SEQUENCE LISTING (70 sequences)

SEQ ID NO: 1 - protein, 110 aa, Homo sapiens
  MISC_FEATURE: Insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 585 aa, Homo sapiens
  MISC_FEATURE: GAD-65
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 3 - protein, 355 aa, Homo sapiens
  MISC_FEATURE: IGRP
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 4 - protein, 86 aa, Homo sapiens
  MISC_FEATURE: proinsulin
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 5 - protein, 70 aa, Homo sapiens
  MISC_FEATURE: human proinsulin 1-70
FVNQHLCGSHLVEALYLVCGERGFFYTPKTRREAEDLQVGQVELGGGPGAGSLQPLALEGSLQKRGIVEQ

SEQ ID NO: 6 - protein, 62 aa, Homo sapiens
  MISC_FEATURE: human proinsulin 9-70
SHLVEALYLVCGERGFFYTPKTRREAEDLQVGQVELGGGPGAGSLQPLALEGSLQKRGIVEQ

SEQ ID NO: 7 - protein, 30 aa, Homo sapiens
  MISC_FEATURE: human proinsulin 9-38
SHLVEALYLVCGERGFFYTPKTRREAEDLQ

SEQ ID NO: 8 - protein, 38 aa, Homo sapiens
  MISC_FEATURE: human proinsulin 1-38
FVNQHLCGSHLVEALYLVCGERGFFYTPKTRREAEDLQ

SEQ ID NO: 9 - protein, 15 aa, Homo sapiens
  MISC_FEATURE: human proinsulin 9-23
SHLVEALYLVCGERG

SEQ ID NO: 10 - protein, 27 aa, Homo sapiens
  MISC_FEATURE: Human Proinsulin 45-71 (C13-A6)
GGGPGAGSLQPLALEGSLQKRGIVEQC

SEQ ID NO: 11 - protein, 11 aa, Homo sapiens
  MISC_FEATURE: Human Proinsulin C24-A1
LALEGSLQKRG

SEQ ID NO: 12 - protein, 18 aa, Homo sapiens
  MISC_FEATURE: Human Proinsulin C19-A3
GSLQPLALEGSLQKRGIV

SEQ ID NO: 13 - protein, 20 aa, Homo sapiens
  MISC_FEATURE: Human Proinsulin C13-32
GGGPGAGSLQPLALEGSLQK

SEQ ID NO: 14 - protein, 28 aa, Homo sapiens
  MISC_FEATURE: Human Proinsulin B9-C4
SHLVEALYLVCGERGFFYTPKTRREAED

SEQ ID NO: 15 - protein, 17 aa, Homo sapiens
  MISC_FEATURE: Human Proinsulin C22-A5
QPLALEGSLQKRGIVEQ

SEQ ID NO: 16 - protein, 65 aa, Homo sapiens
  MISC_FEATURE: Human IA-2 718-782
AYQAEPNTCATAQGEGNIKKNRHPDFLPYDHARIKLKVESSPSRSDYINASPIIEHDPRMPAYIA

SEQ ID NO: 17 - protein, 35 aa, Homo sapiens
  MISC_FEATURE: Human IA-2 785-819
GPLSHTIADFWQMVWESGCTVIVMLTPLVEDGVKQ

SEQ ID NO: 18 - protein, 56 aa, Homo sapiens
  MISC_FEATURE: Human IA-2 828-883
GASLYHVYEVNLVSEHIWCEDFLVRSFYLKNVQTQETRTLTQFHFLSWPAEGTPAS

SEQ ID NO: 19 - protein, 39 aa, Homo sapiens
  MISC_FEATURE: Human IA-2 943-979
EHVRDQRPGLVRSKDQFEFALTAVAEEVNAILKALPQCG

SEQ ID NO: 20 - protein, 304 aa, Homo sapiens
  MISC_FEATURE: Myelin Basic Protein, MBP
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 21 - protein, 247 aa, Homo sapiens
  MISC_FEATURE: Myelin oligodendrocyte glycoprotein (MOG)
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 22 - protein, 277 aa, Homo sapiens
  MISC_FEATURE: Proteolipid Protein
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 23 - protein, 20 aa, Homo sapiens
  MISC_FEATURE: MBP 13-32
KYLATASTMDHARHGFLPRH

SEQ ID NO: 24 - protein, 16 aa, Homo sapiens
  MISC_FEATURE: MBP 83-99
ENPWHFFKNIVTPRTP

SEQ ID NO: 25 - protein, 19 aa, Homo sapiens
  MISC_FEATURE: MBP 111-129
LSRFSWGAEGQRPGFGYGG

SEQ ID NO: 26 - protein, 25 aa, Homo sapiens
  MISC_FEATURE: MBP 146-170
AQGTLSKIFKLGGRDSRSGSPMARR

SEQ ID NO: 27 - protein, 20 aa, Homo sapiens
  MISC_FEATURE: MOG 1-20
GQFRVIGPRHPIRALVGDEV

SEQ ID NO: 28 - protein, 20 aa, Homo sapiens
  MISC_FEATURE: MOG 35-55
MEVGWYRPPFSRWHLYRNGK

SEQ ID NO: 29 - protein, 16 aa, Homo sapiens
  MISC_FEATURE: PLP 39-154
HCLGKWLGHPDKFVGI

SEQ ID NO: 30 - protein, 31 aa, Homo sapiens
  MISC_FEATURE: MOG 30-60
KNATGMEVGWYRSPFSRVVHLYRNGKDQDAE

SEQ ID NO: 31 - protein, 17 aa, Homo sapiens
  MISC_FEATURE: MBP 83-99
ENPVVHFFKNIVTPRTP

SEQ ID NO: 32 - protein, 21 aa, Homo sapiens
  MISC_FEATURE: MOG 35-55
MEVGWYRPPFSRVVHLYRNGK

SEQ ID NO: 33 - protein, 17 aa, Homo sapiens
  MISC_FEATURE: MBP 82-98
DENPVVHFFKNIVTPRT

SEQ ID NO: 34 - protein, 18 aa, Homo sapiens
  MISC_FEATURE: MBP 82-99
DENPVVHFFKNIVTPRTP

SEQ ID NO: 35 - protein, 25 aa, Homo sapiens
  MISC_FEATURE: MBP 82-106
DENPVVHFFKNIVTPRTPPPSQGKG

SEQ ID NO: 36 - protein, 20 aa, Homo sapiens
  MISC_FEATURE: MBP 87-106
VHFFKNIVTPRTPPPSQGKG

SEQ ID NO: 37 - protein, 25 aa, Homo sapiens
  MISC_FEATURE: MBP 131-155
ASDYKSAHKGLKGVDAQGTLSKIFK

SEQ ID NO: 38 - protein, 18 aa, Homo sapiens
  MISC_FEATURE: PLP 41-58
GTEKLIETYFSKNYQDYE

SEQ ID NO: 39 - protein, 18 aa, Homo sapiens
  MISC_FEATURE: PLP 89-106
GFYTTGAVRQIFGDYKTT

SEQ ID NO: 40 - protein, 22 aa, Homo sapiens
  MISC_FEATURE: PLP 95-116
AVRQIFGDYKTTICGKGLSATV

SEQ ID NO: 41 - protein, 20 aa, Homo sapiens
  MISC_FEATURE: PLP 178-197
NTWTTCQSIAFPSKTSASIG

SEQ ID NO: 42 - protein, 20 aa, Homo sapiens
  MISC_FEATURE: PLP 190-209
SKTSASIGSLCADARMYGVL

SEQ ID NO: 43 - protein, 20 aa, Homo sapiens
  MISC_FEATURE: MOG 11-30
PIRALVGDEVELPCRISPGK

SEQ ID NO: 44 - protein, 20 aa, Homo sapiens
  MISC_FEATURE: MOG 21-40
ELPCRISPGKNATGMEVGWY

SEQ ID NO: 45 - protein, 23 aa, Homo sapiens
  MISC_FEATURE: MOG 64-86
EYRGRTELLKDAIGEGKVTLRIR

SEQ ID NO: 46 - protein, 62 aa, Homo sapiens
  MISC_FEATURE: MOG 1-62
GQFRVIGPRHPIRALVGDEVELPCRISPGKNATGMEVGWYRPPFSRVVHLYRNGKDQDGDQA

SEQ ID NO: 47 - protein, 63 aa, Homo sapiens
  MISC_FEATURE: MBP 76-136
SHGRTQDENPVVHFFKNIVTPRTPPPSQGKGRGLSLSRFSWGAEGQRPGFGYGGRASDYKSCG

SEQ ID NO: 48 - protein, 118 aa, Homo sapiens
  MISC_FEATURE: MART1, Melanoma antigen recognized by T cells 1
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 49 - protein, 529 aa, Homo sapiens [sequence omitted]

SEQ ID NO: 50 - protein, 661 aa, Homo sapiens
  MISC_FEATURE: Melanocyte protein PMEL, gp100
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 51 - protein, 323 aa, Homo sapiens
  MISC_FEATURE: Aquaporin-4
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 52 - protein, 405 aa, Homo sapiens
  MISC_FEATURE: S-arrestin
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 53 - protein, 1247 aa, Homo sapiens
  MISC_FEATURE: IRBP
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 54 - protein, 33 aa, Homo sapiens
  MISC_FEATURE: DQ-2 relevant, Alpha-gliadin “33-mer” native
LQLQPFPQPQLPYPQPQLPYPQPQLPYPQPQPF

SEQ ID NO: 55 - protein, 33 aa, Homo sapiens
  MISC_FEATURE: DQ-2 relevant, Alpha-gliadin “33-mer” deamidated
LQLQPFPQPELPYPQPELPYPQPELPYPQPQPF

SEQ ID NO: 56 - protein, 18 aa, Homo sapiens
  MISC_FEATURE: DQ-8 relevant, Alpha-gliadin
QQYPSGQGSFQPSQQNPQ

SEQ ID NO: 57 - protein, 12 aa, Homo sapiens
  MISC_FEATURE: DQ-8 relevant, Omega-gliadin
QPFPQPEQPFPW

SEQ ID NO: 58 - protein, 15 aa, Homo sapiens
  MISC_FEATURE: Alpha-gliadin “15-mer” fragment
ELQPFPQPELPYPQP

SEQ ID NO: 59 - protein, 18 aa, Homo sapiens
  MISC_FEATURE: Gliadin linker
GCRGGGPQPQPFPSQQPY

SEQ ID NO: 60 - protein, 48 aa, Homo sapiens
  MISC_FEATURE: Gliadin extended
GCGPQPQPFPSQQPYLQLQPFPQPQLPYPQPQLPYPQPQLPYPQPQPF

SEQ ID NO: 61 - protein, 48 aa, Homo sapiens
  MISC_FEATURE: Gliadin deamidated extended
GCGPQPQPFPSQQPYLQLQPFPQPELPYPQPELPYPQPELPYPQPQPF

SEQ ID NO: 62 - protein, 140 aa, Homo sapiens
  MISC_FEATURE: alpha synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 63 - protein, 735 aa, adeno-associated virus 2 [sequence omitted]

SEQ ID NO: 64 - protein, 598 aa, adeno-associated virus 2
  MISC_FEATURE: AAV2 VP2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 65 - protein, 533 aa, adeno-associated virus 2
  MISC_FEATURE: AAV2 VP3
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 66 - protein, 736 aa, Adeno-associated Virus Serotype 9
  MISC_FEATURE: AAV9 VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - protein, 599 aa, Adeno-associated Virus Serotype 9
  MISC_FEATURE: AAV9 VP2
TAPGKKRPVEQSPQEPDSSSGIGKSGQQPAKKRLNFGQTGDSESVPDPQPLGEPPEAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 68 - protein, 534 aa, Adeno-associated Virus Serotype 9
  MISC_FEATURE: AAV9 VP3
MASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 69 - protein, 256 aa, Homo sapiens
  MISC_FEATURE: Myeloblastin
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 70 - protein, 745 aa, Homo sapiens
  MISC_FEATURE: Myeloperoxidase
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

(19) United States
(12) Patent Application Publication
(10) Pub. No.: US 2023/0069712 A1
(43) Pub. Date: Mar. 2, 2023

(54) COMPOUNDS FOR THE INDUCTION OF ANTIGEN-SPECIFIC IMMUNE TOLERANCE

(71) Applicant: Anokion SA, Ecublens (CH)

(72) Inventors: Stephan Kontos, Boston, MA (US); Dilrukshi Vitharana, Arlington, MA (US); Sergey A. Isarov, Cambridge, MA (US)

(21) Appl. No.: 17/753,603

(22) PCT Filed: Sep. 18, 2020
(86) PCT No.: PCT/IB2020/058693

§ 371 Date: Mar. 9, 2022

Related U.S. Application Data
(60) Provisional application No. 62/903,609, filed on Sep. 20, 2019.
(60) Provisional application No. 63/062,858, filed on Aug. 7, 2020.

(51) Int. Cl. A61K 39/00; A61K 39/35; A61K 47/54; A61K 47/58; A61P 37/00

(52) U.S. Cl. CPC A61K 39/0008; A61K 39/001; A61K 39/35; A61K 47/549; A61K 47/58; A61P 37/00; A61K 2039/6093; A61K 2039/627

(57) ABSTRACT

Disclosed are compounds for the induction of antigen-specific immune tolerance in a subject, the compounds comprising an antigen, a polymeric linker and a liver targeting moiety, wherein the polymeric linker comprises a terminal end unit lacking each of a dithioester and a dithiobenzoate and wherein the terminal end unit confers improved stability to the compound when in solution.

### CROSS REFERENCE TO RELATED APPLICATIONS

The present application claims the benefit of priority to U.S. Provisional Patent Application No. 63/062,858, filed Aug. 7, 2020, and U.S. Provisional Patent Application No. 62/903,609, filed Sep. 20, 2019, the disclosures of each of which are hereby incorporated by reference herein in their entireties.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is ANOK037WO_ST25.TXT, the date of creation of the ASCII text file is Sep. 16, 2020, and the size of the ASCII text file is 104 KB.

### FIELD

Several embodiments disclosed herein pertain generally to compounds and compositions, including pharmaceutically acceptable compositions, for use in inducing immune tolerance to specific antigens of interest, methods of generating such compositions, and methods/uses of same for induction of antigen specific tolerance.

### BACKGROUND

The liver is involved in a variety of tolerogenic processes. For example, it plays a role in the development of immune tolerance to harmless non-self-antigens absorbed into the blood draining from the gut or to newly formed antigens resulting from hepatic metabolic activities. Harmless antigens such as these fail to induce an immune response in healthy individuals. Antigen-specific immune tolerance and cross-tolerance induction towards CD4+ and CD8+ T cells, respectively, has been attributed to various cell types in the liver including hepatocytes and liver sinusoidal endothelial cells (LSECs). Hepatocytes are the predominant cell type that make up the liver parenchyma, and they can process and present antigens on MHC-I- and MHC-II to signal to CD8+ and CD4+ T cells, respectively. LSECs efficiently scavenge, process and present soluble antigens from the bloodstream on MHC-I and MHC-II to circulating lymphocytes, typically resulting in the induction of CD4+ regulatory T cells or anergic CD8+ T cells.

### SUMMARY

Some embodiments disclosed herein pertain to tolerogenic molecules (e.g., tolerogenic constructs, tolerogenic compounds, etc.) and/or the use of tolerogenic molecules in methods of inducing immune tolerance in a patient. In some embodiments, the tolerogenic molecules show surprisingly improved stability. In some embodiments, the tolerogenic molecules comprise one or more antigens (e.g., a substance that induces an immune response in the body and/or an unwanted immune response). In several embodiments, the antigen is a full-length protein (e.g., a native antigen), fragments (e.g., immunogenic portions) thereof, mimotopes thereof, and the like (collectively, antigens for brevity, unless otherwise indicated as a specific type, e.g., a fragment). In several embodiments, the tolerogenic molecules disclosed herein are configured to induce immune tolerance for an antigen to which immune tolerance is desired. In several embodiments, the tolerogenic molecules comprise an antigen to which immune tolerance is desired. In several embodiments, the tolerogenic molecules comprise one or more targeting moieties (e.g., liver targeting moieties). In several embodiments, the targeting moieties and antigens are bound to each other via a linking group. In several embodiments, the liver targeting moieties are covalently bound (e.g., through the linking group) to the antigens to which tolerance is desired.

In several embodiments, as disclosed elsewhere herein, the construct comprises an antigen to which tolerance is desired, a linker comprising a polymeric portion, one or more liver targeting moieties, and terminal end unit attached to the polymeric portion. In several embodiments, the terminal end unit lacks a dithioester functionality (e.g., a dithiobenzoate (DTB) group). In several embodiments, the antigen is bonded (e.g., covalently) within the construct a one end (e.g., terminus) of the linker and the terminal end unit is bonded (e.g., covalently) to the other end (e.g., terminus) of the linker. In several embodiments, the targeting agents are bonded along and/or decorate the polymeric portion of the linker (e.g., a portion of the polymeric portion of the linker). In several embodiments, the polymeric portion of the linker may be formed through a reversible addition-fragmentation chain transfer reaction (RAFT). These RAFT reactions are performed using RAFT reagent that generates and/or is a chain transfer agent (CTA). In several embodiments, a functionalized dithioester is a CTA fragment (e.g., DTB or the like) and, after the reaction (e.g., polymerization) is complete, it forms an end-capping group within the construct (as a CTA remnant). Surprisingly, though a small feature of the overall construct itself, it has now been found that displacing the CTA remnant (e.g., the dithioester moiety) from the construct using a terminal end unit (that is not a dithioester) stabilizes the entire construct, giving the construct especially advantageous properties for use in, for example, pharmaceutical compositions.

For example, in several embodiments, a construct with a terminal end unit and an antigen book-ending the linker has better stability during storage (e.g., in a buffered solution or some other vehicle for delivery of the construct to a patient) than a molecule having a end-capping group (e.g., a dithioester remnant of a CTA). In several embodiments, where the end groups of the linker are an antigen and a terminal end unit lacking a dithioester (e.g., a terminal end unit lacking a DTB), the construct has enhanced stability. In several embodiments, over a period of 5 days, 14 days, and/or 28 days when stored in a buffered solution, the construct with a terminal end unit and an antigen book-ending the linker degrades less than or equal to about: 0.1%, 0.5%, 1.0%, 2.0%, 2.5%, 5%, or ranges spanning and/or including the aforementioned values. In several embodiments, the degradation of the construct can be measured by the loss in area of a main peak by HPLC or by other conventional means. In several embodiments, over a period of 5 days, 14 days, and/or 28 days, a construct with a terminal end unit and an antigen book-ending the linker has stability (e.g., as measured by the loss in area of a main peak by HPLC, etc.) that is improved by 5%, 10%, 15%, or 20% (or ranges spanning and/or including the aforementioned values) relative to a construct having a dithioester end-capping group (e.g., DTB).

In several embodiments, the polymeric portion of the linker is made up of different repeat units. In several embodiments, as disclosed elsewhere herein, certain repeat units along the length of the polymeric portion of the linker may be bonded to liver targeting moieties. In several embodiments, the liver targeting moieties comprise one or more galactosylating moieties, glucosylating moieties, or combinations thereof. In several embodiments, the liver targeting moieties may comprise a plurality of liver targeting moieties of one type or a plurality of liver targeting moieties of more than one type (e.g., a mixture of 2, 3, 4, or more types of different liver targeting agents). For example, in several embodiments, where the construct comprises one type of liver targeting moiety (e.g., N-acetylgalactosamine), multiple units of the liver targeting moiety can be distributed (e.g., randomly, in blocks, or as a gradient) on repeat units along the polymeric portion of the linker. In several embodiments, where the construct comprises more than one type of liver targeting moiety (e.g., two or more of galactose, glucose, galactosamine, glucosamine, N-acetylgalactosamine, and/or N-acetylglucosamine targeting agents), these different liver targeting moieties can be also distributed (e.g., randomly, in blocks, or as a gradient) on repeat units along the polymeric portion of the linker.

In several embodiments, as disclosed elsewhere herein, the polymeric portion of the linker may comprise a copolymer (e.g., a random copolymer, gradient copolymer, block copolymer, or mixture of the foregoing). As disclosed elsewhere herein, for example, the polymer may comprise more than one type of repeat unit. For example, in several embodiments, the copolymer includes different repeat units bonded to different liver targeting moieties (e.g., more than one type of liver targeting moiety) and/or mixtures of repeat units bonded to liver targeting agents and other spacing repeat units lacking liver targeting agents. To illustrate, in several embodiments, the polymeric portion may comprise and/or be an acrylyl-based polymer (e.g., acrylate-based units or derivatives thereof, acrylamide-based units or derivatives thereof, or the like, or combinations of any of the foregoing). In several embodiments, the acrylyl portion comprises one or more acrylyl units (e.g., acrylyl derivatives, including acrylates, acrylamides, methacrylates, methacrylamides, derivatives of any of the foregoing, or similar acrylyl structures). In some embodiments, some of the acrylyl units are bonded (e.g., covalently) to liver targeting moieties (e.g., have liver targeting agents as pendant side groups) and other acrylyl units may lack (and or may not be bonded to) liver targeting agents. In several embodiments, the liver targeting moieties may be of a single type or multiple types. In some embodiments, the acrylyl groups that lack liver targeting moieties serve as spacing units (e.g., spacers) along the polymer chain, which can change the distance between the liver targeting moieties.

In several embodiments, the copolymer (e.g., a random copolymer, gradient copolymer, block copolymer, or mixture of the foregoing) comprises one or more a first acrylyl unit and a second acrylyl unit. In several embodiments, the first acrylyl unit may be bonded to the liver targeting agent while the second acrylyl unit lacks a liver targeting moiety. In several embodiments, by using a higher ratio of the first acrylyl moiety relative to the second during polymerization, additional density and/or quantity of pendant targeting agents are provided within the polymeric portion of the linker (and the construct). In some embodiments, the acrylyl units are methacrylyl units. In several embodiments, the polymeric portion comprises a first methacrylic unit and a second methacrylic unit. In several embodiments, a first ethylacetamido functionality is connected (e.g., covalently bonded directly or through a connecting group) to the liver-targeting moiety (e.g., to provide a liver targeting repeat unit). In several embodiments, a second ethylacetamido functionality is conjugated (e.g., connected to or covalently bonded) to an aliphatic group, an alcohol, or an aliphatic alcohol (e.g., to provide a spacing unit). In several embodiments, the second ethylacetamido functionality is conjugated (e.g., covalently bonded) to an aliphatic group, an amine, a polyamino, an alcohol, a polyether, or an aliphatic alcohol. In several embodiments, the first methacrylic unit comprises a first ethylacetamido functionality (e.g., a side chain, such as a connecting group) and the second methacrylic unit comprises a second ethylacetamido functionality. In several embodiments, the ethylacetamido groups join the liver targeting moieties and/or spacing groups (e.g., aliphatic group, an alcohol, or an aliphatic alcohol) to the polymeric portion of the linker. In several embodiments, more than one spacing group and/or more than one liver targeting moiety may be added within the polymeric portion of the linker by polymerizing a third, a fourth, a fifth (or additional) methacrylic units during polymerization. In several embodiments, the liver-targeting moiety comprises one or more types of galactosylating moieties, one or more types of glucosylating moieties, or mixtures thereof.

In several embodiments, as noted elsewhere herein, the linker is book-ended on one end by the antigen. In several embodiments, the linker is bonded to the antigen to which tolerance is desired via a degradable bond (e.g., a biodegradable bond and/or a bond that degrades when in a target cell). In several embodiments, the degradable bond is a disulfide bond or a disulfanyl ethyl ester. In several embodiments, the disulfide bond or the disulfanyl ethyl ester are each configured to cleave after administration of the composition to the subject and to release the antigen to which tolerance is desired from the linker. In some embodiments, the antigen is released in its original form (e.g., the form it was in prior to functionalization within the construct). In some embodiments, the antigen is released in its active form (e.g., able to induce immune tolerance). In some embodiments, the antigen is released at a target site.

Several embodiments pertain to an antigen-specific tolerogenic compound. In several embodiments, the compound finds use in the induction of antigen-specific immune tolerance in a subject. In several embodiments, the compound comprises (or consists essentially of) an antigen to which tolerance is desired, a polymeric linker, and a liver-targeting moiety. In several embodiments, the antigen to which tolerance is desired, when presented alone to the subject is capable of inducing an unwanted immune response in the subject. In several embodiments, the polymeric linker comprises (or consists essentially of) a copolymer comprising a first acrylyl unit and a second acrylyl unit, the first acrylyl unit comprising a first C-amido or C-carboxy functionality and the second acrylyl unit comprising a second C-amido or C-carboxy functionality. In several embodiments, the second C-amido or C-carboxy functionality is conjugated to an aliphatic group, an alcohol, or an aliphatic alcohol. In several embodiments, the polymeric linker is bonded (e.g., conjugated) to the antigen to which tolerance is desired via a degradable bond (e.g., disulfide bond or a disulfanyl ethyl ester). In several embodiments, the polymeric linker comprises a terminal end unit lacking each of a dithioester and a dithiobenzoate (DTB). In several embodiments, the terminal end unit confers improved stability to the compound when in solution. In several embodiments, the liver-targeting moiety is connected to the first acrylyl unit through the first C-amido or C-carboxy functionality and a polyether.

In several embodiments, the compound has improved stability relative to a compound that includes a dithioester moiety that is a remnant of a chain transfer agent (CTA).

In several embodiments, the terminal end unit is isobutyronitrile.

In several embodiments, the degradable bond (e.g., disulfide bond or the disulfanyl ethyl ester) is configured to be cleaved upon administration of the compound to the subject and to release the antigen to which tolerance is desired from the polymeric linker.

In several embodiments, the terminal end unit lacks one or more of a trithiocarbonate and a xanthate.

In several embodiments, when in a solution of 10 mM sodium acetate and 274 mM sorbitol at a compound concentration of 1 mg/mL, at a temperature of 23° C. to 27° C., the compound degrades less than 5.0% over a period of 28 days.

In several embodiments, the terminal end unit comprises a carbon atom that is bonded to a carbon of the polymer linker, thereby providing a carbon-carbon bond between the polymeric linker and the terminal end unit.

In several embodiments, the terminal end unit is provided by performing a reaction between an azo compound and a precursor to the polymeric linker, the precursor comprising a dithioester, a dithiobenzoate, a trithiocarbonate, or a xanthate. In several embodiments, the azo compound is azobisisobutyronitrile (AIBN).

In several embodiments, the terminal end unit lacks one or more of a S atom or an aryl group.

In several embodiments, the terminal end unit is provided on the compound through a reaction between an azo compound and a precursor to the compound. In several embodiments, the azo compound is an azobisalkylnitrile.

In several embodiments, the azo compound is azobisisobutyronitrile (AIBN).

In several embodiments, a precursor to the compound comprises a dithioester.

In several embodiments, the the first acrylyl unit comprises a first ethylacetamido functionality and the second acrylyl unit comprising a second ethylacetamido functionality.

In several embodiments, the liver-targeting moiety is bonded to the polymeric linker through the first ethylacetamido functionality. In several embodiments, the liver-targeting moiety is connected to the first acrylyl unit directly or through a connecting group comprising an aliphatic group, a polyether, or a polyamino group. In several embodiments, the second ethylacetamido functionality is connected to an aliphatic group, an alcohol, or an aliphatic alcohol and wherein the acts as a spacer.

Several embodiments pertain to a composition comprising the compound. In several embodiments, the composition comprises a pharmaceutically acceptable carrier.

Several embodiments pertain to the use of a compound as disclosed herein for use in inducing tolerance to the antigen.

Several embodiments pertain to method of inducing tolerance to an antigen to which a subject is capable of developing an unwanted immune response. In several embodiments, the method comprises administering the compound as disclosed herein to the subject. In several embodiments, the compound is administered prior to the subject being exposed to the antigen, after the subject has been exposed to the antigen, or both.

Several embodiments pertain to the use of a compound as disclosed herein for the preparation of a medicament for inducing tolerance to an antigen or for the treatment of an unwanted immune response.

Several embodiments pertain to a compound as disclosed herein for the induction of antigen-specific immune tolerance in a subject, the compound comprising (or consisting essentially of) an antigen, a polymeric linker comprising a polymeric portion, and a liver targeting moiety. In several embodiments, the linker is bonded to the antigen via a degradable bond (e.g., via a disulfide bond or a disulfanyl ethyl ester). In several embodiments, the degradable bond (e.g., the disulfide bond or the disulfanyl ethyl ester) is configured to be cleaved upon administration of the compound to the subject and to release the antigen from the polymeric linker. In several embodiments, the polymeric linker comprises (or consists essentially of) a terminal end unit lacking each of a dithioester and a dithiobenzoate (DTB). In several embodiments, the terminal end unit confers improved stability to the compound when in solution.

In several embodiments, the polymeric portion copolymeric and comprises (or consists essentially of) a first repeat unit and a second repeat unit. In several embodiments, the liver targeting moiety is connected to the first unit directly or through a connecting group of the first unit. In several embodiments, the first unit comprises (or consists essentially of) a first C-amido or first C-carboxy functionality and a connecting group. In several embodiments, the connecting group is an aliphatic group, a polyether, or a polyamino group.

In several embodiments, the first repeat unit is a first acrylyl unit and the second repeat unit is a second acrylyl unit.

Several embodiments pertain to antigen-specific tolerogenic compound. In several embodiments, the compound comprises (or consists essentially of) Formula 1:

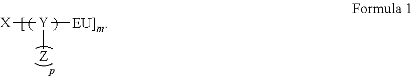

In several embodiments, m is an integer from about 1 to 100. In several embodiments, X comprises an antigen. In several embodiments, Y is a linker moiety. In several embodiments, Y is of a linker moiety having a formula selected from the group consisting of:

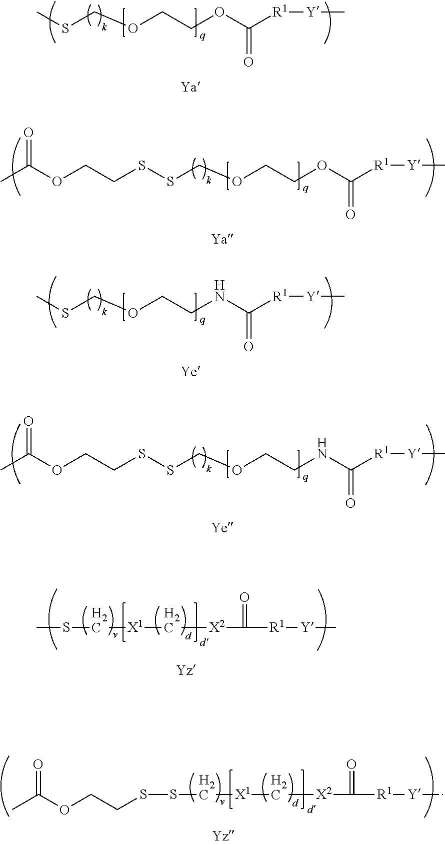

In several embodiments, q is an integer from 0 to 100. In several embodiments, k is an integer from 0 to 10. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, X¹ and X² are independently selected from a direct bond, —NR⁶—, and —O—. In several embodiments, v is an integer from 0 to 10. In several embodiments, d is an integer from 0 to 5. In several embodiments, d′ is an integer from 0 to 50. In several embodiments, R⁶ is H or optionally substituted C₁₋₆ alkyl. In several embodiments, Y′ is a random copolymer, gradient copolymer, or block copolymer of W¹ and W² or of W³ and W⁴, where W¹, W², W³, and W⁴ are as depicted below:

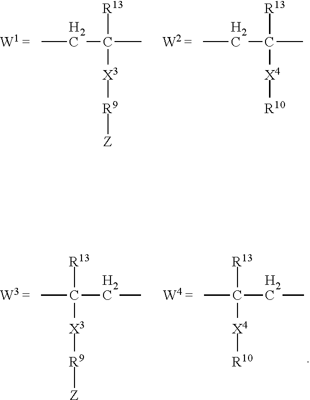

In several embodiments, the number of repeat units of W¹ or W³ in Y is denoted as p. In several embodiments, p is an integer of at least about 1. In several embodiments, the number of repeat units of W² or W⁴ in Y is denoted as r. In several embodiments, r is an integer of at least about 1. In several embodiments, wherein the sum of p and r ranges from 100 to 200. In several embodiments, X³ is selected from —C(O)—NH— or —C(O)O—. In several embodiments, X⁴ is —C(O)—NH—, —C(O)O—, or —C(O)—OH. In several embodiments, R⁹ is a direct bond (e.g., a bond between X³ and Z) or —[((CH₂)ₕX⁵)ₜ—((CH₂)_{h′}X⁶)_{t′}]—. In several embodiments, X⁵ and X⁶ are independently selected from a direct bond, —CNR^{6′}—, and —O—. In several embodiments, each instance of R^{6′} is independently H or optionally substituted —C₁₋₆ alkyl. In several embodiments, R¹⁰ is not present, is H, or is —[((CH₂)_{h″}—X⁷)_{t″}—((CH₂)_{h′″}X⁸)_{t′″}]—H. In several embodiments, where, X⁷ and X⁸ are independently selected from a direct bond, —CNR^{6″}—, and —O—. In several embodiments, where each instance of R^{6″} is independently H or optionally substituted —C₁₋₆ alkyl, In several embodiments, t, t′, h, h′, t″, t′″, h″, and h′″ are each independently an integer of equal to or at least 0, 1, 2, 3, 4, 5, 6, 7, 8, 9, 10, or 20. In several embodiments, each instance of R¹³ is independently H, methyl, or optionally substituted —C₁₋₆ alkyl. In several embodiments, EU is a terminal end unit. In several embodiments, EU comprises a carbon atom that is bonded to a carbon of the linker, thereby forming a carbon-carbon bond between the linker and the terminal end unit. In several embodiments, Z comprises a liver-targeting moiety.

In several embodiments, EU is represented by:

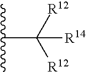

In several embodiments, R¹⁴ is selected from optionally substituted C₁₋₁₁ alkyl; —CN; optionally substituted —C(═NH)NH₂; optionally substituted heterocyclyl; optionally substituted aryl; optionally substituted heteroaryl; optionally substituted aralkyl; optionally substituted heteroaralkyl; C-carboxy (—C(O)OR) where R is —H, optionally substituted C₁₋₆ alkyl, optionally substituted C₁₋₆alkylenyl, or optionally substituted phenyl; optionally substituted C-amido (—C(O)NR_{A}R_{B}) where R_{A} and R_{B} are independently —H or optionally substituted C₁₋₆ alkyl; optionally substituted succinimidyl ester; optionally substituted isoindolin-1,3-dione; an alkyl silane. In several embodiments, each instance of R¹² is independently a hydrogen, an optionally substituted C₁₋₁₀ alkyl, or each instance of R¹² is taken together to provide an optionally substituted C₃₋₆ cycloalkyl.

In several embodiments, each optional substitution on an R¹⁴ or R¹² is selected from the group consisting of C₁₋₃ alkyl, C₁₋₆ alkoxy, C₁₋₆ alkylenyl, hydroxyl, amino, halogen, C-carboxy (where R is —H, C₁₋₆ alkyl, or polyethylene glycol (PEG). In several embodiments, each optional substitution on an R⁴ or R¹² is selected from the group consisting of halogen, C-carboxy, -amino, or —OH.

In several embodiments, EU is selected from the group consisting of

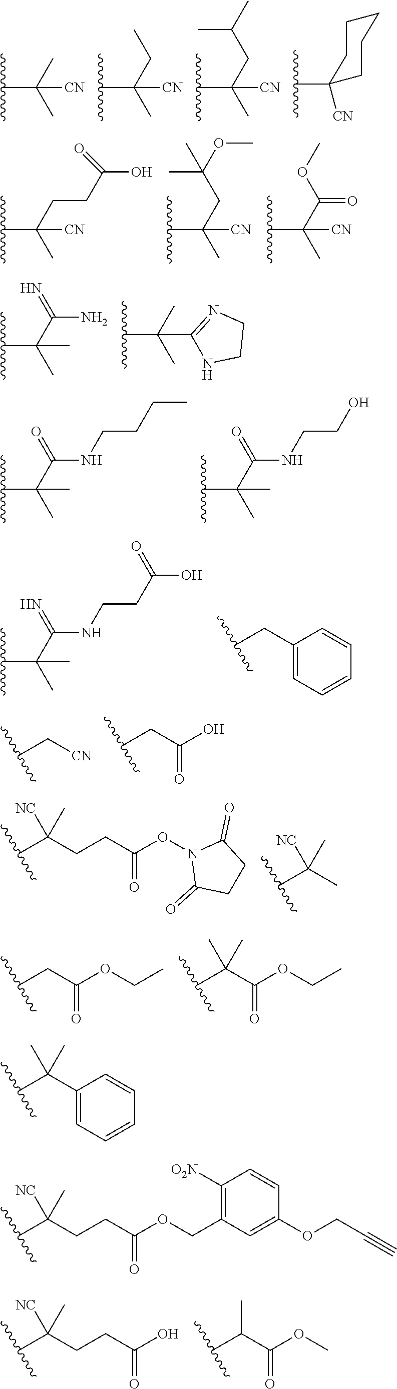

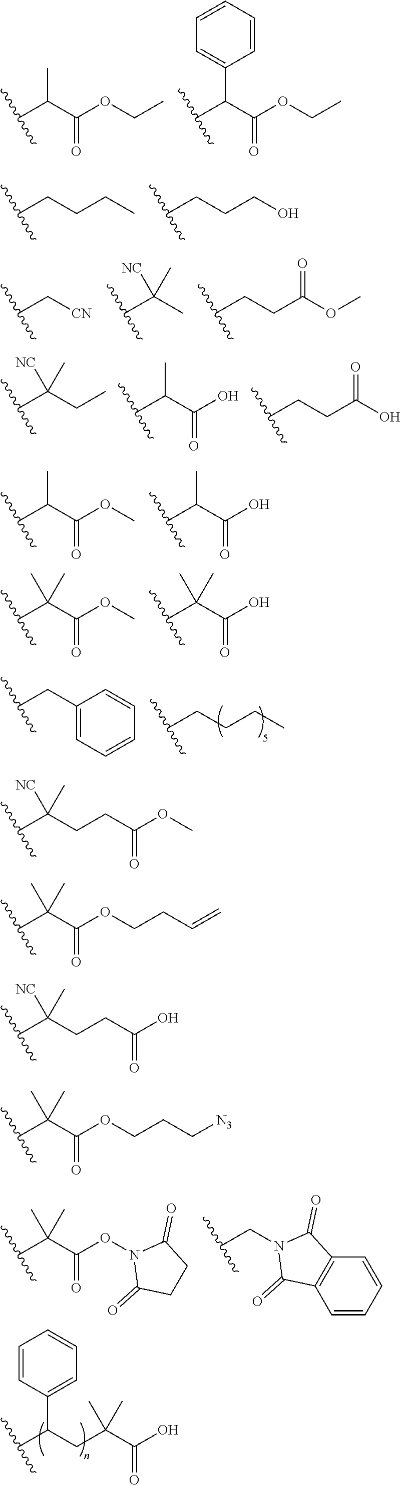

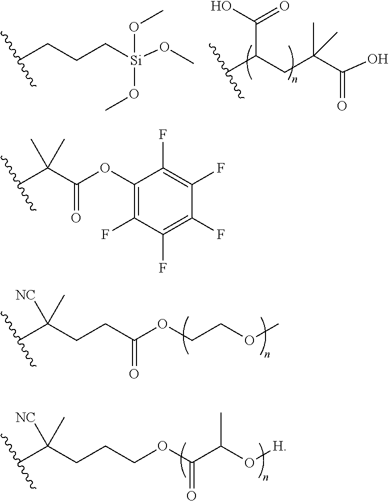

In several embodiments, the ratio of p to r is about 1:1. In several embodiments, the ratio of p to r is about 4:1.

In several embodiments, Y is Ya′. In several embodiments, k is 2. In several embodiments, R¹ is —(CH₂)₂—C(CH₃)(CN)—. In several embodiments, X³ is —C(O)—NH—, h is 2, X⁵ is —O—, t is 2, and t′ is 0, such that X³ and R⁹ taken together is —C(O)—NH—CH₂—CH₂—O—CH₂—CH₂—O—. In several embodiments, q is 0. In several embodiments, q is 3.

In several embodiments, Y is Ye′. In several embodiments, q is 0. In several embodiments, k is 2. In several embodiments, R¹ is —(CH₂)₂—C(CH₃)(CN)—. In several embodiments, X³ and R⁹ taken together is —C(O)—NH—(CH₂)₂—(O—CH₂—CH₂)ₜ—. In several embodiments, t is 1. In several embodiments, Z is one or more of galactose, glucose, galactosamine, glucosamine, N-acetylgalactosamine, or N-acetylglucosamine. In several embodiments, q is 0. In several embodiments, q is 3.

In several embodiments, when n a solution of 10 mM sodium acetate and 274 mM sorbitol at a compound concentration of mg/mL, at a temperature of 23° C. to 27° C., the compound as disclosed above or elsewhere herein degrades less than 2.0% over a period of 28 days.

In several embodiments, the liver targeting moeity is galactose, glucose, galactosamine, glucosamine, N-acetylgalactosamine, and/or N-acetylglucosamine. In several embodiments, the liver targeting moiety (e.g., Z) is bound at its C1, C2 or C6 to the linker (e.g., Y). In several embodiments, Z is N-acetylgalactosamine or N-acetylglucosamine.

In several embodiments, the antigen is a tolerogenic portion of a full-length antigen. In several embodiments, the antigen is a fragment of a full-length antigen. In several embodiments, the antigen is a mimetic (e.g., a mimetope) of a native antigen.

In several embodiments, the antigen comprises (and/or consists of and/or consists essentially of) a food antigen. In several embodiments, the food antigen is selected from the group consisting of conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), α-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1 lS globulin (Sin a 2), lipid transfer protein (Sin a 3), profilin (Sin a 4), profilin (Api g 4), high molecular weight glycoprotein (Api g 5), Pen a 1 allergen (Pen a 1), allergen Pen m 2 (Pen m 2), tropomyosin fast isoform, high molecular weight glutenin, low molecular weight glutenin, alpha-, gamma- and omega-gliadin, hordein, secalin, avenin, major strawberry allergy Fra a 1-E (Fra a 1), profilin (Mus xp 1), a portion of any of said antigens, and a mimetic of any of said antigens. In several embodiments, the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens. In several embodiments, the food antigen is associated with celiac disease.

In several embodiments, the antigen is associated with an autoimmune disease. In several embodiments, the autoimmune disease is selected from the group consisting of Type I diabetes, multiple sclerosis, rheumatoid arthritis, vitiligo, uveitis, pemphis vulgaris, neuromyelitis optica, Goodpasture's Disease, Parkinson's disease, myasthenia gravis, and celiac disease.

In several embodiments, the antigen comprises (and/or consists of and/or consists essentially of) a self antigen. In several embodiments, the self antigen is selected from myelin basic protein, myelin oligodendrocyte glycoprotein and proteolipid protein, a portion of any of said antigens, and a mimetic of any of said antigens. In several embodiments, the self antigen is selected from insulin, proinsulin, preproinsulin, glutamic acid decarboxylase-65 (GAD-65), GAD-67, insulinoma associated protein 2 (IA-2), and insulinoma-associated protein 213 (IA-213), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestinepancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, SST G-protein coupled receptors 1-5, and a portion of any of said antigens, and a mimetic of any of said antigens.

In several embodiments, the antigen comprises (and/or consists of and/or consists essentially of) a therapeutic agent. In several embodiments, the therapeutic agent is selected from Abciximab, Adalimumab, Agalsidase alfa, Agalsidase beta, Aldeslukin, Alglucosidase alfa, Factor VIII, Factor IX, Infliximab, L-asparaginase, Laronidase, Natalizumab, Octreotide, Phenylalanine ammonia-lyase (PAL), Rasburicase (uricase), a gene therapy vector, or AAV, a portion of any of said antigens, and a mimetic of any of said antigens.

In several embodiments, the antigen is not bee venom (e.g., melittin).

In several embodiments, the antigen comprises (and/or consists of and/or consists essentially of) a transplant antigen. In several embodiments, the transplant antigen is selected from the group consisting of subunits of the MHC class I and MHC class II haplotype proteins and their complexes with the antigens they present, and minor blood group antigens RhCE, Kell, Kidd, Duffy and Ss.

In several embodiments, the antigen or a tolerogenic portion thereof is desmoglein-3, -1, and/or -4 (or a portion of any one of the foregoing).

In several embodiments, the antigen or a tolerogenic portion thereof is associated with pemphigus vulgaris.

Several embodiments pertain to a composition comprising the compound as disclosed above or anywhere else herein.

Several embodiments pertain to the use of a compound as disclosed above or anywhere else herein for use in inducing tolerance to the antigen.

Several embodiments pertain to a method of inducing tolerance to an antigen to which a subject is capable of developing an unwanted immune response. In several embodiments, the method comprises (or consists of or consists essentially of) administering the compound as disclosed above or anywhere else herein to the subject. In several embodiments, the compound is administered prior to the subject being exposed to the antigen, after the subject has been exposed to the antigen, or both.

Several embodiments pertain to the use of a compound as disclosed above or anywhere else herein for the preparation of a medicament for inducing tolerance to an antigen.

Several embodiments pertain to a compound or composition as disclosed above or anywhere else herein, wherein the antigen comprises a foreign transplant antigen, a tolerogenic portion thereof, or a mimetic thereof against which transplant recipients develop an unwanted immune response.

Several embodiments pertain to a compound or composition as disclosed above or anywhere else herein, wherein the antigen comprises a foreign food, animal, plant or environmental antigen, a tolerogenic portion of any of thereof, or a mimetic of any of thereof against which induces patients develop an unwanted immune response.

Several embodiments pertain to a compound or composition as disclosed above or anywhere else herein, wherein the antigen comprises a foreign therapeutic agent, a tolerogenic portion thereof, or a mimetic thereof against which patients develop an unwanted immune response.

Several embodiments pertain to a compound or composition as disclosed above or anywhere else herein, wherein the antigen comprises a self-antigen, a tolerogenic portion thereof, or a mimetic thereof against the endogenous version of which patients develop an unwanted immune response or a tolerogenic portion thereof.

Several embodiments pertain to a compound or composition as disclosed above or anywhere else herein, wherein the antigen comprises an antibody, antibody fragment or ligand that specifically binds a circulating protein or peptide or antibody, which circulating protein or peptide or antibody is causatively involved in transplant rejection, immune response against a therapeutic agent, autoimmune disease, hypersensitivity and/or allergy.

Several embodiments pertain to a pharmaceutically acceptable composition for inducing tolerance to a therapeutic protein in a subject having a deficiency in production of a functional analogous native protein, comprising a compound as disclosed above or anywhere else herein. In several embodiments, the pharmaceutically acceptable composition comprises (and/or consists of and/or consists essentially of) a pharmaceutically acceptable carrier. In several embodiments, the pharmaceutically acceptable composition comprises a pharmaceutically acceptable excipient. In several embodiments, the pharmaceutically acceptable composition comprises one or more of dispersion media, coatings, antibacterial and antifungal agents, isotonic and absorption delaying agents and the like. In several embodiments, the compound is provided as a pharmaceutically acceptable salt. In several embodiments, the pharmaceutically acceptable composition comprises a compound that facilitates the incorporation of a compound into cells or tissues. For example, without limitation, dimethyl sulfoxide (DMSO) is a commonly utilized carrier that facilitates the uptake of many organic compounds into cells or tissues of a subject. In several embodiments, the pharmaceutically acceptable composition comprises a diluent. In several embodiments, the pharmaceutically acceptable composition comprises (and/or consists of and/or consists essentially of) a liquid for the dissolution of a compound to be administered by injection, ingestion or inhalation. In several embodiments, the pharmaceutically acceptable composition comprises (and/or consists of and/or consists essentially of) a buffered aqueous solution such as, without limitation, phosphate buffered saline (PBS) that mimics the pH and isotonicity of human blood. In several embodiments, the pharmaceutically acceptable composition comprises (and/or consists of and/or consists essentially of) other buffers.

In several embodiments, the composition (e.g., pharmaceutically acceptable composition) comprises (and/or consists of and/or consists essentially of) a buffer selected from sodium acetate buffer, PBS, or HEPES buffer. HEPES (4-(2-hydroxyethyl)-1-piperazineethanesulfonic acid) is a zwitterionic sulfonic acid buffering agent. In several embodiments, the pharmaceutically acceptable composition comprises (or consists essentially of) 10 mM sodium acetate, containing 274 mM sorbitol (pH of about 5 to 5.5). In several embodiments, the composition comprises a peptide concentration (e.g., the antigen concentration or construct concentration) of 1 mg/mL. In several embodiments, the pharmaceutically acceptable composition comprises (or consists essentially of) HEPES buffered saline (pH 8.04). In several embodiments, the pharmaceutically acceptable composition comprises (or consists essentially of) a solution of PBS (pH 7.2). In several embodiments, the pharmaceutically acceptable composition comprises 1 mg/mL concentration of a construct or of an antigen of the construct. In several embodiments, the pharmaceutically acceptable composition comprises 10 mM reduced glutathione.

Several embodiments pertain to a compound or composition as disclosed above or anywhere else herein for use in treating an unwanted immune response against an antigen. In several embodiments, the unwanted immune response is associated with Type I diabetes, multiple sclerosis, rheumatoid arthritis, vitiligo, uveitis, pemphigus vulgaris, neuromyelitis optica, Parkinson's disease, Goodpasture's disease, celiac disease, or myasthenia gravis.

Several embodiments pertain to a compound or composition as disclosed above or anywhere else herein for manufacturing a medicament for use in treating an unwanted immune response against an antigen.

Several embodiments pertain to a method of manufacturing an antigen-specific tolerogenic compound as disclosed above or anywhere else herein. In several embodiments, the method comprises preparing a polymeric portion of a polymeric linker using reversible addition-fragmentation chain transfer reaction (RAFT). In several embodiments, the method comprises displacing a chain transfer agent remnant from the polymeric portion with a terminal end unit. In several embodiments, the method comprises conjugating an antigen to the polymeric linker.

Several embodiments pertain to a method of manufacturing a method of manufacturing a compound having the following structure:

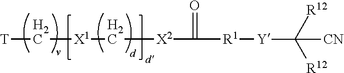

In several embodiments, the method comprises providing a dithiobenzoate containing compound of the following structure:

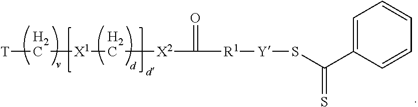

In several embodiments, the method comprises providing a terminal end unit of the following structure:

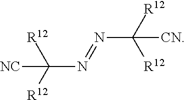

In several embodiments, the method comprises mixing the terminal end unit reagent with the dithiobenzoate containing compound to form the compound. In several embodiments, T, when present, is PDS or a carboxylic acid. In several embodiments, X¹ and X² are independently selected from a direct bond, —NR⁶—, and —O—. In several embodiments, R⁶ is H or C₁₋₆ alkyl optionally substituted with a halogen, C₁₋₃ alkyl or C-carboxy. In several embodiments, v is an integer from 0 to 10. In several embodiments, d is an integer from 0 to 5. In several embodiments, d′ is an integer from 0 to 50. In several embodiments, each instance of R¹² is independently hydrogen or an optionally substituted alkyl. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, Y′ is a random copolymer, gradient copolymer, or block copolymer of W¹ and W² or of W³ and W⁴, where W¹, W², W³, and W⁴ are as depicted below:

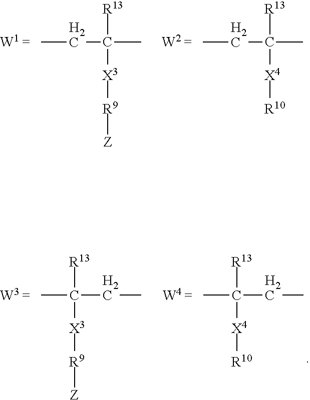

In several embodiments, the number of repeat units of W¹ or W³ in Y′ is denoted as p and wherein p is an integer of at least about 1. In several embodiments, the number of repeat units of W² or W⁴ in Y′ is denoted as r and wherein r is an integer of at least about 1. In several embodiments, X³ is selected from —C(O)—NH— or —C(O)O—. In several embodiments, X⁴ is —C(O)—NH—, —C(O)O—, or —C(O)—OH. In several embodiments, R⁹ is a direct bond or —[((CH₂)ₕX⁵)ₜ—((CH₂)_{h′}X⁶)_{t′}]—. In several embodiments, X⁵ and X⁶ are independently selected from a direct bond, —CNR^{6′}—, and —O—. In several embodiments, each instance of R^{6′} is independently H or —C₁₋₄ alkyl optionally substituted with halogen, C₁₋₃ alkyl or C-carboxy. In several embodiments, R¹⁰ is not present or is —[((CH₂)_{h″}—X⁷)_{t″}—((CH₂)_{h′″}X⁸)_{t′″}]—H. In several embodiments, X⁷ and X⁸ are independently selected from a direct bond, —CNR^{6″}—, and —O—. In several embodiments, each instance of R^{6″} is independently H or —C₁₋₆ alkyl optionally substituted with —OH, halogen, C₁₋₃ alkyl or C-carboxy. In several embodiments, t, t′, h, and h′ are each independently an integer of equal to or at least about: 0, 1, 2, 3, 4, 5, 6, 7, 8, 9, 10, 20. In several embodiments, t″, t′″, h″, and h′″ are each independently an integer of equal to or at least about: 0, 1, 2, 3, 4, 5, 6, 7, 8, 9, 10, 20. In several embodiments, each instance of R¹³ is independently H, methyl, or optionally substituted —C₁₋₆alkyl. In several embodiments, each instance of R¹³ may be optionally substituted with —OH, halogen, C₁₋₃ alkyl or C-carboxy. In several embodiments, Z comprises a liver-targeting moiety.

Several embodiments pertain to a method of manufacturing an antigen-specific tolerogenic compound. In several embodiments, the method comprises coupling a liver targeting moiety to a first acrylyl monomer. In several embodiments, the method comprises preparing a polymeric portion of a polymeric linker using reversible addition-fragmentation chain transfer reaction (RAFT) of at least the first acrylyl monomer and a second acrylyl monomer, the polymeric portion comprising a copolymer comprising a first acrylyl unit and a second acrylyl unit, the first acrylyl unit comprising a first C-amido or C-carboxy functionality and the second acrylyl unit comprising a second C-amido or C-carboxy functionality. In several embodiments, the method comprises displacing a chain transfer agent remnant from the polymeric portion with a terminal end unit wherein the terminal end unit confers improved stability to the compound when in solution. In several embodiments, the method comprises conjugating an antigen to the polymeric linker. In several embodiments, the antigen to which tolerance is desired, when presented alone to the subject is capable of inducing an unwanted immune response in the subject. In several embodiments, the terminal end unit is added to the polymeric portion by reaction using a bis-azo compound. In several embodiments, the second acrylyl monomer is coupled to an aliphatic group, an alcohol, or an aliphatic alcohol.

Several embodiments pertain to a compound for the induction of antigen-specific immune tolerance in a subject. In several embodiments, the compound comprises an antigen, a polymeric linker comprising a polymeric portion, and a liver targeting moiety. In several embodiments, the linker is bonded to the antigen via a degradable bond (e.g., a biodegradable bond such as a disulfide bond or a disulfanyl ethyl ester). In several embodiments, the degradable bond (e.g., disulfide bond or the disulfanyl ethyl ester) is configured to be cleaved upon administration of the compound to the subject and to release the antigen from the polymeric linker. In several embodiments, the polymeric linker comprises a terminal end unit that confers improved stability to the compound when in solution. In several embodiments, the terminal end unit is not —H, a dithioester, a dithiobenzoate, a trithiocarbonate, or a xanthate.

In several embodiments, the antigen as disclosed above or elsewhere herein comprises all or an immunogenic fragment of SEQ ID NO: 54, 55, 56, 57, 60, or 61. In several embodiments, the antigen comprises all or an immunogenic fragment of SEQ ID NO: 21, 27, 28, 30, 32, 43, 44, or 46. In several embodiments, the antigen comprises all or an immunogenic fragment of SEQ ID NO: 1 to SEQ ID NO: 19. In several embodiments wherein the antigen is associated with Celiac disease, the antigen comprises, consists of or consists essentially of an antigen having a sequence according to one of SEQ ID NO: 54, 55, 58, 60 or 61. In several embodiments wherein the antigen is associated with Multiple Sclerosis, the antigen comprises, consists of or consists essentially of an antigen having a sequence according to one of SEQ ID NO: 27, 28, 30, 32, 43, 44, or 46. In several embodiments wherein the antigen is associated with Type I Diabetes, the antigen comprises, consists of or consists essentially of an antigen having a sequence according to one or more of SEQ ID NO: 1 to 19. In several embodiments wherein the antigen is associated with Type I Diabetes, the antigen used in the tolerogenic composition comprises an immunogenic fragment (or fragments) of insulin (e.g., an immunogenic fragment or fragments of SEQ ID NO: 1).

In several embodiments, the construct (e.g., the tolerogenic molecule) may be represented structurally. In several embodiments, the construct is represented by Formula 1:

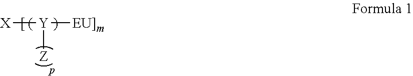

In several embodiments, X comprises (consists of and/or consists essentially of) an antigen, including mimetics of antigens, one or more fragments thereof, or one or more tolerogenic portions thereof. In several embodiments, Y represents (comprises, consists of, and/or consists essentially of) the linker. In several embodiments, EU represents the terminal end unit (e.g., the end unit). The terminal end unit may be a displacing group for a polymeric portion of the linker. In several embodiments, the terminal end unit is provided after polymerization is complete to provide a terminus of the polymeric portion of the linker (e.g., an acrylyl polymer). In several embodiments, as disclosed elsewhere herein, the terminal end unit lacks a dithioester (e.g., DTB). In several embodiments, Z represents the one or more liver targeting moieties. In several embodiments, m is an integer from about 1 to 100.

In several embodiments, Y is of a linker moiety having a formula selected from the group consisting of:

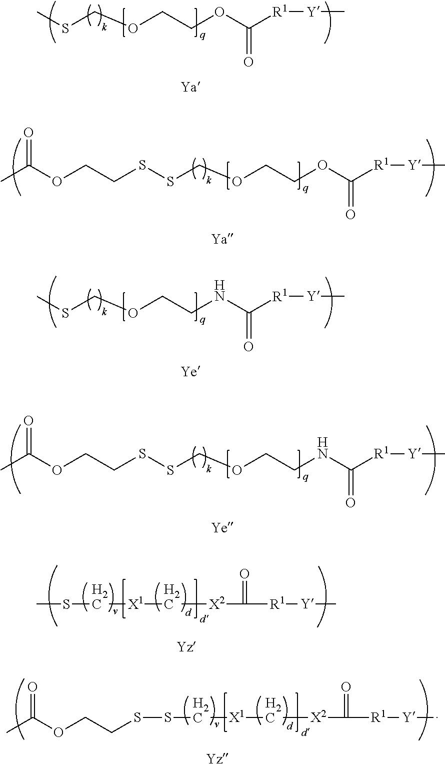

In several embodiments, q is an integer from about 1 to about 100. In several embodiments, q is 0. In several embodiments, q is an integer from about 0 to about 100. In several embodiments, k is an integer from about 1 to about 20. In several embodiments, k is 0. In several embodiments, k is an integer from about 0 to about 20. In several embodiments, R₁ is optionally substituted alkylene. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, X¹ and X² are independently selected from a direct bond, —NR⁶—, and —O—. In several embodiments, X¹ and X² are independently selected from —NR⁶— and —O—. In several embodiments, X¹ and X² are the same while, in other embodiments, X¹ and X² are different. In several embodiments, R⁶ is H or optionally substituted C₁₋₆ alkyl. In some embodiments, R⁶ is optionally substituted with halogen, C₁₋₃ alkyl or C-carboxy. In several embodiments, R⁶ is H or C₁₋₆ alkyl optionally substituted with a halogen, C₁₋₃ alkyl and/or C-carboxy. In several embodiments, each of q, k, v, d, and d′ are independently an integer greater than or equal to about: 0, 1, 2, 3, 4, 5, 6, 7, 8, 9, 10, 15, 20, 40, 44, 50, 75, 100, 150 or ranges including and/or spanning the aforementioned values. In some embodiments, R¹, X¹ and X² (e.g., one or more of the methyl or methylene groups or other groups of these structures) may be optionally substituted. In some embodiments, R¹, X¹ and X² may be optionally substituted with C₁₋₆alkyl or halogen. In several embodiments, Y is Ya′ (or Ya″), k is 2, q is 0, and R¹ is —(CH₂)₂—C(CH₃)(CN)—. In several embodiments, Y is Ya′ (or Ya″), k is 2, q is 3, and R¹ is —(CH₂)₂—C(CH₃)(CN)—. In several embodiments, Y is Ye′ (or Ye″), k is 2, q is 0, and R¹ is —(CH₂)₂—C(CH₃)(CN)—. In several embodiments, Y is Ye′ (or Ye″), k is 2, q is 3, and R¹ is —(CH₂)₂—C(CH₃)(CN)—. In several embodiments, Y is Yz′ (or Yz″), v is 2, X¹ and X² are —O—, d is 2, d′ is 3, and R¹ is —(CH₂)₂—C(CH₃)(CN)—. In several embodiments, Y is Yz′ (or Yz″), v is 2, X¹ and X² are —NR⁶—, R⁶ is H, d is 2, d′ is 3, and R¹ is —(CH₂)₂—C(CH₃)(CN)—. In several embodiments, Y is Yz′ (or Yz″), v is 2, X² is —NR⁶—, R⁶ is H, d′ is 0, and R¹ is —(CH₂)₂—C(CH₃)(CN)—.

In several embodiments, Y′ is a random copolymer, gradient copolymer, or block copolymer of of W¹ and W² or of W³ and W⁴, where W¹, W², W³, and W⁴ are as disclosed elsewhere herein. In several embodiments, W¹, W², W³, and W⁴ are as depicted below:

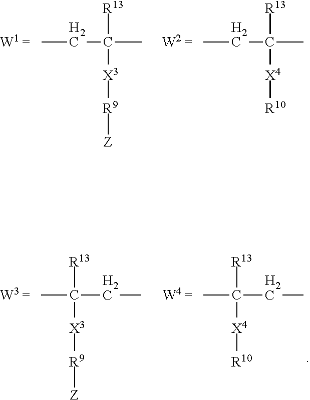

In several embodiments, more than one type of W¹ and/or more than one type of W² can be used in a single instance of Y′. In several embodiments, more than one type of W³ and/or more than one type of W⁴ can be used in a single instance of Y. In several embodiments, Y is a polymer of W¹ or W³ units (e.g., lacking W² or W⁴ units). In several embodiments, where Y is a polymer of W¹ and/or W² units (e.g., lacking W³ and/or W⁴ units) and an antigen may be bound at the α-end of the linker. In several embodiments, where Y′ is a polymer of W³ and/or W⁴ units (e.g., lacking W¹ and/or W² units) and an antigen may be bound at the ω-end of the linker.

In several embodiments, the number of repeat units of W¹ or W³ in Y (or Y′) is denoted as p and p is an integer of at least about 1. In several embodiments, the number of repeat units of W² or W⁴ in Y (or Y′) is denoted as r and wherein r is an integer of at least about 1. In several embodiments, the sum of p and r is equal to or greater than about 100. In several embodiments, the sum of p and r is equal to or greater than about 150. In several embodiments, the sum of p and r is equal to or greater than about 170. In several embodiments, the sum of p and r is equal to or greater than about 200. In several embodiments, the sum of p and r is equal to or greater than about 250. In some embodiments, p is an integer equal to or greater than about: 0, 1, 50, 85, 100, 150, 165, 200, 225, 250, 300, 400, or ranges including and/or spanning the aforementioned values. In some embodiments, r is an integer equal to or greater than about: 0, 1, 50, 85, 100, 150, 165, 200, 225, 250, 300, 400, or ranges including and/or spanning the aforementioned values. In some embodiments, Y′ is a homopolymer of W, W², W³, or W⁴. In some embodiments, p is 0. In some embodiments, r is 0. In some embodiments, the sum of p and r is an integer equal to or greater than about: 1, 50, 75, 80, 85, 100, 150, 165, 170, 200, 225, 250, 300, 400, 600, 800, or ranges including and/or spanning the aforementioned values.

Referring to W¹ or W³, in several embodiments, X³ is selected from —C(O)—NH— or —C(O)O—. In several embodiments, R⁹ is a direct bond or —[((CH₂)ₕX⁵)ₜ—((CH₂)_{h′}X⁶)_{t′}]—. In several embodiments, X⁵ and X⁶ are independently selected from a direct bond, —CNR^{6′}—, and —O—. In some embodiments, each instance of R^{6′} (e.g., in X⁵ or X⁶), where present, is independently selected from H or optionally substituted —C₁₋₆ alkyl. In several embodiments, where R^{6′} is optionally substituted, R^{6′} may be optionally substituted with with halogen, C₁₋₃ alkyl and/or C-carboxy. In several embodiments, t, t′, h, and h′ are each independently an integer of equal to or at least about: 0, 1, 2, 3, 4, 5, 6, 7, 8, 9, 10, 20, or ranges including and/or spanning the aforementioned values. In several embodiments, X³ is —C(O)—NH— and R⁹ is —(CH₂)₂— (e.g., where X⁵ is a bond, h is 2, and t is 1), —((CH₂)₂—O))ₜ— (where X⁵ is a —O—, h is 2), or —((CH₂)₂—O))₂— (where X⁵ is a —O—, h is 2, and t is 2). In several embodiments, t is an integer from 1 to 5 (e.g., 1, 2, 3, 4, or 5). In several embodiments, h is an integer from 1 to 5 (e.g., 1, 2, 3, 4, or 5). In several embodiments, each of t′ and h′ is independently an integer from 0 to 5 (e.g., 0, 1, 2, 3, 4, or 5).

Referring to W² or W⁴, in several embodiments, X⁴ is —C(O)—NH—, —C(O)O— or —C(O)—OH. In several embodiments, R¹⁰ is not present. For example, in several embodiments, where X⁴ is —C(O)—OH in a given unit of W² or W⁴, R¹⁰ is not present. In several embodiments, R¹⁰ is —H, an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol. In several embodiments, R¹⁰ is optionally substituted C₁₋₆alkyl. In several embodiments, where substituted, R¹⁰ is substituted with one or more of —OH, halogen, C₁₋₃ alkyl and/or C-carboxy. In several embodiments, R¹⁰ is —[((CH₂)_{h″}—X⁷)_{t″}—((CH₂)_{h′″}X⁸)_{t′″}]—H. In several embodiments, X⁷ and X⁸ are independently selected from a direct bond, —NR^{6″}—, and —O—. In some embodiments, each instance of R^{6″} is independently H or optionally substituted —C₁₋₆ alkyl. In several embodiments, where R^{6″} is optionally substituted, it may be optionally substituted with halogen, C₁₋₃ alkyl, and/or C-carboxy. In several embodiments, t″, t′″, h″, and h′″ are each independently an integer of equal to or at least about: 0, 1, 2, 3, 4, 5, 6, 7, 8, 9, 10, 20, or ranges including and/or spanning the aforementioned values. In several embodiments, X⁴ is —C(O)—NH— and R¹⁰ is —(CH₂)₂—OH (e.g., where X⁷ is —O—, h″ is 2, and t″ is 1, and t′″ is 0), —((CH₂)₂—O))_{t″}—H (where X⁷ is —O—, h″ is 2, and t′″ is 0). In several embodiments, t″ is an integer from 1 to 5 (e.g., 1, 2, 3, 4, or 5). In several embodiments, h″ is an integer from 1 to 5 (e.g., 1, 2, 3, 4, or 5). In several embodiments, each of t′″ and h′″ is independently an integer from 0 to 5 (e.g., 0, 1, 2, 3, 4, or 5).

In some embodiments, R⁹ and/or R¹⁰ (e.g., one or more of the methyl or methylene groups or other groups of these structures) may be optionally substituted. In some embodiments, R⁹ and/or R¹⁰ may be optionally substituted with C₁₋₆ alkyl, halogen, —OH, amino, or C-carboxy.

In several embodiments, each instance of R¹³ (e.g., in either W¹, W², W³, or W⁴) is independently H, methyl, or optionally substituted —C₁₋₆ alkyl. In several embodiments, R¹³ is methyl. In several embodiments, where R¹³ is optionally substituted, it may be optionally substituted with halogen, —OH, C₁₋₃ alkyl, and/or C-carboxy.

In several embodiments, EU is attached to Y via a carbon-carbon bond. For example, EU comprises a carbon atom that is bonded to a carbon atom of Y. In several embodiments, the carbon of EU (e.g., that is bonded to the carbon of Y or the Y-bonded carbon) has three other positions available for bonding. In several embodiments, each other position (e.g., valence position) of the Y-bonded EU carbon is occupied by a substitutent independently selected from —H, optionally substituted C₁₋₁₁ alkyl, —CN, optionally substituted —C(═NH)NH₂, optionally substituted —C(═NH)NH(C₁₋₃ alkyl), optionally substituted imidazoline, optionally substituted aryl, optionally substituted heteroaryl, optionally substituted aralkyl, optionally substituted heteroaralkyl, optionally substituted C-carboxy (where R is —H, optionally substituted C₁₋₆ alkyl, optionally substituted C₁₋₆ alkylenyl, or optionally substituted phenyl), optionally substituted C-amido where R_{A} and R_{B} are independently —H or C₁₋₆alkyl, optionally substituted succinimidyl ester, optionally substituted isoindolin-1,3-dione, an optionally substituted polystyryl unit, an optionally substituted polyacrylic acid, or two positions on the carbon are taken together to provide an optionally substituted C₃₋₁₀ cycloalkyl or optionally substituted heterocyclyl (having 3 to 10 members in the ring). In several embodiments, a position of the Y-bonded EU carbon is occupied by —CN. In several embodiments, a position of the Y-bonded EU carbon is occupied by a polymeric (or partially polymeric) unit, as disclosed elsewhere herein. In several embodiments, optional substitutions of the Y-bonded EU carbon substituent, where present on an EU group, are independently selected from C₁₋₃ alkyl, C₁₋₆ alkoxy, C₁₋₆ alkylenyl, hydroxyl, amino, halogen, C-carboxy (where R is —H, C₁₋₆ alkyl, or polyethylene glycol (PEG) (e.g., having repeat units numbering from equal to or at least about 1, 2, 3, 4, 5, 10, 15, 20, 30, 40, 50, 75, 100, 150, or ranges including and/or spanning the aforementioned values)), succinimidyl ester, 2-nitro-5-(prop-2-yn-1-yloxy)benzyl 4-cyanopentanoate, azide (N₃), C₁₋₃ alkyl azide, C₁₋₃ alkyl silane (e.g., trimethyl silane, triethyl silane, tripropyl silane), polyethylene glycol (PEG) (e.g., having repeat units numbering from equal to or at least about 1, 2, 3, 4, 5, 10, 15, 20, 30, 40, 50, 75, 100, 150, or ranges including and/or spanning the aforementioned values), and/or combinations of the foregoing. For example, where present the Y-bonded EU carbon may comprise a C₁₋₁₁ alkyl substituted with a C-carboxy where R is H. In several embodiments, the Y-bonded EU carbon substituent may lack optional substituents, may be optionally substituted by one optional substituent, or may be optionally substituted by multiple (2, 3, or more) optional substituents.

In several embodiments, EU is represented by (EU1):

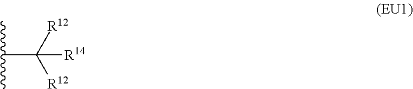

where [symbol] denotes a connection to Y (via a carbon of the Y portion of the construct) and each instance of R¹² and/or R¹⁴ is independently selected from —H, optionally substituted C₁₋₁₁ alkyl, —CN, optionally substituted —C(═NH)NH₂, optionally substituted —C(═NH)NH(C₁₋₃ alkyl), optionally substituted imidazoline, optionally substituted heterocyclyl, optionally substituted aryl, optionally substituted heteroaryl, optionally substituted aralkyl, optionally substituted heteroaralkyl, C-carboxy (where R, as defined elsewhere herein, is —H, optionally substituted C₁₋₆ alkyl, optionally substituted C₁₋₆ alkylenyl, or optionally substituted phenyl), optionally substituted C-amido (where R_{A} and R_{B}, as defined elsewhere herein, are independently —H or optionally substituted C₁₋₆ alkyl), optionally substituted succinimidyl ester, optionally substituted isoindolin-1,3-dione, an alkyl silane, an optionally substituted polystyryl unit, an optionally substituted polyacrylic acid. In some embodiments, R¹⁴ is selected from —H, optionally substituted C₁₋₁₁ alkyl, —CN, optionally substituted —C(═NH)NH₂, optionally substituted —C(═NH)NH(C₁₋₃ alkyl), optionally substituted imidazoline, optionally substituted heterocyclyl, optionally substituted aryl, optionally substituted heteroaryl, optionally substituted aralkyl, optionally substituted heteroaralkyl, C-carboxy (where R, as defined elsewhere herein, is —H, optionally substituted C₁₋₆ alkyl, optionally substituted C₁₋₆ alkylenyl, or optionally substituted phenyl), optionally substituted C-amido (where R_{A} and R_{B}, as defined elsewhere herein, are independently —H or optionally substituted C₁₋₆ alkyl), optionally substituted succinimidyl ester, optionally substituted isoindolin-1,3-dione, an alkyl silane, an optionally substituted polystyryl unit, an optionally substituted polyacrylic acid; and each instance of R¹² is independently hydrogen, optionally substituted C₁₋₆ alkyl, C-carboxy (where R is —H or optionally substituted C₁₋₆ alkyl), C-amido (where R_{A} and R_{B} are independently —H or optionally substituted C₁₋₆ alkyl), or each instance of R¹² is taken together to provide an optionally substituted C₃₋₁₀ cycloalkyl. In some embodiments, R¹⁴ is the carbon of a polymeric (or partially polymeric) terminal end unit, as disclosed elsewhere herein.

In several embodiments, R¹⁴ is selected from optionally substituted C₁₋₁₁ alkyl; —CN; optionally substituted —C(═NH)NH₂; optionally substituted heterocyclyl; optionally substituted aryl; optionally substituted heteroaryl; optionally substituted aralkyl; optionally substituted heteroaralkyl; C-carboxy (—C(O)OR) where R is —H, optionally substituted C₁₋₆ alkyl, optionally substituted C₁₋₆ alkylenyl, or optionally substituted phenyl; optionally substituted C-amido (—C(O)NHR) where R_{A} and R_{B} are independently —H or optionally substituted C₁₋₆ alkyl; optionally substituted succinimidyl ester; optionally substituted isoindolin-1,3-dione; an alkyl silane. In several embodiments, each optional substitution on R¹⁴ is selected from C₁₋₃ alkyl, C₁₋₆ alkoxy, C₁₋₆ alkylenyl, hydroxyl, amino, halogen, C-carboxy (where R is —H, C₁₋₆ alkyl, or polyethylene glycol (PEG). In several embodiments, each instance of R¹² is independently a hydrogen, an C₁₋₁₀ alkyl optionally substituted with halogen, C-carboxy, -amino, or —OH, or each instance of R² is taken together to provide an C₃₋₆ cycloalkyl optionally substituted with one or more of halogen, C-carboxy, -amino, or —OH. In several embodiments, EU is an optionally substituted polystyryl unit or an optionally substituted polyacrylic acid.

In some embodiments, optional substituents, where present on an EU group (e.g., on any one of R¹², R¹², or R¹⁴), are independently selected from C₁₋₃ alkyl, C₁₋₆ alkoxy, C₁₋₆ alkylenyl, hydroxyl, amino, halogen, C-carboxy (where R is —H, C₁₋₆ alkyl, or PEG), succinimidyl ester, 2-nitro-5-(prop-2-yn-1-yloxy)benzyl 4-cyanopentanoate, azide (N₃), C₁₋₃ alkyl azide, C₁₋₃ alkyl silane, PEG, and/or combinations of the foregoing. In some embodiments, R¹⁴ may be optionally substituted with the following:

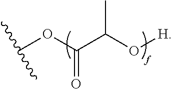

When reference to “each instance” of a variable is indicated as being “independently” one of various structures (e.g., “each instance of R¹² is independently hydrogen or an optionally substituted alkyl”), or where similar language is used, those variables, even though labeled with the same alphanumeric reference (e.g., two instances of the variable “R¹²”), may be the same or different. For example, where each instance of R¹² is independently hydrogen or an optionally substituted alkyl, both instances of R¹² may be H or, alternatively, one instance of R¹² may be —H and the other instance of R¹² may be optionally substituted alkyl. In several embodiments, both instances of R¹² are —H. In several embodiments, one instance of R¹² is —H and the other is an optionally substituted alkyl. In several embodiments, each instance of R¹² is an optionally substituted alkyl and the optionally substituted alkyls are the same. In several embodiments, each instance of R¹² is an optionally substituted alkyl and the optionally substituted alkyls are different. In some embodiments, both instances of R¹² are taken together to provide an optionally substituted cycloalkyl (e.g., C₃₋₁₀ cycloalkyl). In some embodiments, each instance of R¹² is independently optionally substituted C₁₋₆ alkyl.

In several embodiments, EU is represented by (EU2):

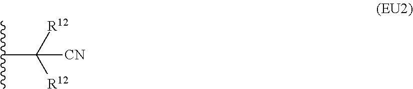

where [symbol] denotes a connection to Y (via a carbon of the Y portion of the construct) and each instance of R¹² is independently selected from —H, optionally substituted C₁₋₁₁ alkyl, optionally substituted —C(═NH)NH₂, optionally substituted —C(═NH)NH(C₁₋₃ alkyl), optionally substituted imidazoline, optionally substituted aryl, optionally substituted heteroaryl, optionally substituted aralkyl, optionally substituted heteroaralkyl, optionally substituted C-carboxy (where R is —H, optionally substituted C₁₋₆ alkyl, optionally substituted C₁₋₆ alkylenyl, or optionally substituted phenyl), optionally substituted C-amido where R_{A} and R_{B} are independently —H or C₁₋₆ alkyl, optionally substituted succinimidyl ester, optionally substituted isoindolin-1,3-dione, an optionally substituted polystyryl unit, an optionally substituted polyacrylic acid, or two positions on the carbon are taken together to provide an optionally substituted C₃₋₁₀ cycloalkyl or optionally substituted heterocyclyl (having 3 to 10 members in the ring). In several embodiments, R¹² is independently hydrogen, optionally substituted C₁₋₆ alkyl, C-carboxy (where R is —H or optionally substituted C₁₋₆ alkyl), C-amido (where R_{A} and R_{B} are independently —H or optionally substituted C₁₋₆ alkyl), or each instance of R¹² is taken together to provide an optionally substituted C₃₋₁₀ cycloalkyl. In several embodiments, each instance of R¹² is independently hydrogen, an optionally substituted alkyl (e.g., a C₁₋₆ alkyl optionally substituted with one or more of a halogen, C-carboxy, -amino, —OH, etc.), or each instance of R¹² is taken together to provide an optionally substituted cycloalkyl.

In several embodiments, EU is selected from the group consisting of:

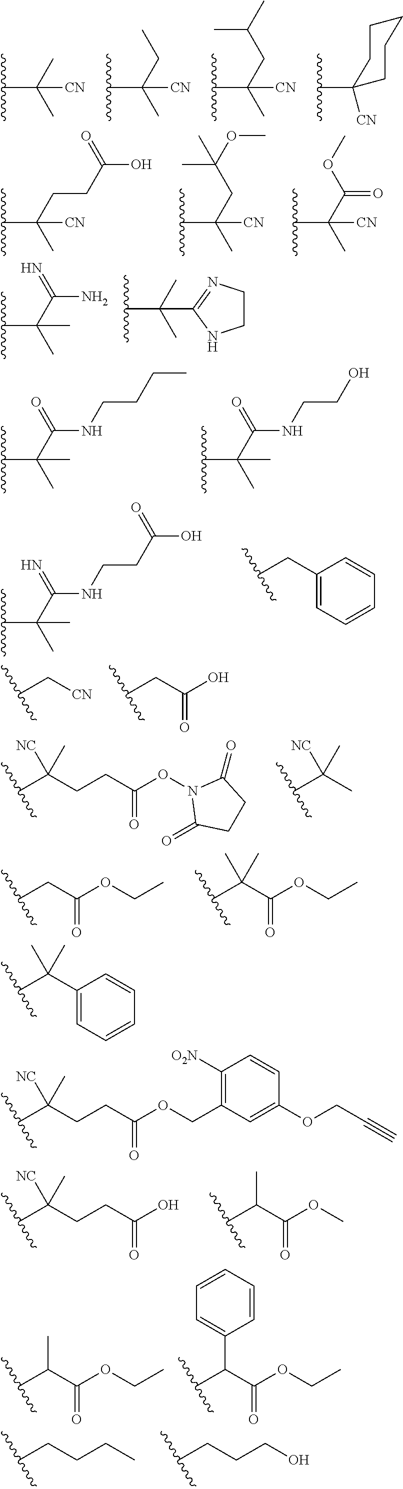

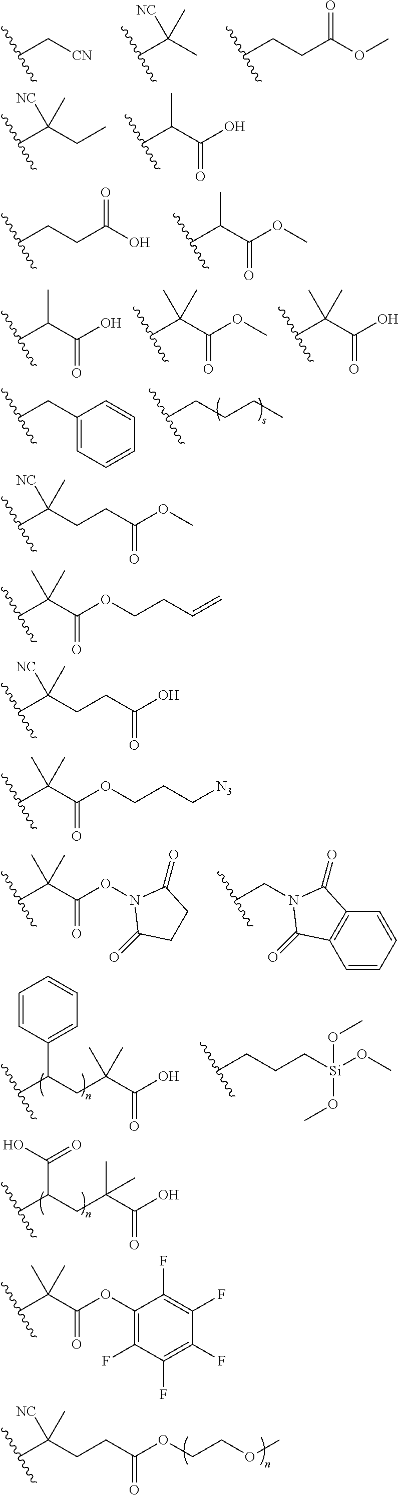

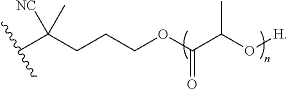

In several embodiments, EU is selected from the group consisting of:

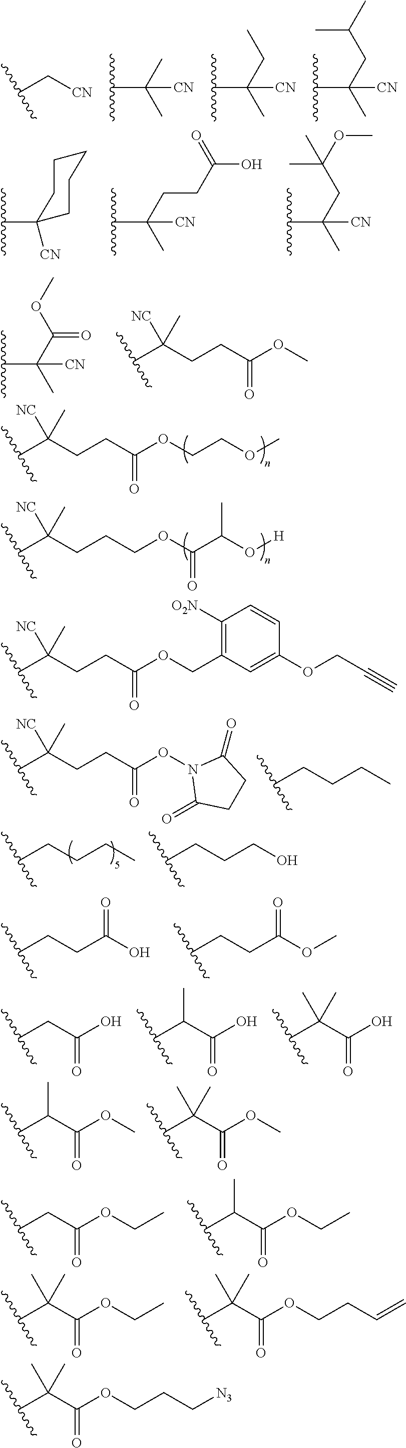

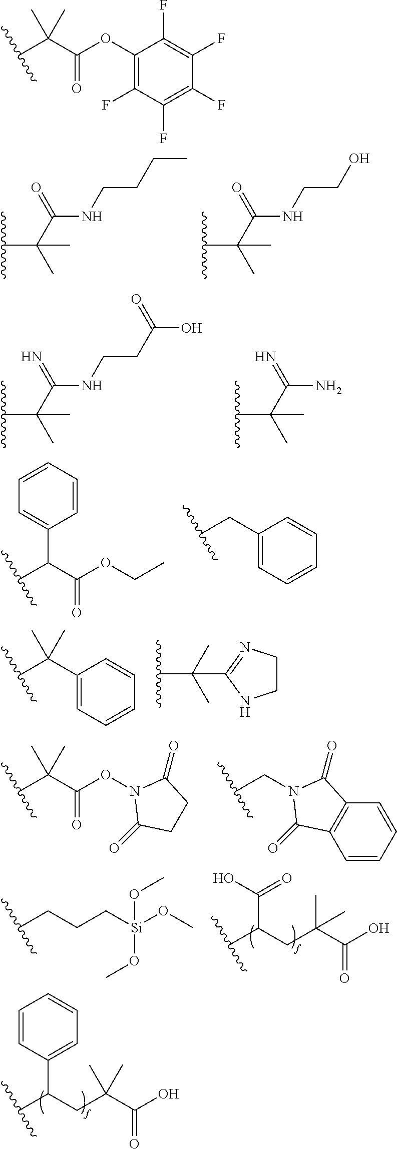

In several embodiments, f, where present, is an integer greater than or equal to about: 0, 1, 2, 3, 4, 5, 10, 20, 40, 50, 100, 150, 200, or ranges spanning and/or including the aforementioned values.

In several embodiments, each instance of R¹² is —CH₃ and EU is the following group:

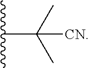 [SMILES: *C(C)(C)C#N]

In several embodiments, Z comprises one or more of galactose, glucose, galactosamine, glucosamine, N-acetylgalactosamine, and/or N-acetylglucosamine. In several embodiments, Z is bound at its C1, C2 or C6 to Y.

In several embodiments, the ratio of p to r is about 1:1. In several embodiments, the ratio of p to r is about 4:1.

In several embodiments, Y is Ya′; k is 2; R¹ is —(CH₂)₂—C(CH₃)(CN)—. In several embodiments, X³ and R⁹ taken together is —C(O)—NH—(CH₂)₂—(O—CH₂—CH₂)ₜ—, In several embodiments, t is 1. In several embodiments, Z is one or more of galactose, glucose, galactosamine, glucosamine, N-acetylgalactosamine, or N-acetylglucosamine. In several embodiments, Z is N-acetylgalactosamine or N-acetylglucosamine. In several embodiments, q is 0. In several embodiments, q is 3.

In several embodiments, Y is Ye′. In several embodiments, q is 0. In several embodiments, k is 2. In several embodiments, R¹ is —(CH₂)₂—C(CH₃)(CN)—. In several embodiments, X³ and R⁹ taken together is —C(O)—NH—(CH₂)₂—(O—CH₂—CH₂)ₜ—. In several embodiments, t is 1. In several embodiments, Z is one or more of galactose, glucose, galactosamine, glucosamine, N-acetylgalactosamine, or N-acetylglucosamine. In several embodiments, Z is N-acetylgalactosamine or N-acetylglucosamine.

Several embodiments pertain to a compound for the induction of antigen-specific immune tolerance in a subject, the compound comprising an antigen, a linker comprising a polymeric portion, and a targeting moiety (e.g., a liver targeting moiety). In several embodiments, the linker is bonded to the antigen via a disulfide bond or a disulfanyl ethyl ester. In several embodiments, the disulfide bond or the disulfanyl ethyl ester are each configured to be cleaved upon administration of the compound to the subject. In several embodiments, the disulfide bond or the disulfanyl ethyl ester are configured to release the antigen from the polymeric linker. In several embodiments, the polymeric linker comprises a terminal end unit. In several embodiments, the terminal end unit improves the stability of the compound in solution.

Several embodiments pertain to a compound for the induction of antigen-specific immune tolerance in a subject. In several embodiments, the compound comprises an antigen to which tolerance is desired or a tolerogenic portion thereof. In several embodiments, the antigen to which tolerance is desired, when presented alone to the subject is capable of inducing an unwanted immune response in the subject. In several embodiments, the compound comprises a polymeric linker (e.g., a linker comprising a polymeric portion). In several embodiments, the polymeric linker comprises a copolymer (e.g., a random copolymer, gradient copolymer, block copolymer, etc.), wherein the copolymer comprises a first acrylyl unit and a second acrylyl unit. In several embodiments, the first acrylyl unit comprises a first C-amido or C-carboxy functionality. In several embodiments, the second acrylyl unit comprises a second C-amido or C-carboxy functionality. In several embodiments, the second C-amido or C-carboxy functionality is conjugated to an aliphatic group, an amine, a polyamino, an alcohol, a polyether, or an aliphatic alcohol. In several embodiments, the polymeric linker is bonded to the antigen to which tolerance is desired or tolerogenic portion thereof via a disulfide bond or a disulfanyl ethyl ester. In several embodiments, the disulfide bond or the disulfanyl ethyl ester are each configured to be cleaved after administration of the compound to the subject and to release the antigen to which tolerance is desired or tolerogenic portion thereof from the polymeric linker. In several embodiments, the polymeric linker comprises a terminal end unit. In several embodiments, the terminal end unit improves the stability of the compound in solution. In several embodiments, the compound comprises a liver-targeting moiety. In several embodiments, the liver targeting moiety is connected to the first acrylyl unit via the first C-amido or C-carboxy functionality. In several embodiments, the first acrylyl unit is covalently linked to the liver targeting moiety through an alkyl group, a polyether, a polyamino group, combinations of any of the foregoing, or the like.

In several embodiments, a terminal end unit as disclosed herein may be a reaction product of an azo compound (e.g., a compound bearing a functionalized diazine functional group) with a dithioester. In several embodiments, the terminal end unit lacks one or more of a sulfur (S) atom or an aryl group. In several embodiments, the terminal end unit lacks a dithioester.

In several embodiments, the liver-targeting moiety comprises a galactosylating or glucosylating moiety.

In several embodiments, the tolerogenic construct is prepared using N-hydroxysuccinamidyl linkers, malaemide linkers, vinylsulfone linkers, pyridyl di-thiol-poly(ethylene glycol) linkers, pyridyl di-thiol linkers, n-nitrophenyl carbonate linkers, NHS-ester linkers, and nitrophenoxy poly(ethylene glycol)ester linkers.

In several embodiments, the antigen, mimetic thereof, fragment thereof, or tolerogenic portion thereof induces an unwanted immune response in a subject.

In several embodiments, the antigen, mimetic thereof, fragment thereof, or tolerogenic portion thereof is associated with an autoimmune disease. In several embodiments, the autoimmune disease is selected from the group consisting of type 1 diabetes, multiple sclerosis, rheumatoid arthritis, vitiligo, uveitis, pemphis vulgaris, neuromyelitis optica, Goodpasture's disease, Parkinson's disease, myasthenia gravis, and celiac disease.

In several embodiments, the antigen, mimetic thereof, fragment thereof, or tolerogenic portion thereof comprises a self antigen. In several embodiments, the self antigen is selected from myelin basic protein, myelin oligodendrocyte glycoprotein and proteolipid protein, a portion of any of said antigens, and a mimetic of any of said antigens. In several embodiments, the self antigen is selected from insulin, proinsulin, preproinsulin, glutamic acid decarboxylase-65 (GAD-65), GAD-67, insulinomaassociated protein 2 (IA-2), and insulinoma-associated protein 213 (IA-213), ICA69, ICA12 (SOX-13), carboxypeptidase H, Imogen 38, GLIMA 38, chromogranin-A. HSP-60, caboxypeptidase E, peripherin, glucose transporter 2, hepatocarcinoma-intestinepancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, SST G-protein coupled receptors 1-5, and a portion of any of said antigens, and a mimetic of any of said antigens.

In several embodiments, the antigen, mimetic thereof, fragment thereof, or tolerogenic portion thereof comprises a food antigen. In several embodiments, the food antigen is selected from the group consisting of conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), α-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1 lS globulin (Sin a 2), lipid transfer protein (Sin a 3), profilin (Sin a 4), profilin (Api g 4), high molecular weight glycoprotein (Api g 5), Pen a 1 allergen (Pen a 1), allergen Pen m 2 (Pen m 2), tropomyosin fast isoform, high molecular weight glutenin, low molecular weight glutenin, alpha-, gamma- and omega-gliadin, hordein, secalin, avenin, major strawberry allergy Fra a I-E (Fra a 1), profilin (Mus xp 1), a portion of any of said antigens, and a mimetic of any of said antigens. In several embodiments, the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens. In several embodiments, the food antigen is associated with celiac disease.

In several embodiments, the antigen, mimetic thereof, fragment thereof, or tolerogenic portion thereof comprises a therapeutic agent. In several embodiments, the therapeutic agent is selected from Abciximab, Adalimumab, Agalsidase alfa, Agalsidase beta, Aldeslukin, Alglucosidase alfa, Factor VIII, Factor IX, Infliximab, L-asparaginase, Laronidase, Natalizumab, Octreotide, Phenylalanine ammonia-lyase (PAL), Rasburicase (uricase), a gene therapy vector, or AAV, a portion of any of said antigens, and a mimetic of any of said antigens.

In several embodiments, the antigen, mimetic thereof, fragment thereof, or tolerogenic portion thereof comprises a transplant antigen. In several embodiments, the transplant antigen is selected from the group consisting of subunits of the MHC class I and MHC class II haplotype proteins and their complexes with the antigens they present, and minor blood group antigens RhCE, Kell, Kidd, Duffy, and Ss.

In several embodiments, the antigen, mimetic thereof, fragment thereof, or tolerogenic portion thereof is desmoglein-3, -1, and/or -4.

In several embodiments, the antigen, mimetic thereof, fragment thereof, or tolerogenic portion thereof is associated with pemphigus vulgaris.

Several embodiments disclosed herein pertain to a composition comprising a compound as disclosed above or elsewhere herein. Several embodiments disclosed herein pertain to the use of a compound (e.g., a construct) as disclosed above or elsewhere herein. In several embodiments, the use is for inducing tolerance to X and/or an antigen, mimetic thereof, fragment thereof, or tolerogenic portion thereof.

Several embodiments disclosed herein pertain to a method of inducing tolerance to an antigen to which a subject is capable of developing an unwanted immune response, comprising administering a compound as disclosed above or elsewhere herein to the subject. In several embodiments, the compound is administered prior to the subject being exposed to the antigen, after the subject has been exposed to the antigen, or both.

Several embodiments disclosed herein pertain to the use of the compound as disclosed above or elsewhere herein for the preparation of a medicament for inducing tolerance to an antigen. In several embodiments, the antigen, mimetic thereof, fragment thereof, or tolerogenic portion thereof comprises a foreign transplant antigen, a tolerogenic portion thereof, or a mimetic thereof against which transplant recipients develop an unwanted immune response.

In several embodiments, the antigen, mimetic thereof, fragment thereof, or tolerogenic portion thereof (e.g., X) comprises a foreign food, animal, plant or environmental antigen, a tolerogenic portion of any of thereof, or a mimetic of any of thereof against which induces patients develop an unwanted immune response.

In several embodiments, the antigen, mimetic thereof, fragment thereof, or tolerogenic portion thereof (e.g., X) comprises a foreign therapeutic agent, a tolerogenic portion thereof, or a mimetic thereof against which patients develop an unwanted immune response.

In several embodiments, the antigen, mimetic thereof, fragment thereof, or tolerogenic portion thereof (e.g., X) comprises comprises a self-antigen, a tolerogenic portion thereof, or a mimetic thereof against the endogenous version of which patients develop an unwanted immune response or a tolerogenic portion thereof.

In several embodiments, the antigen, mimetic thereof, fragment thereof, or tolerogenic portion thereof (e.g., X) comprises an antibody, antibody fragment or ligand that specifically binds a circulating protein or peptide or antibody, which circulating protein or peptide or antibody is causatively involved in transplant rejection, immune response against a therapeutic agent, autoimmune disease, hypersensitivity and/or allergy.

Several embodiments disclosed herein pertain to a pharmaceutically acceptable composition for inducing tolerance to a therapeutic protein in a subject having a deficiency in production of a functional analogous native protein, comprising a compound as disclosed above or elsewhere herein.

Several embodiments disclosed herein pertain to the use of a compound or composition as disclosed above or elsewhere herein for treating an unwanted immune response against an antigen. In several embodiments, the unwanted immune response is associated with type I diabetes, multiple sclerosis, rheumatoid arthritis, vitiligo, uveitis, pemphigus vulgaris, neuromyelitis optica, Parkinson's disease, Goodpasture's disease, celiac disease, or myasthenia gravis.

Several embodiments disclosed herein pertain to manufacturing a medicament for use in treating an unwanted immune response against an antigen.

Several embodiments disclosed herein pertain to a method of manufacturing a compound having the following structure:

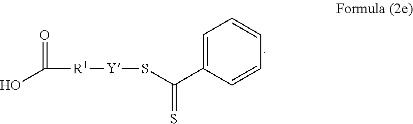

In several embodiments, the method comprises providing a RAFT reagent of the following structure:

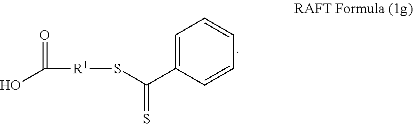

In several embodiments, the method comprises providing providing one or more acrylyl monomers of the following structure:

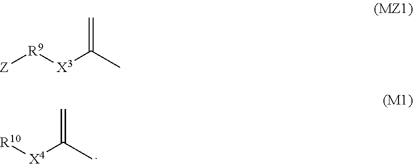

In several embodiments, the method comprises mixing the RAFT reagent with the one or more acrylyl monomers to form the compound. In several embodiments, the variables are as disclosed elsewhere herein. In several embodiments, R₁ is selected from the group consisting of —CH₂—, —(CH₂)₂—C(CH₃)(CN)—, —(CH₂)₂—C(CH₃)CH₃)—, —(CH₂)₂—CH(CH₃)—, and —CH(CH₃)—. In several embodiments, X³ is selected from —C(O)—NH— or —C(O)O—. In several embodiments, X⁴ is —C(O)—NH—, —C(O)O—, or —C(O)—OH. In several embodiments, R⁹ is as defined elsewhere herein. In several embodiments, R⁹ is a direct bond or —[((CH₂)ₕX⁵)ₜ—((CH₂)_{h′}—X⁶)_{t′}]—. In several embodiments, X⁵ and X⁶ are independently selected from a direct bond, —CNR^{6′}—, and —O—. In several embodiments, each instance of R^{6′} is independently H or —C₁₋₆ alkyl optionally substituted with halogen, C₁₋₃ alkyl or C-carboxy. In several embodiments, R¹⁰ is not present or is —[((CH₂)_{h″}X⁷)_{t″}—((CH₂)_{h′″}—X⁸)_{t′″}]—H. In several embodiments, X⁷ and X⁸ are independently selected from a direct bond, —CNR^{6″}—, and —O—. In several embodiments, each instance of R^{6″} is independently H or —C₁₋₆ alkyl optionally substituted with halogen, C₁₋₃ alkyl or C-carboxy. In several embodiments, t, t′, h, and h′ are each independently an integer of equal to or at least about: 0, 1, 2, 3, 4, 5, 6, 7, 8, 9, 10, 20. In several embodiments, Y′ is a random copolymer, gradient copolymer, or block copolymer of W¹ and W² or of W³ and W⁴. In several embodiments, W¹, W², W³, and W⁴ are as depicted below:

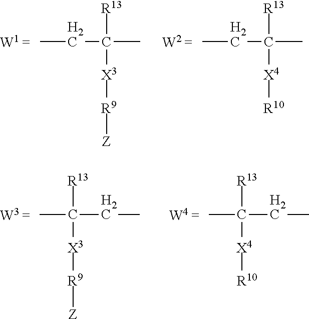

In several embodiments, the number of repeat units of W¹ or W³ in Y′ is denoted as p and wherein p is an integer of at least about 1. In several embodiments, the number of repeat units of W² or W⁴ in Y′ is denoted as r and wherein r is an integer of at least about 1. In several embodiments, the sum of p and r is greater than about 170. In several embodiments, the variables are as disclosed elsewhere herein. In several embodiments, each instance of R¹³ is independently H, methyl, or optionally substituted —C₁₋₆ alkyl. In several embodiments, Z comprises a liver-targeting moiety.

Several embodiments disclosed herein pertain to a method of manufacturing a compound having the following structure:

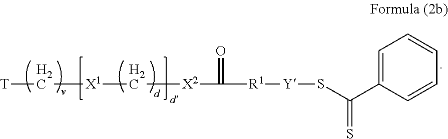

In several embodiments, the method comprises providing a RAFT reagent of the following structure:

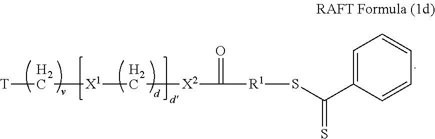

In several embodiments, the method comprises providing one or more acrylyl monomers of the following structure:

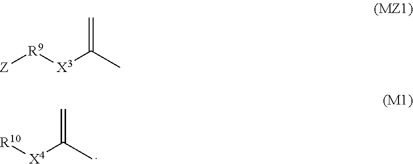

In several embodiments, the method comprises mixing the RAFT reagent with the one or more acrylyl monomers to form the compound. In several embodiments, the variables are as disclosed elsewhere herein. In several embodiments, T is pyridyl disulfide (PDS) or a carboxylic acid. In several embodiments, X¹ and X² are independently selected from a direct bond, —NR⁶—, and —O—. In several embodiments, v, d, and d′ are independently an integer greater than or equal: 0, 1, 2, 3, 4, 5, 10, 15, 20, or 44. In several embodiments, R⁶ is H or C₁₋₆ alkyl optionally substituted with a halogen, C₁₋₃ alkyl or C-carboxy. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, wherein Y′ is a random copolymer, gradient copolymer, or block copolymer of W¹ and W² or of W³ and W⁴, where W¹, W², W³, and W⁴ are as depicted below:

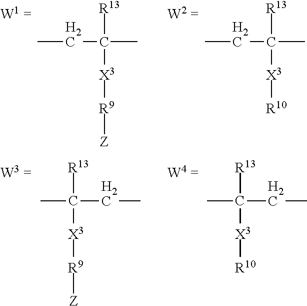

In several embodiments, the number of repeat units of W¹ or W³ in Y′ is denoted as p and wherein p is an integer of at least about 1. In several embodiments, the number of repeat units of W² or W⁴ in Y′ is denoted as r and wherein r is an integer of at least about 1. In several embodiments, the sum of p and r is greater than about 170. In several embodiments, the variables are as disclosed elsewhere herein. In several embodiments, each instance of R¹³ is independently H, methyl, or optionally substituted —C₁₋₆ alkyl. In several embodiments, Z comprises a liver-targeting moiety.

Several embodiments disclosed herein pertain to a method of manufacturing a compound having the following structure:

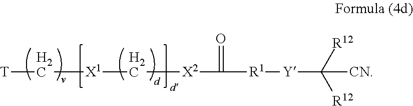

In several embodiments, the method comprises providing a dithiobenzoate containing compound of the following structure:

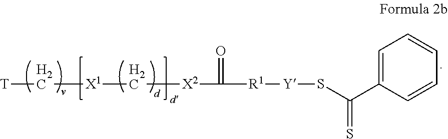

In several embodiments, the variables are as disclosed elsewhere herein. In several embodiments, the method comprises providing a terminal end unit by exposing a structure of Formula (2b) (or another compound comprising R² or DTB) to the following structure:

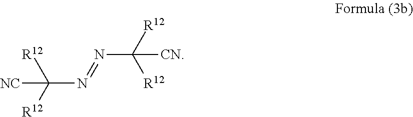

In several embodiments, the method comprises mixing the terminal end unit reagent with the dithiobenzoate containing compound to form the structure of Formula (4d). In several embodiments, the variables are as disclosed elsewhere herein. In several embodiments, T, when present, is PDS or a carboxylic acid. In several embodiments, X¹ and X² are independently selected from a direct bond, —NR⁶—, and —O—. In several embodiments, R⁶ is H or C₁₋₆ alkyl optionally substituted with a halogen, C₁₋₃ alkyl or C-carboxy. In several embodiments, v, d, and d′, when present, are independently an integer greater than or equal to 0, 1, 2, 3, 4, 5, 10, 15, 20, 40, 50, 75, 100, 150. In several embodiments, each instance of R¹² is independently hydrogen or an optionally substituted alkyl. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, wherein Y′ is a random copolymer, gradient copolymer, or block copolymer of W¹ and W² or of W³ and W⁴. In several embodiments, W¹, W², W³, and W⁴ are as depicted below:

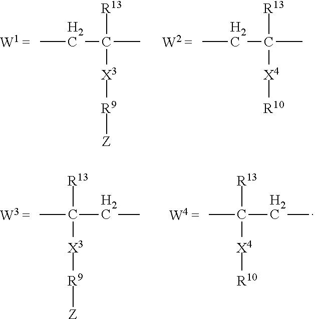

In several embodiments, the number of repeat units of W¹ or W³ in Y′ is denoted as p. In several embodiments, p is an integer of at least about 1. In several embodiments, the number of repeat units of W² or W⁴ in Y′ is denoted as r. In several embodiments, r is an integer of at least about 1. In several embodiments, the sum of p and r is greater than about 170. In several embodiments, the variables are as disclosed elsewhere herein. In several embodiments, X³ is selected from —C(O)—NH— or —C(O)O—. In several embodiments, X⁴ is —C(O)—NH—, —C(O)O—, or —C(O)—OH. In several embodiments, R⁹ is a direct bond or —[((CH₂)ₕX⁵)ₜ—((CH₂)_{h′}X⁶)_{t′}]—. In several embodiments, X⁵ and X⁶ are independently selected from a direct bond, —CNR^{6′}—, and —O—. In several embodiments, each instance of R^{6′} is independently H or —C₁₋₆ alkyl optionally substituted with halogen, C₁₋₃ alkyl or C-carboxy. In several embodiments, R¹⁰ is not present or is —[((CH₂)_{h″}—X⁷)_{t″}—((CH₂)_{h′″}—X⁸)_{t′″}]—H. In several embodiments, X⁷ and X⁸ are independently selected from a direct bond, —CNR^{6″}—, and —O—. In several embodiments, each instance of R^{6″} is independently H or —C₁₋₆ alkyl optionally substituted with halogen, C₁₋₃ alkyl or C-carboxy. In several embodiments, t, t′, h, h′, t″, t′″, h″, and h′″ are each independently an integer of equal to or at least about: 0, 1, 2, 3, 4, 5, 6, 7, 8, 9, 10, 20 (or a range spanning and/or including any of the aforementioned values). In several embodiments, R¹³ is independently H, methyl, or optionally substituted —C₁₋₆ alkyl. In several embodiments, Z comprises a liver-targeting moiety.

Several embodiments disclosed herein pertain to a method of manufacturing a compound having the following structure:

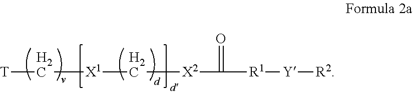

In several embodiments, the variables are as defined elsewhere herein. In several embodiments, R² is an end capping group (e.g., a CTA remnant). In several embodiments, the method comprises providing a carboxylic acid-terminated compound of the following structure:

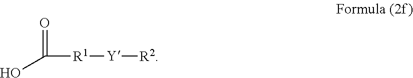

In several embodiments, the method comprises providing a PDS-functionalizing agent having the following structure:

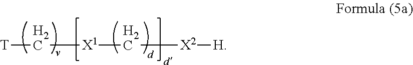

In several embodiments, the method comprises mixing the carboxylic acid-terminated compound with the PDS-functionalizing agent form the compound. In several embodiments, the method comprises mixing the carboxylic acid-terminated compound with the PDS-functionalizing agent in coupling conditions to form the compound. In several embodiments, R² is displaced by EU. In several embodiments, EU is represented by the following structure:

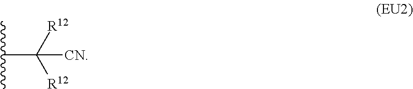

In several embodiments, the variables are as disclosed elsewhere herein. In several embodiments, T is PDS. In several embodiments, X¹ and X² are independently selected from a direct bond, —NR⁶—, and —O—. In several embodiments, v, d, and d′ are independently an integer greater than or equal: 0, 1, 2, 3, 4, 5, 10, 15, 20, or 44. In several embodiments, R⁶ is H or C₁₋₆ alkyl optionally substituted with a halogen, C₁₋₃ alkyl or C-carboxy. In several embodiments, R₁ is selected from the group consisting of —CH₂—, —(CH₂)₂—C(CH₃)(CN)—, —(CH₂)₂—C(CH₃)(CH₃)—, —(CH₂)₂—CH(CH₃)—, and —CH(CH₃)—. In several embodiments, Y′ is a random copolymer, gradient, block copolymer of W¹ and W², where W¹ and W² are as depicted below:

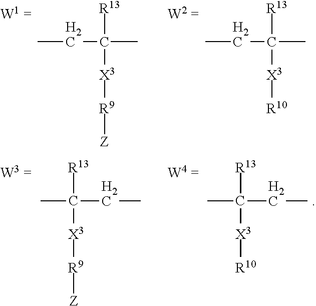

In several embodiments, the number of repeat units of W¹ or W³ in Y′ is denoted as p. In several embodiments, p is an integer of at least about 1. In several embodiments, the number of repeat units of W² or W⁴ in Y′ is denoted as r. In several embodiments, r is an integer of at least about 1. In several embodiments, the sum of p and r is greater than about 170. In several embodiments, each instance of R¹² is independently hydrogen or an optionally substituted alkyl. In several embodiments, each instance of R¹³ is independently H, methyl, or optionally substituted —C₁₋₆alkyl. In several embodiments, Z comprises a liver-targeting moiety.

Several embodiments disclosed herein pertain to a compound for the induction of antigen-specific immune tolerance in a subject. In several embodiments, the compound comprises an antigen to which tolerance is desired or a tolerogenic portion thereof. In several embodiments, the antigen to which tolerance is desired, when presented alone to the subject is capable of inducing an unwanted immune response in the subject. In several embodiments, the compound comprises a polymeric linker that does not include a dithiobenzoate, trithiocarbonate, or a xanthate. In several embodiments, the compound comprises a polymeric linker that does not include a thiocarbonylthio compounds such as dithioesters, dithiocarbamates, trithiocarbonates, and xanthates (e.g., which mediate the polymerization via a reversible chain-transfer process). In several embodiments, the linker comprises a copolymer (e.g., a random copolymer, gradient copolymer, block copolymer, or mixture of the foregoing). In several embodiments, the copolymer (e.g., a random copolymer, gradient copolymer, block copolymer, or mixture of the foregoing) comprises of first acrylyl unit and a second acrylyl unit. In several embodiments, the first acrylyl unit comprises a first C-amido or C-carboxy functionality. In several embodiments, the second acrylyl unit comprises a second C-amido or C-carboxy functionality. In several embodiments, the second C-amido or C-carboxy functionality is conjugated (e.g., bonded) to an aliphatic group, an amine, a polyamino, an alcohol, a polyether, or an aliphatic alcohol. In several embodiments, the polymeric linker is conjugated to the antigen to which tolerance is desired or tolerogenic portion thereof via a disulfide bond or a disulfanyl ethyl ester. In several embodiments, the disulfide bond or the disulfanyl ethyl ester are each configured to be cleaved upon administration of the compound to the subject and to release the antigen to which tolerance is desired or tolerogenic portion thereof from the polymeric linker. In several embodiments, the compound comprises a liver-targeting moiety. In several embodiments, the liver targeting moiety is connected to the first acrylyl unit through the first C-amido or C-carboxy functionality and a polyether. In several embodiments, the polymeric linker comprises a terminal end unit that improves the stability of the compound in solution as compared to a linker that includes a dithioester. In several embodiments, the polymeric linker comprises a terminal end unit that improves the stability of the compound in solution as compared to a polymeric linker that includes a dithiobenzoate-containing, trithiocarbonate-containing, or a xanthate-containing end-capping group. In several embodiments, the polymeric linker comprises a terminal end unit that improves the stability of the compound in solution by at least 20% as compared to a polymeric linker that includes a dithioester-containing, a dithiobenzoate-containing, trithiocarbonate-containing, or a xanthate-containing end-capping group.

Several embodiments disclosed herein pertain to a compound for the induction of antigen-specific immune tolerance in a subject. In several embodiments, the compound comprises an antigen to which tolerance is desired. In several embodiments, the antigen to which tolerance is desired, when presented alone to the subject is capable of inducing an unwanted immune response in the subject. In several embodiments, the antigen comprises one or more of a tolerogenic portion of myelin basic protein, a tolerogenic portion of myelin oligodendrocyte glycoprotein and/or a tolerogenic portion of proteolipid protein. In several embodiments, the compound comprises a polymeric linker that does not include dithioester. In several embodiments, the compound comprises a polymeric linker that does not include a dithiobenzoate, trithiocarbonate, or a xanthate. In several embodiments, the linker comprises a copolymer (e.g., a random copolymer, gradient copolymer, block copolymer, or mixture of the foregoing). In several embodiments, the copolymer (e.g., a random copolymer, gradient copolymer, block copolymer, or mixture of the foregoing) comprises of first acrylyl unit and a second acrylyl unit. In several embodiments, the first acrylyl unit comprises a first C-amido or C-carboxy functionality. In several embodiments, the second acrylyl unit comprises a second C-amido or C-carboxy functionality. In several embodiments, the second C-amido or C-carboxy functionality is conjugated to an aliphatic group, an amine, a polyamino, an alcohol, a polyether, or an aliphatic alcohol. In several embodiments, the polymeric linker is bonded to the antigen to which tolerance is desired or tolerogenic portion thereof via a disulfide bond or a disulfanyl ethyl ester. In several embodiments, the disulfide bond or the disulfanyl ethyl ester are each configured to be cleaved upon administration of the compound to the subject and to release the antigen to which tolerance is desired or tolerogenic portion thereof from the polymeric linker. In several embodiments, the polymeric linker comprises a terminal end unit that improves the stability of the compound in solution by at least 20% as compared to a linker that includes a dithiobenzoate-containing, trithiocarbonate-containing, or a xanthate-containing end-capping group. In several embodiments, the compound comprises a liver-targeting moiety. In several embodiments, the liver-targeting moiety comprises N-acetylgalactosamine. In several embodiments, the liver targeting moiety is connected to the first acrylyl unit through the first C-amido or C-carboxy functionality and a polyether.

Several embodiments disclosed herein pertain to a compound for the induction of antigen-specific immune tolerance in a subject. In several embodiments, the compound comprises an antigen to which tolerance is desired. In several embodiments, the antigen to which tolerance is desired, when presented alone to the subject is capable of inducing an unwanted immune response in the subject. In several embodiments, the antigen comprises a tolerogenic portion of human proinsulin. In several embodiments, the compound comprises a polymeric linker that does not include dithioester. In several embodiments, the compound comprises a polymeric linker that does not include a dithiobenzoate, trithiocarbonate, or a xanthate. In several embodiments, the linker comprises a copolymer. In several embodiments, the copolymer (e.g., a random copolymer, gradient copolymer, block copolymer, or mixture of the foregoing) comprises of first acrylyl unit. In several embodiments, a second acrylyl unit. In several embodiments, the first acrylyl unit comprises a first C-amido or C-carboxy functionality. In several embodiments, the second acrylyl unit comprises a second C-amido or C-carboxy functionality. In several embodiments, the second C-amido or C-carboxy functionality is conjugated to an aliphatic group, an amine, a polyamino, an alcohol, a polyether, or an aliphatic alcohol. In several embodiments, the polymeric linker is bonded to the antigen to which tolerance is desired or tolerogenic portion thereof via a disulfide bond or a disulfanyl ethyl ester. In several embodiments, the disulfide bond or the disulfanyl ethyl ester are each configured to be cleaved upon administration of the compound to the subject and to release the antigen to which tolerance is desired or tolerogenic portion thereof from the polymeric linker. In several embodiments, the polymeric linker comprises a terminal end unit that improves the stability of the compound in solution by at least 20% as compared to a linker that includes a dithiobenzoate-containing, trithiocarbonate-containing, or a xanthate-containing end-capping group. In several embodiments, the compound comprises a liver-targeting moiety. In several embodiments, the liver targeting moiety comprises N-acetylgalactosamine. In several embodiments, the liver targeting moiety is connected to the first acrylyl unit through the first C-amido or C-carboxy functionality and a polyether.

Several embodiments disclosed herein pertain to a compound for the induction of antigen-specific immune tolerance in a subject. In several embodiments, the compound comprises an antigen to which tolerance is desired. In several embodiments, the antigen to which tolerance is desired, when presented alone to the subject is capable of inducing an unwanted immune response in the subject. In several embodiments, the antigen comprises a tolerogenic portion of deamidated alpha-gliadin. In several embodiments, the compound comprises a polymeric linker that does not include dithioester. In several embodiments, the compound comprises a polymeric linker that does not include a dithiobenzoate, trithiocarbonate, or a xanthate. In several embodiments, the linker comprises a copolymer (e.g., a random copolymer, gradient copolymer, block copolymer, or mixture of the foregoing). In several embodiments, the copolymer comprises of first acrylyl unit. In several embodiments, the copolymer comprises a second acrylyl unit. In several embodiments, the first acrylyl unit comprises a first C-amido or C-carboxy functionality. In several embodiments, the second acrylyl unit comprising a second C-amido or C-carboxy functionality. In several embodiments, the second C-amido or C-carboxy functionality is conjugated to an aliphatic group, an amine, a polyamino, an alcohol, a polyether, or an aliphatic alcohol. In several embodiments, the polymeric linker is bonded to the antigen to which tolerance is desired or tolerogenic portion thereof via a disulfide bond or a disulfanyl ethyl ester. In several embodiments, the disulfide bond or the disulfanyl ethyl ester are each configured to be cleaved upon administration of the compound to the subject and to release the antigen to which tolerance is desired or tolerogenic portion thereof from the polymeric linker. In several embodiments, the polymeric linker comprises a terminal end unit that improves the stability of the compound in solution by at least 20% as compared to a linker that includes a dithiobenzoate-containing, trithiocarbonate-containing, or a xanthate-containing end-capping group. In several embodiments, the compound comprises a liver-targeting moiety. In several embodiments, the liver-targeting moiety comprises N-acetylgalactosamine. In several embodiments, the liver targeting moiety is connected to the first acrylyl unit through the first C-amido or C-carboxy functionality and a polyether.

### BRIEF DESCRIPTION OF THE DRAWINGS

FIG. 1 provides an SDS-PAGE gel. The gel demonstrates successful conjugation of an antigen to a polymeric linker comprising a liver-targeting moiety. The conjugation occurs by displacing a 2-thiopyridine group found on the linker moiety (providing an thiol-conjugated construct). A disulfide is formed between a sulfide of the polymeric linker and a sulfide of the antigen. The antigen is coupled to the α-end of the linker.

FIG. 2 provides another SDS-PAGE gel. The gel demonstrates the conjugation of another, different antigen to a linker comprising a liver targeting moiety. The conjugation occurs by displacing a 2-thiopyridine group found on the linker moiety. A disulfide is formed from a sulfide of the linker and a sulfide of the antigen. The antigen is coupled to the α-end of the linker.

FIG. 3 provides another SDS-PAGE gel. The gel demonstrates the conjugation of another, different antigen to a linker comprising a liver targeting moiety. The conjugation occurs by displacing a N-hydroxysuccinimide group found on the polymeric linker (providing an amine-conjugated construct). A disulfanyl ethyl ester unit results. The antigen is coupled to the α-end of the linker.

FIG. 4 provides another SDS-PAGE gel. The gel demonstrates the conjugation of another, different antigen to a linker comprising a liver targeting moiety. The conjugation occurs by displacing a N-hydroxysuccinimide group found on the polymeric linker (providing an amine-conjugated construct). A disulfanyl ethyl ester unit results. The antigen is coupled to the α end of the linker.

FIG. 5 provides an SDS-PAGE gel. The gel demonstrates the conjugation of an antigen to a linker comprising a liver targeting moiety. The conjugation occurs by displacing a 2-thiopyridine group found on the linker moiety. A disulfide is formed between a sulfide of the linker and a sulfide of the antigen. The antigen is coupled to the ω-end of the linker.

FIG. 6 shows the results of OVA01-specific OTI CD8+ T cells after antigen challenge (as a percentage of total live CD3+CD8+ T cells) in the spleen. Mice treated with saline showed a high frequency of OTI CD8+ T cells in the spleen, indicative of maintenance of an inflammatory immune response specific to the OVA01 antigen. Mice treated with DTB-containing poly(GalNAc-co-HEMA)-OVA01 or dithioester-free (e.g., DTB-free) poly(GalNAc-co-HEMA)-OVA01 exhibited significant reductions in OVA01-specific OTI CD8+ T cells in the spleen, at all doses tested.

FIG. 7 shows stability testing data for a compound comprising a terminal end unit versus one comprising a dithioester (e.g., dithiobenzoate) end-capping group. Analytical testing was performed on separate sample vials at the time points of 0, 7, 14, and 28 days, as shown in FIG. 7. The dithioester-free (e.g., DTB-free) conjugates had higher stability than dithioester containing conjugates under the same conditions.

FIG. 8 shows immune tolerance results using a mouse experimental autoimmune encephalomyelitis (EAE) model. These data illustrate that pGal-MOG30-60, as a non-limiting example of a tolerogenic composition, effectively inducted immune tolerance to MOG, and prevented autoimmune pathology of the central nervous system and the associated multiple sclerosis symptomology (EAE disease).

FIG. 9 shows immune tolerance results using a mouse model and P31 (a mimetope of chromogranin-A, which is an autoantigen in type-1 diabetes). As illustrated, pGal-p31 induced prolonged protection against the induction of type-1 diabetes as compared to p31 administered alone and as compared to saline administration. In this mouse model, protection against type-1 diabetes is indicative of effective immune tolerance induction to the autoantigen that drives disease.

FIG. 10 shows immune tolerance results using transgenic mice by measuring the induction of tolerance to subsequent antigen challenge with an adjuvanted tolerogenic peptide derived from human proinsulin. The results of both immunoassays illustrate a marked and statistically significant reduction in the magnitude of proinsulin peptide-specific T cell inflammatory responses induced by administration of pGal-proinsulin peptide (a construct as disclosed herein), and thus effectively demonstrate immune tolerance induction to insulin.

### DETAILED DESCRIPTION

Immune reactions against various antigens can be a significant source of morbidity and mortality. Immune reactions can develop in an individual that lead to adverse impacts on the health and well-being of the individual, reduced efficacy of a treatment being received by an individual, and even reactions to endogenous molecules naturally occurring or existing in the individual. While broad immune suppression is utilized in certain scenarios to address certain types of immune responses, these can lead to generalized susceptibility to infection and sickness. Thus, a more tailored approach, such as those described herein, is advantageous in that antigen-specific immune responses can be targeted. Several embodiments disclosed herein leverage the role of the liver, and its various types of cells, in the development of immune tolerance to specific antigens. For example, in several embodiments, specific antigens, immunogenic fragments thereof, and/or mimetics thereof (collectively, antigens, unless otherwise indicated as a specific type, e.g., a fragment), are linked or coupled to a molecule that is configured to target the liver (or specific cells within or associated with the liver), thereby allowing the specific antigen, immunogenic fragments thereof, and/or mimetics thereof, to be processed and the immune system to be recalibrated to reduce, ameliorate, or otherwise eliminate an immune response against that antigen (or portion of an antigen, or a plurality of antigens). For example, in several embodiments, compositions provided herein are targeted for delivery to (and for uptake by) the liver, particularly hepatocytes, LSECs, Kupffer cells and/or stellate cells, or other cells with scavenger receptors (e.g., asialoglycoprotein receptors (ASGPRs), etc.).

Some embodiments disclosed herein demonstrate that hepatocytes can be manipulated using synthetic constructs, such as those compositions disclosed herein, to actively induce immunologic tolerance of antigen-specific CD8⁺ T cells, for example, by cross-presentation of extracellular antigens. Considered “non-professional antigen-presenting cells,” hepatocytes have not previously been reported to express co-stimulatory molecules under homeostatic physiological conditions that could promote inflammatory T cell responses to antigen, and are thus a promising cellular candidate for antigen presentation to prime tolerogenic T cell responses. Hepatocytes compose up to 80% of the total liver and are in direct contact with circulating T lymphocytes. Hepatocytes do not express immunological co-stimulatory molecules. For that reason, whether hepatocytes contribute to peripheral tolerogenesis by cross-presenting blood-borne antigens was tested. Demonstrated herein, and in accordance with several embodiments, hepatocytes can be manipulated in situ (e.g., through targeting hepatocytes with constructs according to embodiments disclosed herein) to contribute to peripheral tolerogenesis by presenting and cross-presenting antigens to T cells.

Unlike other organs, where circulating lymphocytes only extravasate and gain access to the parenchyma in the case of inflammation, the liver microvasculature has a peculiar fenestrated endothelium devoid of any basal membrane, allowing direct physical contact between circulating CD8+T lymphocytes and liver MHC-I+ parenchymal cells, including hepatocytes. Hepatocytes possess poor cross-presentation capacity in vitro as compared to other liver cells, especially LSECs. Nonetheless, direct antigen expression, obtained by transgenesis and/or viral vector transduction, and subsequent MHC-I-dependent antigen presentation in hepatocytes in vitro and in vivo can result in immune tolerance mainly by suboptimal activation of antigen-specific CD8+T lymphocytes because of a lack of CD28 co-stimulation leading to clonal deletion of the T cells. The induction of CD4+CD25+FoxP3+ Treg cells also occurs upon lentiviral-mediated hepatocyte-dependent antigen presentation, indicating a possible involvement of other antigen-presenting cells (APCs) in hepatocyte-driven tolerogenic mechanisms, since hepatocytes express low levels of MHC-II to interact with CD4+ T cells.

Hepatocytes outnumber other cellular components of the liver and are in close contact with components of the blood. In some embodiments disclosed herein, hepatocytes are used to establish CD8+ T cell peripheral tolerance through mechanisms of extracellular antigen uptake and cross-presentation. In other embodiments, the constructs and compositions disclosed herein are used to induce tolerance through other mechanisms, alone or in conjunction with antigen cross-presentation. Hepatocytes possess lectin receptors (among others), including the asiaoglycoprotein receptor (ASGPR). Apoptotic processes activate neuraminidases that desialylate glycoproteins to expose terminal N-acetylgalactosamine residues, which bind to ASGPR. Given the peripheral tolerogenic nature of apoptotic debris, studies were designed (and are discussed herein) to determine whether hepatocytes might be involved in the collection of exogenous antigens (e.g., N-acetylgalactosaminylated antigens) and might process and present those antigens tolerogenically. Described herein are in vitro and in vivo results of an assessment of the antigen presentation and immune tolerance capabilities of the disclosed constructs in murine models. While the studies herein involve murine models, some embodiments pertain to tolerogenesis in other mammals, including humans. Without being bound to a particular mechanism, the results demonstrate that, in several embodiments, hepatocyte-dependent antigen cross-presentation (among other mechanisms induced by liver-resident immune cells following administration of the constructs disclosed herein [including the induction of regulatory T cells], and related methods) can be used in methods to induce immune tolerance via T cell deletion and/or anergy more generally. In some embodiments, hepatocytes are useful as target cells for tolerogenic prophylactic or therapeutic interventions.

Generally, the compositions provided herein comprise an antigen of interest (e.g., one to which immune tolerance is desired, including antigenic fragments of a larger molecule, or in some embodiments, a plurality of antigens/fragments thereof), a targeting moiety (e.g., a molecule that specifically targets or is recognized by the liver, or a cell type within the liver, or another target organ or cell, e.g., in the lymph nodes and/or spleen), a linker, and a terminal end unit. In some embodiments, mimetics of those antigens may be used instead of the antigen or antigen fragments. As discussed in more detail below, the linkers may vary depending on the embodiment, but in several embodiments are advantageously designed and/or configured to release the antigen (or antigenic fragment(s) thereof or mimetic thereof) in vivo in its native, or substantially native form (e.g., the form in which it was prior to being conjugated to the linker). In some embodiments, the antigenic portion of the molecule is attached to the linker via a degradable bond. Thus, in several embodiments, the antigen of interest is liberated at, in or near the liver (or other target site) and is processed and presented to the immune system in a manner that allows the immune system to recognize the native antigen (or antigenic fragment thereof or mimetic thereof) as self, and reduce or eliminate an immune response against that antigen.

In several embodiments, the antigen can be endogenous (e.g., a self-antigen) or exogenous (e.g., a foreign antigen), including but not limited to: a foreign transplant antigen against which transplant recipients develop an unwanted immune response (e.g., transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease), or a tolerogenic portion (e.g., a fragment or an epitope) thereof; these compositions are useful for inducing tolerization to the antigen. Alternatively, a galactosylating or other liver-targeting moiety can be conjugated to an antibody, antibody fragment, or ligand that specifically binds a circulating protein or peptide or antibody, which circulating protein or peptide or antibody is causatively involved in transplant rejection, immune response against a therapeutic agent, autoimmune disease, and/or allergy (as discussed above); these compositions are useful for clearing the circulating protein, peptide or antibody. Accordingly, the compositions of the present disclosure can be used for treating an unwanted immune response, e.g., transplant rejection, an immune response against a therapeutic agent, an autoimmune disease, and/or an allergy, depending on the embodiment. Also provided are pharmaceutical compositions containing a therapeutically effective amount of a composition or construct of the disclosure. In some embodiments, the construct and/or composition is admixed with at least one pharmaceutically acceptable excipient. In another aspect, the disclosure provides methods for the treatment of an unwanted immune response, such as transplant rejection, response against a therapeutic agent, autoimmune disease or allergy. In some embodiments, methods of manufacturing linkers (including linkers having targeting agents covalently bonded thereto) and/or tolerogenic molecules are provided.

As will be discussed in more detail herein, in several embodiments, liver-targeting facilitates two possible mechanisms of tolerance induction: tolerization and clearance. Tolerization takes advantage of the liver's role in clearing apoptotic cells and processing their proteins to be recognized by the immune system as “self,” as well as the liver's role in sampling peripheral proteins for immune tolerance. Clearance takes advantage of the liver's role in blood purification by rapidly removing and breaking down toxins, polypeptides and the like.

Accordingly, the compositions of the present disclosure (and related methods) can be used for treating an unwanted immune response, e.g., transplant rejection, an immune response against a therapeutic agent, an autoimmune disease, and/or an allergy, depending on the embodiment (e.g., and the antigen). Also provided are pharmaceutical compositions containing a therapeutically effective amount of a composition of the disclosure admixed with at least one pharmaceutically acceptable excipient. In another aspect, the disclosure provides methods for the treatment of an unwanted immune response, such as transplant rejection, response against a therapeutic agent, autoimmune disease or allergy.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

As used herein, term “about” shall have its plain and ordinary meaning as an indication of an approximation. For example, when referring to a measurable value such as an amount of a compound or agent of the current subject matter, dose, time, temperature, efficacy, stability, and the like, is meant to encompass variations of ±20%, ±10%, ±5%, ±1%, ±0.5%, or even ±0.1% of the specified amount. Also included are any values within the disclosed range, including the listed endpoints.

As used herein, an “antigen” shall have its plain and ordinary meaning and shall refer to any substance that serves as a target for the receptors of an innate or adaptive immune response, such as the T cell receptor, major histocompatibility complex class I and H, B cell receptor or an antibody. In some embodiments, an antigen may originate from within the body (e.g., “self,” “auto” or “endogenous”). In additional embodiments, an antigen may originate from outside the body (“non-self,” “foreign” or “exogenous”), having entered, for example, by inhalation, ingestion, injection, or transplantation, transdermally, etc. In some embodiments, an exogenous antigen may be biochemically modified in the body. Foreign antigens include, but are not limited to, food antigens, animal antigens, plant antigens, environmental antigens, therapeutic agents, as well as antigens present in an allograft transplant.

As used herein, the term “conservative changes” shall have its plain and ordinary meaning and refers to changes that can generally be made to an amino acid sequence without altering activity. These changes are termed “conservative substitutions” or mutations; that is, an amino acid belonging to a grouping of amino acids having a particular size or characteristic can be substituted for another amino acid. Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically D-amino acids for L-amino acids for one or more residues of a sequence. Moreover, all of the amino acids in a sequence can undergo a D- to L-isomer substitution. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free —NH₂. Yet another type of conservative substitution constitutes the case where amino acids with desired chemical reactivities are introduced to impart reactive sites for chemical conjugation reactions, if the need for chemical derivatization arises. Such amino acids include but are not limited to Cys (to insert a sulfhydryl group), Lys (to insert a primary amine), Asp and Glu (to insert a carboxylic acid group), or specialized noncanonical amino acids containing ketone, azide, alkyne, alkene, and tetrazine side-chains. Conservative substitutions or additions of free —NH₂ or —SH bearing amino acids can be particularly advantageous for chemical conjugation with the linkers and galactosylating moieties of Formula 1. Moreover, point mutations, deletions, and insertions of the polypeptide sequences or corresponding nucleic acid sequences can in some cases be made without a loss of function of the polypeptide or nucleic acid fragment. Substitutions can include, e.g., 1, 2, 3, 4, 5, 6, 7, 8, 9, 10, 15, 20, 25, 30, 35, 40, 45, 50 or more residues (including any number of substitutions between those listed). A variant usable in the present invention may exhibit a total number of up to 200 (e.g., up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200, including any number in between those listed) changes in the amino acid sequence (e.g., exchanges, insertions, deletions, N-terminal truncations, and/or C-terminal truncations). In several embodiments, the number of changes is greater than 200. Additionally, in several embodiments, the variants include polypeptide sequences or corresponding nucleic acid sequences that exhibit a degree of functional equivalence with a reference (e.g., unmodified or native sequence). In several embodiments, the variants exhibit about 80%, about 85%, about 90%, about 95%, about 97%, about 98%, about 99% functional equivalence to an unmodified or native reference sequence (and any degree of functional equivalence between those listed). The amino acid residues described herein employ either the single letter amino acid designator or the three-letter abbreviation in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

As used herein, the terms “effective amount” or “therapeutically effective amount” shall have its plain and ordinary meaning and shall refer to that amount of a recited compound that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, this amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

As used herein, the term “epitope”, also known as antigenic determinant, shall have its plain and ordinary meaning and shall refer to a segment of a macromolecule (e.g. a protein), which is recognized by the immune system, such as by antibodies, B cells, major histocompatibility complex molecules, or T cells. Epitopes may be recognized by, for example, antibodies or B cells, and may include a part or segment of a macromolecule capable of binding to an antibody or antigen-binding fragment thereof. In this context, the term “binding” in particular relates to a specific binding. In the context of several embodiments of the present invention, it is preferred that the term “epitope” refers to a segment of protein or polyprotein that is recognized by the immune system. In several embodiments, the “antigen” used in the constructs disclosed herein may comprise a one or more epitopes. In some embodiments wherein more than one epitope is included, the additional epitopes may be from the same or a different antigen.

As used herein, the term galactose refers to a monosaccharide sugar that exists both in open-chain form and in cyclic form, having D- and L-isomers. In some embodiments, one or more of the cyclic forms are used, namely the alpha and/or beta anomer. In the alpha form, the C1 alcohol group is in the axial position, whereas in the beta form, the C1 alcohol group is in the equatorial position. In particular, “galactose” refers to the cyclic six-membered pyranose, more in particular the D-isomer and even more particularly the beta-D-form (β-D-galactopyranose) the formal name for which is (2R,3R,4S,5R,6R)-6-(hydroxymethyl)tetrahydro-2H-pyran-2,3,4,5-tetraol. Glucose is an epimer of galactose; the formal name is (2R,3R,4S,5S,6R)-6-(hydroxymethyl)tetrahydro-2H-pyran-2,3,4,5-tetraol. The structure and numbering of galactose and glucose are shown giving two non-limiting examples of stereochemical illustration. As used herein, the term “glucose” refers to a monosaccharide sugar that exists both in open-chain form and in cyclic form, having D- and L-isomers. In some embodiments, one or more of the cyclic forms are used, namely the alpha and/or beta anomer. In the alpha form, the C1 alcohol group is in the axial position, whereas in the beta form, the C1 alcohol group is in the equatorial position. The structure and numbering of galactose and glucose are shown giving two non-limiting examples of stereochemical illustration.

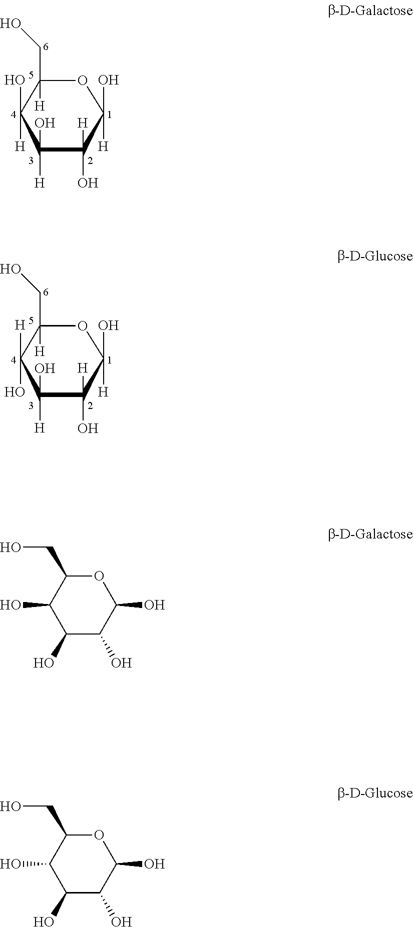

As used herein, the term “galactosylating moiety” refers to a particular type of liver-targeting moiety. Galactosylating moieties include, but are not limited to a galactose, galactosamine and/or N-acetylgalactosamine residue. A “glucosylating moiety” refers to another particular type of liver-targeting moiety and includes, but is not limited to glucose, glucosamine and/or N-acetylglucosamine. In some embodiments, any one of the the available hydroxyl groups of the galactosylating or glucosylating moiety (e.g., the OH bonded to any one of carbons 1, 2, 3, 4, 5, or 6) may be used as an attachment point for functionalization to the linker.

As used herein, the term “liver-targeting moiety” refers to moieties having the ability to direct an agent (e.g., an immune tolerance inducing construct, a polypeptide, etc.) to the liver. The liver comprises different cell types, including but not limited to hepatocytes, sinusoidal epithelial cells, Kupffer cells, stellate cells, and/or dendritic cells. Typically, a liver-targeting moiety directs a polypeptide to one or more of these cells. On the surface of the respective liver cells, receptors are present which recognize and specifically bind the liver-targeting moiety. Liver-targeting can be achieved by chemical conjugation of an antigen or ligand to a galactosylating or glucosylating moiety, desialylation of an antigen or ligand to expose underlying galactosyl or glucosyl moieties, or specific binding of an endogenous antibody to an antigen or ligand, where the antigen or ligand is: desialylated to expose underlying galactosyl or glucosyl moieties, conjugated to a galactosylating or a glucosylating moiety. Naturally occurring desialylated proteins are not encompassed within the scope of certain embodiments of the present disclosure.

The “numerical values” and “ranges” provided for the various substituents are intended to encompass all integers within the recited range. For example, when defining n as an integer representing a mixture including from about 1 to 100, particularly about 8 to 90 and more particularly about 40 to 80 ethylene glycol groups, where the mixture typically encompasses the integer specified as n±about 10% (or for smaller integers from 1 to about 25, ±3), it should be understood that n can be an integer from about 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110, or any between those listed, including the endpoints of the range) and that the disclosed mixture encompasses ranges such as 1-4, 2-4, 2-6, 3-8, 7-13, 6-14, 18-23, 26-30, 42-50, 46-57, 60-78, 85-90, 90-110 and 107-113 ethylene glycol groups. The combined terms “about” and “±10%” or “±3” should be understood to disclose and provide specific support for equivalent ranges wherever used.

As used herein, the term “optional” or “optionally” means that the subsequently described event or circumstance may or may not occur, and that the description includes instances where said event or circumstance occurs and instances in which it does not.

A peptide that specifically binds a particular target is referred to as a “ligand” for that target.

As used herein, a “polypeptide” is a term that refers to a chain of amino acid residues, regardless of post-translational modification (e.g., phosphorylation or glycosylation) and/or complexation with additional polypeptides, and/or synthesis into multisubunit complexes with nucleic acids and/or carbohydrates, or other molecules. Proteoglycans therefore also are referred to herein as polypeptides. A long polypeptide (having over about 50 amino acids) is referred to as a “protein.” A short polypeptide (having fewer than about 50 amino acids) is referred to as a “peptide.” Depending upon size, amino acid composition and three dimensional structure, certain polypeptides can be referred to as an “antigen-binding molecule,” “antibody,” an “antibody fragment” or a “ligand.” Polypeptides can be produced by a number of methods, many of which are well known in the art. For example, polypeptides can be obtained by extraction (e.g., from isolated cells), by expression of a recombinant nucleic acid encoding the polypeptide, or by chemical synthesis. Polypeptides can be produced by, for example, recombinant technology, and expression vectors encoding the polypeptide introduced into host cells (e.g., by transformation or transfection) for expression of the encoded polypeptide.

The term “purified” as used herein with reference to a polypeptide refers to a polypeptide that has been chemically synthesized and is thus substantially uncontaminated by other polypeptides, or has been separated or isolated from most other cellular components by which it is naturally accompanied (e.g., other cellular proteins, polynucleotides, or cellular components). An example of a purified polypeptide is one that is at least 70%, by dry weight, free from the proteins and naturally occurring organic molecules with which it naturally associates. A preparation of a purified polypeptide therefore can be, for example, at least 70%, at least 75%, at least 80%, at least 90%, or at least 99%, by dry weight, the polypeptide. Polypeptides also can be engineered to contain a tag sequence (e.g., a polyhistidine tag, a myc tag, a FLAG® tag, or other affinity tag) that facilitates purification or marking (e.g., capture onto an affinity matrix, visualization under a microscope). Thus, a purified composition that comprises a polypeptide refers to a purified polypeptide unless otherwise indicated. The term “isolated” indicates that the polypeptides or nucleic acids of the disclosure are not in their natural environment. Isolated products of the disclosure can thus be contained in a culture supernatant, partially enriched, produced from heterologous sources, cloned in a vector or formulated with a vehicle, etc.

The term “copolymer” refers to a polymerization of two or more monomers. A copolymer may be a random copolymer, gradient copolymer, block copolymer, or mixture of the foregoing.

The term “random copolymer” refers to the product of simultaneous polymerization of two or more monomers in admixture. In some embodiments, the “random copolymer” is a statistically random copolymer where the probability of finding a given monomeric unit at any given site in a polymer chain is independent of the nature of the neighboring units at that position (Bernoullian distribution). In some embodiments, the “random copolymer” is not statistically random. For instance, where different monomers have different reactivities, the monomer distribution along a polymer chain might not be statistically random and instead may have some gradient or block character. When the variable group identified as Y′ represents a random copolymer, the chain can comprise any sequence from 2 up to about 400 (or more, as disclosed elsewhere herein) W¹ and W² groups, such as: -W¹-W²-W¹-W²—; -W²-W¹-W²-W¹—; -W¹-W¹-W¹-W²-; -W¹-W¹-W²-W²-; -W¹-W²-W²-W¹-; -W¹-W²-W¹-W²-W²-W¹-W²-W¹-; -W¹-W¹-W²-W²-W¹-W²-W²-W¹-; and W²-W²-W¹-W²-W¹-W¹-W¹—W²-W²-W¹-W²-W²-W¹; ad infinitum, where Z attached to the various W¹ groups and the W¹ and W² groups themselves can be the same or different. When the variable group identified as Y′ represents a random copolymer, the chain can comprise any sequence from 2 up to about 400 (or more, as disclosed elsewhere herein) W³ and W⁴ groups, such as: -W¹-W⁴-W³-W⁴-; -W⁴-W³-W⁴-W³-; -W³-W³-W³-W⁴-; -W³-W³-W⁴-W⁴-; -W³-W⁴-W⁴-W³-; -W³-W⁴-W³-W⁴-W⁴—W³-W⁴-W³-; -W³-W³-W⁴-W⁴-W³-W⁴-W⁴-W³-; and W⁴-W⁴-W³-W⁴-W³-W³-W³-W⁴-W⁴-W³—W⁴-W⁴-W³; ad infinitum, where Z attached to the various W³ groups and the W³ and W⁴ groups themselves can be the same or different.

The term “sequence identity” is used with regard to polypeptide (or nucleic acid) sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Particularly, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide. In addition, when a sequence is disclosed as “comprising” a nucleotide or amino acid sequence, such a reference shall also include, unless otherwise indicated, that the sequence “comprises”, “consists of” or “consists essentially of” the recited sequence.

“Specific binding,” as that term is commonly used in the biological arts, refers to a molecule that binds to a target with a relatively high affinity as compared to non-target tissues, and generally involves a plurality of non-covalent interactions, such as electrostatic interactions, van der Waals interactions, hydrogen bonding, and the like. Specific binding interactions characterize antibody-antigen binding, enzyme-substrate binding, and certain protein-receptor interactions; while such molecules might bind tissues besides their specific targets from time to time, to the extent that such non-target binding is inconsequential, the high-affinity binding pair can still fall within the definition of specific binding.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of, for example, Formula (I), those skilled in the art understand that when a salt is formed by protonation of a nitrogen-based group (for example, NH₂), the nitrogen-based group can be associated with a positive charge (for example, —NH₂ can become —NH₃⁺) and the positive charge can be balanced by a negatively charged counterion (such as Cl⁻). One of ordinary skill in the art will understand that the compounds disclosed herein can be provided with one or more pharmaceutically acceptable carriers, diluents or excipients.

The term “physiologically acceptable” or “pharmaceutically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically desirable. For example, a diluent may be used to increase the bulk of a potent compound whose mass is too small for manufacture and/or administration. It may also be a liquid for the dissolution of a compound to be administered by injection, ingestion or inhalation. A common form of diluent in the art is a buffered aqueous solution such as, without limitation, phosphate buffered saline that mimics the pH and isotonicity of human blood. Other buffers can be used as diluents.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In several embodiments, the pharmaceutical composition comprises an anti-oxidant and/or a metal-chelating agent. A “diluent” is a type of excipient.

As used herein, the term “patient” or “subject” includes a human patient, although it is to be understood that the principles of the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

As used herein, the term “treat” or “treating” or “treatment” shall have its plain and ordinary meaning and refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc. In some embodiments, treating can include one or more of preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms. In certain embodiments, treatment of a subject achieves one, two, three, four, or more of the following effects, including, for example: (i) reduction or amelioration the severity of disease state or symptom associated therewith; (ii) reduction in the duration of a symptom associated with a disease or immune response; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalizations of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Administration can be by a variety of routes, including, without limitation, intravenous, intra-arterial, subcutaneous, intramuscular, intrahepatic, intraperitoneal and/or local delivery to an affected tissue.

As used herein, the term “operably linked,” shall be given its ordinary meaning. In some embodiments, as an illustration, where two groups are operably linked, the groups are attached such that one or more of the linked groups is provided without substantial loss in its native reactivity or activity. In some embodiments, the antigens disclosed herein are operably linked to linking agents and targeting agents.

As used herein, the term “unwanted immune response” refers to a reaction by the immune system of a subject, which in the given situation is not desirable. The reaction of the immune system is unwanted if such reaction does not lead to the prevention, reduction, or healing of a disease or disorder but instead causes, enhances or worsens, or is otherwise associated with induction or worsening of a disorder or disease. Typically, a reaction of the immune system causes, enhances or worsens a disease if it is directed against an inappropriate target. For example, an unwanted immune response includes but is not limited to transplant rejection, immune response against a therapeutic agent, autoimmune disease, and allergy or hypersensitivity.

The term “variant” is to be understood as a protein (or nucleic acid) which differs in comparison to the protein (or nucleic acid chain) from which it is derived by one or more changes in its length, sequence, or structure. The polypeptide from which a protein variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence or structure in comparison to the parent molecule. Also encompassed are modified molecules such as but not limited to post-translationally modified proteins (e.g. glycosylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant.” Naturally occurring and artificially constructed variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule (e.g., is functionally active). A variant can be characterized by a certain degree of sequence identity to the parent polypeptide from which it is derived. More precisely, a protein variant in the context of the present disclosure may exhibit at least 80% sequence identity to its parent polypeptide. Preferably, the sequence identity of protein variants is over a continuous stretch of 20, 30, 40, 45, 50, 60, 70, 80, 90, 100 or more amino acids. As discussed above, in several embodiments, variants exhibit about 80%, about 85%, about 90%, about 95%, about 97%, about 98%, about 99% functional equivalence to an unmodified or native reference sequence (and any degree of functional equivalence between those listed).

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, C-amido, N-amido, S-sulfonamido, N-sulfonamido, C-carboxy, O-carboxy, nitro, sulfenyl, sulfinyl, sulfonyl, succinimidyl ester, isoindolin-1,3-dione, haloalkyl, haloalkoxy, an amino, a mono-substituted amine group, a di-substituted amine group, a mono-substituted amine(alkyl), a di-substituted amine(alkyl), a diamino- group, a diether-, a polyamino-, and a polyether-.

As used herein, “C_{a-b}” in which “a” and “b” are integers refer to the number of carbon atoms in a group. The indicated group can contain from “a” to “b”, inclusive, carbon atoms. Thus, for example, a “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C-. If no “a” and “b” are designated, the broadest range described in these definitions is to be assumed. For the avoidance of doubt, where “C_{a-b}” refers to a structure with a heteroatom, a heteroatom may be provided between “a” and “b”. For example, a C₁₋₄ heteroalkyl group refers to all heteroalkyl groups having from 1 to 4 carbons, that is, CH₃O—, CH₃CH₂O—, CH₃OCH₂—, CH₃OCH₂CH₂—, CH₃OCH₂CH₂CH₂—, etc. Likewise, where “C_{a-b},” refers to a structure an alkenyl or an alkynyl group, the double bond(s) or triple bond(s) may be between any of the “a” to “b” carbons.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

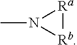

The term “amino” and “amine” refer to nitrogen-containing groups such as NR₃, NH₃, NHR₂, and NH₂R, wherein R can be as described elsewhere herein. Thus, “amino” as used herein can refer to a primary amine, a secondary amine, or a tertiary amine.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

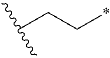 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group

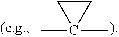

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(S)R” group in which R can be the same as defined with respect to O-carboxy. A thiocarbonyl may be substituted or unsubstituted.

An “O-carbamyl” group refers to a “—OC(O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted. R_{A} and R_{B} as defined here (or elsewhere herein for alternative structures) may be “taken together” as shown elsewhere herein.

An “N-carbamyl” group refers to an “ROC(O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

A “C-amido” group refers to a “—C(O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “R_{C}(O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “R_{C}(O)O—” group in which R can be hydrogen, an alkyl (e.g., Ca-b alkyl), an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A “mono-substituted amine” may be shown as “—NR_{A}R_{B}” where one of R_{A} or R_{B} is —H (just as, in an amine, both R_{A} and R_{B} are —H). A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, —N(phenyl)(methyl), —N(ethyl)methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl)groups include, but are not limited to, —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, the term “diamino-” denotes an a “—NR_{A}(R_{B})N(R_{C})—” group in which R_{B} and R_{C} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{A} connects the two amino groups and can be (independently of R_{B} and R_{C}) an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). For instance, R_{A} may be —(CH₂)_{x′}— where x′ is an integer less than or equal to 1, 2, 3, 4, 5, 6, 10, 15, 20, or ranges including and/or spanning the aforementioned values. R_{A}, R_{B}, and R_{C} can independently be substituted or unsubstituted.

As used herein, the term “ether” denotes an a “—R_{B}—O-R_{A}” group in which R_{A} can be a hydrogen, optionally substituted C₁₋₆ alkyl, as defined herein, R_{B} is a direct bond, or optionally substituted C₁₋₆ alkyl, as defined herein. R_{A} and R_{B} can independently further be substituted or unsubstituted, as defined herein.

As used herein, the term “diether-” denotes an a “—O—R_{D}—O—R_{E}” group in which R_{D} and R_{E} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{D} connects the two O groups. R_{E} can also be —H. In several embodiments, R_{D} may be —(CH₂)_{x″}— where x″ is an integer less than or equal to 1, 2, 3, 4, 5, 6, 10, 15, 20, or ranges including and/or spanning the aforementioned values. R_{D} and R_{E} can be optionally substituted or unsubstituted.

As used herein, the term “polyamino” denotes a repeating —N(R_{B})alkyl- group. For illustration, the term polyamino can comprise —N(R_{B})alkyl-N(R_{B})alkyl-N(R_{B})alkyl-N(R_{B})alkyl-. In some embodiments, the alkyl of the polyamino is as disclosed elsewhere herein. While this example has only 4 repeat units, the term “polyamino” may consist of 1, 2, 3, 4, 5, 6, 7, 8, 9, or 10 repeat units, where R_{B} and alkyl are as defined elsewhere herein. As noted here, the polyamino comprises amine groups with intervening alkyl groups (where alkyl is as defined elsewhere herein). In several embodiments, the alkyl of the polyamino (e.g., separating to N atoms) has 1, 2, 3, 4, or more methylene units (e.g., —CH₂—). A polyamino may terminate with an amine group or as an alkyl where the polyamino is a terminal group, or with as an —N(R_{C})— where the polyamino bridges two atoms. For instance, any one of methylenediamino (—NHCH₂NH—), ethylenediamino (—NH(CH₂)₂NH—), etc. are considered a polyamino groups. In several embodiments, the polyamino terminates with an —N(R_{C})— that is optionally substituted. For instance, a polyamino may terminate with an —N(R_{C})— that is substituted with —H, C₁-C₆ alkyl, etc.

As used herein, the term “polyether” denotes a repeating —Oalkyl- group (e.g., an —O-R_{D}-O— where R_{D} is alkyl as disclosed elsewhere herein). For illustration, the term polyether can comprise —O-alkyl-O-alkyl —O-alkyl-O-alkyl. A polyether may have up to 10 repeat units, comprising —O— (ethers) with intervening alkyl groups (where alkyl is as defined elsewhere herein). A polyether may have 1, 2, 3, 4, 5, 6, 7, 8, 9, 10, or more repeat units. In several embodiments, the alkyl of the ether (e.g., separating two —O— atoms) has 1, 2, 3, 4, or more methylene units (e.g., —CH₂—). The polyether may terminate with a hydroxy group or as an alkyl where the polyether is a terminal group, or with an —O— where the polyether bridges two atoms. A polyether may terminate with an —O—. In several embodiments, the polyether terminates with an —O— that is optionally substituted. For instance, a polyether may terminate with an —O— that is substituted with —H, C₁-C₆ alkyl, etc.

As used herein, the term succinimidyl ester refers to the following structure:

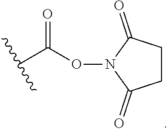 [SMILES: *C(=O)ON1C(=O)CCC1=O]

As used herein, the term isoindolin-1,3-dione refers to the following structure:

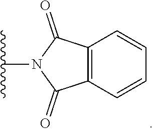 [SMILES: *N1C(=O)c2ccccc2C1=O]

When a range of integers is given, the range includes any number falling within the range and the numbers defining ends of the range. For example, when an “integer from 1 to 20” is used, the integers disclosed in the range are 1, 2, 3, 4, 5, 6, 7, 8, 9, 10, etc., up to and including 20. When the terms “integer from 1 to 100” is used, the integers disclosed include, for example, 1, 2, 3, 4, 5, 6, 7, 8, 9, 10, 50, 51, 52, 53, etc., up to and including 100.

### Compositions for Liver Targeting

Several embodiments of the present disclosure relate to compounds, compositions (e.g., pharmaceutical compositions) or constructs for immune tolerance. In some embodiments, the compounds, compositions, or constructs are polymeric and/or comprise a polymeric region. In some embodiments, immune tolerance can be induced against a variety of antigens, based on the disclosure provided herein. For example, the antigen can be endogenous (e.g., a self-antigen) or exogenous (e.g., a foreign antigen), including but not limited to: a foreign transplant antigen against which transplant recipients develop an unwanted immune response (e.g., transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease), or a tolerogenic portion (e.g., a fragment or an epitope) of any such type of antigen.

In several embodiments, as disclosed herein, a compound (e.g., construct) for the induction of antigen-specific immune tolerance in a subject is provided. In several embodiments, the compound comprises an antigen, a linker, a targeting moiety (e.g., a liver targeting moiety), and a terminal end unit. In several embodiments, the antigen, as disclosed elsewhere herein, is a full length and/or native antigen, a tolerogenic portion of an antigen, and/or a mimetic of an antigen. In several embodiments, the antigen to which tolerance is desired, when presented alone to the subject is capable of inducing an unwanted immune response in the subject. In several embodiments, the linker comprises one or more polymeric portions. In some embodiments, the linker (and/or the polymeric portion thereof) is prepared via living reversible addition-fragmentation chain transfer (RAFT) polymerization. As disclosed elsewhere herein, the RAFT polymerization can be used to polymerize any monomer suited for RAFT polymerization (including but not limited to acrylate (including methacrylates), acrylamides (including but not limited to methacrylamides), or the like) to provide a polymeric portion of the linker. In some embodiments, the linker comprises an acrylyl polymer portion. In some embodiments, the acrylyl portion of the linker comprises an α-end and an ω-end. In some embodiments, the ω-end of the acrylyl polymer portion (at the ω-side of the linker) is the active terminus of the linker where fragmentation and polymer chain extension takes place (or took place in a completed polymerization). In some embodiments, the α-end of the acrylyl polymer portion (at the α-side of the linker) is the distal or opposite end of the RAFT polymer chain (opposite from the ω-side) where the inactive terminus of the growing polymer chain resides. As disclosed elsewhere herein, RAFT reactions are performed using a RAFT reagent that generates a RAFT CTA fragment. In some embodiments, after polymerization, the α-end (the “alpha” end) of the acrylyl polymer comprises a resident portion of the RAFT reagent and the ω-end (the “gamma” end) includes a CTA remnant. In several embodiments, the resident portion of the RAFT reagent at the α-end of the acrylyl polymer includes a reactive group for further functionalization and/or modification (e.g., after polymerization). In other embodiments, the resident portion of the RAFT reagent at the α-end of the acrylyl polymer does not include a reactive group for further functionalization (and instead is the terminal end unit). In some embodiments, one of the α-end or the ω-end may be bonded to and/or may be a terminal end unit of the tolerogenic construct. In some embodiments, either the α-end or the ω-end may be bonded to the antigen. For instance, in some embodiments, the antigen is bonded to the linker at the α-end (and the ω-end is bonded to the terminal end unit). In other embodiments, the antigen is bonded to the linker at the ω-end (and the α-end may be bonded to and/or may include the terminal end unit). In several embodiments, the linker is bonded to the antigen via a cleavable bond. In some embodiments, the cleavable bond is a disulfide bond or a disulfanyl ethyl ester unit. In several embodiments, the disulfide bond or the disulfanyl ethyl ester are each configured to be cleaved upon administration of the compound to the subject and to release the antigen from the linker. In several embodiments, the disulfide is formed using a thiol-reactive linker, and a sulfur of the linker bonds to a sulfur of the antigen to provide the disulfide bond. In several embodiments, the disulfanyl ethyl ester is formed using an amine-reactive linker. In several embodiments, an amine of the antigen reacts with a disulfanyl ethyl ester precursor (e.g., a precursor having a N-hydroxysuccinimide ester or other leaving group) of the linker to provide the disulfanyl ethyl ester.

In several embodiments, as disclosed elsewhere herein, the linker comprises a copolymer. In several embodiments, the copolymer (e.g., a random copolymer, gradient copolymer, or block copolymer, or mixture of the foregoing) comprises one or more acrylyl units. In several embodiments, the linker comprises a plurality of acrylyl units (e.g., 2, 3, 4, or more types of repeat units). In some embodiments, the linker comprises at least a first acrylyl unit and a second acrylyl unit. In several embodiments, the first acrylyl unit comprises a first ethylacetamido functionality. In several embodiments, the second acrylyl unit comprising a second ethylacetamido functionality. In several embodiments, the second ethylacetamido functionality is conjugated to an aliphatic group, an amine, a polyamino, an alcohol, a polyether, or an aliphatic alcohol. In several embodiments, the second ethylacetamido functionality is connected (e.g., bonded or conjugated) to an aliphatic group, an alcohol, or an aliphatic alcohol. In several embodiments, the second ethylacetamido functionality acts as a spacer. In several embodiments, the linker is bonded to the antigen to which tolerance is desired or tolerogenic portion thereof via a disulfide bond or a disulfanyl ethyl ester configured to be cleaved upon administration of the compound to a subject and to release the antigen to which tolerance is desired or tolerogenic portion thereof from the linker. In several embodiments, the liver-targeting moiety comprises a galactosylating or glucosylating moiety. In several embodiments, the liver-targeting moiety is bonded to the linker through the first ethylacetamido functionality. In several embodiments, the first ethylacetamido functionality is connected (e.g., covalently bonded through a connecting group) to the liver-targeting moiety through an amino, a polyamino, an ether, a polyether, or an aliphatic group. In several embodiments, the polyamino or polyether have two to 10 repeat units or more (e.g., 2, 3, 4, 5, 6, 7, 8, 9, 10, or more). In several embodiments, the polyether comprises propylene glycol repeat units (e.g., —(CH₂—CH₂—CH₂—O)—). In several embodiments, the polyether comprises ethylene glycol repeat units (e.g., —(CH₂—CH₂—O)—). In several embodiments, the liver-targeting moiety may be connected to the first ethylacetamido functionality via a chain group (e.g., a connecting group).

As disclosed elsewhere herein, the tolerogenic compounds disclosed herein may be prepared using reversible addition-fragmentation chain transfer (RAFT) polymerization. In some embodiments, an intermediate of a tolerogenic compound as disclosed herein can be depicted structurally. In some embodiments, an intermediate of a tolerogenic compound as disclosed herein can be depicted structurally as Formula (A):

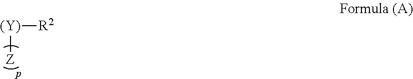

where Y comprises a linker moiety, Z comprises a targeting agent (e.g., a liver targeting agent), and R² is an end capping group (e.g., a CTA remnant), p is an integer from 2 to 250 (or as disclosed elsewhere herein), the left, opening parentheses “(” signifies the location reactive site of Y, the right, closing parentheses “)” signifies the location of the bond between Y and R², and the upper, opening parentheses “ [symbol] ” signifies the location of the bond between Y and a Z unit (of which there are “p” Z units along Y). In several embodiments, the reactive site of Y is thiol-reactive or amine-reactive, as disclosed elsewhere herein.

In several embodiments, as disclosed herein, a portion of the RAFT chain transfer agent remains on the linker intermediate after polymerization as a RAFT remnant. In several embodiments, the RAFT remnant resides at the ω-end (or ω-side) of the linker (and/or polymeric portion of the linker). For example, in some embodiments, R² is a RAFT remnant of a RAFT chain transfer agent used to prepare Y. For instance, R² may be a remnant of a chain transfer agent (CTA) of a RAFT living polymerization.

In some embodiments, R² is any of functional groups I-IV:

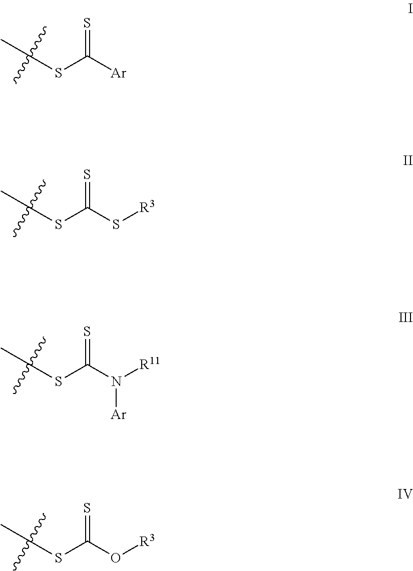

where Ar is a substituted or unsubstituted aromatic group, R³ is any carbon-containing linear or heterocyclic moiety. In some embodiments, R³ is an optionally substituted alkyl, optionally substituted cycloalkyl, optionally substituted aryl, or optionally substituted heteroaryl. In some embodiments, any one of Ar, R³, or R¹¹ is optionally substituted. In some embodiments, any one of Ar, R³, or R¹¹ is optionally substituted with an optionally substituted alkyl, optionally substituted heterocyclyl, optionally substituted aryl, optionally substituted heteroaryl. In some embodiments, R¹¹ is hydrogen or an optionally substituted alkyl. In some embodiments, R² is one of the functional groups:

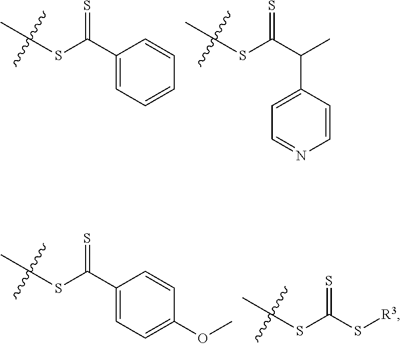

where R³ is as defined above.

It has been found that, surprisingly, displacing the CTA remnant (e.g., R²) after polymerization (with a different group as disclosed elsewhere herein) results in a more stable tolerogenic construct, as disclosed elsewhere herein. In several embodiments, to afford the improved tolerogenic construct, the CTA remnant (e.g., R²) is displaced by a terminal end unit, as disclosed elsewhere herein. In other embodiments, the CTA remnant is displaced by an end group having a reactive site (e.g., a reactive end unit). In some embodiments, the reactive end unit can be further functionalized to couple an antigen to the linker. In some embodiments, the reactive end unit is the terminal end unit. In some embodiments, where the end group having a reactive site is functionalized to the antigen, the resident portion of the RAFT reagent (as disclosed elsewhere herein) provides the terminal end unit (and the RAFT remnant is displaced by the end group that couples the antigen).

In several embodiments, the group used to displace the CTA remnant (e.g., R²) is the product of a reaction with an azo-compound (e.g., a bis-azo compound). In several embodiments, unlike the CTA remnant (which may comprise a dithioester as disclosed elsewhere herein), the terminal end unit or reactive end unit used to displace the CTA remnant comprises a carbon atom (e.g., a carbon atom of a methyl (e.g., CH₃), methylene (—CH₂), a methine (CH), or a quaternary carbon (C)) connecting the terminal end unit (or end-group) to the ω-end of the acrylyl portion of the linker. For example, in several embodiments, the terminal end unit or reactive end unit is bonded to the ω end of the linker via a carbon-carbon bond. For example, a carbon of the terminal end unit or reactive end unit is bonded to a carbon of the linker.

In several embodiments, unlike the CTA remnant, the terminal end unit and/or reactive end unit lacks sulfur (e.g., of a dithioester) that bonds to the carbon of the linker. In several embodiments, the terminal end unit and/or reactive end unit is not —H. In several embodiments, the end-capping group comprises a cyano group. In other embodiments, the end-capping group lacks a cyano group. In some embodiments, as disclosed elsewhere herein, the end-capping group and/or end-group comprises one or more alkyl groups optionally substituted with one or more of a halogen, C-carboxy, -amino, or —OH. In several embodiments, the end-capping group and/or end-group comprises a cycloalkyl group. In several embodiments, as disclosed elsewhere herein, the reactive end unit comprises a reactive moiety that can be functionalized with an antigen. In other embodiments, as disclosed elsewhere herein, the terminal end unit comprises a terminal moiety that is not reacted with an antigen.

In some embodiments, as disclosed elsewhere herein, the tolerogenic compound (e.g., tolerogenic construct) comprises an antigen (e.g., a full length antigen, an antigenic fragment of an antigen, a mimetic of an antigen, etc.) linked via a linker to a targeting agent of the construct. In several embodiments, the tolerogenic compound is prepared using the the intermediate of Formula (A). As disclosed elsewhere herein, certain aspects of the disclosure are directed towards constructs and/or compositions that may be represented by a compound having the structure of Formula (1):

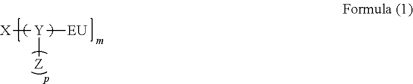

where X comprises an antigen (e.g., a full length antigen, a tolerogenic portion or portions thereof, a mimetic thereof, etc.), Y comprises a linker moiety, Z comprises a liver targeting agent, and EU comprises a terminal end unit (each variable of which is disclosed in more detail elsewhere herein). In some embodiments, p is an integer from 2 to 250 (or as disclosed elsewhere herein), m is an integer from 1 to 100, the left, opening parentheses “(” signifies the location of the bond between X and Y, the right, closing parentheses “)” signifies the location of the bond between Y and EU, and the upper, opening parentheses “ [symbol] ” signifies the location of the bond between Y and a Z unit (of which there are “p” Z units along Y). In some embodiments, multiple Z units (e.g., “p” Z units) can be bonded along the polymeric portion of the Y linker. For instance, where the Y linker has a polymeric portion with one or more repeat units, some or all of those repeat units may comprise a pendant Z group.

As shown, each instance of Z can be a moiety that is pendant from the Y linker moiety (e.g., along the length of an acrylyl polymer portion of the linker, as disclosed elsewhere herein). Where a plurality of Z groups is present, together the pendant Z groups can provide a comb or bottlebrush structure along the length of Y. In some embodiments, Formula (1) can be written as X—[Y(—Z)ₚ-EU]ₘ. As shown, each antigen can have m units of —Y(—Z)ₚ-EU. In several embodiments, m is an integer equal to or greater than about: 1, 2, 3, 4, 5, 10, 25, 50, 75, 100, or ranges including and/or spanning the aforementioned values.

### Linking Groups

As disclosed in greater detail below (and elsewhere herein), in several embodiments, linker moieties are used to join an antigen (against which tolerance is desired or an immunogenic fragment thereof) to a moiety configured to target the liver (or a specific liver cell subtype). In several embodiments, the linker is also bonded to a terminal end unit (EU) as disclosed elsewhere herein. In several embodiments, the antigen is joined with the linker (or linkers) in a manner that allows for the antigen to be liberated from the linker in vivo. In several embodiments, the linker (or linkers) is configured to release the antigen in substantially its native format (or the form it was in when conjugated to the linker, though not necessarily a format found in nature, as the antigen could be a fragment, a recombinant antigen or the like). In several embodiments, the linker (or linkers) is configured to release the antigen in substantially its active format (e.g., a form that induces an immune response) and/or in an active format. In several embodiments, the linker and antigenic portion of the construct are bonded by a degradable bond and/or a bond that is cleaved at a target site (e.g., by reduction of a disulfide bond, cleavage of a disulfanyl ethyl ester, etc.).

In several embodiments, as disclosed elsewhere herein, the linker comprises a polymeric portion (e.g., the linker is a polymeric linker). As shown below and elsewhere herein, the polymeric portion of Y can be bound to pendant liver targeting moieties that decorate the polymeric portion (e.g., a polymeric chain portion). In some embodiments, the polymeric portion comprises, consists essentially of, or consists of Y′ as disclosed elsewhere herein. In some embodiments, Y comprises, consists essentially of, or consists of Y′ as disclosed elsewhere herein. In several embodiments, the polymeric portion comprises an acrylyl-based polymer portion (e.g., acrylate-based units or derivatives thereof, acrylamide-based units or derivatives thereof, copolymers thereof, or the like). In several embodiments, the acrylyl portion comprises one or more acrylyl units (e.g., acrylyl derivatives, including acrylates, acrylamides, methacrylates, methacrylamides, derivatives of anyone thereof, or similar acrylyl structures) comprising a pendant liver targeting agent. In several embodiments, the acrylyl derivatives, including acrylates, acrylamides, methacrylates, methacrylamides, derivatives of anyone thereof, or similar acrylyl structures may be optionally substituted.

In several embodiments, the linker comprises a hydrophilic portion and/or region. In several embodiments, the linker comprises a plurality of hydrophilic regions. In several embodiments, the acrylyl portion is hydrophilic. In several embodiments, a hydrophilic portion may be separate from the acrylyl portion (though the acrylyl portion may nonetheless also be hydrophilic). In several embodiments, the hydrophilic portion comprises a length of one or more regions having —(CH₂CH₂O)ₛ—. In several embodiments, s is an integer greater than or equal to about: 1, 2, 3, 4, 5, 10, 15, 20, 30, 40, 44, 50, 75, 100, 150, or ranges including and/or spanning the aforementioned values. For example, s may be an integer from 1 to 44, from 1 to 150, etc. In some embodiments, the hydrophilic portion comprises one or more polyethylene glycol (PEG) regions. In several embodiments, the PEG is optionally substituted. In some embodiments, the PEG may have polydispersity as measured by the weight average molecular weight in g/mol (M_{W}) of the PEG divided by the number average molecular weight in g/mol (M_{N}) of the PEG (e.g., M_{W}/M_{N}). In some embodiments, the PEG chains have a number average or weight average molecular weight (g/mol) of equal to or at least about: 50, 200, 300, 500, 1000, 2000, 5000, 10000, or ranges including and/or spanning the aforementioned values. In several embodiments, the hydrophilic portion comprises a polymeric chain separate from the acrylyl portion. In several embodiments, the hydrophilic portion comprises a polymeric chain interspersed within acrylyl portions. In several embodiments, the polymeric chain is optionally substituted. In some embodiments, the polymeric chain comprises pendant hydrophilic groups such as a —OH, —SO(OH)₂, optionally substituted polyether, optionally substituted polyamino, and the like.

In some embodiments, the linker may be represented structurally by Formula (AI):

[symbol]

polymeric chain^{a}-Y′ [symbol] Formula (AI)

where the left, opening parentheses “(” signifies the location of the bond between X and Y, the right, closing parentheses “)” signifies the location of the bond between Y and EU, Y′ is a polymeric portion (as disclosed elsewhere herein) which may comprise a homopolymer or is a random copolymer, gradient copolymer, or block copolymer of two or more different types of repeat units (as disclosed elsewhere herein), wherein at least one type of repeat unit comprises a pendant targeting group, (or plurality of different types of pendant targeting groups are provided). In some embodiments, as disclosed elsewhere herein, Z comprises the targeting moiety. In some embodiments, as disclosed elsewhere herein, Z comprises a liver targeting moiety. In some embodiments, the targeting moiety (e.g., Z) comprises one or more of galactose, galactosamine, N-acetylgalactosamine, glucose, glucosamine, N-acetylglucosamine, a galactose, galactosamine, N-acetylgalactosamine, glucose, glucosamine, N-acetylglucosamine receptor-targeting moiety, and/or moieties that exhibit affinity to receptors that bind any one of galactose, galactosamine, N-acetylgalactosamine, glucose, glucosamine, N-acetylglucosamine (or any combination thereof). In some embodiments, the targeting moiety (e.g., Z) comprises moieties that exhibit affinity to receptors of any one of galactose, galactosamine, N-acetylgalactosamine, glucose, glucosamine, N-acetylglucosamine (or any combination thereof). In some embodiments, a targeting moiety comprising moieties that exhibit affinity to receptors of any one of galactose, galactosamine, N-acetylgalactosamine, glucose, glucosamine, N-acetylglucosamine (or any combination thereof) need not be a galactosylating or glucosylating moiety.

In some embodiments, Y′ is a random copolymer, gradient copolymer, or block copolymer of W¹ and W² or of W³ and W⁴, where W¹, W², W³, and W⁴ are as depicted below:

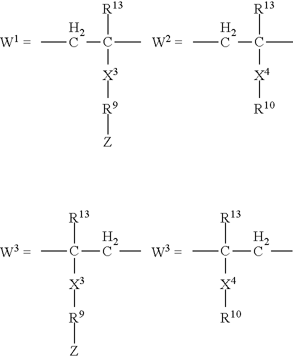

where Z is a targeting moiety (e.g., including but not limited to any one or more of galactose, a galactose receptor-targeting moiety, glucose, a glucose receptor-targeting moiety, galactosamine, a galactosamine receptor-targeting moiety, glucosamine, a glucosamine receptor-targeting moiety, N-acetylgalactosamine, a N-acetylgalactosamine receptor-targeting moiety, N-acetylglucosamine, and/or a N-acetylgalactosamine receptor-targeting moiety); each instance of R¹³ (e.g., in either W¹, W², W³, or W⁴) is independently H, methyl, or optionally substituted —C₁₋₆alkyl; X³ is (or each instance of X³ independently is) a direct bond, —C(O)—NH—, or —C(O)O—; R⁹ is a direct bond or —[((CH₂)ₕX⁵)ₜ—((CH₂)_{h′}—X⁶)_{t′}]—; X⁴ is (or each instance of X⁴ is independently) a direct bond, —C(O)—NH— or —C(O)O—; and R¹⁰ is —H or —[((CH₂)_{h″}—X⁷)_{t″}—((CH₂)_{h′″}—X⁸)_{t′″}]—R^{6″}. In several embodiments, X⁵ and X⁶ are independently selected from a direct bond, —NR^{6′}—, and —O—. In some embodiments, each instance of R^{6′} (e.g., in X⁵ or X⁶) is independently H or optionally substituted —C₁₋₆alkyl. In several embodiments, t, t′, h, and h′ are each independently an integer of equal to or at least about: 0, 1, 2, 3, 4, 5, 6, 7, 8, 9, 10, 20, or ranges including and/or spanning the aforementioned values. In several embodiments, X⁷ and X⁸ are independently selected from a direct bond, —NR^{6″}—, and —O—. In some embodiments, each instance of R^{6″} is independently H or optionally substituted —C₁₋₆ alkyl. In several embodiments, t″, t′″, h″, and h′″ are each independently an integer of equal to or at least about: 0, 1, 2, 3, 4, 5, 6, 7, 8, 9, 10, 20, or ranges including and/or spanning the aforementioned values. In several embodiments, R⁹ is a connecting group (as disclosed elsewhere herein). In several embodiments, the structures for R⁹ or R¹⁰ may be updated accordingly where any one of t, t′, h, h′, t″, t′″, h″, and h′″ is defined. For example, R⁹ may be expressed as —[(CH₂)₂O]₂— where h is 2, X⁵ is —O—, t is 2, and t′ is 0. Alternatively, R⁹ may be expressed as —CH₂—CH₂—O—CH₂—CH₂—O— where h is 2, X⁵ is —O—, t is 2, and t′ is 0. Similarly, R¹⁰ may be expressed as —[(CH₂)₂O]—H, where h″ is 2, X⁷ is —O—, t″ is 1, t′″ is 0, and R^{6″} is —H. Alternatively, R¹⁰ may be expressed as —CH₂—CH₂—OH, where h″ is 2, X⁷ is —O—, t″ is 1, t′″ is 0, and R^{6″} is —H. In several embodiments, R¹⁰ is an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol. In several embodiments, R¹⁰ is optionally substituted. In several embodiments, R¹⁰ is optionally substituted C₁₋₆ alkyl. In several embodiments, where substituted, R¹⁰ is substituted with one or more of —OH, halogen, C₁₋₃ alkyl and/or C-carboxy. In several embodiments, optional substitutions of the W¹, W², W³, and W⁴ may be optionally substituted. In several embodiments, optional substitutions of the W¹, W², W³, and W⁴ (e.g., of any variable within W¹, W², W³, and W⁴), where present, may be independently selected from C₁₋₃ alkyl, C₁₋₆ alkoxy, hydroxyl, amino, halogen, and/or combinations thereof.

In several embodiments, X³ is —C(O)—NH— and R⁹ is —(CH₂)₂— or —(CH₂)₂—(O—CH₂—CH₂)ₜ—. In some embodiments, t is an integer of equal to or at least about: 0, 1, 2, 3, 4, 5, 10, 20, or ranges including and/or spanning the aforementioned values. In some embodiments, t is an integer of equal to or at least about: 0, 1, 2, 3, 4, 5, 10, 20, or ranges including and/or spanning the aforementioned values. In several embodiments, t is an integer from 1 to 5 (e.g., 1, 2, 3, 4, or 5). In some embodiments, X³ and R⁹ taken together are a direct bond, optionally substituted —C(O)—NH—(CH₂)₂— (an ethylacetamido group or “EtAcN”) or optionally substituted —C(O)—NH—(CH₂)₂—(O—CH₂—CH₂)— (a pegylated ethylacetamido group or “Et-PEGₜ-AcN”), t is an integer from 1 to 5.

In several embodiments, X⁴ is a direct bond, —C(O)—NH—, —C(O)O— (or combinations thereof where more than one type of W² or W⁴ is present). In some embodiments, X⁴ and R¹⁰ taken together are —C(O)—NH₂ or —C(O)—OH in a given unit of W² or W⁴. In several embodiments, R¹⁰ is —[((CH₂)_{h″}—X⁷)_{t″}—((CH₂)_{h′″}—X⁸)_{t′″}—]—R^{6″}. In some embodiments, X⁸ and R^{6″} together provide a reactive group (e.g., —NH₂, —C(O)OH, —OH, maleic anhydride, etc.) that may be functionalized after polymerization (e.g., with a liver targeting moiety). In some embodiments, each instance of R^{6″} is independently H or optionally substituted —C₁₋₆ alkyl.

In some embodiments, Y′ is a random copolymer, gradient copolymer, or block copolymer of W¹ and W² or of W³ and W⁴, where W¹, W², W³, and W⁴ are as depicted below:

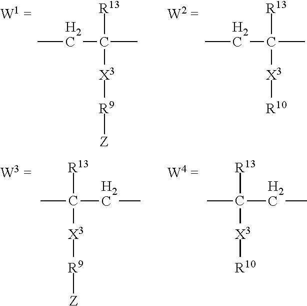

In several embodiments, the variables are as disclosed elsewhere herein. In several embodiments, Z is a liver targeting moiety (e.g., galactose, and/or glucose and/or a galactose and/or glucose receptor-targeting moiety, including, but not limited to, one or more of galactosamine, glucosamine, N-acetylgalactosamine, or N-acetylglucosamine or its receptor-targeting moiety, and/or moieties that exhibit affinity to receptors thereof), X³ is selected from a direct bond, —C(O)—NH— or —C(O)O—, and R⁹ is a direct bond or —[((CH₂)ₕX⁵)ₜ—((CH₂)_{h′}—X⁶)_{t′}]—. In several embodiments, X⁵ and X⁶ are independently selected from a direct bond, —CNR^{6′}—, and —O—. In some embodiments, each instance of R^{6′} (e.g., in X⁵ or X⁶) is independently H or optionally substituted —C₁₋₆alkyl. In several embodiments, t, t′, h, and h′ are each independently an integer of equal to or at least about: 0, 1, 2, 3, 4, 5, 6, 7, 8, 9, 10, 20, or ranges including and/or spanning the aforementioned values. In several embodiments, X³ is —C(O)—NH— and R⁹ is —(CH₂)₂— or —(CH₂)₂—(O—CH₂—CH₂)ₜ—. In several embodiments, t is an integer from 1 to 5. In some embodiments, X³ and R⁹ together are a direct bond, optionally substituted —C(O)—NH—(CH₂)₂— (an ethylacetamido group or “EtAcN”) or optionally substituted —C(O)—NH—(CH₂)₂—(O—CH₂—CH₂)ₜ— (a pegylated ethylacetamido group or “Et-PEGₜ-AcN”), t is an integer from 1 to 5. In some embodiments, t is an integer of equal to or at least about: 0, 1, 2, 3, 4, 5, 10, 20, or ranges including and/or spanning the aforementioned values. In some embodiments, t is an integer of equal to or at least about: 0, 1, 2, 3, 4, 5, 10, 20, or ranges including and/or spanning the aforementioned values.

In several embodiments, X⁴ is a direct bond, —C(O)—NH—, or —C(O)O—. In several embodiments, R¹⁰ is —H or —[((CH₂)_{h″}—X⁷)_{t″}—((CH₂)_{h′″}—X⁸)_{t′″}—]—R^{6″}. In some embodiments, where X⁴ and R¹⁰ are taken together to provide —C(O)—OH in a given unit of W². In several embodiments, X⁷ and X⁸ are independently selected from a direct bond, —NR^{6″}—, and —O—. In some embodiments, X⁸ and R^{6″} are taken together to provide a reactive group (e.g., —NH₂, —C(O)OH, —OH, maleic anhydride, etc.) that may be functionalized after polymerization (e.g., with a liver targeting moiety). In some embodiments, each instance of R^{6″} is independently H or optionally substituted —C₁₋₆ alkyl. In some embodiments, each instance of R^{6″} is independently H or optionally substituted —C₁₋₆ alkyl. In several embodiments, t″, t′″, h″, and h′″ are each independently an integer of equal to or at least about: 0, 1, 2, 3, 4, 5, 6, 7, 8, 9, 10, 20, or ranges including and/or spanning the aforementioned values. In several embodiments, R¹⁰ is an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol.

In some embodiments, R⁹ or R¹⁰ are independently optionally substituted alkyl, an optionally substituted polyether, or optionally substituted polyamino. In some embodiments, R¹⁰ is an optionally substituted C_{f} alkyl, optionally substituted C_{f} alkylOH_{g}, or an optionally substituted —(C_{f} alkyl(OH)_{g})—O)ₑ—H where f represents the number of carbons in the alkyl group and is an integer between 0 and 10, g represents the number of hydroxyl groups present on the alkyl group and is an integer between 0 and 10, and e represents the number of alkyl/ether repeat units and is an integer between 0 and 10. In some embodiments, e, f, and g are independently selected integers of equal to or at least about: 0, 1, 2, 3, 4, 5, 10, or ranges including and/or spanning the aforementioned values. In some embodiments, R¹⁰ is a 2-hydroxyethyl (e.g., —CH₂CH₂OH). In some embodiments, R¹⁰ is an optionally substituted 2-hydroxyethyl. In some embodiments, R¹⁰ is an optionally substituted polyether.

In some embodiments, Y′ is represented as -W¹ₚ-W²ᵣ, where -W¹ₚ-W²ᵣ— represents a block copolymer, gradient copolymer, or a random copolymer of W¹ and W² monomers having p repeat units of W¹ and r repeat units of W². In some embodiments, p is an integer equal to or greater than about: 0, 1, 50, 85, 100, 150, 165, 200, 225, 250, 300, 400, or ranges including and/or spanning the aforementioned values. For example, p may range from 1 to 150, from 50 to 100, from 50 to 150, etc. In some embodiments, r is an integer equal to or greater than about: 0, 1, 50, 85, 100, 150, 165, 200, 225, 250, 300, 400, or ranges including and/or spanning the aforementioned values. For example, r may range from 0 to 150, from 50 to 100, from 50 to 150, etc. In some embodiments, Y′ is a homopolymer of W¹ or W². In some embodiments, p is 0. In some embodiments, r is 0. In some embodiments, the sum of p and r is an integer equal to or greater than about: 1, 50, 75, 80, 85, 100, 150, 165, 170, 200, 225, 250, 300, 400, 600, 800, or ranges including and/or spanning the aforementioned values. For example, the number of p and r units may range from 1 to 150, from 50 to 250, from 100 to 300, etc. As another illustration, the number of p and r units may range be equal to or at least 100, 150, 200, etc.

In some embodiments, Y′ is represented as -W³ₚ-W⁴ᵣ—, where -W³ₚ-W⁴ᵣ— represents a block copolymer, gradient copolymer, or a random copolymer of W³ and W⁴ monomers having p repeat units of W³ and r repeat units of W⁴. In some embodiments, p is an integer equal to or greater than about: 0, 1, 50, 85, 100, 150, 165, 200, 225, 250, 300, 400, or ranges including and/or spanning the aforementioned values. For example, p may range from 1 to 150, from 50 to 100, from 50 to 150, etc. In some embodiments, r is an integer equal to or greater than about: 0, 1, 50, 85, 100, 150, 165, 200, 225, 250, 300, 400, or ranges including and/or spanning the aforementioned values. For example, r may range from 0 to 150, from 50 to 100, from 50 to 150, etc. In some embodiments, Y′ is a homopolymer of W³ or W⁴. In some embodiments, p is 0. In some embodiments, r is 0. In some embodiments, the sum of p and r is an integer equal to or greater than about: 1, 50, 75, 80, 85, 100, 150, 165, 170, 200, 225, 250, 300, 400, 600, 800, or ranges including and/or spanning the aforementioned values. For example, the number of p and r units may range from 1 to 150, from 50 to 250, from 100 to 300, etc. As another illustration, the number of p and r units may range be equal to or at least 100, 150, 200, etc.

In some embodiments, polymeric chain^{a} is present or optionally not present. In some embodiments, the polymeric chain^{a} is optionally substituted. In some embodiments, where present, polymeric chain^{a} can comprise a hydrophilic polymer. In some embodiments, where present, polymeric chain^{a} can comprise one or more optionally substituted —(CH₂CH₂O)ₛ—, optionally substituted —(CH₂)ᵤ—, or optionally substituted alkylene. In several embodiments, u is an integer less than or equal to about: 1, 5, 10, 20, or ranges including and/or spanning the aforementioned values. In some embodiments, polymeric chain^{a} can comprise or consist of one or more of the following structures, or a portion thereof. For example, in some embodiments, the polymeric chain^{a} comprises more than of these structural segments:

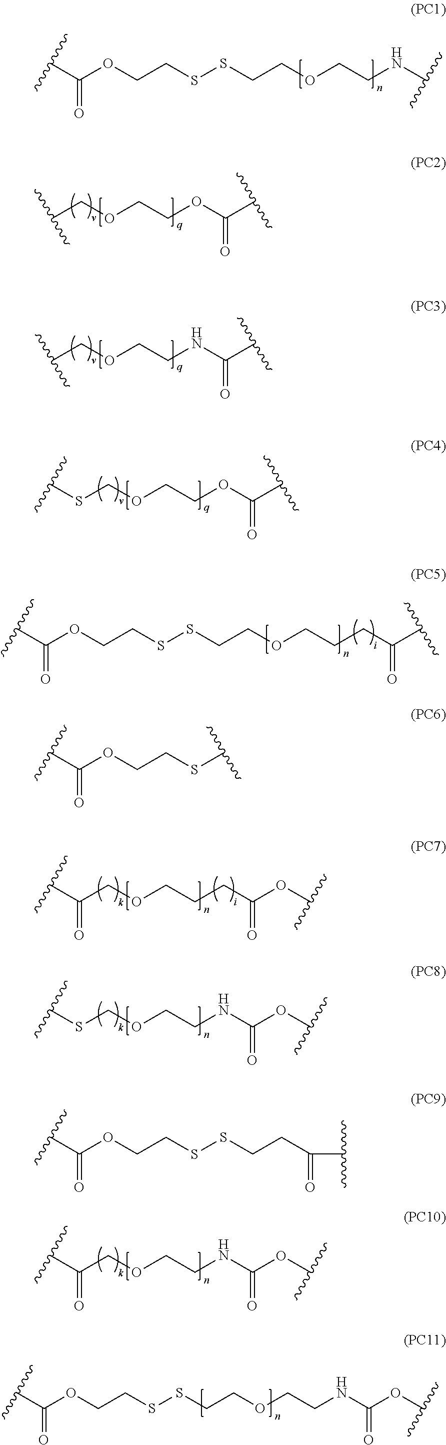

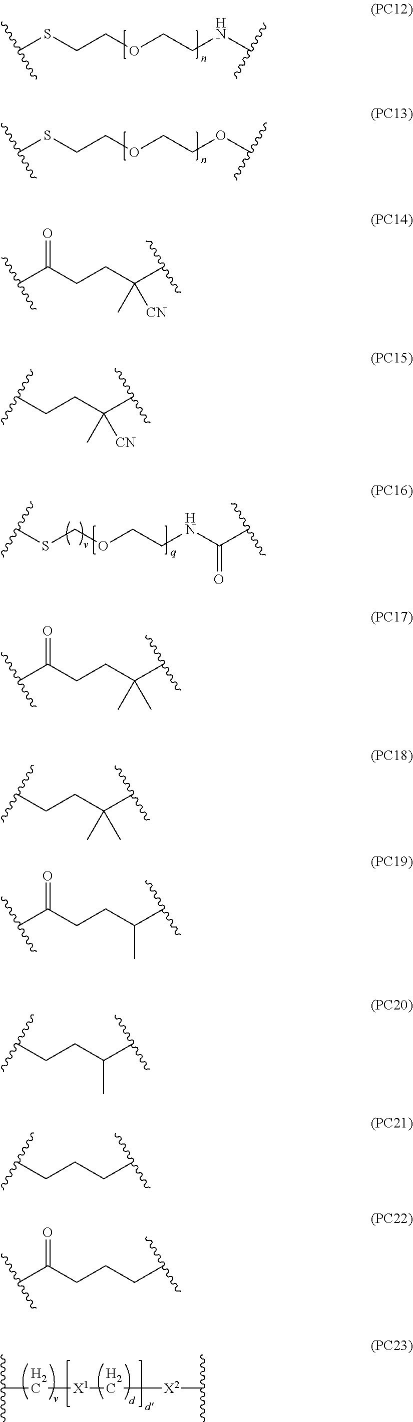

wherein the variables (e.g., i, k, n, q, v, d, d′, X¹, and X²) are as disclosed elsewhere herein. In several embodiments, i, k, n, q, v, d, and d′ are each independently an integer that is greater than or equal to about: 0, 1, 2, 3, 4, 5, 10, 15, 20, 30, 40, 44, 50, 75, 100, 150, or ranges including and/or spanning the aforementioned values. In several embodiments, i is an integer that is greater than or equal to about: 0, 1, 2, 3, 4, 5, 10, 15, 20, 30, 40, or ranges including and/or spanning the aforementioned values. In several embodiments, k is an integer that is greater than or equal to about: 0, 1, 2, 3, 4, 5, 10, 15, 20, 30, 40, or ranges including and/or spanning the aforementioned values. In several embodiments, n is an integer that is greater than or equal to about: 0, 1, 2, 3, 4, 5, 10, 15, 20, 30, 40, 50, or ranges including and/or spanning the aforementioned values. In several embodiments, q is an integer that is greater than or equal to about: 0, 1, 2, 3, 4, 5, 10, 15, 20, 30, 40, 50, or ranges including and/or spanning the aforementioned values. In several embodiments, v is an integer that is greater than or equal to about: 0, 1, 2, 3, 4, 5, 10, 15, 20, 30, 40, or ranges including and/or spanning the aforementioned values. In several embodiments, d is an integer that is greater than or equal to about: 0, 1, 2, 3, 4, 5, 10, 15, 20, 30, 40, or ranges including and/or spanning the aforementioned values. In several embodiments, d′ is an integer that is greater than or equal to about: 0, 1, 2, 3, 4, 5, 10, 15, 20, 30, 40, or ranges including and/or spanning the aforementioned values. In several embodiments, X¹ and X² are independently selected from —NR⁶— (e.g., —NH—) and —O—, where R⁶ is H or optionally substituted C₁₋₆alkyl.

To illustrate, an embodiment of polymer chain^{a} comprising one or more of the structural segments found above, when polymer chain^{a} comprises the below structure:

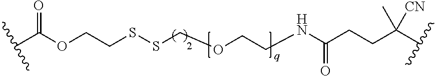

that polymer chain^{a} structure can be described as comprising any one of the following, or all of the following:

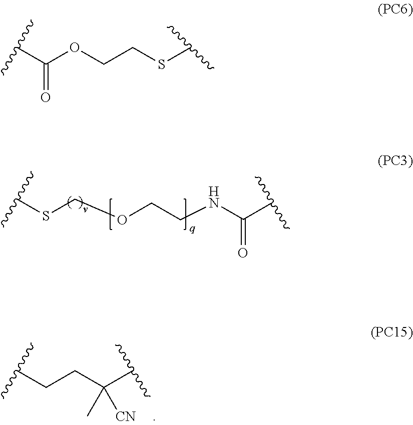

where v is 2. To further illustrate, an embodiment of polymer chain^{a} comprising one or more of the structural segments found above, when polymer chain^{a} comprises the below structure:

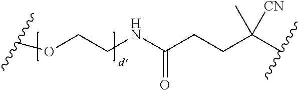

that polymer chain^{a} structure can be described as comprising any one of the following, or all of the following:

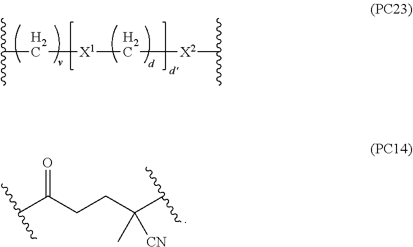

where v is 0, d is 2, X¹ is —O—, and X² is NR. In some embodiments, Formula (AI) comprises any of the following combinations: (PC6), (PC3), and (PC15); (PC3) and (PC15); (PC6), (PC4), and (PC15); (PC4) and (PC15); (PC13) and (PC14); (PC23) and (PC14); or other combinations of any one or more of (PC1) to (PC23).

In several embodiments, n is an integer from about 1 to about 100 (or n is 0), q is an integer from about 1 to about 100 (or q is 0), k is an integer from about 1 to about 20 (or k is 0), i is an integer from about 0 to about 20, and v is an integer from about 1 to about 20 (or v is 0). In several embodiments, n is an integer greater than or equal to about: 0, 1, 10, 20, 40, 50, 75, 100, 150 or ranges including and/or spanning the aforementioned values. In several embodiments, n is an integer greater than or equal to about: 0, 1, 2, 3, 4, 5, 10, 15, 20, 30, 40, 50, or ranges including and/or spanning the aforementioned values. In several embodiments, q is an integer greater than or equal to about: 0, 1, 10, 20, 40, 50, 75, 100, 150 or ranges including and/or spanning the aforementioned values. In several embodiments, q is an integer greater than or equal to about: 0, 1, 2, 3, 4, 5, 10, 15, 20, 30, 40, 50, 75, 100, 150, or ranges including and/or spanning the aforementioned values. In several embodiments, i is an integer greater than or equal to about: 0, 1, 2, 3, 4, 5, 10, 15, 20, 30, 40, 50, or ranges including and/or spanning the aforementioned values. In several embodiments, k is an integer greater than or equal to about: 0, 1, 2, 3, 4, 5, 10, 15, 20, or ranges including and/or spanning the aforementioned values. In several embodiments, v is an integer greater than or equal to about: 0, 1, 2, 3, 4, 5, 10, 15, 20, or ranges including and/or spanning the aforementioned values. In several embodiments, k is 2. In several embodiments, v is 2. In several embodiments, n is 4. In several embodiments, n is 43 or 44. In several embodiments, q is 3. As used herein, variables disclosed as having structure, a value, or a range of values for one embodiment, may also have those values when the variable is used in another embodiment (even where the variable is not defined with respect to that other embodiment). In several embodiments, n or q represents the number of repeat units in a PEG chain. In some embodiments, the PEG chain may have some polydispersity. In some embodiments, n and q do not indicate a number of repeat units but instead independently indicate the presence of a PEG polymer chain having a M_{N} (in g/mol) or M_{W} (in g/mol) of equal to or at least about: 50, 200, 300, 500, 1000, 2000, 5000, 10000, or ranges including and/or spanning the aforementioned values. In some embodiments, k, i, and v can each independently comprise an optionally substituted alkylene.

In some embodiments, the compound comprises the following configuration, where the variables (e.g., X, Y, Z, EU, m, p, etc.) are as disclosed elsewhere herein:

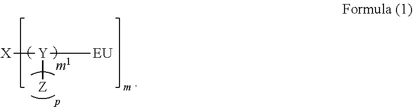

In Formula (1′), Y can include, for example, a “box car” motif with m¹ box cars. Two embodiments of boxcar motifs are shown below with m¹ equal to 3 in each instance. In some embodiments, as shown, the box car arrangement may include a single polymeric chain^{a} (as disclosed elsewhere herein) and different Y′ portions (as shown below in Formula (P1)). In some embodiments, the box car arrangement may include more than one polymeric chain^{a} portions (that are different or the same) along with separate Y′ segments (see Formula (P2)). Where more than one polymeric chain^{a} is provided (e.g., polymeric chain^{a1}, polymeric chain^{a2}, polymeric chain^{a3}) with each may be independently defined as polymeric chain^{a} above (and each instance may be the same or different).

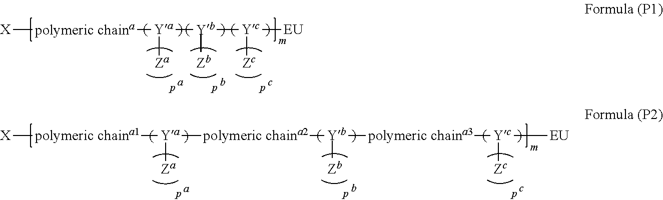

In some embodiments, as indicated above, Y′^{a}, Y′^{b}, and Y′^{c} independently comprise polymeric or copolymeric portions (e.g., acrylyl portions as disclosed elsewhere herein as Y′) that may be the same or different, Z^{a}, Z^{b}, and Z^{c} independently comprise targeting moieties that may be the same or different, and p^{a}, p^{b}, and p^{c} each indicate the number of Z^{a}, Z^{b}, and Z^{c} repeat units present, respectively. Each different Y′ portion (e.g., Y′^{a}, Y′^{b}, and Y′^{c}) may comprise various W¹ structures, W² structures, W³ structures, W⁴ structures, or mixtures thereof, as disclosed elsewhere herein. In some embodiments, m¹ is an integer equal to or greater than about: 1, 2, 3, 4, 5, 10, or ranges including and/or spanning the aforementioned values. In some embodiments, where a Y′ portion (e.g., Y′^{a}, Y′^{b}, and/or Y′^{c}) lacks a liver targeting moiety, p (e.g., p^{a}, p^{b}, and/or p^{c}) may be 0 in that portion. In some embodiments, where a Y′ portion (e.g., Y′^{a}, Y′^{b}, and/or Y′^{c}) lacks a spacer, r (e.g., r^{a}, r^{b}, and/or r^{c}, not shown) may be 0 in that portion.

In several embodiments, —[Y(Z)ₚ]— is a group represented by any one or more of of the following Formulae (e.g., (Ya′) to (Yz″)):

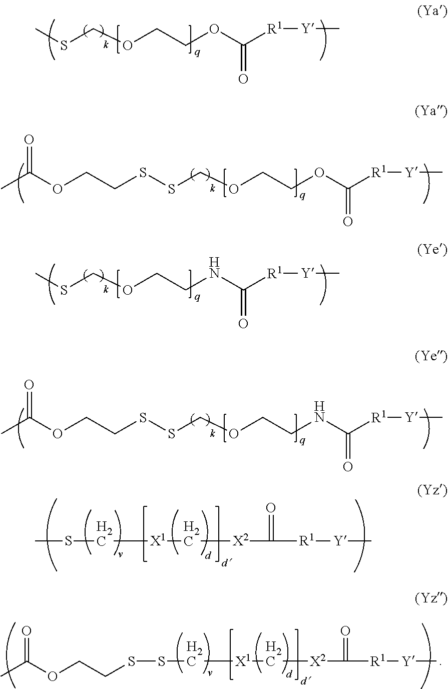

wherein the variables (e.g., Y′, R¹, v, d, d′, X¹, X², k, and q) are as disclosed elsewhere herein. In several embodiments, q is an integer greater than or equal to about: 0, 1, 2, 3, 4, 5, 10, 15, 20, 30, 40, 50, 75, 100, 150, or ranges including and/or spanning the aforementioned values. In several embodiments, k is an integer greater than or equal to about: 0, 1, 2, 3, 4, 5, 10, 15, 20, or ranges including and/or spanning the aforementioned values. In several embodiments, q is 0. In several embodiments, q is 3. In several embodiments, k is 2. In several embodiments, v is 2. In several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—. In several embodiments, X¹ and X² are independently selected from —NR⁶— and —O—. In some embodiments, v, d, and d′ are independently an integer greater than or equal to about: 0, 1, 2, 3, 4, 5, 10, 15, 20, 40, 50, 75, 100, 150 or ranges including and/or spanning the aforementioned values. In some embodiments, each instance of R⁶ is H or optionally substituted C₁₋₆ alkyl. In some embodiments, Y′ is a copolymer of W¹ and W² having p repeat units of W¹ and r repeat units of W². In some embodiments, Y′ is a copolymer of W³ and W⁴ having p repeat units of W³ and r repeat units of W⁴. In several embodiments, Y is Ya′. In several embodiments, Y is Ye′. In several embodiments, Y is Yz′. In several embodiments, Formula (AI) may be expressed as any one of Formulae (Ya′) to (Yz″).

In some embodiments, as disclosed elsewhere herein, Y′ comprises (or consists of, or consists essentially of) W¹ and W² or W³ and W⁴, where W¹, W², W³, and W⁴ are as depicted below, and as described elsewhere herein:

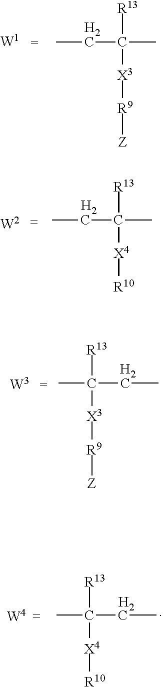

where the variable as disclosed elsewhere herein. In several embodiments, Z is a targeting moiety (e.g., including but not limited to any one or more of galactose, a galactose receptor-targeting moiety, glucose, a glucose receptor-targeting moiety, galactosamine, a galactosamine receptor-targeting moiety, glucosamine, and/or moieties that exhibit affinity to receptors thereof including a glucosamine receptor-targeting moiety, N-acetylgalactosamine, a N-acetylgalactosamine receptor-targeting moiety, N-acetylglucosamine, and/or a N-acetylgalactosamine receptor-targeting moiety); each instance of R³ (e.g., in either W¹, W², W³, or W⁴) is independently H, methyl, or optionally substituted —C₁₋₆ alkyl; X³ is (or each instance of X³ is independently) a direct bond, —C(O)—NH—, or —C(O)O—; R⁹ is a direct bond or —[((CH₂)^{h}X⁵)ₜ—((CH₂)_{h′}X⁶)_{t′}—]—; X⁴ is (or each instance of X⁴ is independently) a direct bond, —C(O)—NH— or —C(O)O—; and R¹⁰ is —H or —[((CH₂)_{h″}—X⁷)_{t″}—((CH₂)_{h′″}X⁸)_{t′″}]—R^{6″}. In several embodiments, X⁵ and X⁶ are independently selected from a direct bond, —NR^{6′}—, and —O—. In some embodiments, each instance of R^{6′} (e.g., in X⁵ or X⁶) is independently H or optionally substituted —C₁₋₆alkyl. In several embodiments, t, t′, h, and h′ are each independently an integer of equal to or at least about: 0, 1, 2, 3, 4, 5, 6, 7, 8, 9, 10, 20, or ranges including and/or spanning the aforementioned values. In several embodiments, X⁷ and X⁸ are independently selected from a direct bond, —NR^{6″}—, and —O—. In some embodiments, each instance of R^{6″}— is independently H or optionally substituted —C₁₋₆ alkyl. In several embodiments, t″, t′″, h″, and h′″ are each independently an integer of equal to or at least about: 0, 1, 2, 3, 4, 5, 6, 7, 8, 9, 10, 20, or ranges including and/or spanning the aforementioned values. In several embodiments, R¹⁰ is an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol. In several embodiments, optional substitutions of W¹, W², W³, and W⁴ (e.g., of any variable within W¹, W², W³, and W⁴), where present, may be independently selected from C₁₋₃ alkyl, C₁₋₆ alkoxy, hydroxyl, amino, halogen, and/or combinations thereof. In several embodiments, optional substitutions of any variable of Y′, where present, may be independently selected from C₁₋₃ alkyl, C₁₋₆ alkoxy, hydroxyl, amino, halogen, and/or combinations thereof.

In several embodiments, Z comprises a galactose and/or glucose receptor-targeting moiety. In several embodiments, each instance of R¹³ (e.g., in either W¹, W², W³, or W⁴) is independently H, methyl, or optionally substituted —C₁₋₆ alkyl. In several embodiments, optional substitutions of R¹³, where present, may be independently selected from C₁₋₃ alkyl, C₁₋₆alkoxy, hydroxyl, amino, halogen, and/or combinations thereof. In several embodiments, each instance of R¹³ is methyl. In several embodiments, R⁹ is a direct bond or —[((CH₂)ₕX⁵)ₜ—((CH₂)_{h′}X⁶)_{t′}]—. In several embodiments, X⁵ and X⁶ are independently selected from a direct bond, —CNR^{6′}—, and —O—. In some embodiments, each instance of R^{6′} (e.g., in X⁵ or X⁶) is independently H or optionally substituted —C₁₋₆ alkyl. In several embodiments, t, t′, h, and h′ are each independently an integer of equal to or at least about: 0, 1, 2, 3, 4, 5, 6, 7, 8, 9, 10, 20, or ranges including and/or spanning the aforementioned values. In several embodiments, X³ is —C(O)—NH— and R⁹ is —(CH₂)₂— or —(CH₂)₂—(O—CH₂—CH₂)—. In several embodiments, t is an integer from 1 to 5. In several embodiments, X⁴ is —C(O)—NH—, —C(O)O—, or —C(O)—OH. In some embodiments, where X⁴ is —C(O)—OH in a given unit of W² or W⁴ and R¹⁰ is not present. In several embodiments, R¹⁰ is an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol. In several embodiments, R¹⁰ is —[((CH₂)_{h″}—X⁷)_{t″}—((CH₂)_{h′″}—X⁸)_{t′″}]—R^{6″}. In several embodiments, X⁷ and X⁸ are independently selected from a direct bond, —NR^{6″}—, and —O—. In some embodiments, each instance of R^{6″} is independently H or optionally substituted —C₁₋₆ alkyl. In several embodiments, t″, t′″, h″, and h′″ are each independently an integer of equal to or at least about: 0, 1, 2, 3, 4, 5, 6, 7, 8, 9, 10, 20, or ranges including and/or spanning the aforementioned values.

In some embodiments, X³ and R⁹ together form a direct bond, —C(O)—NH—(CH₂)₂—, or —C(O)—NH—(CH₂)₂—(O—CH₂—CH₂)ₜ—, where t is an integer from 1 to 5. In several embodiments, p is an integer from 2 to 250. In several embodiments, X⁴ is a direct bond and R¹⁰ is an aliphatic group, an alcohol, an aliphatic amine-containing group, or an aliphatic alcohol. In several embodiments, R¹⁰ is an aliphatic group, an amine, a polyamino, an alcohol, a polyether, or an aliphatic alcohol. In several embodiments, r is an integer from 0 to 250. In several embodiments, R¹⁰ is a C_{f}alkyl or C_{f}alkylOH_{g}, where f represents the number of carbons in the alkyl group and is an integer between 0 and 10, and g represents the number of hydroxyl groups present on the alkyl group and is an integer between 0 and 10. In some embodiments, X⁴ is —C(O)NH— and R¹⁰ is 2-hydroxyethyl.

In some aspects, as disclosed elsewhere herein, -W¹ₚ-W²ᵣ— and/or -W³ₚ-W⁴ᵣ— represents a block copolymer, gradient copolymer, or a random copolymer of W¹ and W² repeat units, or W³ and W⁴ repeat units, respectively. In several embodiments, Y′ may comprise different acrylyl units represented by different W¹ or W³ structures. For example, with respect to W¹, in some embodiments, Y′ comprises a first acrylyl unit and a second acrylyl unit that are different but that can both be represented structurally by the structure of W¹. By way of illustration, in several embodiments, the first acrylyl unit comprises W¹ where X³ is —C(O)O—, R⁹ is —[((CH₂)ₕX⁵)ₜ—((CH₂)_{h′}X⁶)_{t′}]—, where h is 3, X⁵ is —O—, t is 2, h′ is 3, X⁶ is —O—, and t′ is 1, and Z is a first galactosylating moiety. In several embodiments, the second acrylyl unit comprises W¹ where X³ is —C(O)NH—, R⁹ is —[((CH₂)ₕX⁵)ₜ—((CH₂)_{h′}—X⁶)_{t′}]—, where h is 2, X⁵ is —O—, t is 4, h′ is 1, X⁶ is —O—, and t′ is 1 and Z is a second galactosylating moiety different or the same as the first. In several embodiments, a third acrylyl unit may be present and may comprise W¹ where X³ is —C(O)NH—, R⁹ is —[((CH₂)ₕX⁵)ₜ—((CH₂)_{h′}X⁶)_{t′}]—, where h is 2, X⁵ is —O—, t is 2, h′ is 1, and t′ is 1 and Z is a third glucosylating moiety different or the same as the first and/or second. In several embodiments a fourth acrylyl unit may be represented by W² (e.g., a spacer). In several embodiments, multiple types of acrylyl spacers (a fifth acrylyl unit, sixth acrylyl unit, etc.) may be used. In several embodiments, similarly, Y′ may comprise different acrylyl units that are represented by W² or W⁴ (e.g., a first W² or W⁴ acrylyl units and a second W² or W⁴ acrylyl unit, respectively).

In several embodiments, as shown elsewhere herein, the targeting portion comprises one or more pendant liver targeting moieties decorating a portion of the linker. In several embodiments, the portion of the linker is a polymeric chain with pendant targeting agents attached randomly, in a gradient, and/or in blocks along the chain. In some embodiments, the polymeric chain comprises an acrylyl portion (e.g., acrylyl polymers and/or acrylyl copolymers). In several embodiments, the acrylyl portion comprises an acrylyl unit comprising a pendant liver targeting agent. In several embodiments, the acrylyl portion further comprises an acrylyl unit not comprising a pendant liver targeting agent.

In some embodiments, the tolerogenic construct results from one or more reactions involving at least one of the following: N-hydroxysuccinamidyl (NHS) linker, NHS ester linker, PEG linker, maleimide linker, vinylsulfone linker, pyridyl di-thiol-poly(ethylene glycol) linker, pyridyl di-thiol linker, n-nitrophenyl carbonate linker, or a nitrophenoxy poly(ethylene glycol)ester linker. The linker may have one or more galactose and/or glucose moieties and/or galactose and/or glucose receptor-targeting moieties bound to it. In embodiments, Y comprises an antibody, an antibody fragment, a peptide, or a disulfanyl ethyl ester to which one or more galactose and/or glucose moieties and/or galactose and/or glucose receptor-targeting moieties are bound.

Di-thiol-containing linkers and disulfanyl ethyl carbamate-containing linkers (named including a free amine of the antigen, otherwise named a “disulfanyl ethyl ester” without including the free amine of an antigen (e.g., X)) are advantageous in the present constructs as having the ability to cleave and release an antigen in its original form once inside a cell (or at a target area). For example as illustrated below (where X′ indicates the remaining portion of the antigen and the disulfanyl ethyl ester is part of the antigen).

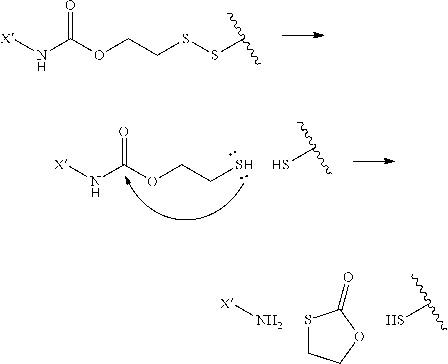

### Targeting

According to several embodiments, targeting of compositions disclosed herein (e.g., to the liver) is accomplished by one or more types of moiety that binds to receptors on liver cells (or a subtype of liver cell). In several embodiments, the targeting agent (e.g., liver targeting moiety) is operably linked to the linker. In several embodiments, a targeting agent that binds a galactose receptor is used. In several embodiments, a galactosylating moiety (e.g., galactose, galactosamine, and N-acetylgalactosamine) is used. In several embodiments, such a moiety can be conjugated to a linker at any of the carbon molecules of the sugar. In several embodiments, the conjugation of the galactosylating moiety is at the C1, C2 or C6 position. In several embodiments, a targeting agent that binds a glucose receptor is used. In several embodiments, a glucosylating moiety (e.g., glucose, glucosamine and N-acetylglucosamine) is used. In several embodiments, glucosylating moieties may be glucose receptor targeting moieties. In several embodiments, such a moiety can be conjugated to a linker at any of the carbon molecules of the sugar. In several embodiments, the conjugation of the glucosylating moiety is at the C1, C2 or C6 position. Combinations of glucose receptor and galactose receptor targeting moieties may also be used, depending on the embodiment. In several embodiments, where multiple targeting agents are used, specific ratios of glucose receptor-based (e.g., targeting) to galactose receptor-based (e.g., targeting) moieties are used, for example, about 500:1, about 250:1, about 100:1, about 50:1, about 25:1, about 10:1, about 5:1, about 2:1, about 1:1, about 1:2, about 1:5, about 1:10, about 1:25, about 1:50 about 1:100, about 1:250, about 1:500, and any ratio in between those listed, including endpoints. In additional embodiments, a polypeptide for which such liver-targeting is desired can be desialylated to facilitate targeting. Depending on the embodiment, the galactosylating or glucosylating moiety (or galactose receptor targeting moiety and/or glucose receptor targeting moiety) can be chemically conjugated or recombinantly fused to an antigen, whereas desialylation exposes a galactose-like moiety on an antigen polypeptide.

In several embodiments, various ratios of W¹ to W² or W³ to W⁴ are used. In some embodiments, of the Y′ repeat units, a majority comprise W¹. In some embodiments, the ratio of W¹ to W² is equal to or greater than about about 50:1, about 25:1, about 10:1, about 5:1, about 4:1, about 2:1, about 1:1, about 1:2, about 1:4, about 1:5, about 1:10, about 1:25, about 1:50, and any ratio in between those listed, including endpoints. In some embodiments, of the Y′ repeat units a majority comprise W³ repeat units. In some embodiments, the ratio of W³ to W⁴ is equal to or greater than about about 50:1, about 25:1, about 10:1, about 5:1, about 4:1, about 2:1, about 1:1, about 1:2, about 1:4, about 1:5, about 1:10, about 1:25, about 1:50, and any ratio in between those listed, including endpoints. In some embodiments, the ratio of p to r is (for W¹ and W² or W³ and W⁴) equal to or greater than about about 50:1, about 25:1, about 10:1, about 5:1, about 4:1, about 2:1, about 1:1, about 1:2, about 1:4, about 1:5, about 1:10, about 1:25, about 1:50, and any ratio in between those listed, including endpoints. In some embodiments, a homopolymer of W¹ is provided without a W² portion. In some embodiments, a homopolymer of W³ is provided without a W⁴ portion.

### Antigens

In several embodiments, the antigen to which tolerance is desired is an agent that is capable of inducing an unwanted immune response in the subject. The antigen employed as X in the compounds or compositions of Formula (1), or in any of the compounds, compositions, or methods of the current disclosure, can be a protein or a peptide, e.g. the antigen may be a complete or partial therapeutic agent, a full-length transplant protein or peptide thereof, a full-length autoantigen or peptide thereof, a full-length allergen or peptide thereof, and/or a nucleic acid, or a mimetic of an aforementioned antigen. Combinations of multiple fragments may also be used, depending on the embodiment. For example, if a longer peptide identified as P has antigenic regions A, B, C, and D, compositions disclosed herein for induction of tolerance to P can comprise any combination of A, B, C, and D, and repeats of any of A, B, C, and D. A listing of any particular antigen in a category or association with any particular disease or reaction does not preclude that antigen from being considered part of another category or associated with another disease or reaction.

In several embodiments, the antigen comprises one or more therapeutic agents that are proteins, peptides, antibodies, and antibody-like molecules (including antibody fragments and fusion proteins with antibodies and antibody fragments), and gene therapy vectors. These include human, non-human (such as mouse) and non-natural (e.g., engineered) proteins, antibodies, chimeric antibodies, humanized antibodies, viruses and virus-like particles, and non-antibody binding scaffolds, such as fibronectins, DARPins, knottins, and the like. In several embodiments, human allograft transplantation antigens against which transplant recipients develop an unwanted immune response are used. In several embodiments, the antigen comprises one or more self-antigens that cause an unwanted, autoimmune response. While self-antigens are of an endogenous origin in an autoimmune disease patient, according to several embodiments, the polypeptides employed in the disclosed compositions are, depending on the embodiment, synthesized exogenously (as opposed to being purified and concentrated from a source of origin).

In several embodiments, the antigen to which tolerance is desired comprises one or more foreign antigens, such as food, animal, plant, and environmental antigens against which a patient experiences an unwanted immune response. While a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, considered foreign antigens. In the interest of simplicity no attempt will be made to describe distinguish and define all of such potentially overlapping groups, as those skilled in the art can appreciate the antigens that can be employed in the compositions of the disclosure, particularly in light of the detailed description and examples.

In several embodiments, X is selected from the group consisting of insulin, proinsulin, preproinsulin, gluten, gliadin, myelin basic protein, myelin oligodendrocyte glycoprotein and proteolipid protein, desmoglein-3, desmoglein-1, alpha-synulein, acetylcholine receptor, Factor VIII, Factor IX, asparaginase, uricase, adeno-associated viruses (AAV), and fragments of any of the preceding. In several embodiments, the antigen X is not a full-length protein. For example, in some embodiments, the antigen is not full-length gliadin, insulin, or proinsulin. In several embodiments, the antigen is not full-length myelin basic protein, not full-length myelin oligodendrocyte protein, or not full-length proteolipid protein. In several embodiments, the antigen X is not a fragment of a protein. As discussed in more detail below, there exist a variety of antigens to which tolerance may be desired. These may include, but are not limited to, exogenous antigens that result in an adverse immune response when a subject is exposed to the antigen. In several embodiments, the adverse immune response could be a result of ingestion of the antigen, e.g., orally, nasally, or via some other mucosal route. These routes could be the case, for example, with food antigens. In some embodiments, the antigen may be purposefully administered to a subject, for example, with the administration of a therapeutic composition to treat a disease or condition that the subject is affected by. In still additional embodiments, the antigen may be produced by the subject, e.g., an autoimmune antigen. For example, in several embodiments, X comprises a foreign transplant antigen against which transplant recipients develop an unwanted immune response or a tolerogenic portion thereof. In several embodiments, X comprises a foreign food, animal, plant or environmental antigen against which patients develop an unwanted immune response or a tolerogenic portion thereof. In several embodiments, X comprises a foreign therapeutic agent against which patients develop an unwanted immune response or a tolerogenic portion thereof. In several embodiments, X comprises a synthetic self-antigen against the endogenous version of which patients develop an unwanted immune response or a tolerogenic portion thereof.

In further detail to the above, there are provided in several embodiments, compounds where X is a food antigen. In some such embodiments, X is one or more of conarachin (Ara h 1), allergen II (Ara h 2), arachis agglutinin, conglutin (Ara h 6), α-lactalbumin (ALA), lactotransferrin, Pen a 1 allergen (Pen a 1), allergen Pen m 2 (Pen m 2), tropomyosin fast isoform, high molecular weight glutenin, low molecular weight glutenin, alpha-gliadin, gamma-gliadin, omega-gliadin, hordein, seclain, and avenin. Fragment of any of these antigens and/or mimotopes of any of these antigens are also used, in several embodiments. In several embodiments, X is selected from the group consisting of gluten, high molecular weight glutenin, low molecular weight glutenin, alpha-gliadin, gamma-gliadin, omega-gliadin, hordein, seclain, and avenin and fragments thereof. In several embodiments, X is selected from the group consisting of gluten, high molecular weight glutenin, low molecular weight glutenin, alpha-gliadin, gamma-gliadin, and omega-gliadin and fragments thereof. In several embodiments, X is gluten or fragment thereof. In several embodiments, X is gliadin or fragment thereof.

In several embodiments, there are provided compounds where X is a therapeutic agent. In several embodiments, X is selected from the group consisting of Factor VII, Factor IX, asparaginase, and uricase, adeno-associated viruses (AAV), and fragments of any one thereof. In several embodiments, X is a therapeutic agent selected from the group consisting of Factor VII and Factor IX and fragments thereof. In several embodiments, X is a therapeutic agent selected from the group consisting of Factor VIII or fragment thereof. In several embodiments, when X is a therapeutic agent, the compound can be used in the treatment, prevention, reduction, or otherwise amelioration of an immune response developed against a therapeutic agent for hemophilia. As discussed herein, mimotopes of any antigenic portion of the antigens above can be used in several embodiments.

In several embodiments, X comprises asparaginase or a fragment thereof. In several embodiments, X comprises uricase or a fragment thereof. In several such embodiments, the compound can be used in the treatment, prevention, reduction, or otherwise amelioration of an immune response developed against an anti-neoplastic agent. As discussed herein, mimotopes of any antigenic portion of the antigens above can be used in several embodiments.

In several embodiments, X is associated with an autoimmune disease. For example, in several embodiments, the associated autoimmune disease is one or more of Type I diabetes, multiple sclerosis, rheumatoid arthritis, vitiligo, uveitis, pemphigus vulgaris, celiac disease, myasthenia gravis, and neuromyelitis optica.

In several embodiments, the autoimmune disease is Type I diabetes and X comprises insulin or a fragment thereof. In several embodiments, the autoimmune disease is Type I diabetes and X comprises proinsulin or a fragment thereof. In several embodiments, the autoimmune disease is Type I diabetes and X comprises preproinsulin or a fragment thereof. As discussed herein, mimotopes of any antigenic portion of the antigens above can be used in several embodiments. In several embodiments, combinations of these antigens can be incorporated into the tolerogenic compound which may aid in reducing immune responses to self-antigens at multiple points along the insulin pathway.

In several embodiments, the autoimmune disease is multiple sclerosis and X comprises myelin basic protein or a fragment thereof. In several embodiments, the autoimmune disease is multiple sclerosis and X comprises myelin oligodendrocyte glycoprotein or a fragment thereof. In several embodiments, the autoimmune disease is multiple sclerosis and X comprises proteolipid protein or a fragment thereof. As discussed herein, mimotopes of any antigenic portion of the antigens above can be used in several embodiments. In several embodiments, combinations of these antigens can be incorporated into the tolerogenic compound (e.g., a mixture of antigens or fragments of MOG, MBP and/or PLP) which may aid in reducing immune responses to self-antigens at multiple points along the enzymatic pathways that control myelination or myelin repair.

As discussed herein, mimotopes of any antigenic portion of the self-antigens above (or otherwise disclosed herein) can be used in several embodiments.

In several embodiments, the pharmaceutically acceptable composition consists of, or consists essentially of a compound wherein X is a food antigen, therapeutic agent, a self antigen, or fragment thereof, a linker Y, and a liver targeting moiety Z. In several embodiments, a galactosylating moiety (e.g., galactose, galactosamine, and N-acetylgalactosamine) is used as the targeting moiety Z (e.g., liver targeting moiety). In several embodiments, a glucosylating moiety (e.g., glucose, glucosamine and N-acetylglucosamine) is used as the targeting moiety Z (e.g., liver-targeting moiety).

The tolerogenic antigen can be a complete protein, a portion of a complete protein, a peptide, or the like, and can be derivatized (as discussed above) for attachment to a linker and/or antigen-binding moiety, can be a variant and/or can contain conservative substitutions, particularly maintaining sequence identity, and/or can be desialylated.

In the embodiments where the antigen is a therapeutic protein, peptide, antibody or antibody-like molecule, specific antigens can be selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

Particular therapeutic protein, peptide, antibody or antibody-like molecules include, but are not limited to, Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Some embodiments employ hemostatic agents (e.g., Factor VIII and IX), Insulin (including rHu Insulin), and the therapeutic molecules uricase, PAL and asparaginase (which may be non-human in origin).

In several embodiments, therapeutic agents are delivered through the use of, e.g., a gene therapy vector. In some such embodiments, an immune response may be developed against a portion of such vectors and/or their cargo (e.g., the therapeutic agent). Thus, in several embodiments, the antigen to which tolerance is desired comprises a gene therapy vector, including, but are not limited to: adenoviruses and adeno-associated virus (and corresponding variants −1, −2, −5, −6, −8, −9, and/or other parvoviruses), lentiviruses, and retroviruses.

Unwanted immune response in hematology and transplant includes autoimmune aplastic anemia, transplant rejection (generally), and Graft vs. Host Disease (bone marrow transplant rejection). In the embodiments where the tolerogenic antigen is a human allograft transplantation antigen, specific sequences can be selected from: subunits of the various MHC class I and MHC class II haplotype proteins and associated complexes with the peptide antigens present (for example, donor/recipient differences identified in tissue cross-matching), and single-amino-acid polymorphisms on minor blood group antigens including RhCE, Kell, Kidd, Duffy and Ss. Such compositions can be prepared individually for a given donor/recipient pair.

In Type-1 diabetes (e.g., type 1 diabetes mellitus), antigens include, but are not limited to: insulin, proinsulin, preproinsulin, glutamic acid decarboxylase-65 (GAD-65 or glutamate decarboxylase 2), GAD-67, glucose-6 phosphatase 2 (IGRP or islet-specific glucose 6 phosphatase catalytic subunit related protein), insulinoma-associated protein 2 (IA-2), and insulinoma-associated protein 2β (IA-2p); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5, or immunogenic fragments or portions of any of such antigens. It should be noted that insulin is an example of an antigen that can be characterized both as a self-antigen and a therapeutic protein antigen. For example, rHu Insulin and bovine insulin are therapeutic protein antigens (that are the subject of unwanted immune attack), whereas endogenous human insulin is a self-antigen (that is the subject of an unwanted immune attack). Because endogenous human insulin is not available to be employed in a pharmaceutical composition, a recombinant form is employed in certain embodiments of the compositions of the disclosure.

Human insulin, including an exogenously obtained form useful in the compositions of the disclosure, has the following sequence (UNIPROT P01308):

(SEQ ID NO: 1)
MALWMRLLPLLALLALWGPDPAAAFVNQHLCGSHLVEALYLVCGERGFF
YTPKTRREAEDLQVGQVELGGGPGAGSLQPLALEGSLQKRGIVEQCCTS
ICSLYQLENYCN.

GAD-65, including an exogenously obtained form useful in the compositions of the disclosure, has the following sequence (UNIPROT Q05329):

(SEQ ID NO: 2)
MASPGSGFWSFGSEDGSGDSENPGIARAWCQVAQKETGGIGNKLCALLY
GDAEKPAESGGSQPPRAAARKAACACDQKPCSCSKVDVNYAFLHATDLL
PACDGERPTLAFLQDVMNILIQYVVKSFDRSTKVIDFHYPNELLQEYNW
ELADQPQNLEEILMHCQTTLKYAIKTGHPRYFNQLSTGLDMVGLAADWL
TSTANTNMFTYEIAPVFVLLEYVTLKKMREIIGWPGGSGDGIFSPGGAI
SNMYAMMIARFKMFPEVKEKGMAALPRLIAFTSEHSHFSLKKGAAALGI
GTDSVILIKCDERGKMIPSDLERRILEAKQKGFVPFLVSATAGTTVYGA
FDPLLAVADICKKYKIWMHVDAAWGGGLLMSRKHKWKLSGVERANSVTW
NPHKMMGVPLQCSALLVREEGLMQNCNQMHASYLFQQDKHYDLSYDTGD
KALQCGRHVDVFKLWLMWRAKGTTGFEAHVDKCLELAEYLYNIIKNREG
YEMVFDGKPQHTNVCFWYIPPSLRTLEDNEERMSRLSKVAPVIKARMME
YGTTMVSYQPLGDKVNFFRMVISNPAATHQDIDFLIEEIERIGQDL.

IGRP, including an exogenously obtained form useful in the compositions of the disclosure, has the following sequence (UNIPROT QN9QR9):

(SEQ ID NO: 3)
MDFLHRNGVLHQHLQKDYRAYYTFLNFMSNVGDPRNIFFIYFPLCFQFN
QTVGTKMIWVAVIGDWLNLIFKWILFGHRPYWWVQETQIYPNHSSPCLE
QFPTTCETGPGSPSGHAMGASCVWYVMVTAALSHTVCGMDKFSITLHRL
TWSFLWSVFWLIQISVCISRVFIATHFPHQVILGVIGGMLVAEAFEHTP
GIQTASLGTYLKTNLFLFLFAVGFYLLLRVLNIDLLWSVPIAKKWCANP
DWIHIDTTPFAGIVRNLGVLFGLGFAINSEMFLLSCRGGNNYTLSFRLL
CALTSLTILQLYHFLQIPTHEEHLFYVLSFCKSASIPLTVVAFIPYSVH
MLMKQSGKKSQ.

In several embodiments, human proinsulin, including an exogenously obtained form useful in the tolerogenic compositions of the disclosure, has the following sequence:

(SEQ ID NO: 4)
FVNQHLCGSHLVEALYLVCGERGFFYTPKTRREAEDLQVGQVELGGGPG
AGSLQPLALEGSLQKRGIVEQCCTSICSLYQLENYCN.

Depending on the embodiment, peptides/epitopes useful in the tolerogenic compositions of the disclosure for treating type 1 diabetes include some or all of the following sequences, individually in a tolerogenic composition or together in a cocktail of tolerogenic compositions:

Human Proinsulin 1-70:
(SEQ ID NO: 5)
FVNQHLCGSHLVEALYLVCGERGFFYTPKTRREAEDLQVGQVELGGGPG
AGSLQPLALEGSLQKRGIVEQ;
Human Proinsulin 9-70:
(SEQ ID NO: 6)
SHLVEALYLVCGERGFFYTPKTRREAEDLQVGQVELGGGPGAGSLQPLA
LEGSLQKRGIVEQ;
Human Proinsulin 9-38:
(SEQ ID NO: 7)
SHLVEALYLVCGERGFFYTPKTRREAEDLQ;
Human Proinsulin 1-38:
(SEQ ID NO: 8)
FVNQHLCGSHLVEALYLVCGERGFFYTPKTRREAEDLQ;
Human Proinsulin 9-23:
(SEQ ID NO: 9)
SHLVEALYLVCGERG;
Human Proinsulin 45-71 (C13-A6):
(SEQ ID NO: 10)
GGGPGAGSLQPLALEGSLQKRGIVEQC;
Human Proinsulin C24-A1:
(SEQ ID NO: 11)
LALEGSLQKRG;
Human Proinsulin C19-A3:
(SEQ ID NO: 12)
GSLQPLALEGSLQKRGIV;
Human Proinsulin C13-32:
(SEQ ID NO: 13)
GGGPGAGSLQPLALEGSLQK;
Human Proinsulin B9-C4:
(SEQ ID NO: 14)
SHLVEALYLVCGERGFFYTPKTRREAED;
Human Proinsulin C22-A5:
(SEQ ID NO: 15)
QPLALEGSLQKRGIVEQ;
Human IA-2 718-782:
(SEQ ID NO: 16)
AYQAEPNTCATAQGEGNIKKNRHPDFLPYDHARIKLKVESSPSRSDYIN
ASPIIEHDPRMPAYIA;
Human IA-2 785-819:
(SEQ ID NO: 17)
GPLSHTIADFWQMVWESGCTVIVMLTPLVEDGVKQ;
Human IA-2 828-883:
(SEQ ID NO: 18)
GASLYHVYEVNLVSEHIWCEDFLVRSFYLKNVQTQETRTLTQFHFLSWP
AEGTPAS;
Human IA-2 943-979:
(SEQ ID NO: 19)
EHVRDQRPGLVRSKDQFEFALTAVAEEVNAILKALPQCG.

In autoimmune diseases of the thyroid, including Hashimoto's thyroiditis and Graves' disease, main antigens include, but are not limited to, thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor.

In Addison's Disease, main antigens include, but are not limited to, 21-hydroxylase, 17α-hydroxylase, and P450 side chain cleavage enzyme (P450scc); other antigens include ACTH receptor, P450c21 and P450c17.

In premature ovarian failure, main antigens include, but are not limited to, FSH receptor and α-enolase.

In autoimmune hypophysitis, or pituitary autoimmune disease, main antigens include, but are not limited to, pituitary gland-specific protein factor (PGSF) 1a and 2; another antigen is type 2 iodothyronine deiodinase.

In multiple sclerosis, main antigens include, but are not limited to, myelin basic protein (“MBP”), myelin oligodendrocyte glycoprotein (“MOG”) and myelin proteolipid protein (“PLP”).

MBP, including an exogenously obtained form useful in the compositions of the disclosure, has the following sequence (UNIPROT P02686):

(SEQ ID NO: 20)
MGNHAGKRELNAEKASTNSETNRGESEKKRNLGELSRTTSEDNEVFGEA
DANQNNGTSSQDTAVTDSKRTADPKNAWQDAHPADPGSRPHLIRLFSRD
APGREDNTFKDRPSESDELQTIQEDSAATSESLDVMASQKRPSQRHGSK
YLATASTMDHARHGFLPRHRDTGILDSIGRFFGGDRGAPKRGSGKDSHH
PARTAHYGSLPQKSHGRTQDENPVVHFFKNIVTPRTPPPSQGKGRGLSL
SRFSWGAEGQRPGFGYGGRASDYKSAHKGFKGVDAQGTLSKIFKLGGRD
SRSGSPMARR.

MOG, including an exogenously obtained form useful in the compositions of the disclosure, has the following sequence (UNIPROT Q16653):

(SEQ ID NO: 21)
MASLSRPSLPSCLCSFLLLLLLQVSSSYAGQFRVIGPRHPIRALVGDEV
ELPCRISPGKNATGMEVGWYRPPFSRVVHLYRNGKDQDGDQAPEYRGRT
ELLKDAIGEGKVTLRIRNVRFSDEGGFTCFFRDHSYQEEAAMELKVEDP
FYWVSPGVLVLLAVLPVLLLQITVGLIFLCLQYRLRGKLRAEIENLHRT
FDPHFLRVPCWKITLFVIVPVLGPLVALIICYNWLHRRLAGQFIEELRN
PF.

PLP, including an exogenously obtained form useful in the compositions of the disclosure, has the following sequence (UNIPROT P60201):

(SEQ ID NO: 22)
MGLLECCARCLVGAPFASLVATGLCFFGVALFCGCGHEALTGTEKLIET
YFSKNYQDYEYLINVIHAFQYVIYGTASFFFLYGALLLAEGFYTTGAVR
QIFGDYKTTICGKGLSATVTGGQKGRGSRGQHQAHSLERVCHCLGKWLG
HPDKFVGITYALTVVWLLVFACSAVPVYIYFNTWTTCQSIAFPSKTSAS
IGSLCADARMYGVLPWNAFPGKVCGSNLLSICKTAEFQMTFHLFIAAFV
GAAATLVSLLTFMIAATYNFAVLKLMGRGTKF.

Peptides/epitopes useful in the compositions of the disclosure for treating multiple sclerosis include some or all of the following sequences, individually in a tolerogenic composition as disclosed herein or together in a combination (e.g., a cocktail) of tolerogenic compositions:

MBP 13-32:
(SEQ ID NO: 23)
KYLATASTMDHARHGFLPRH;
MBP 83-99:
(SEQ ID NO: 24)
ENPWHFFKNIVTPRTP;
MBP 111-129:
(SEQ ID NO: 25)
LSRFSWGAEGQRPGFGYGG;
MBP 146-170:
(SEQ ID NO: 26)
AQGTLSKIFKLGGRDSRSGSPMARR;
MOG 1-20:
(SEQ ID NO: 27)
GQFRVIGPRHPIRALVGDEV;
MOG 35-55:
(SEQ ID NO: 28)
MEVGWYRPPFSRWHLYRNGK;
and
PLP 139-154:
(SEQ ID NO: 29)
HCLGKWLGHPDKFVGI
MOG 30-60:
(SEQ ID NO: 30)
KNATGMEVGWYRSPFSRVVHLYRNGKDQDAE
MBP 83-99:
(SEQ ID NO: 31)
ENPWHFFKNIVTPRTP
MOG 35-55:
(SEQ ID NO: 32)
MEVGWYRPPFSRVVHLYRNGK
MBP 82-98:
(SEQ ID NO: 33)
DENPVVHFFKNIVTPRT
MBP 82-99:
(SEQ ID NO: 34)
DENPVVHFFKNIVTPRTP
MBP 82-106:
(SEQ ID NO: 35)
DENPVVHFFKNIVTPRTPPPSQGKG
MBP 87-106:
(SEQ ID NO: 36)
VHFFKNIVTPRTPPPSQGKG
MBP 131-155:
(SEQ ID NO: 37)
ASDYKSAHKGLKGVDAQGTLSKIFK
PLP 41-58:
(SEQ ID NO: 38)
GTEKLIETYFSKNYQDYE
PLP 89-106:
(SEQ ID NO: 39)
GFYTTGAVRQIFGDYKTT
PLP 95-116:
(SEQ ID NO: 40)
AVRQIFGDYKTTICGKGLSATV
PLP 178-197:
(SEQ ID NO: 41)
NTWTTCQSIAFPSKTSASIG
PLP 190-209:
(SEQ ID NO: 42)
SKTSASIGSLCADARMYGVL
MOG 11-30:
(SEQ ID NO: 43)
PIRALVGDEVELPCRISPGK
MOG 21-40:
(SEQ ID NO: 44)
ELPCRISPGKNATGMEVGWY
MOG 64-86:
(SEQ ID NO: 45)
EYRGRTELLKDAIGEGKVTLRIR
MOG 1-62:
(SEQ ID NO. 46)
GQFRVIGPRHPIRALVGDEVELPCRISPGKNATGMEVGWYRPPFSRVVH
LYRNGKDQDGDQA
MBP 76-136:
(SEQ ID NO: 47)
SHGRTQDENPVVHFFKNIVTPRTPPPSQGKGRGLSLSRFSWGAEGQRPG
FGYGGRASDYKSCG

In rheumatoid arthritis, main antigens include, but are not limited to, collagen II, immunoglobulin binding protein, the fragment crystallizable region of immunoglobulin G, double-stranded DNA, and the natural and cirtullinated forms of proteins implicated in rheumatoid arthritis pathology, including fibrin/fibrinogen, vimentin, collagen I and II, and alpha-enolase.

In autoimmune gastritis, a main antigen is H+, K+-ATPase.

In pernicious angemis, a main antigen is intrinsic factor.

In celiac disease, main antigens include, but are not limited to, tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that while the main antigen of celiac disease is alpha gliadin, alpha gliadin turns more immunogenic in the body through deamidation by tissue glutaminase converting alpha gliadin's glutamines to glutamic acid. Thus, while alpha gliadin is originally a foreign food antigen, once it has been modified in the body to become more immunogenic it can be characterized as a self-antigen, depending on the embodiment.

In vitiligo, a main antigen is tyrosinase, and tyrosinase related protein 1 and 2.

MART1, Melanoma antigen recognized by T cells 1, Melan-A, including an exogenously obtained form useful in the compositions of the disclosure, has the following sequence (UNIPROT Q16655):

(SEQ ID NO: 48)
MPREDAHFIYGYPKKGHGHSYTTAEEAAGIGILTVILGVLLLIGCWYCR
RRNGYRALMDKSLHVGTQCALTRRCPQEGFDHRDSKVSLQEKNCEPVVP
NAPPAYEKLSAEQSPPPYSP,

Tyrosinase, including an exogenously obtained form useful in the compositions of the disclosure, has the following sequence (UNIPROT P14679):

(SEQ ID NO: 49)
MLLAVLYCLLWSFQTSAGHFPRACVSSKNLMEKECCPPWSGDRSPCGQL
SGRGSCQNILLSNAPLGPQFPFTGVDDRESWPSVFYNRTCQCSGNFMGF
NCGNCKFGFWGPNCTERRLLVRRNIFDLSAPEKDKFFAYLTLAKHTISS
DYVIPIGTYGQMKNGSTPMFNDINIYDLFVWMHYYVSMDALLGGSEIWR
DIDFAHEAPAFLPWHRLFLLRWEQEIQKLTGDENFTIPYWDWRDAEKCD
ICTDEYMGGQHPTNPNLLSPASFFSSWQIVCSRLEEYNSHQSLCNGTPE
GPLRRNPGNHDKSRTPRLPSSADVEFCLSLTQYESGSMDKAANFSFRNT
LEGFASPLTGIADASQSSMHNALHIYMNGTMSQVQGSANDPIFLLHHAF
VDSIFEQWLRRHRPLQEVYPEANAPIGHNRESYMVPFIPLYRNGDFFIS
SKDLGYDYSYLQDSDPDSFQDYIKSYLEQASRIWSWLLGAAMVGAVLTA
LLAGLVSLLCRHKRKQLPEEKQPLLMEKEDYHSLYQSHL.

Melanocyte protein PMEL, gp100, including an exogenously obtained form useful in the compositions of the disclosure, has the following sequence (UNIPROT P40967):

(SEQ ID NO: 50)
MDLVLKRCLLHLAVIGALLAVGATKVPRNQDWLGVSRQLRTKAWNRQLY
PEWTEAQRLDCWRGGQVSLKVSNDGPTLIGANASFSIALNFPGSQKVLP
DGQVIWVNNTIINGSQVWGGQPVYPQETDDACIFPDGGPCPSGSWSQKR
SFVYVWKTWGQYWQVLGGPVSGLSIGTGRAMLGTHTMEVTVYHRRGSRS
YVPLAHSSSAFTITDQVPFSVSVSQLRALDGGNKHFLRNQPLTFALQLH
DPSGYLAEADLSYTWDFGDSSGTLISRALVVTHTYLEPGPVTAQVVLQA
AIPLTSCGSSPVPGTTDGHRPTAEAPNTTAGQVPTTEVVGTTPGQAPTA
EPSGTTSVQVPTTEVISTAPVQMPTAESTGMTPEKVPVSEVMGTTLAEM
SIPEATGMTPAEVSIVVLSGTTAAQVTTTEWVETTARELPIPEPEGPDA
SSIMSTESIIGSLGPLLDGTATLRLVKRQVPLDCVLYRYGSFSVTLDIV
QGIESAEILQAVPSGEGDAFELTVSCQGGLPKEACMEISSPGCQPPAQR
LCQPVLPSPACQLVLHQILKGGSGTYCLNVSLADTNSLAVVSTQLIMPG
QEAGLGQVPLIVGILLVLMAVVLASLIYRRRLMKQDFSVPQLPHSSSHW
LRLPRIFCSCPIGENSPLLSGQQV.

In myasthenia gravis, a main antigen is acetylcholine receptor. Other myasthenia gravis antigens may include MuSK (muscle-specific kinase) and LRP4 (lipoprotein receptor-related protein 4).

In pemphigus vulgaris and variants, main antigens include, but are not limited to, desmoglein 3, 1 and 4; other antigens include pemphaxin, desmocollins, plakoglobin, perplakin, desmoplakins, and acetylcholine receptor.

In bullous pemphigoid, main antigens include BP180 and BP230; other antigens include plectin and laminin 5.

In dermatitis herpetiformis Duhring, main antigens include, but are not limited to, endomysium and tissue transglutaminase.

In epidermolysis bullosa acquisita, a main antigen is collagen VII.

In systemic sclerosis, main antigens include, but are not limited to, matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, I and III, PM-Slc, fibrillarin, and B23.

In mixed connective tissue disease, a main antigen is U1snRNP.

In Sjogren's syndrome, the main antigens include, but are not limited to, nuclear antigens SS-A and SS-B; other antigens include fodrin, poly(ADP-ribose) polymerase and topoisomerase, muscarinic receptors, and the Fc-gamma receptor IIIb.

In systemic lupus erythematosus, main antigens include nuclear proteins including the “Smith antigen,” SS-A, high mobility group box 1 (HMGB1), nucleosomes, histone proteins and double-stranded DNA (against which auto-antibodies are made in the disease process).

In Goodpasture's syndrome, main antigens include, but are not limited to, glomerular basement membrane proteins including collagen IV.

In rheumatic heart disease, a main antigen is cardiac myosin.

In autoimmune polyendocrine syndrome type 1 antigens include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501 A2 and 2A6, SOX-9, SOX-10, calcium-sensing receptor protein, and the type 1 interferons interferon alpha, beta and omega.

In neuromyelitis optica, a main antigen is aquaporin-4 (AQP4).

Aquaporin-4, including an exogenously obtained form useful in the compositions of the disclosure, has the following sequence (UNIPROT P55087):

(SEQ ID NO: 51)
MSDRPTARRWGKCGPLCTRENIMVAFKGVWTQAFWKAVTAEFLAMLIFV
LLSLGSTINWGGTEKPLPVDMVLISLCFGLSIATMVQCFGHISGGHINP
AVTVAMVCTRKISIAKSVFYIAAQCLGAIIGAGILYLVTPPSVVGGLGV
TMVHGNLTAGHGLLVELIITFQLVFTIFASCDSKRTDVTGSIALAIGFS
VAIGHLFAINYTGASMNPARSFGPAVIMGNWENHWIYWVGPIIGAVLAG
GLYEYVFCPDVEFKRRFKEAFSKAAQQTKGSYMEVEDNRSQVETDDLIL
KPGVVHVIDVDRGEEKKGKDQSGEVLSSV.

In uveitis, main antigens include Retinal S-antigen or “S-arrestin” and interphotoreceptor retinoid binding protein (IRBP) or retinol-binding protein 3.

S-arrestin, including an exogenously obtained form useful in the compositions of the disclosure, has the following sequence (UNIPROT P10523):

(SEQ ID NO: 52)
MAASGKTSKSEPNHVIFKKISRDKSVTIYLGNRDYIDHVSQVQPVDGVV
LVDPDLVKGKKVYVTLTCAFRYGQEDIDVIGLTFRRDLYFSRVQVYPPV
GAASTPTKLQESLLKKLGSNTYPFLLTFPDYLPCSVMLQPAPQDSGKSC
GVDFEVKAFATDSTDAEEDKIPKKSSVRLLIRKVQHAPLEMGPQPRAEA
AWQFFMSDKPLHLAVSLNKEIYFHGEPIPVTVTVTNNTEKTVKKIKAFV
EQVANVVLYSSDYYVKPVAMEEAQEKVPPNSTLTKTLTLLPLLANNRER
RGIALDGKIKHEDTNLASSTIIKEGIDRTVLGILVSYQIKVKLTVSGFL
GELTSSEVATEVPFRLMHPQPEDPAKESYQDANLVFEEFARHNLKDAGE
AEEGKRDKNDVDE.

IRBP, including an exogenously obtained form useful in the compositions of the disclosure, has the following sequence (UNIPROT P10745):

(SEQ ID NO: 53)
MMREWVLLMSVLLCGLAGPTHLFQPSLVLDMAKVLLDNYCFPENLLGMQ
EAIQQAIKSHEILSISDPQTLASVLTAGVQSSLNDPRLVISYEPSTPEP
PPQVPALTSLSEEELLAWLQRGLRHEVLEGNVGYLRVDSVPGQEVLSMM
GEFLVAHVWGNLMGTSALVLDLRHCTGGQVSGIPYIISYLHPGNTILHV
DTIYNRPSNTTTEIWTLPQVLGERYGADKDVVVLTSSQTRGVAEDIAHI
LKQMRRAIVVGERTGGGALDLRKLRIGESDFFFTVPVSRSLGPLGGGSQ
TWEGSGVLPCVGTPAEQALEKALAILTLRSALPGVVHCLQEVLKDYYTL
VDRVPTLLQHLASMDFSTVVSEEDLVTKLNAGLQAASEDPRLLVRAIGP
TETPSWPAPDAAAEDSPGVAPELPEDEAIRQALVDSVFQVSVLPGNVGY
LRFDSFADASVLGVLAPYVLRQVWEPLQDTEHLIMDLRHNPGGPSSAVP
LLLSYFQGPEAGPVHLFTTYDRRTNITQEHFSHMELPGPRYSTQRGVYL
LTSHRTATAAEEFAFLMQSLGWATLVGEITAGNLLHTRTVPLLDTPEGS
LALTVPVLTFIDNHGEAWLGGGVVPDAIVLAEEALDKAQEVLEFHQSLG
ALVEGTGHLLEAHYARPEVVGQTSALLRAKLAQGAYRTAVDLESLASQL
TADLQEVSGDHRLLVFHSPGELVVEEAPPPPPAVPSPEELTYLIEALFK
TEVLPGQLGYLRFDAMAELETVKAVGPQLVRLVWQQLVDTAALVIDLRY
NPGSYSTAIPLLCSYFFEAEPRQHLYSVFDRATSKVTEVWTLPQVAGQR
YGSHKDLYILMSHTSGSAAEAFAHTMQDLQRATVIGEPTAGGALSVGIY
QVGSSPLYASMPTQMAMSATTGKAWDLAGVEPDITVPMSEALSIAQDIV
ALRAKVPTVLQTAGKLVADNYASAELGAKMATKLSGLQSRYSRVTSEVA
LAEILGADLQMLSGDPHLKAAHIPENAKDRIPGIVPMQIPSPEVFEELI
KFSFHTNVLEDNIGYLRFDMFGDGELLTQVSRLLVEHIWKKIMHTDAMI
IDMRFNIGGPTSSIPILCSYFFDEGPPVLLDKIYSRPDDSVSELWTHAQ
VVGERYGSKKSMVILTSSVTAGTAEEFTYIMKRLGRALVIGEVTSGGCQ
PPQTYHVDDTNLYLTIPTARSVGASDGSSWEGVGVTPHVVVPAEEALAR
AKEMLQHNQLRVKRSPGLQDHL.

In the embodiments where the tolerogenic antigen is a foreign antigen against which an unwanted immune response can be developed, such as food antigens, specific antigens include, but are not limited to:

from peanut: conarachin (Ara h 1), allergen II (Ara h 2), arachis agglutinin, conglutin (Ara h 6);

conarachin, for example has the sequence identified as UNIPROT Q6PSU6

from apple: 31 kda major allergen/disease resistance protein homolog (Mal d 2), lipid transfer protein precursor (Mal d 3), major allergen Mal d 1.03D (Mal d 1);

from milk: α-lactalbumin (ALA), lactotransferrin; from kiwi: actinidin (Act c 1, Act d 1), phytocystatin, thaumatin-like protein (Act d 2), kiwellin (Act d 5);

from egg whites: ovomucoid, ovalbumin, ovotransferrin, and lysozyme;

from egg yolks: livetin, apovitillin, and vosvetin;

from mustard: 2S albumin (Sin a 1), 11 S globulin (Sin a 2), lipid transfer protein (Sin a 3), profilin (Sin a 4);

from celery: profilin (Api g 4), high molecular weight glycoprotein (Api g 5);

from shrimp: Pen a 1 allergen (Pen a 1), allergen Pen m 2 (Pen m 2), tropomyosin fast isoform;

from wheat and/or other cereals: high molecular weight glutenin, low molecular weight glutenin, alpha-, gamma- and omega-gliadin, hordein, secalin and/or avenin;

peptides/epitopes useful in the compositions of the disclosure for treating Celiac Disease include some or all of the following sequences, individually in a composition of Formula (1) or together in a cocktail of compositions of Formula (1):

HLA-DQ-2.5 relevant, Alpha-gliadin “33-mer”
native:
(SEQ ID NO: 54)
LQLQPFPQPQLPYPQPQLPYPQPQLPYPQPQPF
HLA-DQ-2.5 relevant, Alpha-gliadin “33-mer”
deamidated:
(SEQ ID NO: 55)
LQLQPFPQPELPYPQPELPYPQPELPYPQPQPF
HLA-DQ-8 relevant, Alpha-gliadin:
(SEQ ID NO: 56)
QQYPSGQGSFQPSQQNPQ
HLA-DQ-8 relevant, Omega-gliadin (wheat,
U5UA46):
(SEQ ID NO: 57)
QPFPQPEQPFPW
Alpha-gliadin “15-mer” fragment:
(SEQ ID NO: 58)
ELQPFPQPELPYPQP
Gliadin linker:
(SEQ ID NO: 59)
GCRGGGPQPQPFPSQQPY
Gliadin extended:
(SEQ ID NO: 60)
GCGPQPQPFPSQQPYLQLQPFPQPQLPYPQPQLPYPQPQLPYPQPQPF
Gliadin deamidated extended:
(SEQ ID NO: 61)
GCGPQPQPFPSQQPYLQLQPFPQPELPYPQPELPYPQPELPYPQPQPF

from strawberry: major strawberry allergy Fra a 1-E (Fra a 1); and

from banana: profilin (Mus xp 1).

In the embodiments where the antigen is a foreign antigen against which an unwanted immune response is developed, such as to animal, plant and environmental antigens, specific antigens can, for example, be: cat, mouse, dog, horse, bee, dust, tree and goldenrod, including the following proteins or peptides derived from:

weeds, (including ragweed allergens amb a 1, 2, 3, 5, and 6, and Amb t 5; pigweed Che a 2 and 5; and other weed allergens Par j 1, 2, and 3, and Par o 1);

grass (including major allergens Cyn d 1, 7, and 12; Dac g 1, 2, and 5; Hol I 1.01203: Lol p 1, 2, 3, 5, and 11; Mer a 1; Pha a 1; Poa p 1 and 5);

pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut);

dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blo t 1, 2, 3, 4, 5, 6, 10, 11, 12, 13, 19, and 21; also allergens Eur m 2 from Euroglyphus maynei, Tyr p 13 from Tyrophagus putrescentiae, and allergens Bla g 1, 2, and 4; Per a 1, 3, and 7 from cockroach);

pets (including cats, dogs, rodents, and farm animals; major cat allergens include Fel d 1 through 8, cat IgA, BLa g 2, and cat albumin; major dog allergens include Can f 1 through 6, and dog albumin);

bee stings, including major allergens Api m 1 through 12; and

fungus, including allergens derived from, species of Aspergillus and Penicillium, as well as the species Alternaria alternata, Davidiella tassiana, and Trichophyton rubrum.

In Parkinson's disease, the main antigen is alpha synuclein. Alpha synuclein, including an exogenously obtained form useful in the tolerogenic compositions of the disclosure, has the following sequence (UNIPROT P37840):

(SEQ ID NO: 62)
MDVFMKGLSKAKEGVVAAAEKTKQGVAEAAGKTKEGVLYVGSKTKEGVV
HGVATVAEKTKEQVTNVGGAVVTGVTAVAQKTVEGAGSIAAATGFVKKD
QLGKNEEGAPQEGILEDMPVDPDNEAYEMPSEEGYQDYEPEA.

In several embodiments, the antigen to which tolerance is desired is a viral antigen, for example a viral antigen derived from a therapeutic viral vector, such as an adeno-associated viral vector (AAV). In several embodiments, the antigen to which tolerance is desired comprises is or is an immunogenic fragment derived from the AAV serotype 2 capsid protein 1 (SEQ ID NO: 63). In several embodiments, the antigen to which tolerance is desired comprises is or is an immunogenic fragment derived from the AAV serotype 2 capsid protein 2 (SEQ ID NO: 64). In several embodiments, the antigen to which tolerance is desired comprises is or is an immunogenic fragment derived from the AAV serotype 2 capsid protein 3 (SEQ ID NO: 65). In several embodiments, the antigen to which tolerance is desired comprises is or is an immunogenic fragment derived from the AAV serotype 9 capsid protein 1 (SEQ ID NO: 66). In several embodiments, the antigen to which tolerance is desired comprises is or is an immunogenic fragment derived from the AAV serotype 9 capsid protein 2 (SEQ ID NO: 67). In several embodiments, the antigen to which tolerance is desired comprises is or is an immunogenic fragment derived from the AAV serotype 9 capsid protein 3 (SEQ ID NO: 68).

In anti-neutrophil cytoplasmic antibody-associated vasculitis (ANCA-V), main antigens include, but are not limited to, myeloperoxidase (MPO) and proteinase-3/myeloblastin (PR3). In several embodiments, the antigen to which tolerance is desired comprises is or is an immunogenic fragment derived from Myeloblastin (SEQ ID NO: 69). In several embodiments, the antigen to which tolerance is desired comprises is or is an immunogenic fragment derived from Myeloperoxidase (SEQ ID NO: 70).

The antigen can be a complete protein, a portion of a complete protein, a peptide, or the like, and can be derivatized (as discussed above) for attachment to a linker and/or a galactosylating moiety, and/or a glucosylating moiety, can be a variant and/or can contain conservative substitutions, particularly maintaining sequence identity, and/or can be desialylated.

### Terminal End Unit

As disclosed elsewhere herein, in several embodiments, the antigen is bonded (e.g., covalently) to one end of the linker, the terminal end unit is bonded (e.g., covalently) to another end of the linker. In several embodiments, as disclosed elsewhere herein, the terminal end unit is attached to the linker via a carbon-carbon bond. For example, the terminal end unit comprises a carbon that is bonded to a carbon of the linker. In several embodiments, the carbon of the terminal end unit (e.g., that is bonded to the carbon of the linker) has three other valencies (e.g., positions) available for bonding to other substituents. In several embodiments, any one or more of the other valencies (e.g., positions) of the linker-bonded terminal end unit carbon is occupied by a substitutent independently selected from —H, optionally substituted C₁₋₁₁ alkyl, —CN, optionally substituted —C(═NH)NH₂, optionally substituted —C(═NH)NH(C₁₋₃ alkyl), optionally substituted imidazoline, optionally substituted aryl, optionally substituted heteroaryl, optionally substituted aralkyl, optionally substituted heteroaralkyl, optionally substituted C-carboxy (where R is —H, optionally substituted C₁₋₆ alkyl, optionally substituted C₁₋₆ alkylenyl, or optionally substituted phenyl), optionally substituted C-amido (where R_{A} and R_{B} are independently —H or optionally substituted C₁₋₆ alkyl), optionally substituted succinimidyl ester, optionally substituted isoindolin-1,3-dione, an optionally substituted polystyryl unit, an optionally substituted polyacrylic acid, or two positions on the carbon are taken together to provide an optionally substituted C₃₋₁₀ cycloalkyl or optionally substituted heterocyclyl (having 3 to 10 members in the ring). In several embodiments, a position of the linker-bonded terminal end unit carbon is occupied by —CN. In several embodiments, a position of the linker-bonded terminal end unit is occupied by a polymeric (or partially polymeric) unit. In several embodiments, optional substitutions of substituents on the linker-bonded terminal end unit carbon (e.g., optional substitutions of substituents on the Y-bonded EU carbon), where present, are independently selected from C₁₋₃ alkyl, C₁₋₆ alkoxy, C₁₋₆ alkylenyl, hydroxyl, amino, halogen, C-carboxy (where R is —H, C₁₋₆ alkyl, or polyethylene glycol (PEG) (e.g., having repeat units numbering from equal to or at least about 1, 2, 3, 4, 5, 10, 15, 20, 30, 40, 50, 75, 100, 150, or ranges including and/or spanning the aforementioned values)), succinimidyl ester, 2-nitro-5-(prop-2-yn-1-yloxy)benzyl 4-cyanopentanoate, azide (N₃), C₁₋₃ alkyl azide, C₁₋₃ alkyl silane (e.g., trimethyl silane, triethyl silane, tripropyl silane), polyethylene glycol (PEG) (e.g., having repeat units numbering from equal to or at least about 1, 2, 3, 4, 5, 10, 15, 20, 30, 40, 50, 75, 100, 150, or ranges including and/or spanning the aforementioned values), and/or combinations of the foregoing. For example, where present the linker-bonded (e.g., Y-bonded) terminal end unit (e.g., EU) carbon may comprise a C₁₋₁₁ alkyl substituted with a C-carboxy where R is H. In several embodiments, the Y-bonded EU carbon substituent may lack optional substituents, may be optionally substituted by one optional substituent, or may be optionally substituted by multiple (2, 3, or more) optional substituents.

In several embodiments, a position of the linker-bonded terminal end unit carbon is occupied by —CN. In several embodiments, one or more positions of the linker-bonded terminal end group unit is occupied by C₁₋₆ alkyl that is unsubstituted. In several embodiments, one or more positions of the linker-bonded terminal end unit carbon is occupied by C₁₋₁₁ alkyl that is unsubstituted. In several embodiments, one or more positions of the linker-bonded terminal end unit carbon is occupied by C₁₋₆alkyl that is substituted with one or more of —OH, —OCH₃, C-carboxy (where R is —H, optionally substituted C₁₋₆alkyl, optionally substituted C₁₋₆ alkylenyl, succinimidyl ester, 2-nitro-5-(prop-2-yn-1-yloxy)benzyl 4-cyanopentanoate, or PEG), or C-amido (where R_{A} and R_{B} are independently —H or optionally substituted C₁₋₆ alkyl). In several embodiments, one or more positions of the linker-bonded terminal end unit carbon is occupied by C-carboxy (where R is —H, optionally substituted C₁₋₆ alkyl, optionally substituted C₁₋₆ alkylenyl, or phenyl optionally substituted with one or more halogens). In several embodiments, one or more positions of the linker-bonded terminal end unit carbon is occupied by C-amido (where R_{A} and R_{B} are independently —H or optionally substituted C₁₋₆ alkyl). In several embodiments, one or more positions of the linker-bonded terminal end unit carbon is occupied by C-amido (R_{A} and R_{B} are independently —H, C₁₋₆ alkyl optionally substituted with an azide, or C₁₋₆ alkylenyl). In several embodiments, one or more positions of the linker-bonded terminal end unit carbon is occupied by —C(═NH)NH₂ (optionally substituted with C₁₋₆ alkyl). In several embodiments, one or more positions of the linker-bonded terminal end unit carbon is occupied by optionally substituted phenyl. In several embodiments, one or more positions of the linker-bonded terminal end unit carbon is occupied by an optionally substituted heterocyclyl. In several embodiments, one or more positions of the linker-bonded terminal end unit carbon is occupied by succinimidyl ester. In several embodiments, one or more positions of the linker-bonded terminal end unit carbon is occupied by isoindolin-1,3-dione. In several embodiments, one or more positions of the linker-bonded terminal end unit carbon is occupied by an alkyl silane. In several embodiments, optional substitutions, where present, are as disclosed elsewhere herein. In several embodiments, optional substitutions, where present, are independently selected from halogen, —OH, —N₃, C-carboxy (where R is H or C₁₋₃ alkyl). In several embodiments, two positions of the linker-bonded terminal end unit carbon is occupied by groups selected from the group consisting of —H and methyl.

In several embodiments, the carbon-carbon bond between the linker and the terminal end unit may be described using Formula (1). For example, EU is attached to Y via a carbon-carbon bond. In several embodiments, any one or more of the other positions of the Y-bonded EU carbon is occupied by a substitutent independently selected from R¹² and R¹⁴. In several embodiments, EU is represented by (EU1):

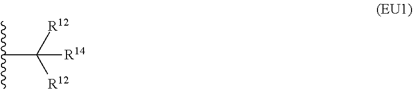

where the variables are as disclosed elsewhere herein. For example, in some embodiments, [symbol] denotes a connection to Y (via a carbon of the Y portion of the construct); R¹⁴ is selected from optionally substituted C₁₋₁₁ alkyl, —CN, optionally substituted —C(═NH)NH-2, optionally substituted heterocyclyl, optionally substituted aryl, optionally substituted heteroaryl, optionally substituted aralkyl, optionally substituted heteroaralkyl, C-carboxy (where R is —H, optionally substituted C₁₋₆ alkyl, optionally substituted C₁₋₆ alkylenyl, or optionally substituted phenyl), optionally substituted C-amido (where R_{A} and R_{B} are independently —H or optionally substituted C₁₋₆ alkyl), optionally substituted succinimidyl ester, optionally substituted isoindolin-1,3-dione, an alkyl silane, an optionally substituted polystyryl unit, an optionally substituted polyacrylic acid; and each instance of R¹² is independently hydrogen, optionally substituted C₁₋₆alkyl, C-carboxy (where R is —H or optionally substituted C₁₋₆alkyl), C-amido (where R_{A} and R_{B} are independently —H or optionally substituted C₁₋₆ alkyl), or each instance of R¹² is taken together to provide an optionally substituted C₃₋₁₀ cycloalkyl. In some embodiments, optional substitutions, where present on an EU group substituent (e.g., on any one of R², R¹², or R¹⁴), are independently selected from C₁₋₃ alkyl, C₁₋₆alkoxy, C₁₋₆alkylenyl, hydroxyl, amino, halogen, C-carboxy (where R is —H, C₁₋₆ alkyl, or PEG), succinimidyl ester, 2-nitro-5-(prop-2-yn-1-yloxy)benzyl 4-cyanopentanoate, —N₃, C₁₋₃ alkyl azide, C₁₋₃ alkyl silane, PEG, and/or combinations of the foregoing. In several embodiments, R¹² and/or each instance of R¹⁴ is independently selected from —H, optionally substituted C₁₋₁₁ alkyl, —CN, optionally substituted —C(═NH)NH₂, optionally substituted —C(═NH)NH(C₁₋₃ alkyl), optionally substituted imidazoline, optionally substituted heterocyclyl, optionally substituted aryl, optionally substituted heteroaryl, optionally substituted aralkyl, optionally substituted heteroaralkyl, C-carboxy (where R, as defined elsewhere herein, is —H, optionally substituted C₁₋₆ alkyl, optionally substituted C₁₋₆ alkylenyl, or optionally substituted phenyl), optionally substituted C-amido (where R_{A} and R_{B}, as defined elsewhere herein, are independently —H or optionally substituted C₁₋₆ alkyl), optionally substituted succinimidyl ester, optionally substituted isoindolin-1,3-dione, an alkyl silane, an optionally substituted polystyryl unit, an optionally substituted polyacrylic acid.

In several embodiments, any instance of R¹² or R¹⁴ (of EU) may independently be selected from the following:

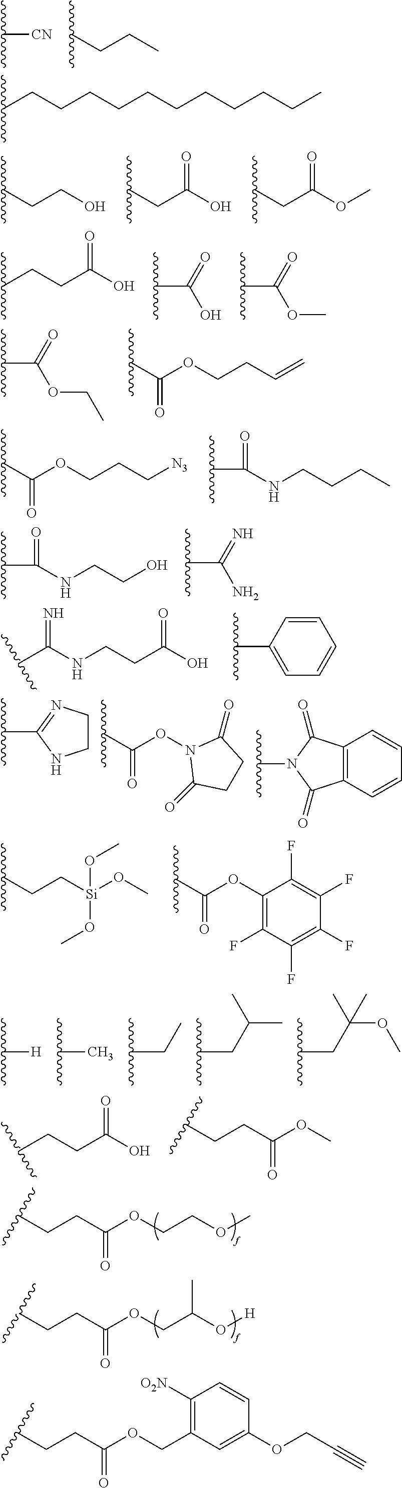

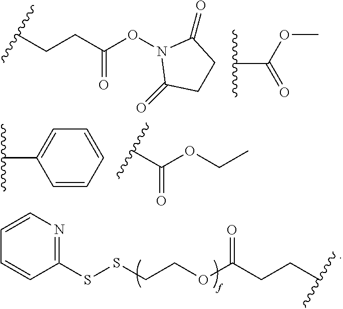

In several embodiments, f, where present, is an integer greater than or equal to about: 0, 1, 2, 3, 4, 5, 10, 20, 40, 50, 100, 150, 200, or ranges spanning and/or including the aforementioned values.

In several embodiments, each instance of R¹⁴ is selected from the group consisting of:

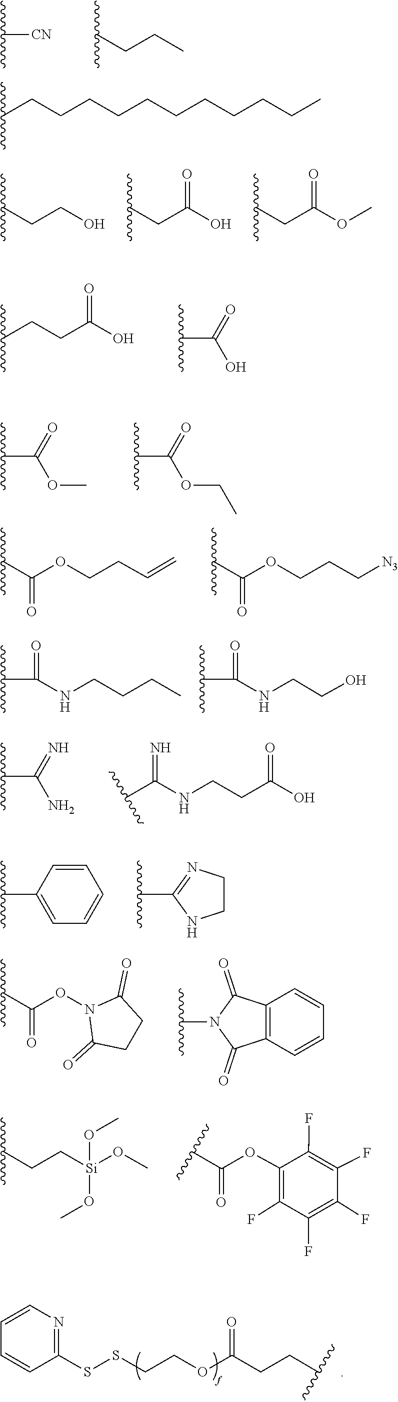

In several embodiments, f, where present, is an integer greater than or equal to about: 0, 1, 2, 3, 4, 5, 10, 20, 40, 50, 100, 150, 200, or ranges spanning and/or including the aforementioned values.

In several embodiments, each instance of R¹² is independently selected from the group consisting of:

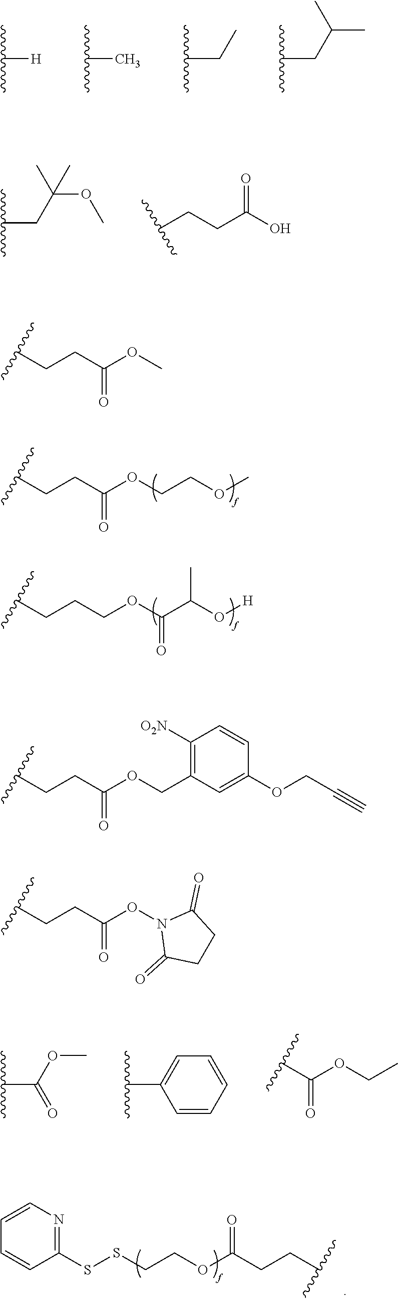

In several embodiments, f, where present, is an integer greater than or equal to about: 0, 1, 2, 3p 4, 5, 10, 20, 40, 50, 100, 150, 200, or ranges spanning and/or including the aforementioned values.

In several embodiments, EU is represented by (EU2):

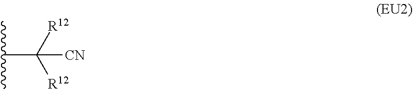

where the variables are as disclosed elsewhere herein. For example, in some embodiments, [symbol] denotes a connection to Y (via a carbon of the Y portion of the construct) and each instance of R¹² is independently hydrogen, optionally substituted C₁₋₆ alkyl, C-carboxy (where R is —H or optionally substituted C₁₋₆ alkyl), C-amido (where R_{A} and R_{B} are independently —H or optionally substituted C₁₋₆ alkyl), or each instance of R¹² is taken together to provide an optionally substituted C₃₋₁₀ cycloalkyl. In several embodiments, each instance of R¹² is independently hydrogen, an optionally substituted alkyl (e.g., a C₁₋₆ alkyl optionally substituted with one or more of a halogen, C-carboxy, -amino, —OH, etc.), or each instance of R¹² is taken together to provide an optionally substituted cycloalkyl.

In several embodiments, EU is selected from the group consisting of:

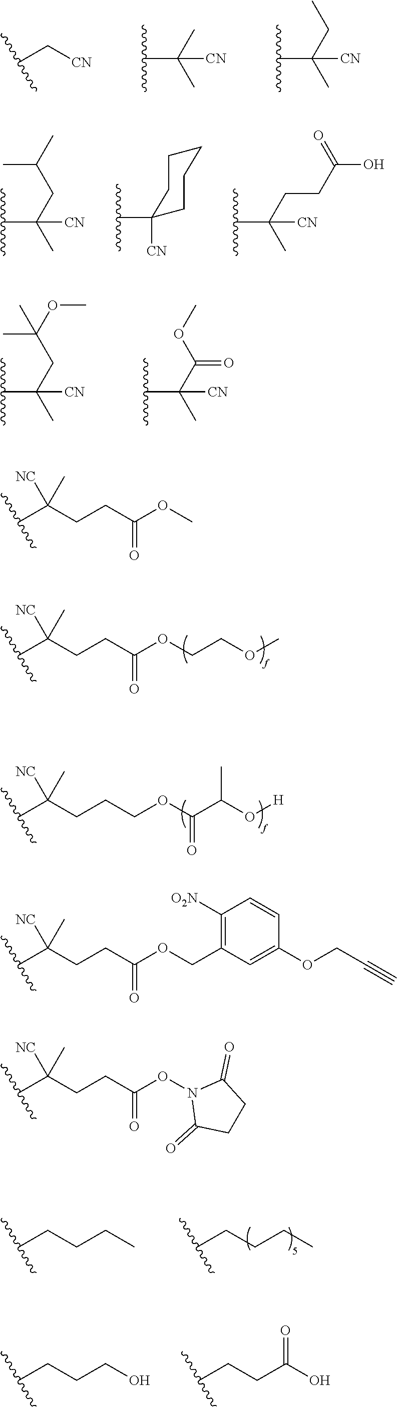

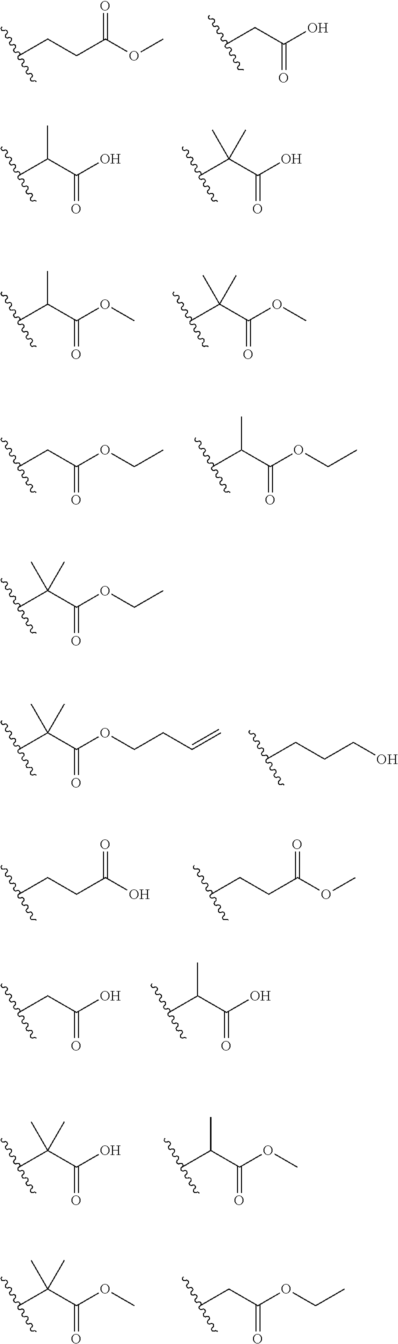

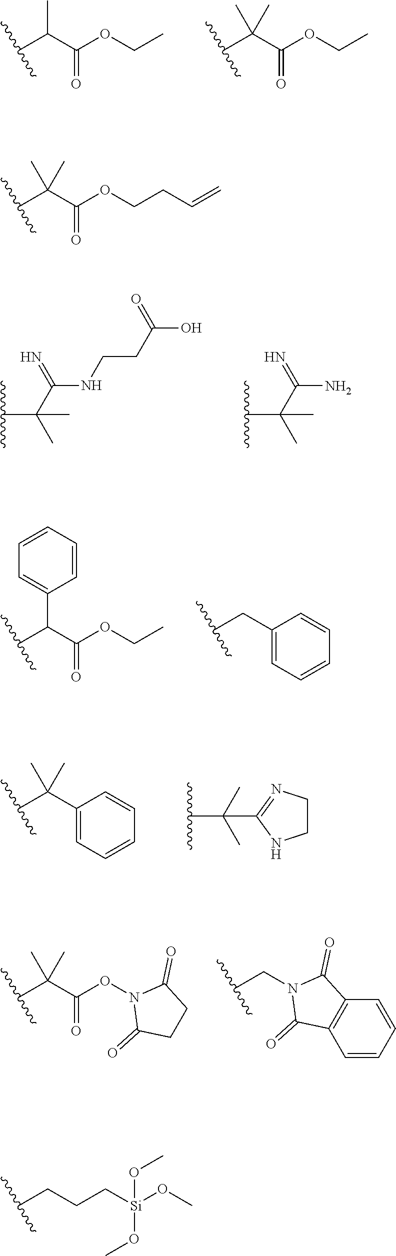

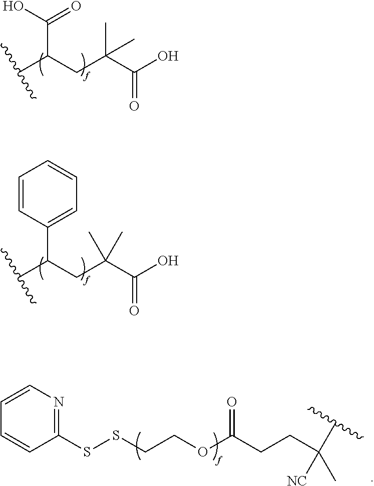

In several embodiments, f, where present, is an integer greater than or equal to about: 0, 1, 2, 3, 4, 5, 10, 20, 40, 50, 100, 150, 200, or ranges spanning and/or including the aforementioned values.

In some embodiments, R¹⁴ is —CN and each instance of R¹² is —CH₃ and EU is the following group:

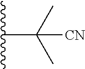 [SMILES: *C(C)(C)C#N]

In several embodiments, the terminal end unit (e.g., EU) lacks a dithioester (e.g., —S—C(═S)—). In some embodiments, the terminal end unit (e.g., EU) is not a dithiobenzoate, the terminal end unit (e.g., EU) is not a trithiocarbonate, and the terminal end unit (e.g., EU) is not a xanthate. In some embodiments, R² (e.g., the RAFT remnant) is replaced by —H. In several embodiments, the terminal end unit is not —H.

In several embodiments, as disclosed elsewhere herein, the terminal end unit is used to displace the CTA remnant. In several embodiments, equal to or at least 50%, 70%, 80%, 90%, 95%, 99%, or 99.9% (or ranges spanning and/or including the aforementioned values) of the construct comprises the terminal end unit (e.g., EU). In several embodiments, less than or equal to 30%, 25%, 20%, 15%, 10%, 5%, 1%, or 0.1% (or ranges spanning and/or including the aforementioned values) of the CTA remnant remains on the construct after it is displaced (e.g., by an terminal end unit).

Several embodiments pertain to a composition (e.g., a pharmaceutical composition) comprising a construct where the CTA remnant has been removed completely, has been substantially removed, or has been partially removed. Several embodiments pertain to a composition (e.g., a pharmaceutical composition) comprising a construct where the CTA remnant has been displaced by a terminal end unit. In several embodiments, the composition comprises a compound where equal to or at least 50%, 70%, 80%, 90%, 95%, 99%, or 99.9% (or ranges spanning and/or including the aforementioned values) of the CTA remnant is removed from the construct. In several embodiments, equal to or at least 50%, 70%, 80%, 90%, 95%, 99%, or 99.9% (or ranges spanning and/or including the aforementioned values) of the construct in the composition comprises the terminal end unit (e.g., EU). In several embodiments, equal to or at least 50%, 70%, 80%, 90%, 95%, 99%, or 99.9% (or ranges spanning and/or including the aforementioned values) of the construct in the composition lacks the CTA remnant. In several embodiments, less than or equal to 30%, 25%, 20%, 15%, 100, 5%, 1%, or 0.1% (or ranges spanning and/or including the aforementioned values) of the CTA remnant remains on the construct of the composition after the CTA remnant is displaced (e.g., by an terminal end unit). In several embodiments, equal to or at least 50%, 70%, 80%, 90%, 95%, 99%, or 99.9% (or ranges spanning and/or including the aforementioned values) of the CTA remnant is removed from the construct of the composition.

### Methods of Inducing Tolerance

Also provided herein are methods of inducing tolerance to antigens which, when administered alone (e.g., without the presently disclosed compositions) would result in an adverse immune response. In several embodiments, the compositions provided herein are used in the treatment, prevention, reduction or otherwise alter an immune response to an antigen. In several embodiments, the immune response has, or is occurring in an ongoing manner, while in some embodiments, the treatment and use of the compositions is in a prophylactic manner. For instance, in some embodiments, the administration (e.g., to a subject) is performed before, after, or before and after exposure to the antigen. In several embodiments, administration prior to exposure serves a prophylactic effect, which in several embodiments essentially avoids or significantly reduces in the immune response. Administration of the compositions can be via a variety of methods, including, but not limited to intravenous, infusion, intramuscular, oral, transdermal, intradermal, or other administration route. In several embodiments, the compositions are delivered in a therapeutically effective amount, for example, by a systemic or local route (e.g., intravenous, intraarterially, locally, intramuscular, subcutaneous, etc.). Administration can be performed at time points that are less frequent or that are substantially equal to yearly, monthly, daily, weekly, multiple times per day, or on an as needed basis (e.g., prior to an anticipated exposure).

In some embodiments, uses of compositions according to Formula (1) are provided for the treatment or prevention of unwanted effects due to exposure to antigens. In some embodiments, the method involve administration of one or more compounds according to Formula (1) comprising one or more antigens, tolerogenic portions thereof, fragments thereof, or mimetics thereof. The compositions disclosed herein are suitable for administration to a subject in conjunction with such use, for example by oral, IV, IM, or other suitable route. Uses of the compositions disclosed herein, in several embodiments, unexpectedly result in the reduction, elimination or amelioration of adverse immune responses to antigens of interest.

In several embodiments, the amount of the composition administered is an amount sufficient to result in induction of clonal deletion and/or anergy of T cells that are specific to the antigen of interest. In several embodiments, the composition is configured to target primarily hepatocytes and/or LSEC. In several embodiments, the composition is configured to induce expansion of certain populations, or sub-populations, of regulatory T cells. For example, in several embodiments, CD4⁺CD25⁺FOXP3⁺ regulatory T cells are induced.

In some embodiments, the method of treatment of an unwanted immune response against an antigen is accomplished by administering to a mammal (e.g., a patient or subject) in need of such treatment an effective amount of a composition comprising a compound of Formula (1) as disclosed herein. In some such methods the composition can be administered for clearance of a circulating protein or peptide or antibody that specifically binds to antigen moiety X, which circulating protein or peptide or antibody is causatively involved in transplant rejection, immune response against a therapeutic agent, autoimmune disease, hypersensitivity and/or allergy. The composition can be administered in an amount effective to reduce a concentration of the antibodies that are causatively involved in transplant rejection, immune response against a therapeutic agent, autoimmune disease, hypersensitivity and/or allergy in blood of the patient by at least 50% w/w, as measured at a time between about 12 to about 48 hours after the administration. The composition can be administered for tolerization of a patient with respect to antigen moiety (e.g., X).

The pharmaceutical compositions described herein can be administered to a human patient per se, or in pharmaceutical compositions where they are mixed with other active ingredients, as in combination therapy, or carriers, diluents, excipients, or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping, or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In several embodiments, a compound of Formula (1), or a pharmaceutically acceptable salt thereof, can be administered intravenously.

The compositions may, if desired, be presented in a dispenser device which may contain one or more unit dosage forms containing the active ingredient. The dispenser device may be accompanied by instructions for administration. The dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

In several embodiments, the composition (e.g., a pharmaceutically acceptable composition) is provided as a unit dose. In several embodiments, the method of treating includes administering a unit dose to a patient or subject. In several embodiments, the unit dose includes 1 μg/kg to 10 mg/kg of a tolerogenic construct (e.g., compositions as disclosed herein comprising a targeting moiety, a linker, and an antigen to which tolerance is desired) to body weight of a subject. In several embodiments, the tolerogenic construct to body weight per administration in a single dose is equal to or less than: about 10 μg/kg, about 50 μg/kg, about 75 μg/kg, about 0.1 mg/kg, about 0.2 mg/kg, about 0.5 mg/kg, about 0.75 mg/kg, about 1.0 mg/kg, about 1.5 mg/kg, about 2.0 mg/kg, about 2.5 mg/kg, about 4.0 mg/kg, about 5.0 mg/kg, 10.0 mg/kg, or ranges spanning and/or including the aforementioned values. In some embodiments, the quantity of tolerogenic construct that is administered is at a unit dose that is less than or equal less or equal to 10, 5, 2, 1, 0.5, 0.1, 0.05, 0.01, 0.005, 0.001, or 0.0005 mg per kg of bodyweight. In several embodiments, a dosing regimen is provided for, wherein a subject receives at least one dose of a composition according to embodiments disclosed herein. In several embodiments, the subject receives at least two, at least three, at least four, at least five, or more doses of a composition according to embodiments disclosed herein. In several embodiments, a given subsequent dose is provided at a concentration that is less than or equal to the prior dose. For example, when receiving a second dose, if the concentration of the first dose was 0.5 mg/kg, the second dose may be provided at about 0.25 mg/kg. In additional embodiments, the doses are held constant over time. Depending on the severity of the underlying immune response (or potential immune response), the doses optionally escalate over time.

### Methods of Manufacture

Some embodiments pertain to a method of manufacturing tolerogenic compounds (e.g., compounds of Formula (1) and/or (1′)) and/or any intermediate and/or precursor compounds used to synthesize the same. In some embodiments, one or more of monomers (e.g., acrylic) are polymerized to provide a block copolymer, gradient copolymer, random copolymer, or mixtures thereof (e.g., of W¹ and W²). In some embodiments, a single monomer (e.g., W¹ or W³) is used to make a homopolymer. In some embodiments, a linker and targeting portion of the construct (e.g., [Y(—Z)ₚ]ₘ-EU) is synthesized and coupled to an antigen (e.g., X) via a disulfide bond or via a disulfanyl ethyl ester. In some embodiments, a linker and targeting portion of the construct (e.g., [Y(—Z)ₚ]ₘ-EU) is synthesized and coupled to an antigen (e.g., X) via an amide or ester coupling (e.g., using an amine or alcohol from the linker and a carboxylic acid from the antigen or using an amine or alcohol from the antigen and a carboxylic acid from the linker). In some embodiments, the antigen (e.g., X) is functionalized with an alkyne containing substitutent and is coupled to a portion of the linker and targeting portion of the construct (e.g., [Y(—Z)ₚ]ₘ-EU) via a pendant azide linkage of Y. In some embodiments, the antigen (e.g., X) is functionalized with a reactive group and the W¹ and/or W² polymer or copolymer is grown from the reactive group of X. In some embodiments, various degrees of polymerization of W¹ and W² are provided. In some embodiments, the degree of polymerization (e.g., the number of W¹ and/or W² units) is equal to or at least about 10, 30, 50, 100, 150, 200, 250, 300, or ranges including and/or spanning the aforementioned values. In several embodiments, degree of polymerization unexpectedly increases the tolerogenic effect of the constructs disclosed herein.

In several embodiments, an acrylyl containing monomer is prepared. In some embodiments, the acrylyl containing monomer is one that is functionalized with a liver-targeting agent (e.g., as shown below).

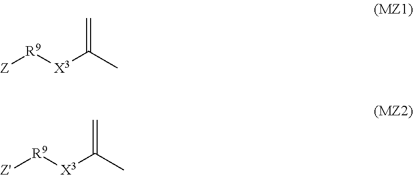

where the variables are as disclosed elsewhere herein. In some embodiments, the acrylyl containing monomer comprises a precursor of a targeting agent (e.g., a protected liver targeting moiety). The precursor is shown above as Z′. In some embodiments, Z′ can be a protected liver targeting moiety (such as OAc protected sugars as shown on Compound 8 in the examples). In some embodiments, X³ and R⁹ are as disclosed elsewhere herein.

In some embodiments, the acrylyl monomer is one that is not functionalized with a targeting agent (e.g., a liver-targeting agent). An embodiment of an acrylyl monomer that is not functionalized with a liver-targeting agent is shown below, where X⁴ and R¹⁰ are as disclosed elsewhere herein.

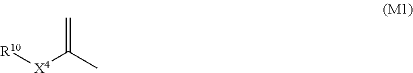

In some embodiments, the acrylyl monomer that is not functionalized with a liver-targeting agent is as follows (where R¹⁰ is as disclosed elsewhere herein):

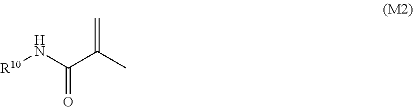

In some embodiments, one or more acrylyl monomers that are not functionalized with a liver-targeting agent is copolymerized with one or more acrylyl monomers that are functionalized with a liver-targeting agent (and/or the acrylyl that is functionalized with a liver-targeting agent precursor). In some embodiments, the acrylyl monomer(s) that is not functionalized with a liver-targeting agent acts as a spacer for the acrylyl monomer(s) that is functionalized with a liver-targeting agent. In some embodiments, employing a spacer may increase or decrease the liver targeting efficacy. In several embodiments, the liver targeting efficacy of the construct is modulated by varying the spacer acrylyl units with the non-spacer acrylyl units. In several embodiments, the liver targeting efficacy of the construct is modulated with the by varying the spacing of the targeting (e.g., liver targeting) acrylyl units. In some embodiments, the one or more acrylyl monomers that are functionalized with a liver-targeting agent can comprise acrylyl monomers that are galactosylated and/or glucosylated.

In some embodiments, the acrylyl monomers are polymerized using a reversible addition-fragmentation chain-transfer agent (e.g., RAFT reagent) in a reversible addition-fragmentation chain-transfer (RAFT) polymerization. In some embodiments, the RAFT reagent is terminated with pyridyl disulfide (PDS). In some embodiments the RAFT reagent is terminated with a carboxylic acid. In several embodiments, the polymerization is performed in the presence of an initiator.

In some embodiments, the RAFT reagent is terminated with a different end group. In some embodiments, the RAFT reagent is a structure as shown by RAFT Formula (1):

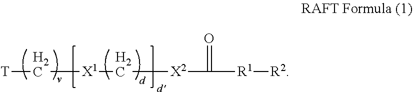

where the variables are as disclosed elsewhere herein. In several embodiments, T is a terminal group (e.g., PDS, maleimide, vinyl sulfone, iodoacetyl, an NHS ester, or a carboxylic acid, or any group reactive toward bioconjugation), R² is any of functional groups I-IV, X¹ and X² are independently selected from —NR⁶—, —NR⁶H, —O—, and —OH, and R¹ and v are as disclosed elsewhere herein. For example, in several embodiments, R₁ is —CH₂—, —(CH₂)₂—C(CH₃)(CN)—, —(CH₂)₂—C(CH₃)(CH₃)—, —(CH₂)₂—CH(CH)— or —CH(CH₃)—. For example, as disclosed elsewhere herein, in several embodiments, v is an integer greater than or equal to about: 0, 1, 2, 3, 4, 5, 10, 15, 20, 40, 50, 75, 100, 150 or ranges including and/or spanning the aforementioned values. In some embodiments, d and d′ are independently an integer greater than or equal to about: 0, 1, 2, 3, 4, 5, 10, 15, 20, 40, 50, 75, 100, 150 or ranges including and/or spanning the aforementioned values. In some embodiments, R⁶ is H or optionally substituted C₁₋₆alkyl. Where X² is —OH, X¹ and T are not present and each of v, d, and d′ are 0. In several embodiments, where the antigen is coupled at the ω side of the polymeric portion, T may be defined as EU (as disclosed elsewhere herein). In several embodiments, where the antigen is coupled at the ω side of the polymeric portion, EU (as disclosed elsewhere herein) may be provided by the RAFT reagent.

In some embodiments, the RAFT Formula (1) reagent (e.g., RAFT reagent) may be expressed as one of the following structures:

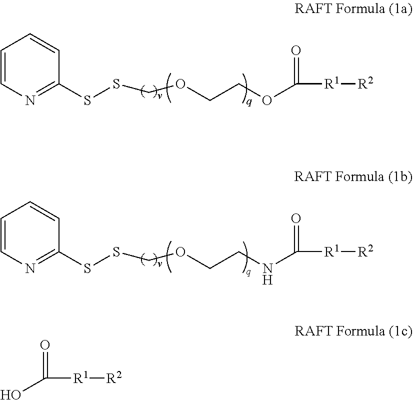

In several embodiments, the variables are as disclosed elsewhere herein. For example, as disclosed elsewhere herein, in several embodiments, q is an integer greater than or equal to about: 0, 1, 2, 3, 4, 5, 10, 15, 20, 40, 50, 75, 100, 150 or ranges including and/or spanning the aforementioned values. As disclosed elsewhere herein, in several embodiments, v is an integer greater than or equal to about: 0, 1, 2, 3, 4, 5, 10, 15, 20, 40, 50, 75, 100, 150 or ranges including and/or spanning the aforementioned values. In several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—.

In some embodiments, the RAFT reagent of Formula (1) is a structure as represented by RAFT Formula (l d) (where R² is DTB):

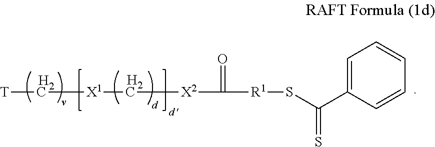

In several embodiments, the variables are as disclosed elsewhere herein. In several embodiments, for example, T is a terminal group or is not present (e.g., PDS, maleimide, vinyl sulfone, iodoacetyl, an NHS ester, or a carboxylic acid), X¹ and X² are independently selected from —NR⁶—, —NR⁶H, —O—, and —OH, and R¹ and v are as disclosed elsewhere herein. For example, in several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—. For example, as disclosed elsewhere herein, in several embodiments, v is an integer greater than or equal to about: 0, 1, 2, 3, 4, 5, 10, 15, 20, 40, 50, 75, 100, 150 or ranges including and/or spanning the aforementioned values. In some embodiments, where v is 0, d′ is 0, and X² is OH, T is not present. In some embodiments, d and d′ are independently an integer greater than or equal to about: 0, 1, 2, 3, 4, 5, 10, 15, 20, 40, 50, 75, 100, 150 or ranges including and/or spanning the aforementioned values. In some embodiments, R⁶ is H or optionally substituted C₁₋₆ alkyl. Where X² is —OH, X¹ and T are not present and each of v, d, and d′ are 0.

In some embodiments, the RAFT Formula (1) reagent may be expressed as one of the following structures:

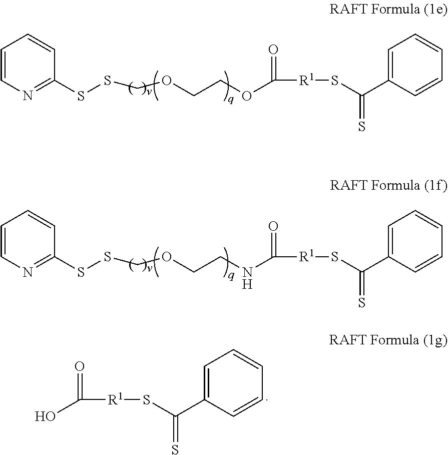

In several embodiments, the variables are as disclosed elsewhere herein. In some embodiments, q, v, and R¹ are as disclosed elsewhere herein. For example, as disclosed elsewhere herein, in several embodiments, q is an integer greater than or equal to about: 0, 1, 2, 3, 4, 5, 10, 15, 20, 40, 50, 75, 100, 150 or ranges including and/or spanning the aforementioned values. In several embodiments, R₁ is —CH₂—, —(CH₂)₂—C(CH₃)(CN)—, —(CH₂)₂—C(CH₃)(CH₃)—, —(CH₂)₂—CH(CH₃)— or —CH(CH₃)—.

In some embodiments, the RAFT Formula (1) structure may be expressed as one of the following (where q and v are as disclosed elsewhere herein):

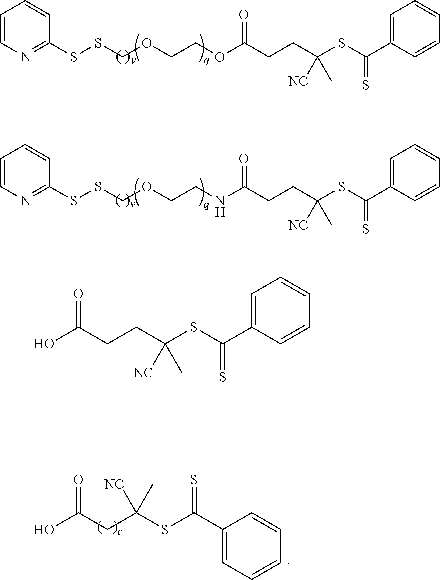

In several embodiments, the variables are as disclosed elsewhere herein. In several embodiments, c is an integer greater than or equal to about: 0, 1, 2, 3, 4, 5, 10, 15, 20, 40, 50, 75, 100, 150 or ranges including and/or spanning the aforementioned values.

In some embodiments, the RAFT Formula (1) structure may be expressed as one of the following:

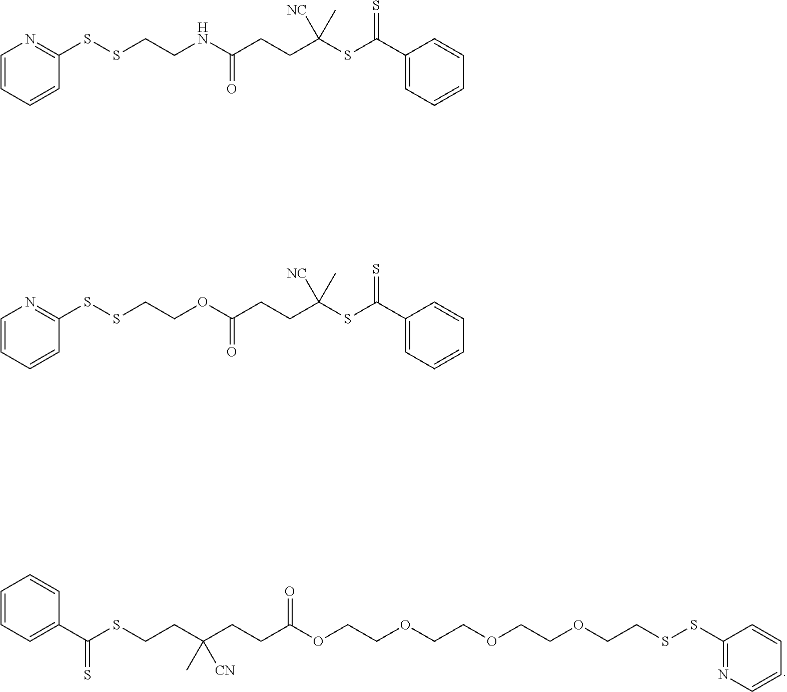 [SMILES: CC(C#N)(CCC(=O)NCCSSc1ccccn1)SC(=S)c1ccccc1 ; CC(C#N)(CCC(=O)OCCSSc1ccccn1)SC(=S)c1ccccc1 ; CC(C#N)(CCSC(=S)c1ccccc1)CCC(=O)OCCOCCOCCOCCSSc1ccccn1]

In several embodiments, as disclosed elsewhere herein, the antigen is bonded to the ω-side of the construct. In several embodiments, where the antigen is bonded to the ω-side of the construct, the terminal end unit may be provided as part of the RAFT reagent. In some embodiments, the RAFT reagent is a structure represented by RAFT Formula (1h):

EU-R² RAFT Formula (1h).

where EU is as defined elsewhere herein. In some embodiments, the RAFT reagent (e.g., the RAFT reagent of Formula (1a)) is a structure represented by RAFT Formula (1i):

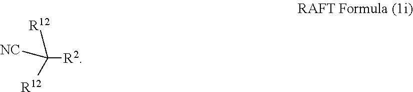

where each instance of variable is as defined elsewhere herein. In some embodiments, the RAFT reagent (e.g., the RAFT reagent of Formula (1h)) is a structure represented by RAFT Formula (1j) (where R² is DTB):

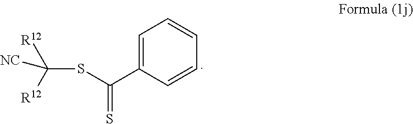

In some embodiments, one or more acrylyl monomers that are functionalized with one or more different liver-targeting agents are mixed with the RAFT reagent to polymerize the acrylyl monomers into a polymer. For example, a N-acetylgalactosamine functionalized monomer could be mixed with a N-acetylglucosamine functionalized monomer and a RAFT reagent. In some embodiments, the acrylyl monomer that is functionalized with a liver-targeting agent (or different types of monomers functionalized with one or more different liver-targeting agents) is mixed with one or more acrylyl monomers that are not functionalized with a liver-targeting agent and the RAFT reagent to form a copolymer of liver targeting units and spacer units as disclosed elsewhere herein. In some embodiments, an acrylyl monomer that is not functionalized with a targeting agent (or one or more different types of monomers not functionalized with liver-targeting agents) is mixed with the RAFT reagent to polymerize the acrylyl monomer that is not functionalized with a targeting agent into a polymer. In some embodiments, an acrylyl monomer that is not functionalized with a liver-targeting agent may be functionalized after polymerization with a liver-targeting agent by reacting it with, for example, a reactive liver targeting agent (see, e.g., Compound 4). In some embodiments, where the liver-targeting agent is added after polymerization, the reactive liver targeting agent may be protected by one or more protecting groups that can be removed after the polymer chain is functionalized.

In some embodiments, the acrylyl monomer(s) and the RAFT reagent (e.g., RAFT Formula 1) form a polymer as shown below (as Formula (2a)):

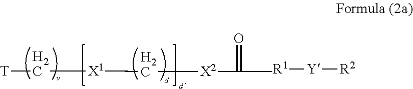

where T, v, d, d′, X¹, X², R¹, and Y′ are as disclosed elsewhere herein. In some embodiments, for example, Y′ is a random copolymer, a gradient copolymer, or block copolymer of W¹ and W², as disclosed elsewhere herein. An embodiment of a scheme showing the reaction of one or more acrylyl-based monomers with the RAFT reagent is shown below. In some embodiments, a carboxylic acid of the R²-terminated polymer may be be further functionalized with, for example, a functionalized reagent to provide additional functionalized polymers, as shown in the following scheme:

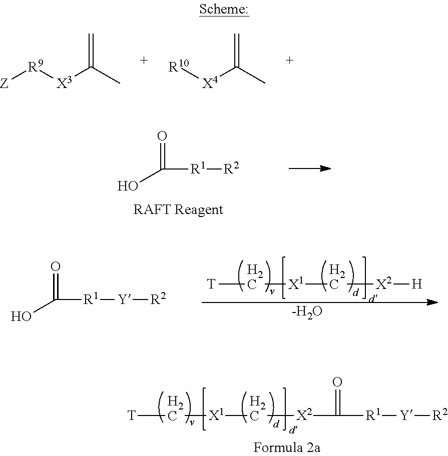

where variables are as disclosed elsewhere herein. In some embodiments, a carboxylic acid of the DTB-terminated polymer may be be further functionalized with, for example, a functionalized reagent to provide additional functionalized polymers, as shown in the following scheme:

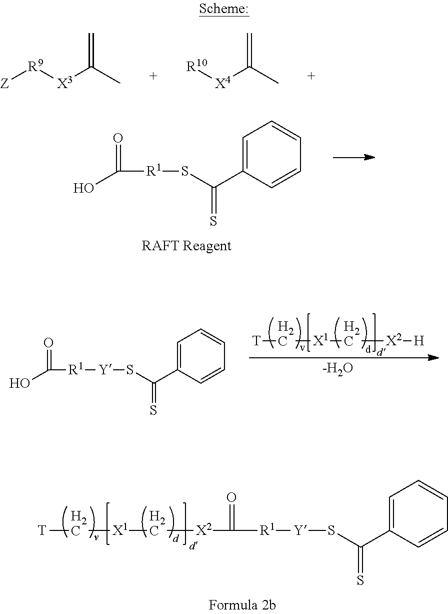

where T, v, d, d′, X¹, X², R¹, and Y are as disclosed elsewhere herein. As noted elsewhere herein, in some embodiments, the RAFT reagent may be terminated with PDS or, alternatively, a carboxylic acid (e.g., as T). An embodiment of a reaction scheme showing the reaction of one or more acrylyl-based monomers with a carboxylate-terminated RAFT reagent is shown above. Also noted in the above scheme, the carboxylate may be terminated with an activating group (e.g., it may be provided instead as a PDS, maleimide, vinyl sulfone, an NHS ester, or iodoacetyl) after polymerization.

In some embodiments, the reaction of the one or more acrylyl monomers and the RAFT reagent provides a polymeric structure as follows (Formulae (2c) to (2f)):

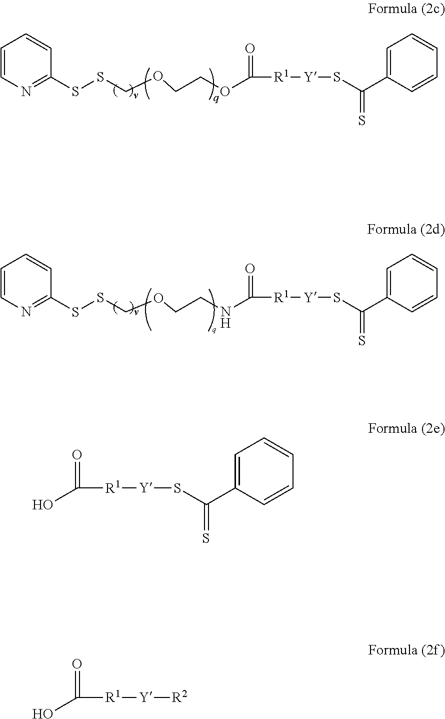

where v, q, R¹, R² and Y′ are as disclosed elsewhere herein.

In some embodiments, the following structures may be used as RAFT reagents:

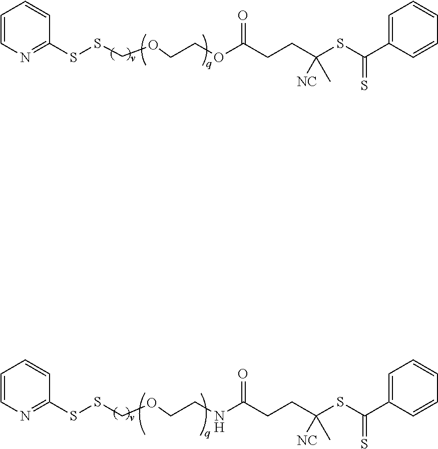

where q and v are as disclosed elsewhere herein.

In several embodiments, where a ω-end antigen construct is provided, the acrylyl monomer(s) and the RAFT reagent (e.g., RAFT Formula (1a)) form a polymer as shown below:

EU-Y′—R² Formula (2g)

where variables are as disclosed elsewhere herein.

In some embodiments, the R² (e.g., a dithiobenzoate group (DTB), etc.) can be exchanged with an EU using an azo-containing compound, as shown below (Formula (3)):

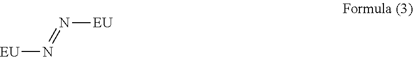

where EU is as disclosed elsewhere herein. In some embodiments, the R² (e.g., a dithiobenzoate group (DTB), etc.) can be exchanged with a terminal end unit using an azo-containing compound (Formula (3a)), as shown below:

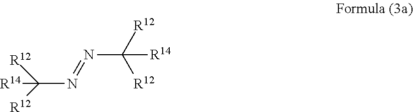

where R¹⁴ and R¹² are as disclosed elsewhere herein. In several embodiments, Formula (3) is represented by Formula (3a). In several embodiments, R² is displaced with a terminal end unit to provide an α-end antigen construct. In other embodiments, in the case of preparing a ω-end antigen construct, R² may be displaced with a group having a functional unit (e.g., a reactive end unit). The functional unit may be further functionalized to provide a tolerogenic construct, by adding an antigen (e.g., where the antigen is provided on the ω-side of the polymer). In several embodiments, the R² group (e.g., dithiobenzoate group (DTB)) can be exchanged through reaction with various terminal end units such as nucleophiles, alkyl boranes, alkyl silanes, heat, and other radical sources. In some embodiments, the terminal end unit is the reaction product of an azonitrile, as shown below:

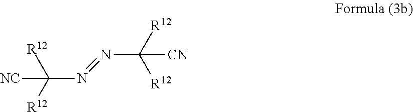

In several embodiments, the azo-containing compound (e.g., of Formula (3) or Formulae (3a) or (3b)) may be represented by a structure selected from the group consisting of the following:

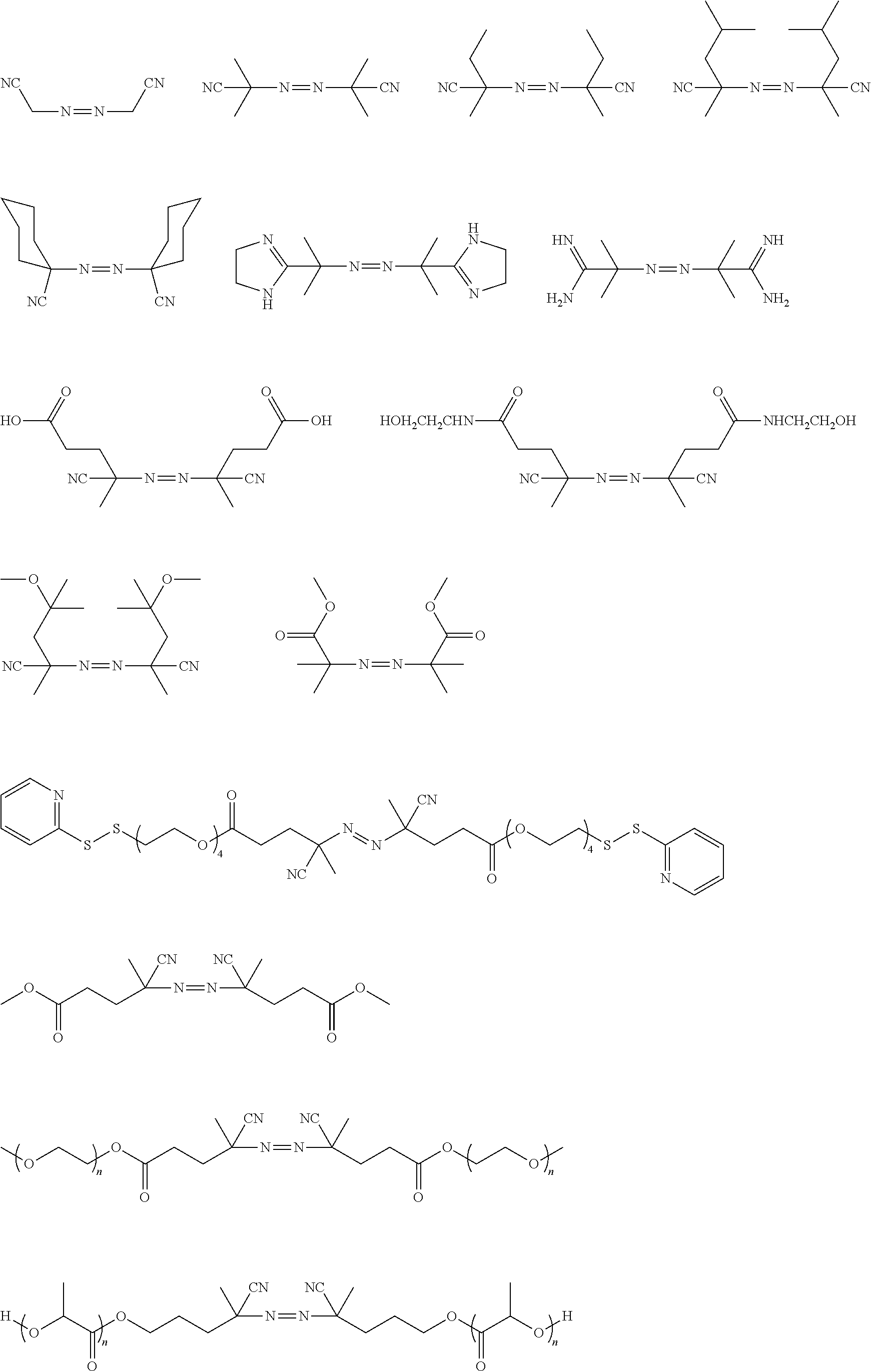

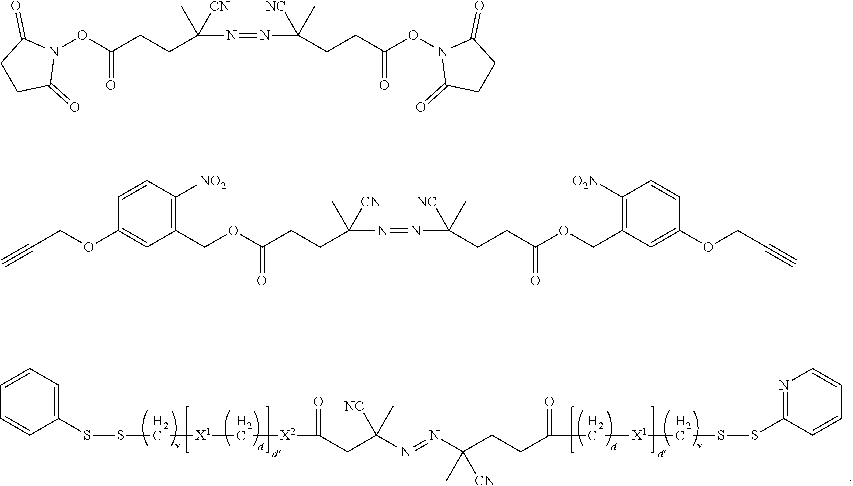

where the variables are as disclosed elsewhere herein. In several embodiments, f, v, d, d′, where present, are independently an integer greater than or equal to about: 0, 1, 2, 3, 4, 5, 10, 20, 40, 50, 100, 150, 200, or ranges spanning and/or including the aforementioned values.

In some embodiments, the exchange of, for example, an R² group (having a thioester and/or an aryl group (e.g., DTB-group), etc.) through reaction with one of the above structures (e.g., of Formula (3)) leads to unexpectedly improved properties for the polymer, including but not limited to improved stability. Exemplary reaction schemes that can be used to provide an α-end antigen constructs are shown below:

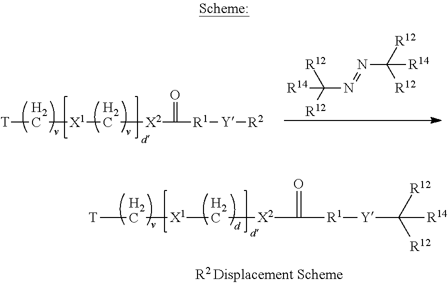

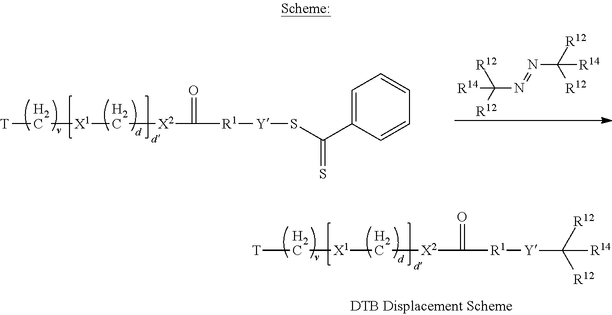

In several embodiments, the variables are as disclosed elsewhere herein. These syntheses can be performed to provide, for example, the following structures:

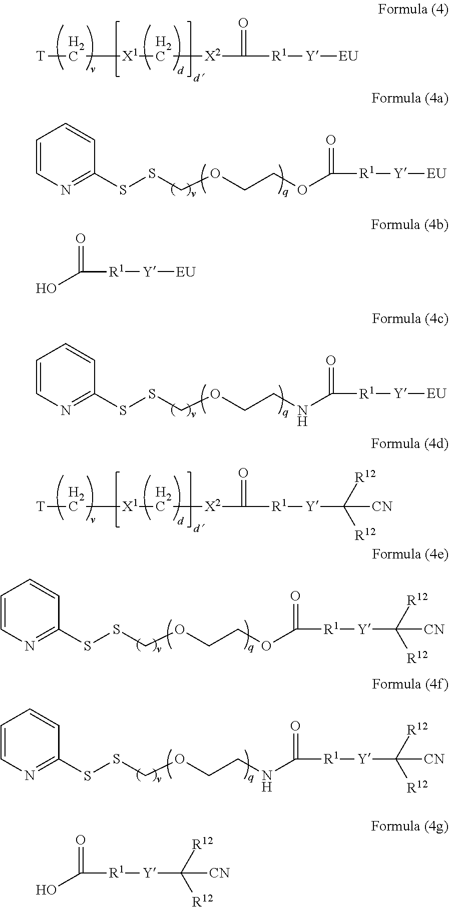

where the variables are as disclosed elsewhere herein. In some embodiments, the azobisalkylnitrile is isobutyronitrile (IBN) and each R¹² is methyl. In some embodiments the DTB-terminated polymer is reacted with azobisisobutyronitrile (ATBN) or another IBN donating group to provide the IBN-terminated polymer, as shown here:

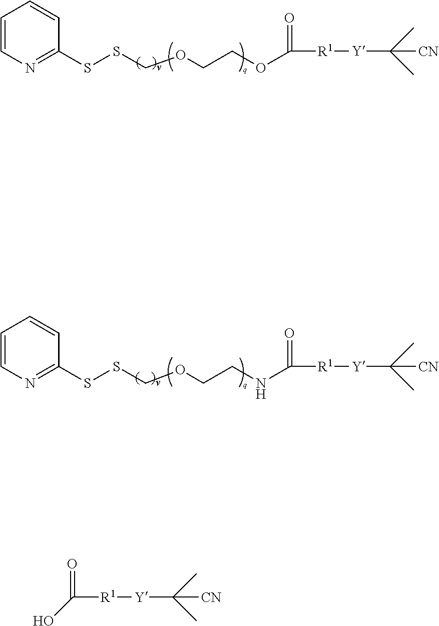

In some embodiments, a carboxylic acid-terminated polymer (shown in the below schemes) may be be further functionalized with, for example, a PDS reagent to provide additional functionalized polymers:

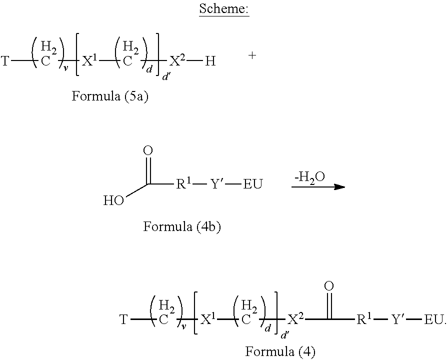

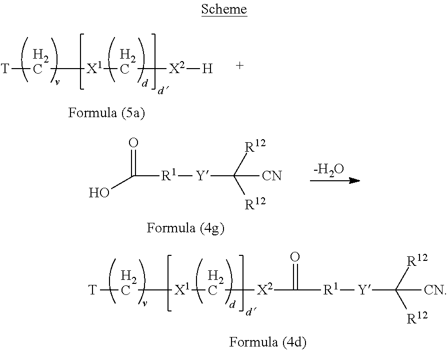

In several embodiments, the variables are as disclosed elsewhere herein. In several embodiments, where T provides a carboxylic acid group, the following PDS-functionalizing reagents may be used:

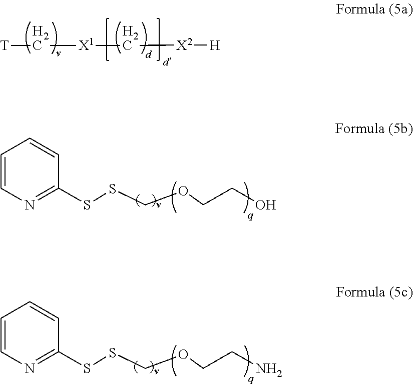

where variables are as disclosed elsewhere herein.

In some embodiments, the following structures may be provided through this reaction:

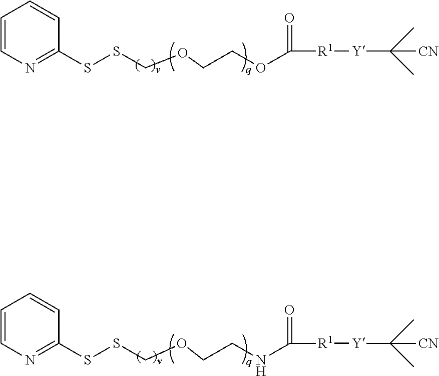

where q and v are as disclosed elsewhere herein.

In several embodiments, as disclosed elsewhere herein, R² may be displaced with a group having a functional group. This functional group may be further reacted with an antigen to provide a ω-end antigen construct. An exemplary reaction scheme that can be used to provide a ω-end antigen constructs is shown below, where R¹² or R¹⁴ comprises a reactive group (and an antigen construct precursor is provided):

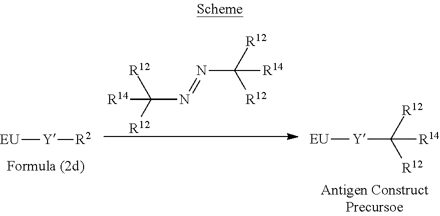

where the variables are as disclosed elsewhere herein. In several embodiments, the antigen construct precursor may be functionalized with a PDS-containing entity or a precursor to a disulfanyl ethyl ester. The PDS-containing entity and/or the precursor to the disulfanyl ethyl ester can then be reacted to provide a ω-end antigen construct (as the following reaction scheme demonstrates):

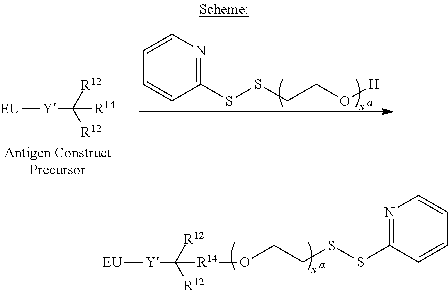

where the variables are as disclosed elsewhere herein and x^{a} is 0, 1, 2, 3, 4, 5, 10, 15, 20, or 44. In several embodiments, the antigen construct precursor is represented by the following structure and the PDS-containing entity is formed as follows:

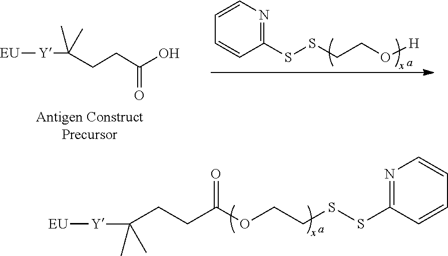

where the variables are as disclosed elsewhere herein.

In several embodiments, the antigen construct precursor comprises the PDS containing entity, as shown by the following synthetic scheme:

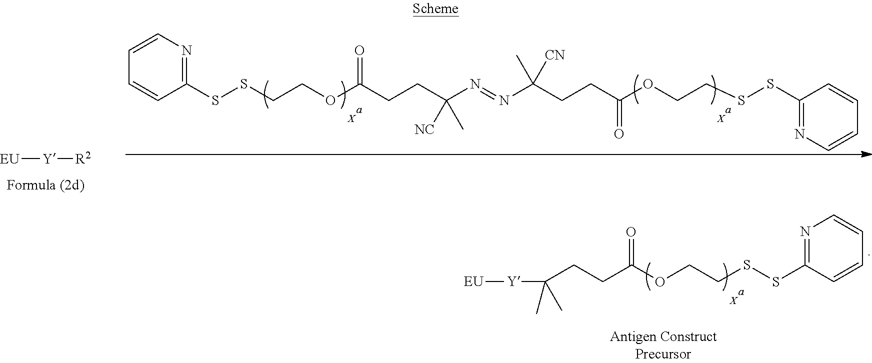

In several embodiments, in embodiments having a PDS group (which comprises a thiopyridine), the thiopyridine may be displaced by an antigen having a thiol functionality to provide the construct (e.g., of Formula (1)). In several embodiments, in embodiments having a PDS group, the thiopyridine may be displaced by a mercapto C₂-C₆ alkanol (e.g., 2-mercaptoethanol) to provide an amide functionalizable precursor (e.g., a linker that may bond with an antigen via an amine of the antigen). The following demonstrates (using 2-mercaptoethanol) and (e.g., Formula 2 where T is PDS):

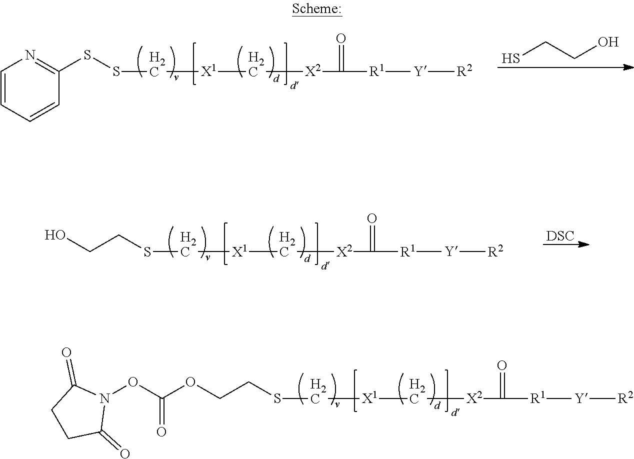

where the variables are as described elsewhere herein. In several embodiments, the NHS ester is added using N,N′-disuccinimidyl carbonate (DSC). In several embodiments, the NHS ester is displaced by mixing with an antigen to provide a structure of Formula (1). In several embodiments, one or more of the following steps may be performed (using 2-mercaptoethanol) and (e.g., where T is PDS):

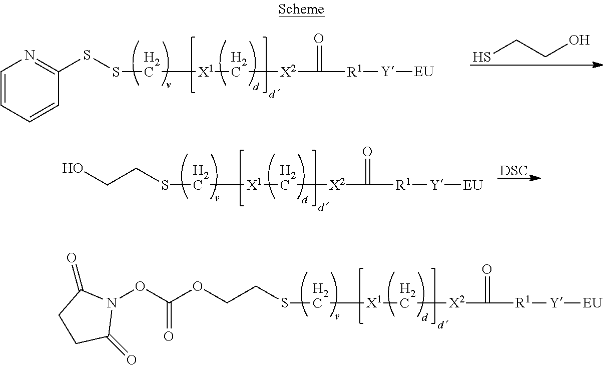

where the variables are as described elsewhere herein. In several embodiments, the NHS ester is displaced by mixing with an antigen to provide a structure of Formula (1).

In several embodiments, increased (or changed) degree of polymerization unexpectedly increases the tolerogenic effect of the constructs disclosed herein. In several embodiments, increased degree of polymerization increases induction of T cell anergy and binding to target cells. In several embodiments, one or more properties of the constructs disclosed herein unexpectedly increases the tolerogenic effect, induction of T cell anergy, binding to target cells, and/or other properties.

In several embodiments, the antigen is conjugated to a full-length linker (e.g., comprising a linker, a terminal end unit, and a targeting agent). In several embodiments, the linker is a thiol-reactive linker. In several embodiments, the thiol-reactive linker comprises a reactive disulfide that reacts with a thiol of the antigen to provide a disulfide bond (e.g., between the antigen and the linker). In several embodiments, the linker is an amine-reactive linker. In several embodiments, the amine-reactive linker comprises a functional group that reacts with an amine of the antigen to provide conjugation to the linker. In several embodiments, the amine-reactive linker comprises a N-hydroxysuccinimide that is displaced by an amine of the antigen to provide a bond between the antigen and linker (e.g., an amide bond, a carbamate, a carbamide, etc.). In several embodiments, the link between the amine reactive linker and the amine-containing antigen provides a disulfanyl ethyl ester linkage.

### Improved Stability

In several embodiments, as disclosed elsewhere herein, constructs comprising terminal end unit having a carbon bond to the linker (and/or lacking a dithioester) have surprisingly improved stability versus dithioester-containing constructs (or those containing trithiocarbonate and xanthates). In several embodiments, by using terminal end units that lack a dithioester group and/or by using constructs that are a reaction product of and/or are produced using a reaction between dithioester terminated linker and an azo-compound (e.g., a bis-azo compound), stability is improved. In several embodiments, stability of the resultant reaction product is enhanced (relative to a dithioester containing construct) by about 2%, 5%, about 10%, about 15%, about 20%, or about 25%.

For example, after a period of about 10 days in storage (e.g., in buffered solution at room temperature), the peak corresponding to the intact construct as disclosed herein loses no more than about 1% of its area at 220 nm as measured by HPLC. Alternatively, after a period of about 10 days in storage (e.g., in buffered solution at room temperature), the peak corresponding to the intact construct comprising a dithioester group loses about 2% of its area at 220 nm as measured by HPLC. After a period of about 20 days in storage (e.g., in buffered solution at room temperature), the peak corresponding to the intact construct as disclosed herein loses no more than about 1% of its area at 220 nm as measured by HPLC. Alternatively, after a period of about 20 days in storage (e.g., in buffered solution at room temperature), the peak corresponding to the intact construct comprising a dithioester loses about 6% of its area at 220 nm as measured by HPLC. After a period of about 28 days in storage (e.g., in buffered solution at room temperature), the peak corresponding to the intact construct as disclosed herein loses no more than about 2% of its area at 220 nm as measured by HPLC. Alternatively, after a period of about 28 days in storage (e.g., in buffered solution at room temperature), the peak corresponding to the intact construct comprising a dithioester loses about 10% of its area at 220 nm as measured by HPLC. In several embodiments, the buffered solution comprises 10 mM sodium acetate, containing 274 mM sorbitol at a compound concentration of 1 mg/mL (pH of about 5 to 5.5). In some embodiments, the temperature is 23-27° C.

In several embodiments, such increases in stability result in a functional improvement of the compositions disclosed herein (e.g., increased efficacy), with respect to induction of antigen-specific immune tolerance. For example, in several embodiments, more antigen per unit dose is delivered to a patient because less construct has degraded. In several embodiments, the improved stability results in improved life of the composition in vivo. In several embodiments, the improved stability allows for the more rapid, more efficient, more robust, or otherwise improved induction of tolerance to an antigen.

In several embodiments, the constructs as disclosed herein (e.g., those lacking a dithioester) show improved stability under various conditions (e.g., during storage, accelerated degradation conditions, etc.). In several embodiments, a 1 mg/mL concentration of a construct as disclosed herein in reducing conditions (10 mM reduced glutathione) in a solution of PBS (pH 7.2) at a temperature of 60° C. shows less than 10% degradation (e.g., area loss at a product peak in HPLC, etc.) after a period of greater than or equal to about: 48 hours, 1 week, one month, 2 months, 6 months, 9 months, 12 months, or ranges including and/or spanning the aforementioned values. In several embodiments, a 1 mg/mL concentration of a construct as disclosed herein in reducing conditions (10 mM reduced glutathione) in a solution of HEPES buffered saline (pH 8.04) at a temperature of 60° C. show less than 10% degradation after a period of greater than or equal to about: 48 hours, 1 week, one month, 2 months, 6 months, 9 months, 12 months, or ranges including and/or spanning the aforementioned values. In several embodiments, a 1 mg/mL concentration of a construct as disclosed herein in a solution of PBS (pH 7.2) at a room temperature show less than 10% degradation after a period of greater than or equal to about: 48 hours, 1 week, one month, 2 months, 6 months, 9 months, 12 months, or ranges including and/or spanning the aforementioned values. In several embodiments, a 1 mg/mL concentration of a construct as disclosed herein in a solution of HEPES-buffered saline (pH 8.04) show less than 10% degradation after a period of greater than or equal to about: 48 hours, 1 week, one month, 2 months, 6 months, 9 months, 12 months, or ranges including and/or spanning the aforementioned values. In several embodiments, a 1 mg/mL concentration of a construct as disclosed herein in a solution of 10 mM sodium acetate, 274 mM sorbitol show less than 10% degradation after a period of greater than or equal to about: 48 hours, 1 week, one month, 2 months, 6 months, 9 months, 12 months, or ranges including and/or spanning the aforementioned values.

In several embodiments, the stability of dithioester-free embodiments (versus compounds comprising a dithioester) is improved over a given period of time by equal to or at least about: 1.0%, 2.5%, 5%, 10%, 15%, 20%, or ranges including and/or spanning the aforementioned values. In several embodiments, dithioester-free embodiments degrade at a reduced rate, for example, their stability decreases by less than or equal to about: 0.1%, 0.5%, 1.0%, 2.0%, 2.5%, 5% over a period of 5, 10, 14, 20, 15, 28 days, or longer. In several embodiments, testing for stability may be performed using the conditions provided in Example 11. In several embodiments, stability testing may be performed over a period of equal to or at least about: 7 days, 14 days, 28 days, or ranges including and/or spanning the aforementioned values. In several embodiments, testing for stability may be performed using a solution comprising sodium acetate buffer. In several embodiments, testing for stability may be performed using a solution comprising sorbitol. In several embodiments, testing for stability may be performed using an aqueous solution at a pH of about 5. In several embodiments, testing for stability may be performed at a temperature of about 23-27° C. In some embodiments, testing is performed using a solution that is 10 mM sodium acetate and 274 mM sorbitol at a peptide concentration of 1 mg/mL (pH of about 5). In some embodiments, testing is performed for a period of 14 days or 28 days at 23-27° C. In several embodiments, over a period of 14 days or 28 days, the stability of dithioester-free embodiments (versus compounds comprising a dithioester) is improved by equal to or at least about: 1.0%, 2.5%, 5%, 10%, 15%, 20%, or ranges including and/or spanning the aforementioned values. In several embodiments, the peptide concentration may refer to either the antigen concentration (e.g., when reduced-off the construct) or construct concentration.

The various studies described in more detail below provide additional evidence that the compositions and methods disclosed herein are useful for the induction of antigen-specific immune tolerance, in accordance with several embodiments herein.

### EXAMPLES

Additional embodiments are disclosed in further detail in the following examples, which are not in any way intended to limit the scope of the claims. The methods and materials represent non-limiting embodiments of how the compounds disclosed herein can be made.

### Materials and Instrumentation.

Exemplary vendors and instrumentation are disclosed here. Unless otherwise indicated reagents were purchased from Sigma-Aldrich. Methylene chloride (reagent grade) 2-propanol (ACS grade), and N,N-dimethylformamide (reagent grade) were obtained from Fisher Scientific. Acetone (reagent grade) was obtained from BDH. Methanol (reagent grade) was obtained from EMD Millipore. Methylene chloride (anhydrous) was obtained from Acros Organics. Pyridine was obtained from VWR N,N-dimethylformamide (HPLC grade) and ethyl acetate (HPLC grade) were obtained from Honeywell. N,N-dimethylformamide (anhydrous) was obtained from Millipore. D-Galactosamine HCl was obtained from Carbosynth. Methacryloyl chloride was obtained from BTC. Acetic anhydride, 4-dimethylaminopyridine, Diglycolamine, Dithiodipyridine, NaOMe (30% wt/wt in MeOH), Potassium thioacetate, Triethylamine, Tetraethylene glycol, and Lithium Bromide (anhydrous) were obtained from Alpha Aesar. 1,2-DCE, Molecular Sieves, Amberlite IR120 (H+) resin, D-Glucosamine HCl, N,N′-dicyclohexylcarbodiimide, Ethanolamine, 4-ethylbenzene-1-sulfonyl chloride, Potassium carbonate, Trimethylsilyl trifluoromethanesulfonate (TMSOTf), 2,2′-azobis(2-methylpropionitrile) (AIBN, recrystallized, 99% purity), 4,4′-azobis(4-cyanovaleric acid) (ACVA, 98.0% purity), N,N′-disuccinimidyl carbonate, 2-mercaptoethanol, 4-nitrophenyl chlorofomate, BCN-NHS, and human insulin protein were obtained from Sigma Aldrich. 4-cyano-4-(thiobenzoylthio)pentanoic acid was obtained from Strem Chemical. 11-Azido-3,6,9-trioxaundecanol, NHS-DTP (SPDP) and S-DBCO-Amine were obtained from BroadPharm. DIBO-OH was obtained from AstaTech, Inc. HS-PEG2K-NH₂HCl was obtained from Jenchem. 2-(Pyridin-2-yldisulfanyl)ethanol was obtained from Synnovator, Inc. Ovalbumin protein (EndoGrade) was obtained from Worthington Biochemical Corporation. Unless otherwise specified, all reagents were used directly, without further purification. All reactions were performed under an atmosphere of nitrogen, unless otherwise stated.

Instrumentation. ¹H- and ¹³C-NMR spectra were obtained using a Varian 400 spectrometer energized to 399.85 MHz or a Varian 500 spectrometer energized to 499.9 MHz. All NMR spectra were analyzed at 25° C. and evaluated against residual solvent peaks. Gel permeation chromatography (GPC) was performed on a Shimadzu Prominence i-Series Plus instrument equipped with a Shimadzu RID20A differential refractometer detector maintained at 50° C. GPC stationary phase was a single Shodex KD-804 size exclusion column packed with styrene-divinylbenzene resin maintained at 50° C. GPC mobile phase was HPLC-grade N,N-dimethylformamide (Honeywell) containing 25 mM Lithium Bromide (Alpha Aesar) at a flow rate of 1.0 mL/min. Liquid chromatography-mass spectrometry (LC-MS) was performed on a Waters single quadrupole TOF spectrometer equipped with a Phenomenex Luna C-8 3μ 30×2.0 mm column. LC-MS mobile phase was a water-acetonitrile gradient containing 0.1% formic acid at a flow rate of 0.7 mL/min. Cation exchange chromatography (CEX) and size exclusion chromatography (SEC) were performed on an ÄKTA pure 25 L chromatography system. For CEX, the stationary phase was a single GE Healthcare 1.0 mL HiTrap Sp High Performance column. CEX mobile phase was 20 mM sodium acetate at pH 4.2 with a gradient of 0-100% of 20 mM sodium acetate pH 4.2 with 1.0 M NaCl at a flow rate of 1.0 mL/min. For SEC, the stationary phase was a single GE Healthcare HiLoad 16/600 Superdex 200 pg (16 mm×600 mm) column. SEC mobile phase was 1.0 M PBS buffer (pH 7.4) at a flow rate of 1.0 mL/min. SDS polyacrylamide gel electrophoresis (PAGE) was performed on Bolt 12% Bis-Tris protein gels (Invitrogen, 1.0 mm×12-well) (23 minutes, 180 V, 20× Bolt MES SDS PAGE running buffer, pH 7.0). Gels were stained with Coomassie SimplyBlue SafeStain (Life Technologies). Matrix-assisted laser desorption-ionization time-of-flight mass spectrometry (MALDI-TOF-MS) spectra were obtained on an Applied Biosystems Voyager-DE Pro instrument in linear positive mode. All MALDI samples were analyzed via 2,5-Dihydroxybenzoic acid (DHB) matrix. Size-exclusion chromatography (SEC) was done on a GE Healthcare life sciences ÄKTA pure 25 L system, using PBS as the mobile phase and a GE HiLoad 16/600 Superdex 200 prep grade column.

### Example 1: Chemical Synthesis of Monomers

The following provides exemplary procedures for the syntheses of various monomers for preparing certain embodiments as disclosed herein.

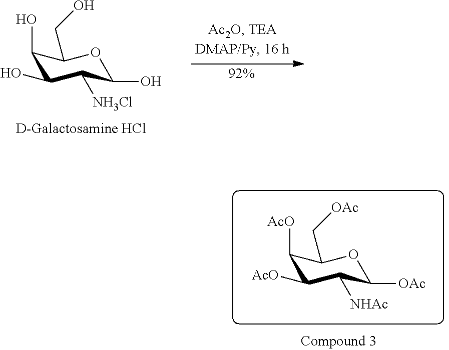

Galactosamine pentaacetate (3). D-Galactosamine HCl (6.73 g, 31.2 mmol) was suspended in pyridine (30 mL) and acetic anhydride (22 mL, 0.23 mol). The flask was cooled to 0° C. in an ice bath and DMAP and triethylamine were charged into the mixture. The contents of the flask were allowed to warm to room temperature under N₂ atmosphere. After stirring for 16 hours, the reaction mixture was diluted with EtOAc at which point additional solids were evident. The solid product 3 was collected by filtration on a fritted glass filter and placed on high vacuum (11.23 g, 92%). This material was sufficiently pure by NMR and used directly in the next procedural step.

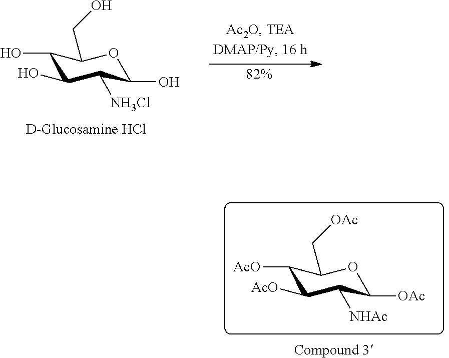

Glucosamine pentaacetate (3′). D-Glucosamine HCl (20 g, 92.7 mmol) was suspended in pyridine (125 mL). Acetic anhydride (123 mL, 1.3 mol) was added followed by a catalytic amount of DMAP and triethylamine (13 mL, 93 mmol, 1 eq.). The reaction mixture turned a pale-yellow color with minimal white solid precipitate. Stirring continued at room temperature under N₂ atmosphere. TLC analysis showed reaction completion and the mixture was filtered through a glass filter to remove some white solids (presumably a salt). The filtrate was diluted with ethyl acetate, washed with saturated NaHCO₃ and brine, dried over Na₂SO₄, filtered and concentrated. The crude material was dissolved in boiling absolute EtOH (350 mL) cooled to room temperature and placed in freezer for 16 hours. The white solids were collected on a Buchner funnel and washed with cold EtOH. The mother liquor was concentrated, recrystallized and dried on high vacuum (29.2 mg, 82%).

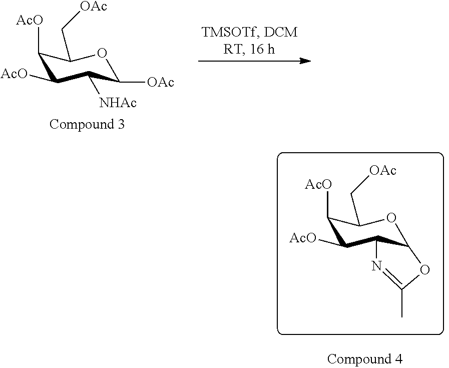

Triacetyl D-Galactose Oxazoline (4). Galactosamine pentaacetate 3 (21.86 g, 56 mmol) was dissolved in anhydrous dichloromethane (40 mL) under N₂ atmosphere in a flask equipped with a stir bar. 12.2 mL (1.2 eq., 67 mmol) of TMSOTf was added to the reaction mixture and stirring continued for 16 hours at room temperature. Reaction completion was confirmed by TLC analysis (70% EtOAc:Hex). The reaction solution was quenched by pouring into a saturated aqueous NaHCO₃/ice mixture followed by stirring for 30 minutes. The reaction mixture was then separated, and the aqueous layer was extracted twice with DCM. The combined organic layers were dried over anhydrous Na₂SO₄, filtered, and concentrated under vacuum resulting in 4 as a crude oil (18.4 g). Compound 4 was used without further purification in the next step.

Alternatively, the following procedures were used to provide 4 (e.g., 2-Methyl-(3,4,6-tri-O-acetyl-1,2-dideoxy-α-D-galactopyrano)[1,2-d]-1,3-oxazoline). D-galactosamine penta-acetate 15 (2.0 g, 5.15 mmol) was dissolved in dichloroethane (DCE) (20 mL). Then trimethylsilyl trifluoromethanesulfonate (TMSOTf) (1 mL, 5.53 mmol) was added, and the mixture was stirred at 50° C. for 9 h. The mixture was then removed from the heat and stirred for 7 hours. Triethylamine (2 mL) was added to the mixture at room temperature. The mixture was then washed with a saturated solution of NaHCO₃ and then dried with sodium sulfate. The organic phase was then filtered, and the solvent was removed via rotary evaporation and the residue was loaded onto silica gel. The product was purified via column chromatography on silica gel with EtOAc (100) to yield 16 as a yellow viscus solid (Yield: 64%). ¹H-NMR: (400 MHz, CDCl₃-d₆): δ (ppm), 5.97 (d, J=6.9 Hz, 1H, H-4); 5.45 (t, J=3.0 Hz, 1H, H-5); 4.92 (dd, J=7.6 Hz, 3.4 Hz, 1H, H-4); 4.26 (td, J=6.7 Hz, 2.8 Hz, 1H); 4.25-4.13 (m, 1H, H-3); 3.99 (s, 1H); 2.13 (s, 3H); 2.07 (s, 6H); 2.05 (s, 3H). ¹³C-NMR: (125 MHz, DMSO-d6): δ (ppm), 170.0; 169.55; 168.11; 165.21; 100.9; 70.66; 68.2; 65.02, 63.00, 61.8, 20.5, 20.44, 20.42, 13.91. MS m/z: [M+H]+ 330.12.

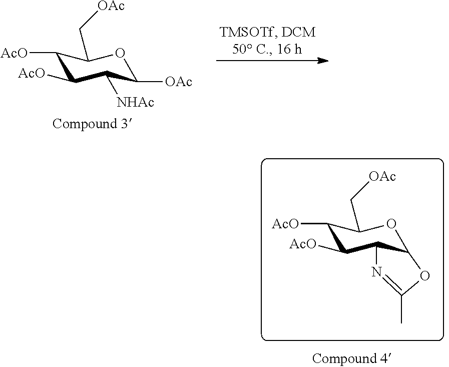

Triacetyl D-Glucose Oxazoline (4′). Glucosamine pentaacetate 3′ (19.64 g, 50.44 mmol) was dissolved in anhydrous dichloroethane (500 mL) under N₂ atmosphere. 17.0 g of activated AW-300 molecular sieves were added, and the solution heated to 50° C. while stirring. TMSOTf (1.1 eq., 55.5 mmol, 10 mL) was slowly added to the reaction mixture and heating and stirring continued for 16 hours. TLC analysis showed the reaction was complete and the mixture was quenched by pouring into ice-cold saturated NaHCO₃. The reaction mixture was filtered through a glass frit and the layers of filtrate separated. The aqueous layer was extracted twice with DCM and the combined organic layers were dried over Na₂SO₄, filtered and concentrated under vacuum resulting in crude 4′ as a yellow oil (16.09 g). ¹H-NMR showed a 5:1 ratio of product 4′ and starting material 3′. This material was used directly without purification in the next step.

Alternatively, the following procedures were used to provide 4′ (e.g., 2-Methyl-(3,4,6-tri-O-acetyl-1,2-dideoxy-α-D-glucopyrano)[1,2-d]-1,3-oxazoline). D-glucosamine penta-acetate (10 g, 25.6 mmol) was dissolved in dichloroethane (DCE) (150 mL). Then trimethylsilyl trifluoromethanesulfonate (TMSOTf) (5.5 mL, 30 mmol) was added, and the mixture was stirred at 50° C. for 1 hour. The mixture was then removed from the heat and stirred for 16 h. Triethylamine (4 mL) was added to the mixture at room temperature. The mixture was then stirred for 10 min then the solvent was removed via rotary evaporation. The crude material was loaded onto silica gel and purified via flash chromatography, EtOAC (100) to give 14 as a pink oil (Yield: 61%). ¹H-NMR: (400 MHz, CDCl₃-d₆): δ (ppm), 5.86 (d, J=7.4 Hz, 1H, H-4); 5.22 (t, J=2.1 Hz, 1H, H-5); 4.87 (d, J=9.3 Hz, 1H, H-4); 4.12-4.05 (m, 3H, H-2, H-6, H-6′); 3.54-3.57 (m, 1H, H-3); 2.06 (s, 3H); 2.03 (s, 6H); 2.01 (s, 3H). ¹³C-NMR: (125 MHz, CDCl₃-d₆): δ (ppm), 170.41; 169.55; 169.18; 166.34; 99.27; 70.04; 68.17; 67.43, 64.98, 63.12, 20.55, 20.34, 20.42, 13.91. MS m/z: [M+H]+ 330.12.

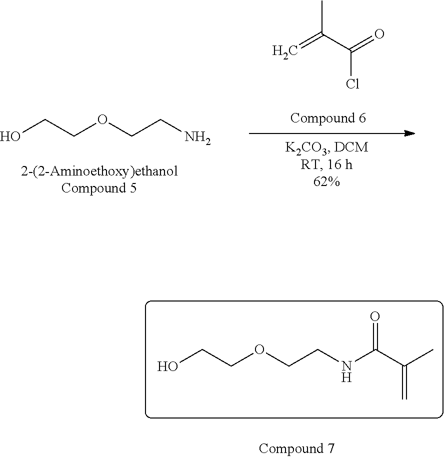

N-(2-(2-hydroxyethoxy)ethyl)methacrylamide (7). 2-(2-aminoethoxy)ethanol 5 (5.0 g, 47.6 mmol) was dissolved in 200 mL of anhydrous dichloromethane and 30 g of K₂CO₃ (217 mmol) was added. The suspension was stirred at 0° C. for 30 minutes followed by dropwise addition of methacryloyl chloride 6 (5.6 mL, 57.1 mmol). After stirring at room temperature for 16 hours, the reaction mixture was filtered through a pad of Celite to remove potassium carbonate and the filtrate concentrated below 30° C. providing a crude oil. Purification was conducted on a silica gel pad using 0-5% MeOH:DCM as an eluent. Fractions containing product were combined and evaporated under vacuum below 30° C. resulting in a pale-yellow oil 7 (5.08 g, 62%). Compound 7 was stored under nitrogen at −20° C. before use.

Alternatively, the following procedures were also performed to provide Compound 7. To 200 mL of an ice-cold solution of 5 2-(2-aminoethoxy ethanol) (24 mL, 240 mmol) and potassium carbonate (15 g) in DCM was slowly added a solution of methacryloyl chloride 6 (24 mL, 250 mmol) in DCM (50 mL). The reaction was allowed to come to room temperature and stirred for another 4 hours. After 4 hours the reaction mixture was filtered through celite and the solvent was removed via rotary evaporation. The crude product was loaded onto silica gel and purified via flash chromatography, Ethyl Acetate (EtOAc):Hexanes (90:10), to give 7 as a colorless oil (Yield: 72%). ¹H-NMR: (400 MHz, CDCl₃-d₆): δ (ppm), 6.53 (s, 1H); 5.66 (m, 1H); 5.29 (m, 1H); 3.71 (s, 2H); 3.56 (m, 4H); 3.48 (m, 2H); 1.91 (m, 3H). ¹³C-NMR (75 MHz, CDCl₃-d₆): δ (ppm), 169.34; 141.72; 120.37; 72.43; 69.82; 61.63; 39.81; 18.86. MS m/z: [M+H]+ 174.11.

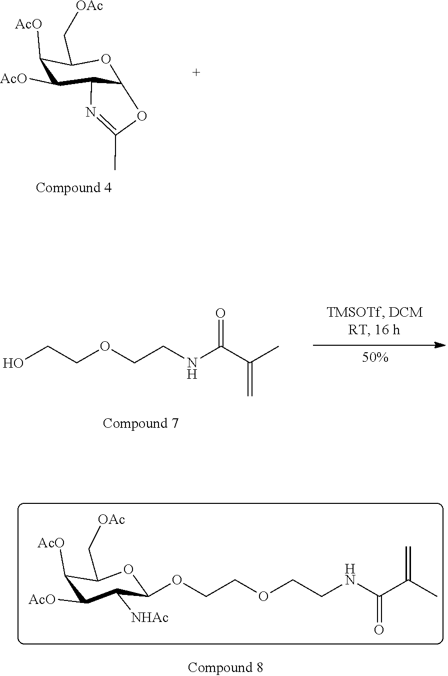

(2-(2-hydroxyethoxy)ethyl)methacrolyl 2-acetamido-3,4,6-O-acetyl D-galactoside (8). Donor 4 (15.1 g, 46.1 mmol) and acceptor 7 (12.5 g, 72.2 mmol, 1.5 eq.) were combined and placed under high vacuum for 30 minutes and subsequently solubilized in anhydrous DCM (180 mL) under N₂ atmosphere. Flame dried AW-300 molecular sieves (15.0 g) were added and the mixture was stirred at room temperature for 30 minutes. The flask was then cooled to 0° C. and TMSOTf (6.3 mL, 34.6 mmol, 0.75 eq.) was slowly added to the reaction mixture over 10 minutes. The reaction was stirred for 16 hours and allowed to warm to room temperature. TLC analysis (60% acetone:hexane) showed minimal donor remaining and the reaction was filtered through a pad of Celite. The resulting filtrate was extracted with saturated NaHCO₃, water, and brine, and dried over anhydrous Na₂SO₄. The crude was purified on a 120 g silica flash cartridge using a 0-100% acetone:hexane gradient. Less pure fractions were combined, concentrated, and re-purified. Fractions containing pure product were combined and evaporated under vacuum providing 8 as an off-white foam (11.44 g, 50%).

Alternatively, the following procedures were also used to prepare 8 (e.g., 2-(2-Hydroxyethoxy)ethyl methacrylamide-2-acetamido-3,4,6-tri-O-acetyl-2-deoxy-α-D-galactopyranoside). A flask was charged with compound 4 (2.0 g, 6.0 mmol), 2-(2-Aminoethoxyethanol) methacrylamide 7 (1.1 g, 6.6 mmol), 4 Å molecular sieves (2.5 g), and DCE (20 mL). The solution was stirred for 30 min. TMSOTf (464 μL, 2.6 mmol) was added and the mixture was stirred at room temperature for 19 hours, then TMSOTf (464 μL, 2.6 mmol) was added again and the reaction was allowed to stir for an additional 8 hours. Triethylamine was then added to the reaction and the reaction was stirred for another 10 min. The solvents were removed via rotary evaporation and the crude product was loaded onto silica gel and purified via column chromatography, hexanes: EtOAc (80:20), to yield 8 as a viscous solid (Yield: 43%). ¹H-NMR: (500 MHz, CD3OD): δ (ppm), 5.72 (s, 1H), 5.35 (s, 1H), 4.67 (m, 1H), 4.65 (m, 1H), 4.32 (d, J=8.5 Hz, 1H), 4.27 (dd, J=5.0, 10.5 Hz, 1H), 4.17-3.69 (m, 6H). 2.01 (s. 3H), 1.99 (s. 3H). 1.97 (s. 3H). 1.89 (s X 2, 6H). MS m/z: [M+H]+ 503.22.

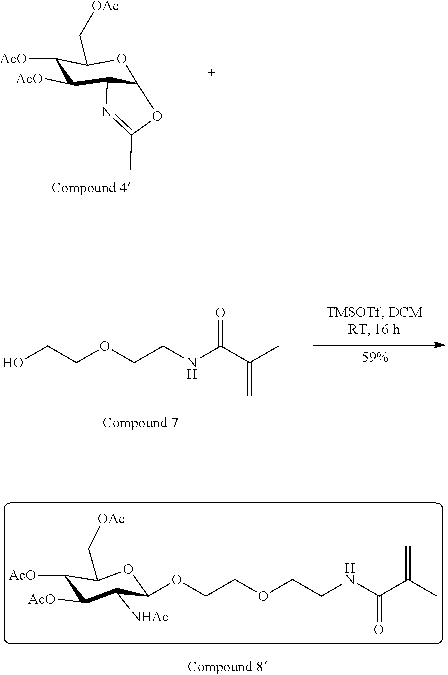

(2-(2-hydroxyethoxy)ethyl)methacrolyl 2-acetamido-3,4,6-O-acetyl D-glucoside (8′). Compound 4′ (13.33 g, 40.5 mmol) and acceptor 7 (8.2 g, 47.3 mmol, 1.2 eq.) were combined and dried under high vacuum for one hour. The starting materials were solubilized in anhydrous DCM (125 mL) under nitrogen overlay and the contents of the flask stirred with flame-dried AW-300 molecular sieves (15 g) for 30 minutes. The reaction mixture was then cooled to 0° C. on an ice bath and TMSOTf (5.5 mL, 0.75 eq.) was added dropwise over a period of 15 minutes. After 4 hours and with equilibration to room temperature, a large amount of starting materials was observed by TLC. 1.0 mL of additional TMSOTf (0.14 eq.) was added. The reaction was complete by TLC analysis (50% acetone:hexane) after stirring for 16 hours at room temperature. The mixture was filtered through a pad of Celite, the filtrate washed with saturated NaHCO₃ and brine, dried over Na₂SO₄, filtered and concentrated. The crude oil was purified on a 120 g HP silica gel column using a 0-100% acetone:hexane gradient. Less pure fractions by TLC were combined, concentrated then re-purified. All fractions containing pure product by TLC were combined and evaporated under vacuum to providing product 8′ as an oil (12.2 g, 59%).

Alternatively, the following procedures were also used to prepare 8′ (e.g., 2-(2-Hydroxyethoxy)ethyl methacrylamide-2-acetamido-3,4,6-tri-O-acetyl-2-deoxy-α-D-glucopyranoside). A flask was charged with compound 4′ (2.0 g, 6.0 mmol), 7 (1.1 g, 6.6 mmol), 4 Å molecular sieves (2.5 g), and DCE (20 mL). The solution was stirred for 30 min. TMSOTf (464 μL, 2.6 mmol) was added and the mixture was stirred at room temperature for 19 hours, then TMSOTf (464 μL, 2.6 mmol) was added again and the reaction was allowed to stir for an additional 8 hours. Triethylamine was then added to the reaction and the reaction was stirred for another 1 hour. The solvents were removed via rotary evaporation and the crude product was loaded onto silica gel and purified via column chromatography, hexane: EtOAc (80:20), to yield 8′ as a viscous solid (Yield: 51%). ¹H-NMR: (500 MHz, CD3OD): δ (ppm), 5.7 (s, 1H), 5.45 (s, 1H), 4.97 (dd, J=10.5, 10.5 Hz, 1H), 4.65 (d, J=8.5 Hz, 1H), 4.32 (d, J=8.5 Hz, 1H), 4.27 (dd, J=5.0, 10.5 Hz, 1H), 4.17-3.69 (m, 6H), 2.01 (s, 3H), 1.99 (s, 3H), 1.97 (s, 3H), 1.89 (s X 2, 6H). MS m/z: [M+H]+ 503.31.

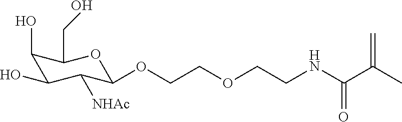 [SMILES: C=C(C)C(=O)NCCOCCOC1OC(CO)C(O)C(O)C1NC(C)=O]

GalNAc Monomer (9). To a solution of compound 8 (14.13 g, 28.2 mmol) in anhydrous MeOH (160 mL) at 0° C. under N₂ was added a solution of NaOMe (4.5 M in MeOH, 6.8 mL). The reaction was warmed to room temperature and monitored by TLC (10% MeOH/DCM). After 2 hours, the reaction was complete and neutralized with Amberlite IR-120 (H+) resin. The reaction mixture was filtered, the resin washed with MeOH, and the combined filtrates evaporated providing a yellow syrup. The crude oil was purified by flash chromatography (0-25% MeOH:DCM) resulting in GalNAc monomer 9 (9.3 g, 90%) as a pale-yellow syrup. The GalNAc monomer 9 was stored in DMF at 40% wt/wt concentration, under nitrogen overlay at −20° C. 1H-NMR (499.9 MHz, D₂O, 25° C., ppm): δ=5.69 (s, 1H), 5.44 (s, 1H), 4.46 (d, J=8.5 Hz, 1H), 3.92 (d, J=3.2 Hz, 1H), 3.90-3.63 (m, 10H), 3.45 (J=10.1 Hz, J=6.5 Hz), 2.01 (s, 3H), 1.91 (s, 3H). ¹³C-NMR: (125 MHz, D₂O, 25° C., ppm): δ=176.2; 169.34; 141.72; 120.37; 103.0; 76.5; 73.6; 72.43; 72.5; 69.82; 69.3; 61.63; 62.4; 53.9; 39.81; 23.5; 18.86; 11.0. MS m/z: [M+H]+ 377.19.

Alternatively, the following procedures were used to provide 9 (e.g., 2-(2-ethoxy)ethyl methacrylamide 2-acetamido-2-deoxy-pi-D-galactopyranoside). Compound 8 (2.0 g, 3.98 mmol) was dissolved in 10 mL of MeOH and stirred at room temperature. Sodium methoxide (4 mmol) was added to the reaction and the reaction was stirred at room temperature. After 6 h, the solution was neutralized with Amberlite IR120 and then filtered. The solvent was removed via rotary evaporation and loaded on to silica gel. The products was purified via column chromatography using DCM:MeOH (83:17) to give 9 as a clear solid (Yield: 78%). ¹H-NMR: (400 MHz, D₂O): δ (ppm), 5.69 (s, 1H), 5.44 (s, 1H), 4.46 (d, J=8.5 Hz, 1H), 3.92 (d, J=3.2 Hz, 1H), 3.90-3.63 (m, 10H), 3.45 (J=10.1 Hz, J=6.5 Hz), 2.01 (s, 3H), 1.91 (s, 3H). ¹³C-NMR: (125 MHz, D₂O): δ (ppm), 176.2; 169.34; 141.72; 120.37; 103.0; 76.5; 73.6; 72.43; 72.5; 69.82; 69.3; 61.63; 62.4; 53.9; 39.81; 23.5; 18.86; 11.0. MS m/z: [M+H]+ 377.19.

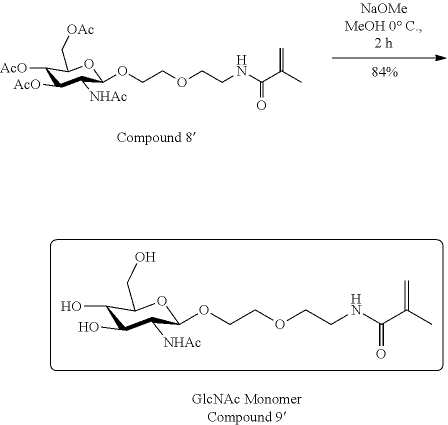

GlcNAc Monomer (9′). To a solution of compound 8′ (12.2 g, 24.3 mmol) in anhydrous MeOH (160 mL) at 0° C. under N₂ was added a solution of NaOMe (4.5 M in MeOH, 6.5 mL). The reaction was warmed to room temperature and monitored by TLC (20% MeOH/DCM). After 2 hours, the reaction was complete and neutralized with Amberlite IR-120 (H+) resin. The resin was removed by filtration, washed with MeOH and the combined filtrate was evaporated and purified by flash chromatography (0-25% MeOH:DCM) to obtain GlcNAc monomer 9′ as a pale-yellow syrup (7.5 g, 84%). The GlcNAc monomer 9′ was stored in DMF at 58% wt/wt concentration, under nitrogen overlay at −20° C. ¹H-NMR (499.9 MHz, D₂O, 25° C., ppm): δ=5.7 (s, 1H), 5.45 (s, 1H), 4.44 (d, J=8.5 Hz, 1H), 3.83-3.66 (m, 5H), 3.60-3.36 (m, 6H), 2.01 (s, 3H), 1.91 (s, 3H). ¹³C-NMR: (125 MHz, D₂O, 25° C., ppm): δ=176.2; 169.34; 141.72; 120.37; 103.0; 76.5; 72.43; 72.5; 69.82; 69.3; 61.63; 62.4; 53.9; 39.81; 18.86; 11.0. MS m/z: [M+H]+ 377.18.

Alternatively, the following procedures were used to provide 9′ (e.g., 2-(2-ethoxy)ethyl methacrylamide 2-acetamido-2-deoxy-β-D-glucopyranoside). Compound 8′ (2.0 g, 3.98 mmol) was dissolved in 10 mL of MeOH and stirred at room temperature. Sodium methoxide (4 mmol) was added to the reaction and the reaction was stirred at room temperature. After 6 hours, the solution was neutralized with Amberlite IR120 and then filtered. The solvent was removed via rotary evaporation and loaded on to silica gel. The product was purified via column chromatography using DCM:MeOH (83:17) to give 9′ as a clear solid. ¹H-NMR: (400 MHz, D₂O): δ (ppm), 5.7 (s, 1H), 5.45 (s, 1H), 4.44 (d, J=8.5 Hz, 1H), 3.83-3.66 (m, 5H), 3.60-3.36 (m, 6H), 2.01 (s, 3H), 1.91 (s, 3H). ¹³C-NMR: (125 MHz, D₂O): δ (ppm), 176.2; 169.34; 141.72; 120.37; 103.0; 76.5; 72.43; 72.5; 69.82; 69.3; 61.63; 62.4; 53.9; 39.81; 18.86; 11.0. MS m/z: [M+H]+ 377.18.

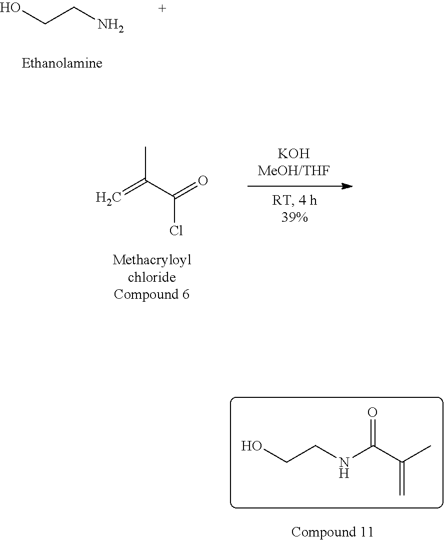

N-(2-hydroxyethyl)methacrylamide (HEMA) (11). To an ice-cold solution of ethanolamine (5.0 g, 82 mmol) in 70 mL of methanol was slowly added methacryloyl chloride (9.4 g, 90 mmol, 1.1 eq.) in THF (75 mL) under N₂ overlay. Potassium hydroxide (1.0 M, aqueous) was added to maintain a pH of 8-9 throughout the reaction. The mixture was warmed to room temperature over a period of 4 hours. The pH was adjusted to 5.0 with 1.0 M hydrochloric acid and the product was concentrated to minimum volume in the absence of light. The crude material was diluted with EtOAc, the layers separated, the aqueous layer was extracted with EtOAc three times. The combined organics were dried over Na₂SO₄, filtered, and concentrated at room temperature. Purification was done on 120 g silica gel column using a gradient of acetone:hexane (0-60%). Fractions containing pure product were combined and concentrated in the absence of light resulting in pale yellow oil 11 (4.0 g, 39%). Compound 11 was diluted 72% wt/wt in DMF and stored under nitrogen at −20° C. ¹H-NMR (499.9 MHz, CDCl₃, 25° C., ppm): δ=6.87 (m, 1H), 5.7 (m, 1H), 5.3 (m, 1H), 4.29 (s, 1H), 3.66 (t, J=5.1 Hz, 2H), 3.4 (dt, J=5.3, 5.1 Hz, 2H), 1.96 (s, 3H). ¹³C-NMR: (125 MHz, CDCl₃-d₆, 25° C., ppm): δ=166.5, 139.2, 120.1, 61.2, 42.3, 18.4. MS m/z: [M+H]+ 130.08.

Alternatively, the following procedures were used to prepare compound 11 (N-(2-Hydroxyethyl) methacrylamide). To 200 mL of an ice-cold solution of ethanolamine (12 mL) and potassium carbonate (15 g) in DCM was slowly added a solution of methacryloyl chloride (6) (9 mL) in DCM (50 mL). The reaction was allowed to come to room temperature and stirred for another 4 hours. After 4 hours the reaction mixture was filtered through celite and the solvent was removed via rotary evaporation. The crude product was loaded onto silica gel and purified via flash chromatography, Ethyl Acetate (EtOAc):Hexanes (90:10), to give 11 as a colorless oil (Yield: 75%). ¹H-NMR: (400 MHz, CDCl₃-d₆): 6.87 (m, 1H), 5.7 (m, 1H), 5.3 (m, 1H), 4.29 (s, 1H), 3.66 (t, J=5.1 Hz, 2H), 3.4 (dt, J=5.3, 5.1 Hz, H2), 1.96 (s, H3). ¹³C-NMR (125 MHz, CDC-d): δ (ppm), 166.5, 139.2, 120.1, 61.2, 42.3, 18.4. MS m/z: [M+H]+ 130.08.

### Example 2: RAFT Reagent Synthesis

The following provides exemplary procedures for the synthesis of certain RAFT reagents.

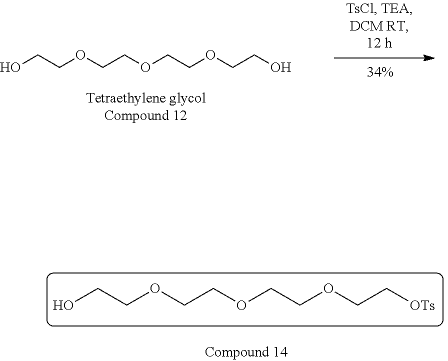 [SMILES: OCCOCCOCCOCCO ; Cc1ccc(S(=O)(=O)OCCOCCOCCOCCO)cc1]

Tetra (ethylene glycol) mono p-toluenesulfonate (14). To a solution of tetraethylene glycol 12 (26.6 g, 137 mmol) in CH₂Cl₂ (400 mL) was added 29.0 mL of triethylamine. The reaction was cooled to 0° C. and 4-methylbenzene-1-sulfonyl chloride (24.8 g, 130 mmol, 0.95 eq.) was added. The reaction was allowed to warm to room temperature and stirred for an additional 12 hours. The reaction mixture was washed with saturated NaHCO₃, brine, dried over Na₂SO₄, filtered, and concentrated in vacuo. The crude material was purified on silica gel column, eluent 0-60% acetone:hexane system. Pure product 14 was collected as a light-yellow oil (16.3 g, 34%).

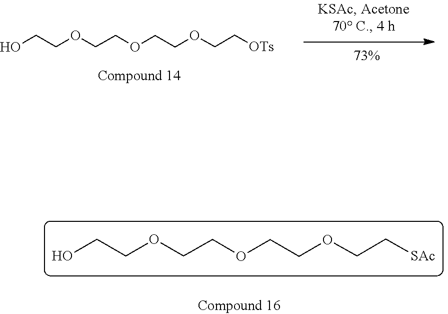

Tetra (ethylene glycol) monothioacetate (16). To a suspension of potassium thioacetate (10.7 g, 93.6 mmol, 2 eq.) in 680 mL of acetone was added a solution of mono p-toluenesulfonate 14 (16.3 g, 46.8 mmol) in 100 mL of acetone. The mixture was stirred at room temperature for 1 hour and then refluxed at 68° C. for 4 hours under a stream of nitrogen and a condenser. The reaction mixture was cooled to room temperature and filtered through a pad of Celite. The filtrate was concentrated to minimum volume, diluted with EtOAc (300 mL), washed with saturated NaHCO₃, brine, dried over Na₂SO₄ and evaporated under reduced pressure. The crude material was purified on a 120 g silica gel cartridge using an acetone:hexane gradient (0-35%), affording the desired product 16 as a brown syrup (8.65 g, 73%).

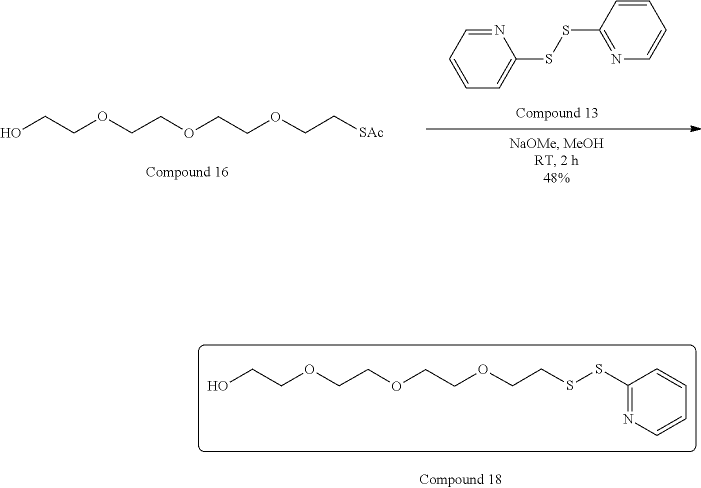

2-(2-(2-(2-(pyridin-2-yldisulfanyl)ethoxy)ethoxy)ethoxy)ethan-1-ol (18). Under nitrogen overlay, sodium methoxide (100 mL of 0.5 M in methanol) was slowly added into a stirred methanolic solution of monothioacetate 16 (5.2 g, 20.6 mmol) and 2,2-dithiodipyridine (5.44 g, 24.7 mmol, 1.2 eq.). After 2 hours, the reaction was concentrated and loaded onto a 120 g HP silica flash column and eluted with a gradient of acetone:hexane (0-50%) to afford desired product 18 as a dark yellow oil (3.15 g, 48%).

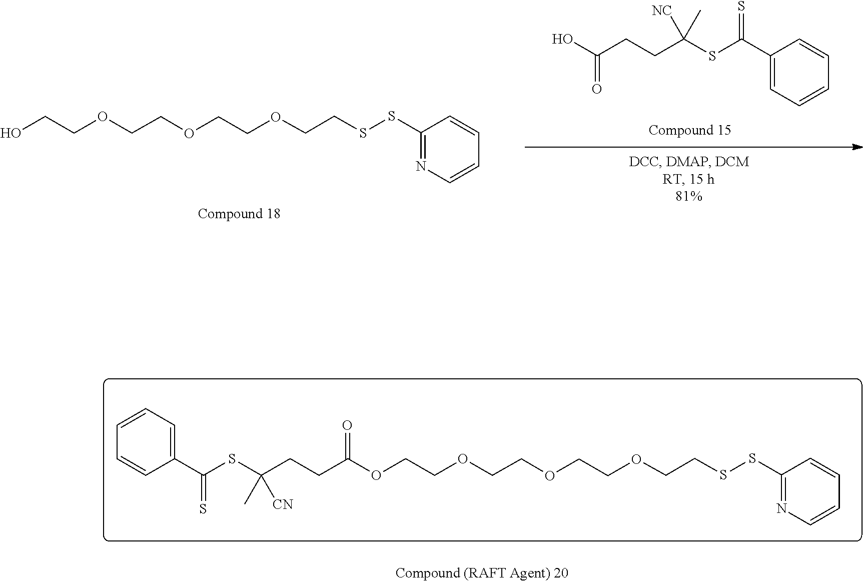 [SMILES: CC(C#N)(CCC(=O)O)SC(=S)c1ccccc1 ; OCCOCCOCCOCCSSc1ccccn1 ; CC(C#N)(CCC(=O)OCCOCCOCCOCCSSc1ccccn1)SC(=S)c1ccccc1]

Thiol-Reactive μRAFT Agent (20). Disulfide compound 18 (355 mg, 1.11 mmol) and 4-cyano-4-(thiobenzoylthio)pentanoic acid 19 (345 mg, 1.24 mmol, 1.1 equivalents) were dissolved in anhydrous DCM (7.0 mL), resulting in a pink solution. 5.0 mol % of 4-dimethylaminopyridine (DMAP) was added into solution and the flask cooled to 0° C. and stirred for 30 minutes. N,N′-dicyclohexylcarbodiimide (DCC, 230 mg, 1.1 mmol, 1 eq.) in 5.0 mL of DCM was added slowly. The reaction was stirred and allowed to equilibrate to room temperature over 5 hours. The reaction was complete by TLC but was allowed to stir for 16 hours before work-up. The pink suspension was filtered through a pad of Celite, and the filtrate concentrated. Purification was done on a 25 g HP silica gel flash column using a 0-40% acetone:hexane gradient. The fractions containing pure product were combined and concentrated resulting in RAFT agent 20 (0.53 g, 81%) as a pink oil. RAFT agent 20 was diluted to 100 mg/mL in DMF for direct use in polymerization. ¹H-NMR (499.9 MHz, D₂O, 25° C., ppm): δ=1.60 (br. S, 2H), 1.93 (s, 3H, methyl), 2.99 (t, 2H, methylene), 3.66 (m, 12H, PEG methylene), 4.27 (t, 2H, methylene), 7.08 (t, 1H, aromatic), 7.39 (t, 2H, aromatic), 7.57 (t, 1H, aromatic), 7.65 (t, 1H, aromatic), 7.77 (d, 1H, aromatic), 7.90 (d, 2H, aromatic), 8.45 (d, 1H, aromatic).

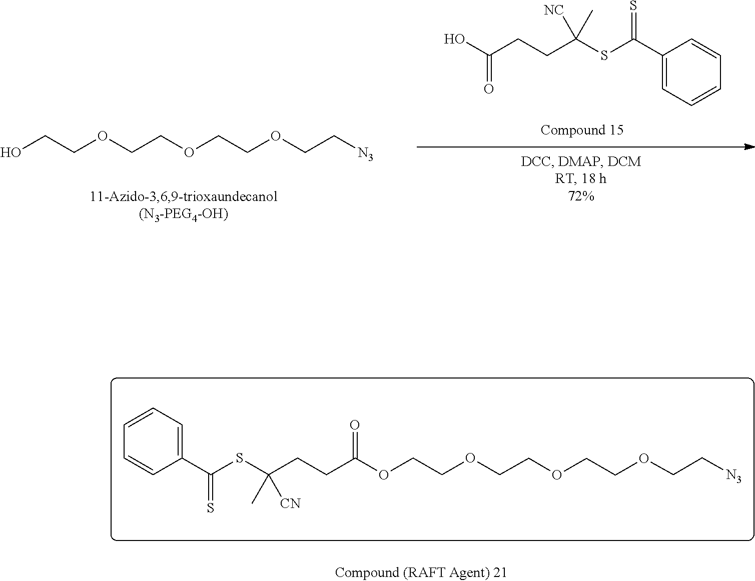 [SMILES: CC(C#N)(CCC(=O)O)SC(=S)c1ccccc1 ; [N-]=[N+]=NCCOCCOCCOCCO ; CC(C#N)(CCC(=O)OCCOCCOCCOCCN=[N+]=[N-])SC(=S)c1ccccc1]

Alkyne-Reactive μRAFT Agent (21). To 4-cyano-4-(phenylcarbonothioylthio)pentanoic acid (505.1 mg, 1.81 mmol) and 4-dimethylaminopyridine (16.4 mg, 0.13 mmol) was added anhydrous dichloromethane (7.0 mL) under and atmosphere of dry N₂ gas with stirring, giving a red solution. To the flask was added a solution of 11-Azido-3,6,9-trioxaundecanol (358.4 mg, 1.64 mmol) in anhydrous dichloromethane (7.0 mL). The mixture was cooled in a water/ice bath under an atmosphere of dry N₂ gas for 20 minutes. Using a gas-tight syringe, a solution of dicyclohexyl carbodiimide (374 mg, 1.81 mmol) in anhydrous dichloromethane (7.0 mL) was slowly added to the reaction mixture over 15 minutes. A precipitate was observed to slowly form turning the reaction mixture dark pink in color. The mixture was maintained in a water/ice bath for 3 hours and then allowed to come to ambient temperature over 18 hours. The reaction mixture was then filtered through a Celite pad and the pad was rinsed with dichloromethane (3×15.0 mL) when all the red color was removed from the Celite. The solution was concentrated under vacuum at 20° C. to 1.0 mL and chromatographed on a silica gel column (12.0 g) using a gradient of acetone:hexane (0-40%). TLC (hexane:acetone 2:1 v/v) showed a major product with R_{f}=0.30. Fractions containing the product R_{f}=0.30 were pooled and concentrated to a red oil. The sample was then dried under vacuum for 30 hours to yield a dark red oil (563.0 mg, 72%). The final product and was then stored at 2° C. in a light resistant container. ¹H-NMR (499.9 MHz, CDCl₃, 25° C., ppm): δ=1.93 (s, 3H, CH3-C—(CN)—S); 3.37 (t, 2H, CH2-CH2—C(O)—O); 3.6-3.75 (m, 16H, —C(O)—O—[CH2-CH2-O]3-CH2-CH2-N3); 4.26 (t, 2H, —CH2-(CH3)-CN); 7.39 (m, 2H); 7.56 (m, 1H); 7.90 (m, 2H).

Alternatively, the following procedures were used to provide 21 (N₃TEG-RAFT). Azido-tetraethylene glycol 11 (219 mg, 1.0 mmol), DMAP (12 mg, 0.1 mmol) and RAFT agent 15 (279.0 mg, 1.0 mmol) were added to 10 mL of DCM and stirred on ice for 30 min. A solution of DCC (206 mg, 1.0 mmol) in DCM was added dropwise to the reaction mixture. The reaction mixture was allowed to come to room temperature and stirred for another 3 hours. The reaction was filtered, and the solvent was removed via rotary evaporation. The product was loaded onto silica gel and separated via column chromatography using EtOAC to yield 21 as a pink liquid. (Yield: 23%). ¹H-NMR: (400 MHz, CDCl₃-d₆): δ (ppm), 7.76 (m, 2H), 7.43 (m, 1H), 7.28 (m, 2H), 4.11 (m, 2H), 3.57 (m, 2H), 3.51 (m, 12H), 3.23 (m, 2H), 2.75-2.45 (m, 4H), 1.79 (s, 3H). ¹³C-NMR: (125 MHz, CDCl₃-d₆): δ (ppm), 221.2; 171.34; 144.72; 135.37; 129.0; 126.5; 119.6; 68.43; 65.5; 44.82; 31.3; 29.64; 24.5; 12.4. MS m/z: [M+H]+ 481.17.

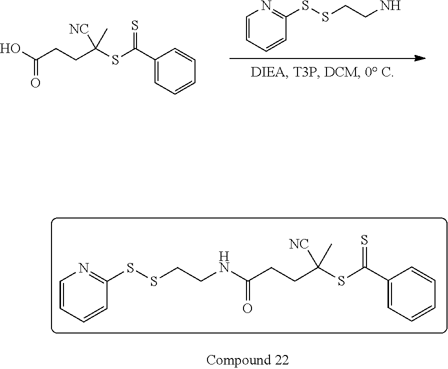 [SMILES: CC(C#N)(CCC(=O)O)SC(=S)c1ccccc1 ; NCCSSc1ccccn1 ; CC(C#N)(CCC(=O)NCCSSc1ccccn1)SC(=S)c1ccccc1]

Pyridyl disulfide pentanamide RAFT reagent (22): To 4-cyano-4-dithiobenzoyl pentanoic acid (0.6920 g, 2.47 mmol) under argon was added dry CH₂Cl₂ (10 mL) using a syringe and stirred for 10 min until the red solid had dissolved. The solution was cooled in an ice bath for 5 min and then DIEA (1.2 mL, 6.9 mmol) was added. The solution was stirred for an additional 10 min under argon. To the solution was then added T3P (1.6 mL, 2.5 mmol) slowly over ˜5 minutes using a syringe. The solution was allowed to stir in an ice bath for 15 min. The septum was removed and 2-(2-pyridylthio)cysteamine hydrochloride (0.5041 g, 2.27 mmol) was added. The septum was replaced, and the mixture stirred under argon. The mixture was allowed to come to ambient temperature as the ice melted. After 4 hours the solution was concentrated to 30% in vacuo and diluted with isopropyl acetate (60 mL). The solution was then extracted with water (50 mL), 1M hydrochloric acid (50 mL), saturated sodium hydrogen carbonate (50 mL), and water (50 mL). The organic layers were collected and dried over anhydrous sodium sulfate. The solid was filtered off and the solution concentrated in vacuo to a red oil. The oil was purification via column chromatography using silica gel (12 g) and hexanes/ethyl acetate gradient 0-45% v/v with TLC monitoring. Fractions containing the product were pooled and concentrated to a red oil and then dried in vacuo to afford a clear dark red oil.

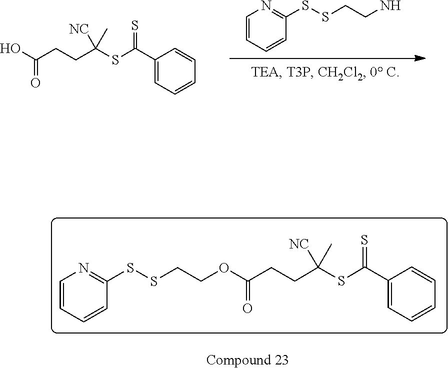 [SMILES: CC(C#N)(CCC(=O)O)SC(=S)c1ccccc1 ; NCCSSc1ccccn1 ; CC(C#N)(CCC(=O)OCCSSc1ccccn1)SC(=S)c1ccccc1]

Pyridyl disulfide pentanoate RAFT reagent (23): To 4-cyano-4-dithiobenzoylpentanoic acid (0.5070 g, 1.81 mmol) under argon was added dry CH₂Cl₂ (5 mL) using a syringe and stirred for 10 minutes until the red solid had dissolved. The solution was cooled in an ice bath for 5 min and then TEA (0.6 mL, 4.31 mmol) was added. The solution was stirred for an additional 5 minutes under argon. To the solution was then added T3P (1.05 mL, 1.81 mmol) slowly over 5 min using a syringe. The solution was allowed to stir in the ice bath for 20 min. A solution of 2-(2-pyridyl) disulfaneyl ethanol (0.308 g, 1.64 mmol, in 0.6 mL anhydrous CH₂Cl₂ was slowly added to the reaction mixture over 5 min. An additional 1.0 mL of anhydrous CH₂Cl₂ was then added and the mixture was allowed to come to ambient temperature as the ice melted. After stirring for 16 hours the solution was concentrated under reduced pressure and dissolved in isopropyl acetate (30 mL). The solution was extracted with saturated sodium hydrogen carbonate (25 mL), and twice with 10% w/v sodium chloride (25 and 20 mL). The organic layers were collected, dried over anhydrous sodium carbonate, filtered, and the solution concentrated in vacuo to a red oil.

### Example 3: poly(GalNAc-co-HEMA)-PDS and Dithioester-Free (e.g., DTB-Free) poly(GalNAc-co-HEMA)-PDS Polymer Synthesis

The following provides exemplary procedures for the synthesis of certain thiol-terminated Y(Z)-EU units (e.g., disulfide terminated polymers).

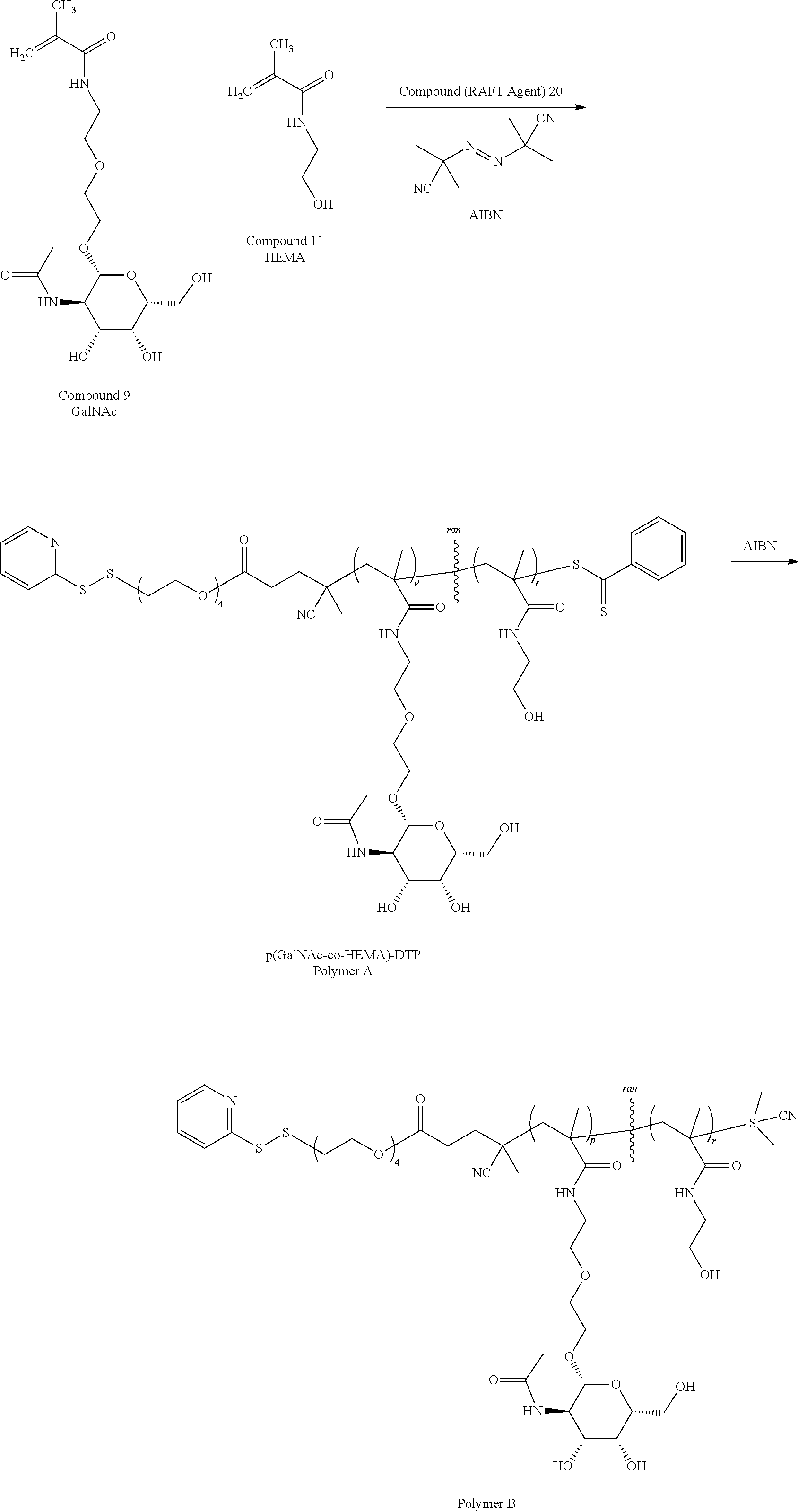

### Synthesis of Polymer A

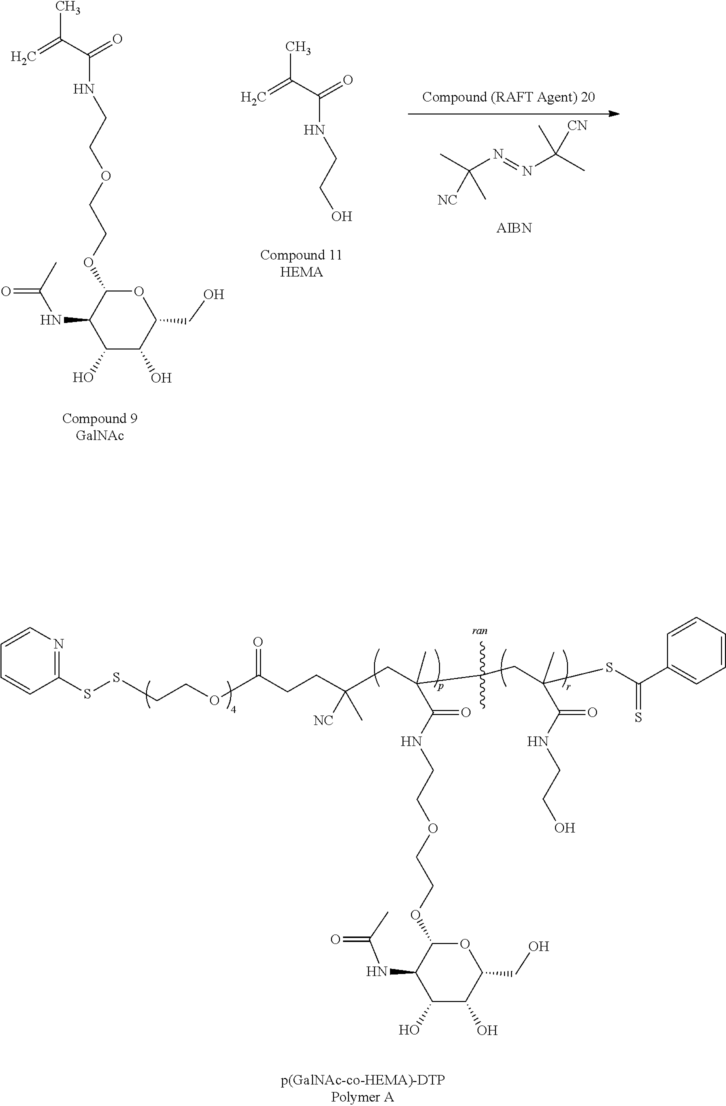

poly(GalNAc-co-HEMA)-PDS (Polymer A). A typical example synthesis of a poly(GalNAc-co-HEMA)-PDS with a target molecular weight of 21.0 kDa, a target degree of polymerization of 100 monomers, and a target GalNAc:HEMA monomer composition of 30:70 is as follows: A 10 mL single-neck Schlenk flask equipped with a PTFE valve and situated in a low-light area was purged with ultra-high-purity Argon (Grade 5), placed in an ice bath, and charged with a magnetic stir bar, compound 9 (300 mg, 0.80 mmol, solid), compound 11 (240 mg, 1.86 mmol, added as 240 μL neat oil), compound 20 (15.4 mg, 30 μmol added as 283 μL of stock solution at 54.53 mg/mL), 2,2′-azobis(2-methylpropionitrile) (1.09 mg, 6.6 μmol, added as 270 μL of stock solution at 4.04 mg/mL), and N,N-dimethylformamide (827 μL). The flask was sealed with a rubber septum, the septum reinforced with parafilm, and the solution was sparged on ice with ultra-high-purity (Grade 5) Argon for 2 hours. Following sparging, the solution was subjected to five freeze-pump-thaw cycles over liquid Nitrogen, each cycle consisted of a 3-minute freeze step, a 15-minute pump step, and a 2-minute thaw step. The solution was then overlaid with ultra-high-purity (Grade 5) Argon and allowed to stir at 800 rpm in a pre-heated oil bath at 68° C. for 18 hours. The RAFT polymerization was quenched by submerging the flask in an ice bath, exposing the solution to air, and allowing the solution to stir on ice at 500 rpm for 15 minutes. The crude polymer solution was then precipitated dropwise into 45 mL anhydrous ethyl acetate at room temperature and the resultant precipitate was pelleted via centrifugation at 4300-G for 10 minutes. The supernatant was then decanted, replaced with fresh anhydrous ethyl acetate, the pellet was re-suspended via vortex, re-pelleted via centrifugation, and the supernatant decanted again, affording a resultant pellet which was dried under high vacuum at room temperature for 2 hours affording a pink powder. The dried crude polymer was re-dissolved in 8.0 mL Milli-Q water, charged into a Slide-a-Lyzer dialysis cassette (3.5 kDa MWCO) and dialyzed against 500 volumes of Milli-Q water for 24 hours during which solvent exchanges were performed at t=4 hours and t=20 hours. The dialyzed aqueous solution was then dried via lyophilization for 4 days to yield poly(GalNAc-co-HEMA)-PDS as a flaky light-pink solid (222.7 mg, 40.1%). GPC: Mₙ=22.2 kDa, M_{w}=24.7 kDa, Mₚ=21.9 kDa, Ð=1.11. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer A′

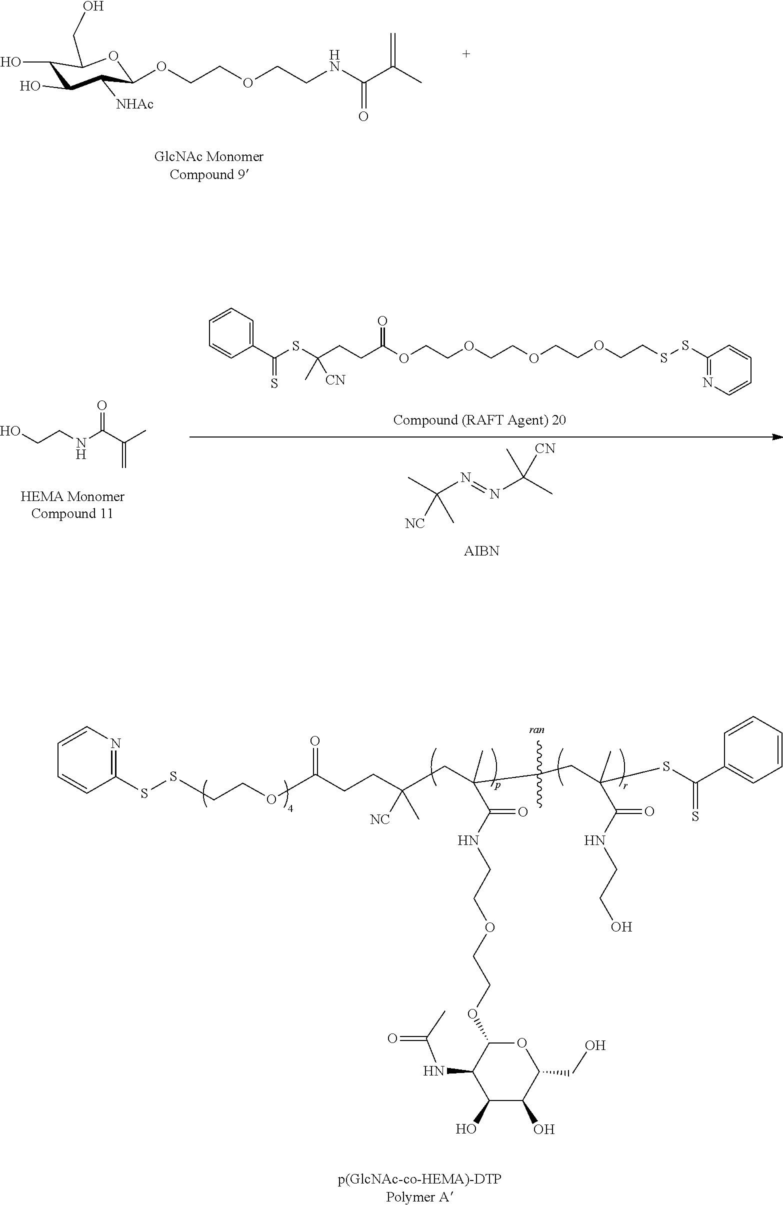

A similar procedure for that used to prepare poly(GalNAc-co-HEMA)-PDS (shown above) was used to produce poly(GlcNAc-co-HEMA)-PDS. Certain embodiments of thiol-reactive polymers produced with the above procedure are described in Table 1. Structure and molecular weight was confirmed by NMR and GPC, respectively.

TABLE 1
Thiol-reactive polymers produced using procedures as disclosed
in Example 3 using different monomer ratios.

| Polymer | Sugar:HEM A Ratio | Mₙ | PDI |
|---|---|---|---|
| poly(GalNAc-co-HEMA)-PDS | 4:1 | 14.9 | 1.10 |
| poly(GalNAc-co-HEMA)-PDS | 3:7 | 21.9 | 1.11 |
| poly(GalNAc-co-HEMA)-PDS | 1:4 | 13.2 | 1.10 |
| poly(GlcNAc-co-HEMA)-PDS | 4:1 | 21.5 | 1.09 |
| poly(GlcNAc-co-HEMA)-PDS | 1:1 | 19.3 | 1.11 |
| poly(GlcNAc-co-HEMA)-PDS | 1:4 | 14.5 | 1.12 |

### Synthesis of Polymer B

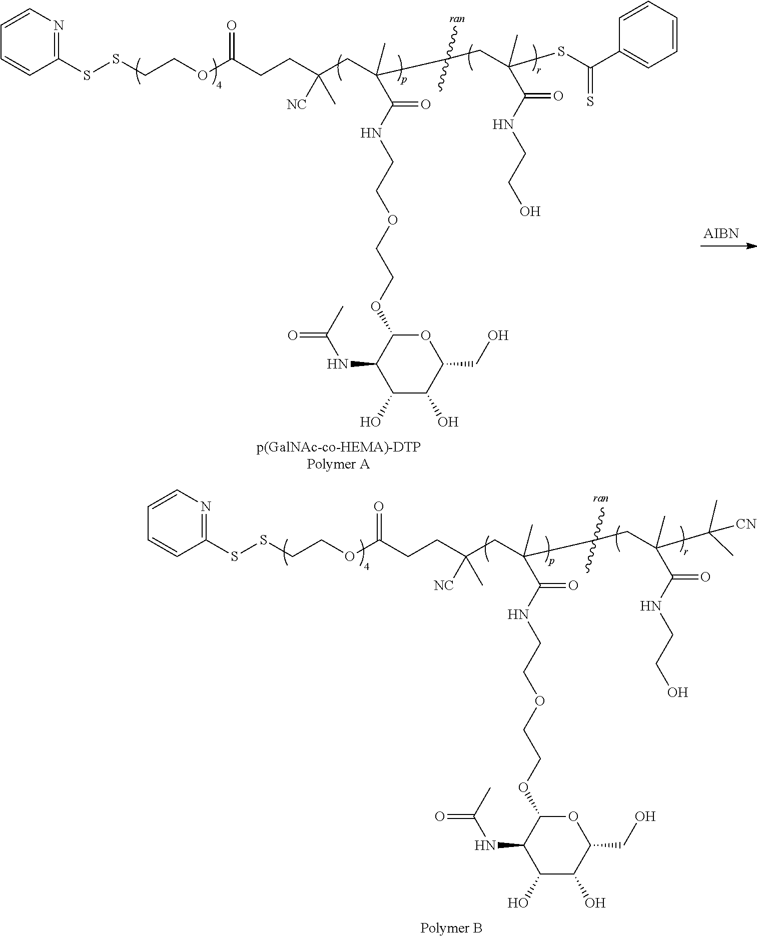

Dithioester-Free (e.g., DTB-Free) poly(GalNAc-co-HEMA)-PDS (Polymer B). A 500 mL single-neck Schlenk flask equipped with a PTFE valve and situated in a low-light area was purged with high-purity Argon (Grade 6) and charged with a magnetic stir bar, poly(GalNAc-co-HEMA)-PDS intermediate (19.4 kDa, 40.0 g, 2.0619 mmol, 1.0 Eq., solid), 2,2′-azobis(2-methylpropionitrile) (AIBN) (17.0 g, 103.09 mmol, 50 Eq., solid), and N,N-dimethylformamide (333 mL, anhydrous). The order of addition was: AIBN, pGal, DMF. The flask was sealed with a rubber septum, the septum reinforced with parafilm, and the suspension was allowed to stir in an ice bath at 700 rpm while being sparged with Argon for 60 minutes. Following sparge, the solution was degassed according to the following procedure: The contents of the flask are stirred at 700 rpm and exposed to high vacuum for 3 minutes. The vacuum is then turned off and the contents are back-filled with argon. This process is repeated 10 times. After the final pump cycle, the degassed solution is overlaid with argon and allowed to stir at 700 rpm in a pre-heated oil bath at 75° C. for 2 hours. The reaction was quenched according to the following procedure: The flask was removed from the oil bath, immediately submerged in an ice bath, and allowed to stir on ice at 700 rpm for 10 minutes. The septum and Schlenk valve were then removed from the flask, and the crude reaction solution was allowed to stir on ice at 700 rpm for an additional 15 minutes. The crude reaction solution was then precipitated into 2000 mL EtOAc at room temperature. The resultant precipitate was filtered via disposable polyethylene fritted funnel (40-micron, 2000 mL capacity), washed four times with EtOAc, isolated, and dried in vacuo. The dried precipitate was re-dissolved in 750 mL Milli-Q water, charged into two Slide-a-Lyzer dialysis cassettes (250 mL capacity, 10.0 kDa MWCO) and dialyzed against 16 volumes of Milli-Q water for 48 hours during which solvent exchanges were performed at t=4, 12, 24 and 36 hours. The dialyzed aqueous solution was then dried via lyophilization to afford poly(GalNAc-co-HEMA)-PDS final product as a flaky off-white solid (35.8 g, 89.5%). GPC: Mₙ=16.5 kDa, M_{w}=19.2 kDa, Mₚ=19.5 kDa, Ð=1.17. ¹H-NMR (499.9 MHz, D²O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.2-8.6 (m, 4H, end-group aromatic). The DTB content of Polymer B was measured using reversed phase HPLC (at 500 nm using a UV detector). For five different lots of Polymer B, the dithioester content after DTB removal was 5.1%, 4.5%, 4.8%, 3.8%, and 6.4%. Prior to removal, the DTB content of Polymer A was between 70% and 80%.

### Example 4: poly(GalNAc-co-HEMA)-PDS Ethyl Ester and a Dithioester-Free (e.g., DTB-Free) poly(GalNAc-co-HEMA)-PDS Ethyl Ester Polymer Synthesis

The following example describes an embodiment of the synthesis of a poly(GalNAc-co-HEMA)-PDS ethyl ester (Polymer C) and a Dithioester-Free (e.g., DTB-Free) poly(GalNAc-co-HEMA)-PDS ethyl ester (Polymer D), as shown in the following scheme.

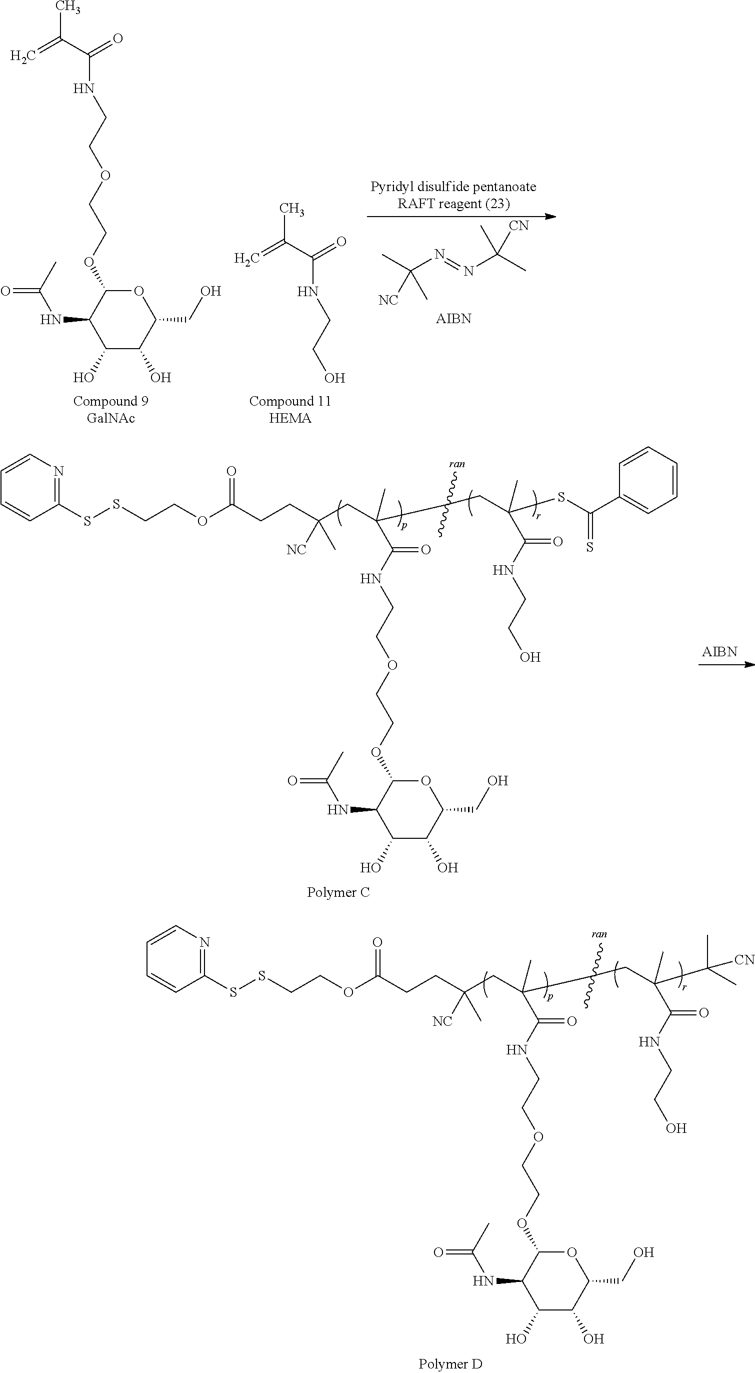

### Synthesis of Polymer C

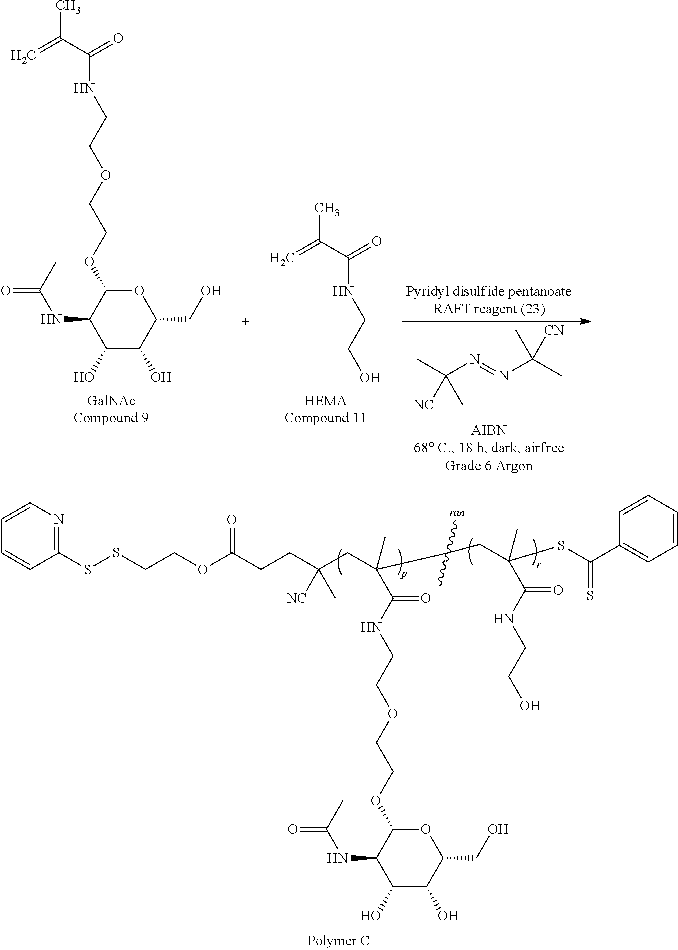

poly(GalNAc-co-HEMA)-PDS Ethyl Ester (Polymer C). A 10 mL single-neck Schlenk flask equipped with a PTFE valve and situated in a low-light area was purged with Argon (Grade 6), placed in an ice bath, and charged with a magnetic stir bar, compound 9 (400 mg, 1.06 mmol, solid), compound 11 (106 mg, 0.82 mmol, added as 106 μL neat oil), pyridyl disulfide pentanoate RAFT reagent 23 (13.6 mg, 30.4 μmol added as 418 μL of stock solution at 32.49 mg/mL), 2,2′-azobis(2-methylpropionitrile) (1.50 mg, 9.1 μmol, added as 374 μL of stock solution at 4.0 mg/mL), and N,N-dimethylformamide (662 μL). The flask was sealed with a rubber septum, the septum reinforced with parafilm, and the solution was sparged on ice with argon for 2 hours. Following sparging, the solution was subjected to five freeze-pump-thaw cycles over liquid Nitrogen, each cycle consisted of a 3-minute freeze step, a 15-minute pump step, and a 2-minute thaw step. The solution was then overlaid with argon and allowed to stir at 800 rpm in a pre-heated oil bath at 68° C. for 18 hours. The RAFT polymerization was quenched by submerging the flask in an ice bath, exposing the solution to air, and allowing the solution to stir on ice at 500 rpm for 15 minutes. The crude polymer solution was then precipitated dropwise into 45 mL anhydrous ethyl acetate at room temperature and the resultant precipitate was pelleted via centrifugation at 4300-G for 10 minutes. The supernatant was then decanted, replaced with fresh anhydrous ethyl acetate, the pellet was re-suspended via vortex, re-pelleted via centrifugation, and the supernatant decanted again, affording a resultant pellet which was dried under high vacuum at room temperature for 2 hours affording a pink powder. The dried crude polymer was re-dissolved in 8.0 mL Milli-Q water, charged into a Slide-a-Lyzer dialysis cassette (3.5 kDa MWCO) and dialyzed against 500 volumes of Milli-Q water for 24 hours during which solvent exchanges were performed at t=4 hours and t=20 hours. The dialyzed aqueous solution was then dried via lyophilization for 4 days to yield poly(GalNAc-co-HEMA)-PDS ethyl ester as a flaky light-pink solid (typically, 272.9 mg). GPC: Mₙ=21.8 kDa, M_{w}=24.2 kDa, Mp=22.3 kDa, Ð=1.11. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer D

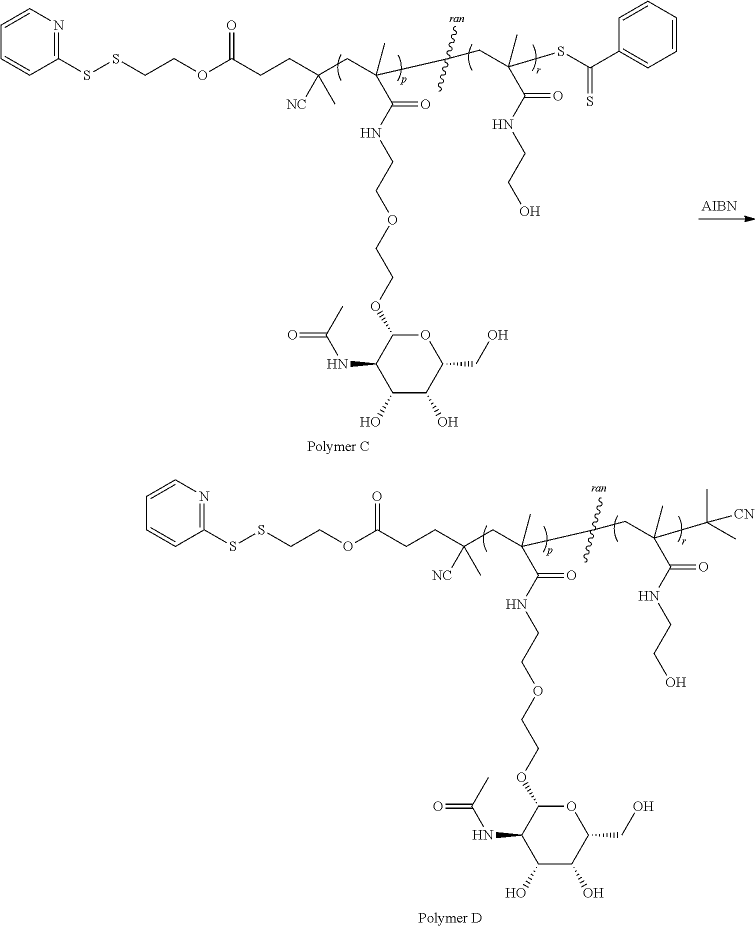

Dithioester-Free (e.g., DTB-Free) poly(GalNAc-co-HEMA)-PDS ethyl ester (Polymer D). It is believed that dithioester-free (e.g., DTB-Free) poly(GalNAc-co-HEMA)-PDS ethyl ester can be synthesized using conditions similar to those from Example 3. This is a prophetic example.

### Example 5: poly(GalNAc-co-HEMA)-PDS Ethyl Amide and a Dithioester-Free (e.g., DTB-Free) poly(GalNAc-co-HEMA)-PDS Ethyl Amide Polymer Synthesis

The following describes an embodiment of the synthesis of a poly(GalNAc-co-HEMA)-PDS ethyl amide (Polymer E) and a dithioester-free (e.g., DTB-Free) poly(GalNAc-co-HEMA)-PDS ethyl amide (Polymer F), as shown in the following scheme.

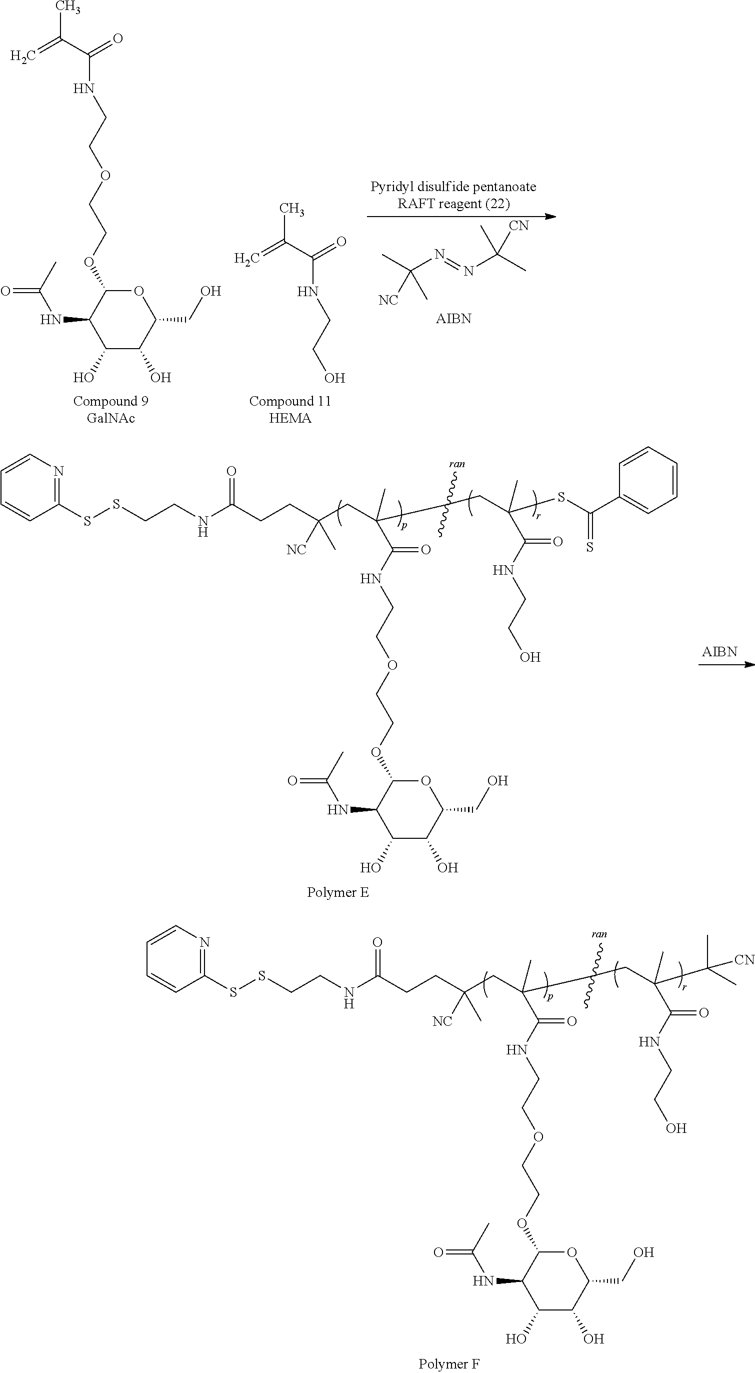

### Synthesis of Polymer E

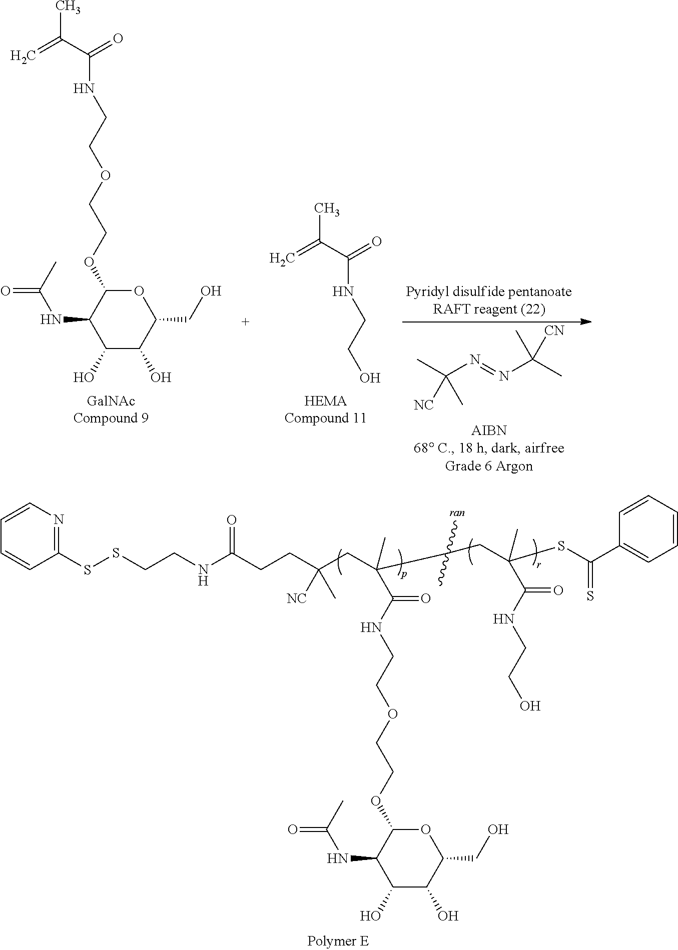

poly(GalNAc-co-HEMA)-PDS ethyl amide (Polymer E). A 10 mL single-neck Schlenk flask equipped with a PTFE valve and situated in a low-light area was purged with Argon (Grade 6), placed in an ice bath, and charged with a magnetic stir bar, compound 9 (400 mg, 1.06 mmol, solid), compound 11 (106 mg, 0.82 mmol, added as 106 μL neat oil), pyridyl disulfide pentanamide RAFT reagent (22) (13.6 mg, 30.4 μmol added as 384 μL of stock solution at 35.31 mg/mL), 2,2′-azobis(2-methylpropionitrile) (1.50 mg, 9.1 μmol, added as 374 μL of stock solution at 4.0 mg/mL), and N,N-dimethylformamide (696 μL). The flask was sealed with a rubber septum, the septum reinforced with parafilm, and the solution was sparged on ice with argon for 2 hours. Following sparging, the solution was subjected to five freeze-pump-thaw cycles over liquid Nitrogen, each cycle consisted of a 3-minute freeze step, a 15-minute pump step, and a 2-minute thaw step. The solution was then overlaid with argon and allowed to stir at 800 rpm in a pre-heated oil bath at 68° C. for 18 hours. The RAFT polymerization was quenched by submerging the flask in an ice bath, exposing the solution to air, and allowing the solution to stir on ice at 500 rpm for 15 minutes. The crude polymer solution was then precipitated dropwise into 45 mL anhydrous ethyl acetate at room temperature and the resultant precipitate was pelleted via centrifugation at 4300-G for 10 minutes. The supernatant was then decanted, replaced with fresh anhydrous ethyl acetate, the pellet was re-suspended via vortex, re-pelleted via centrifugation, and the supernatant decanted again, affording a resultant pellet which was dried under high vacuum at room temperature for 2 hours affording a pink powder. The dried crude polymer was re-dissolved in 8.0 mL Milli-Q water, charged into a Slide-a-Lyzer dialysis cassette (3.5 kDa MWCO) and dialyzed against 500 volumes of Milli-Q water for 24 hours during which solvent exchanges were performed at t=4 hours and t=20 hours. The dialyzed aqueous solution was then dried via lyophilization for 4 days to yield poly(GalNAc-co-HEMA)-PDS ethyl amide or ethyl ester as a flaky light-pink solid (typically, 267.3 mg). GPC: Mₙ=21.3 kDa, M_{w}=24.0 kDa, Mp=22.1 kDa, Ð=1.12. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer F

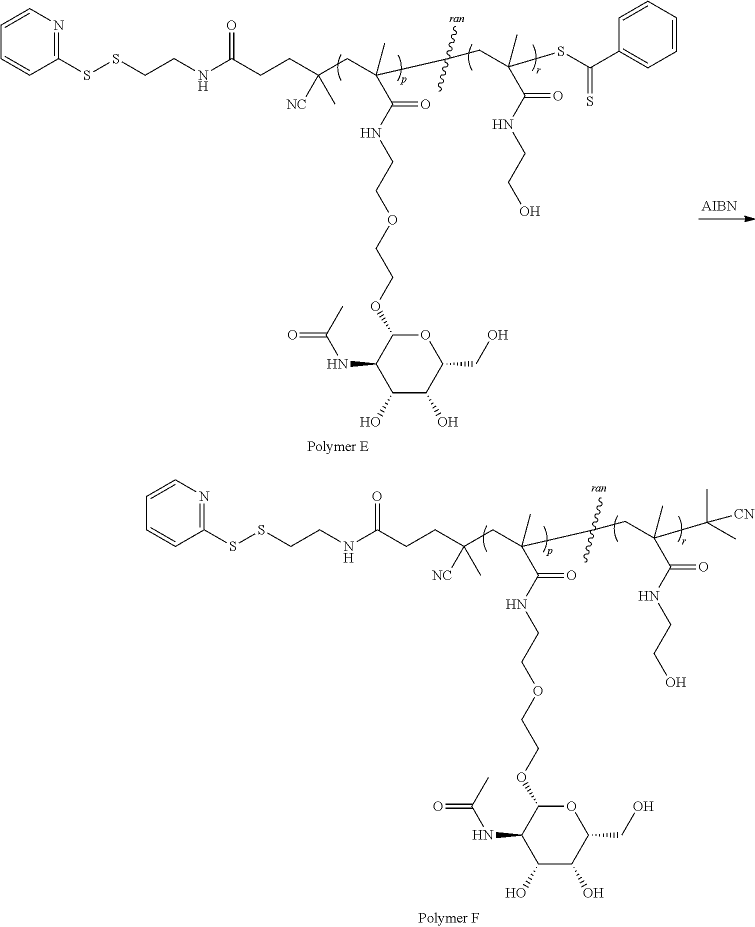

Dithioester-Free (e.g., DTB-Free) poly(GalNAc-co-HEMA)-PDS ethyl amide (Polymer F). It is believed that dithioester-free (e.g., DTB-Free) poly(GalNAc-co-HEMA)-PDS ethyl amide can be synthesized using conditions similar to those from Example 3. This is a prophetic example.

### Example 6: Alternative Synthesis of Dithioester-Free (e.g., DTB-Free) poly(GalNAc-co-HEMA)-PDS, Dithioester-Free (e.g., DTB-Free) poly(GalNAc-co-HEMA)-PDS Ethyl Amide, and Dithioester-Free (e.g., DTB-Free) poly(GalNAc-co-HEMA)-PDS Ethyl Amide Polymer Synthesis

The following describes synthetic methods for providing a carboxylic acid-terminated polymer, a carboxylic acid-terminated dithioester-free (e.g., DTB-free) polymer, and Polymers B, D, and F therefrom, as shown in the following schemes. The carboxylic acid-terminated polymer and the carboxylic acid-terminated dithioester-free (e.g., DTB-free) polymer are Polymer G and Polymer H, respectively.

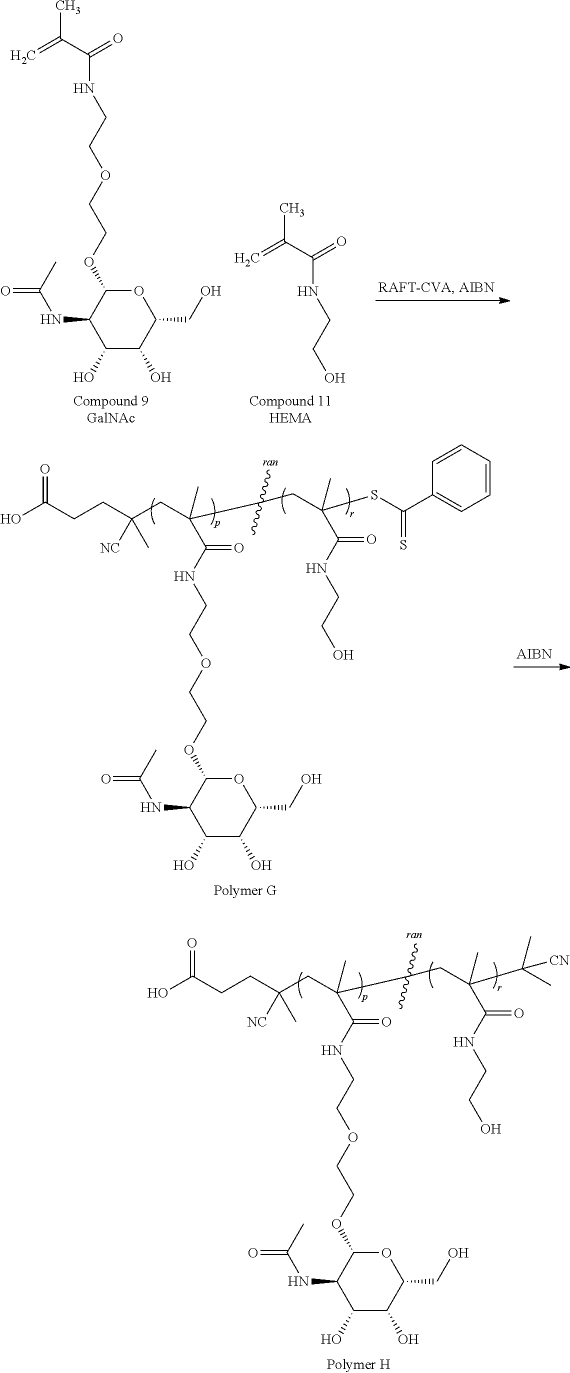

As mentioned above, the following shows an embodiment of a synthetic scheme for preparing dithioester-free (e.g., DTB-free) polymers, for example, Polymer B, Polymer D, and Polymer F. As shown, in some embodiment, these polymers can be prepared using Polymer H as a starting material.

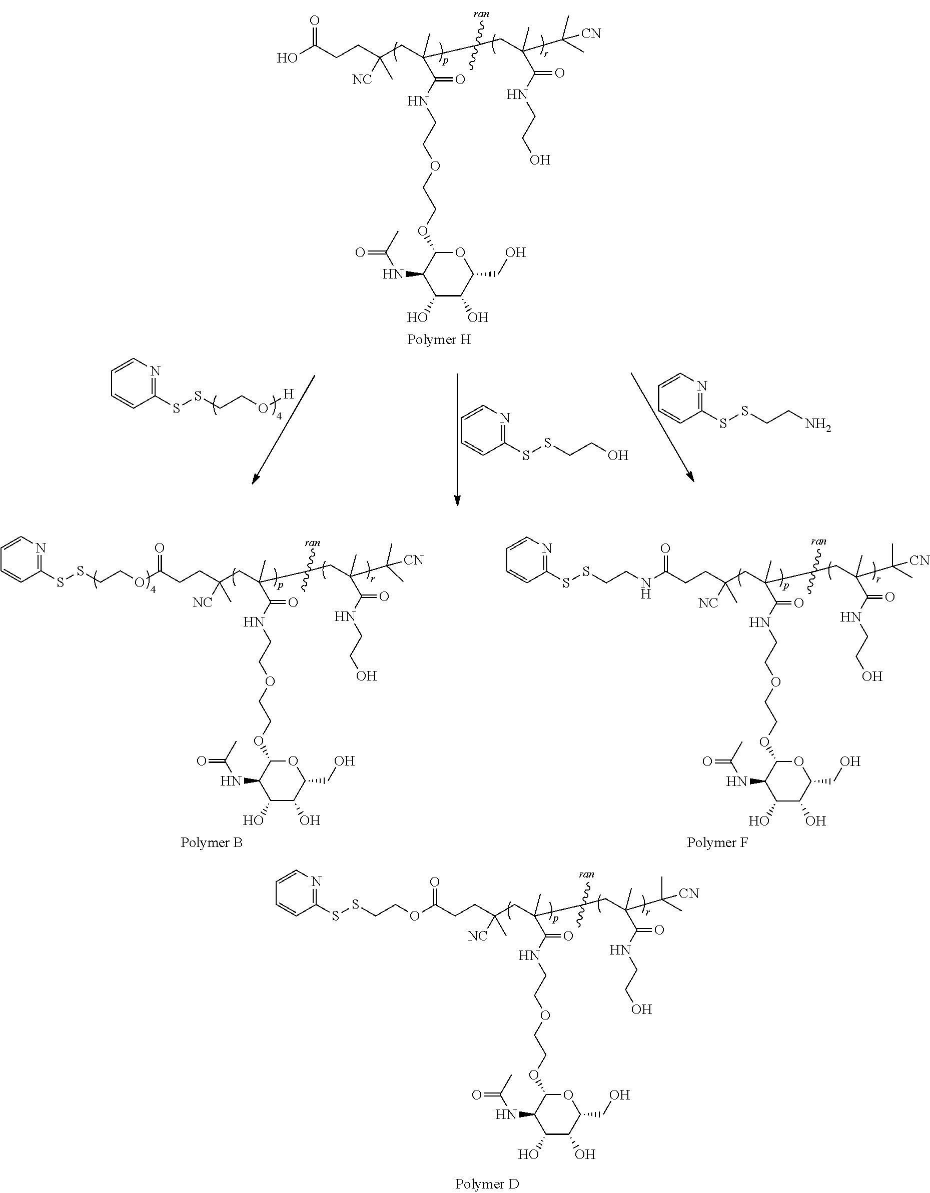

### Synthesis of Polymer G

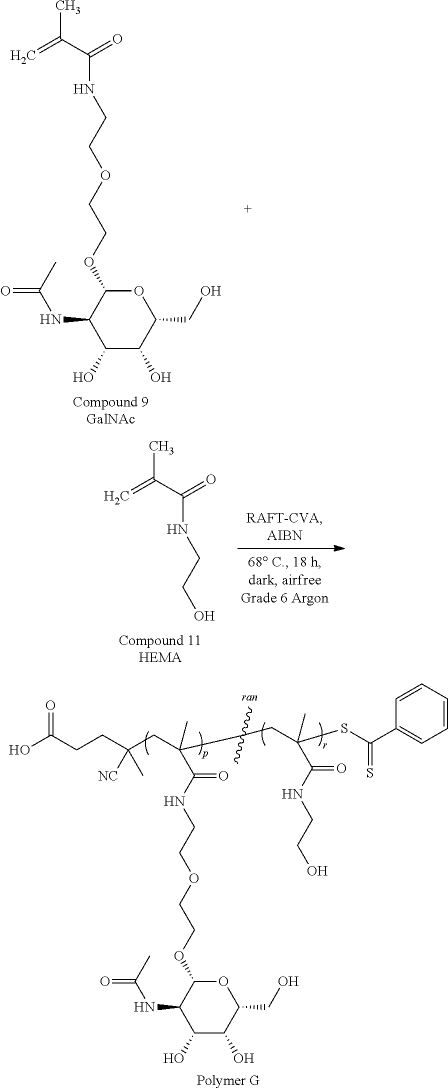

poly(GalNAc-co-HEMA)-CVA (Polymer G). A 10 mL single-neck Schlenk flask equipped with a PTFE valve and situated in a low-light area was purged with Argon (Grade 6), placed in an ice bath, and charged with a magnetic stir bar, compound 9 (1000 mg, 2.66 mmol, solid), compound 11 (291 mg, 2.26 mmol, added as 292 μL neat oil), 4-Cyano-4-(phenylcarbonothioylthio) pentanoic acid RAFT reagent (RAF T-CVA) (18.56 mg, 66.4 μmol added as 386 μL of stock solution at 48.10 mg/mL), 2,2′-azobis(2-methylpropionitrile) (5.45 mg, 33.2 μmol, added as 1.4 mL of stock solution at 4.0 mg/mL), and N,N-dimethylformamide (1.9 mL). The flask was sealed with a rubber septum, the septum reinforced with parafilm, and the solution was sparged on ice with argon for 2 hours. Following sparging, the solution was subjected to five freeze-pump-thaw cycles over liquid Nitrogen, each cycle consisted of a 3-minute freeze step, a 15-minute pump step, and a 2-minute thaw step. The solution was then overlaid with argon and allowed to stir at 800 rpm in a pre-heated oil bath at 68° C. for 18 hours. The RAFT polymerization was quenched by submerging the flask in an ice bath, exposing the solution to air, and allowing the solution to stir on ice at 500 rpm for 15 minutes. The crude polymer solution was then precipitated dropwise into 45 mL anhydrous ethyl acetate at room temperature and the resultant precipitate was pelleted via centrifugation at 4300-G for 10 minutes. The supernatant was then decanted, replaced with fresh anhydrous ethyl acetate, the pellet was re-suspended via vortex, re-pelleted via centrifugation, and the supernatant decanted again, affording a resultant pellet which was dried under high vacuum at room temperature for 2 hours affording a pink powder. The dried crude polymer was re-dissolved in 8.0 mL Milli-Q water, charged into a Slide-a-Lyzer dialysis cassette (3.5 kDa MWCO) and dialyzed against 500 volumes of Milli-Q water for 24 hours during which solvent exchanges were performed at t=4 hours and t=20 hours. The dialyzed aqueous solution was then dried via lyophilization for 4 days to yield poly(GalNAc-co-HEMA)-CVA as a flaky light-pink solid (typically, 702.6 mg). GPC: Mₙ=19.4 kDa, M_{w}=21.4 kDa, Mₚ=23.7 kDa, Ð=1.10. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer H

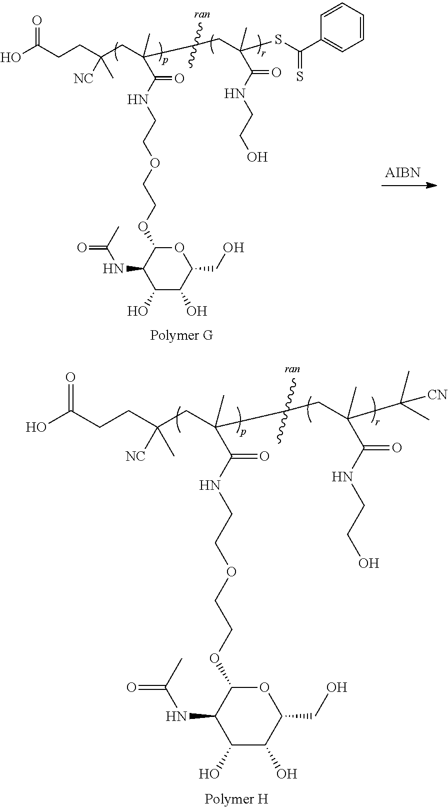

Dithioester-Free (e.g., DTB-Free) poly(GalNAc-co-HEMA)-CVA (Polymer H) was synthesized using conditions similar to those from Example 3.

### Synthesis of Polymer B

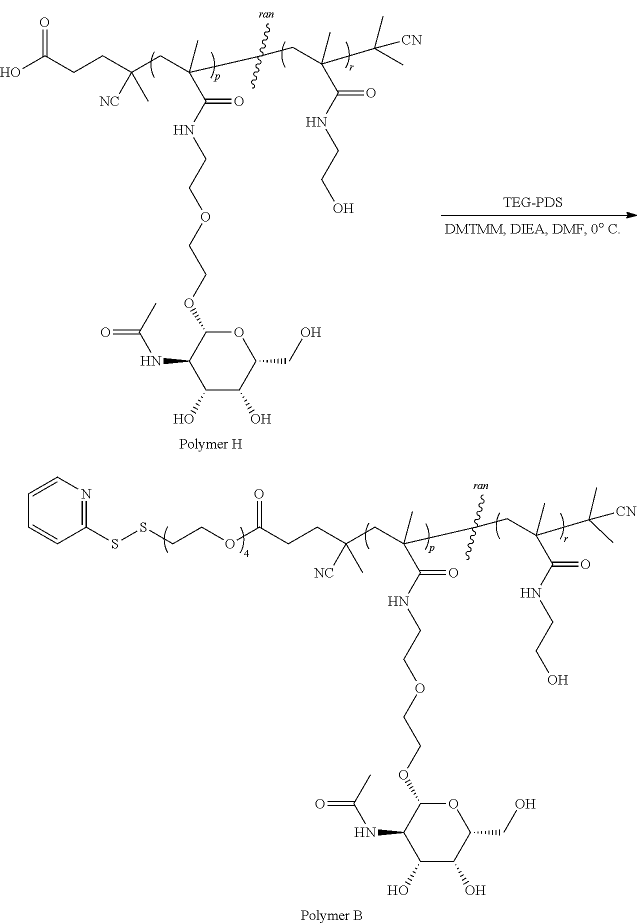

Dithioester-Free (e.g., DTB-free) poly(GalNAc-co-HEMA)-PDS (Polymer B). To a solution of dithioester-free (e.g., DTB-free) poly(GalNAc-co-HEMA)-CVA (26 mg, 1.3 μmol) in DMF (1.0 mL) at 0° C. is added 2-(2-(2-(2-(pyridin-2-yldisulfanyl)ethoxy)ethoxy)ethoxy)ethan-1-ol (TEG-PDS, 100 mg, 0.3 mmol), 4-(4,6-dimethoxy-1,3,5-triazin-2-yl)-4-methyl-morpholinium chloride (DMTMM, 5 mg, 54.3 μmol) and N, N-diisopropylethylamine (DIEA, 100 μL, 0.6 mmol). The reaction is warmed to room temperature while stirring for 15 hours. The crude product is then precipitated dropwise into EtOAc (45 mL). The precipitate and solvent mixture is centrifuged at 2000-G for 30 min with careful removal of supernatant. The solid is re-dissolved in 2.0 mL Milli-Q water, charged into a Slide-a-Lyzer dialysis cassette (10 kDa MWCO) and dialyzed against 4 L of Milli-Q water for 24 hours during which solvent exchanges are performed at t=4 hours and t=8 hours. The dialyzed aqueous solution is then dried via lyophilization to yield poly(GalNAc-co-HEMA)-PDS as a solid. This is a prophetic example.

### Synthesis of Polymer D

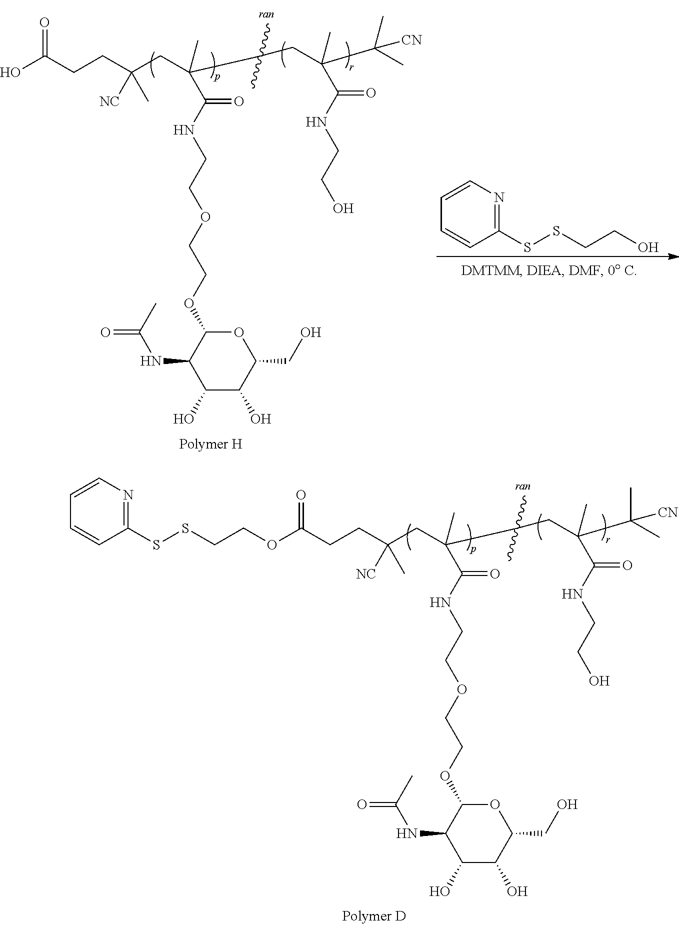

Dithioester-Free (e.g., DTB-free) poly(GalNAc-co-HEMA)-PDS (Polymer D). To a solution of dithioester-free (e.g., DTB-free poly(GalNAc-co-HEMA)-CVA (26 mg, 1.3 μmol) in DMF (1.0 mL) at 0° C. is added 2-(2-(2-(2-(pyridin-2-yldisulfanyl)ethoxy)ethoxy)ethoxy)ethan-1-ol (TEG-PDS, 100 mg, 0.3 mmol), 4-(4,6-dimethoxy-1,3,5-triazin-2-yl)-4-methyl-morpholinium chloride (DMTMM, 15 mg, 54.3 μmol) and N, N-diisopropylethylamine (DIEA, 100 μL, 0.6 mmol). The reaction is warmed to room temperature while stirring for 15 hours. The crude product is then precipitated dropwise into EtOAc (45 mL). The precipitate and solvent mixture is centrifuged at 2000-G for 30 min with careful removal of supernatant. The solid is re-dissolved in 2.0 mL Milli-Q water, charged into a Slide-a-Lyzer dialysis cassette (10 kDa MWCO) and dialyzed against 4 L of Milli-Q water for 24 hours during which solvent exchanges are performed at t=4 hours and t=8 hours. The dialyzed aqueous solution is then dried via lyophilization to yield poly(GalNAc-co-HEMA)-PDS as a solid. This is a prophetic example.

### Synthesis of Polymer F

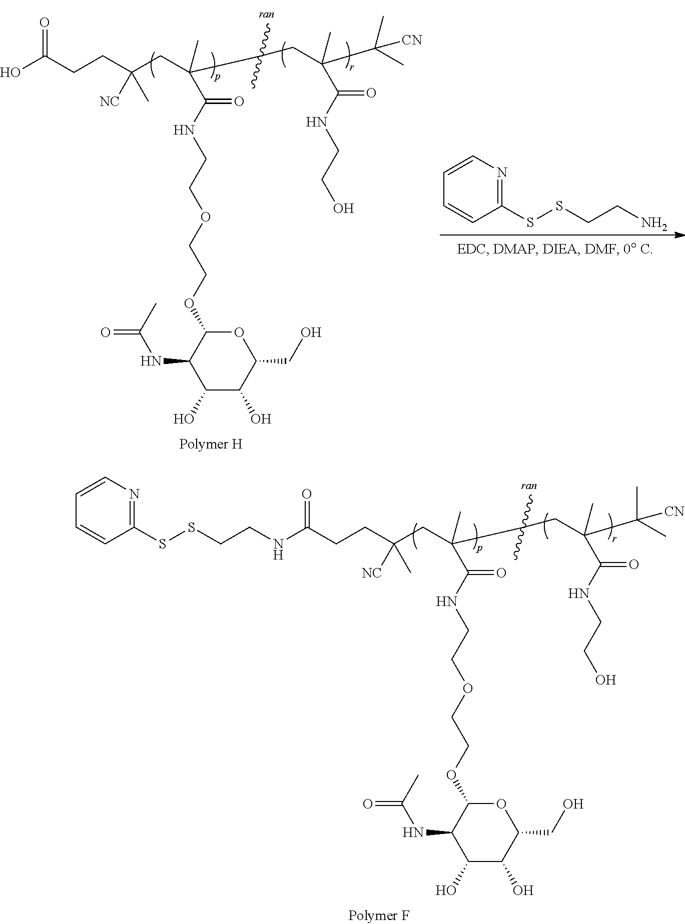

Dithioester-Free (e.g., DTB-free) poly(GalNAc-co-HEMA)-Ethyl Amide (Polymer F). A solution of dithioester-free (e.g., DTB-free) poly(GalNAc-co-HEMA)-CVA (300 mg, 15.9 μmol) in DMF (4.0 mL) is stirred at room temperature for 5 hours. To this solution is added 2-(2-Pyridinyldithio)ethylamine hydrochloride (ethylamine-PDS, 10.6 mg, 47.6 μmol) and N, N-diisopropylethylamine (DIEA, 13.8 μL, 7.9 μmol). Above mixture is cooled to 0° C. and EDC (7.6 mg, 39.6 μmol) and DMAP (0.5 mg, 4.1 μmol) are added. The reaction is warmed to room temperature while stirring for 15 hours. The crude product is then precipitated dropwise into EtOAc (45 mL). The precipitate and solvent mixture is centrifuged at 2000-G for 30 min with careful removal of supernatant. The solid is re-dissolved in 2.0 mL Milli-Q water, charged into a Slide-a-Lyzer dialysis cassette (7 kDa MWCO) and dialyzed against 4 L of Milli-Q water for 24 hours during which solvent exchanges are performed at t=4 hours and t=20 hours. The dialyzed aqueous solution is then dried via lyophilization to yield poly(GalNAc-co-HEMA)-Ethyl Amide as a solid. This is a prophetic example.

### Example 7: Additional Method for Synthesis of Dithioester-Free (e.g., DTB-Free) poly(GalNAc-co-HEMA)-PDS. Dithioester-Free (e.g., DTB-Free) poly(GalNAc-co-HEMA)-PDS Ethyl Amide, and Dithioester-Free (e.g., DTB-Free) poly(GalNAc-co-HEMA)-PDS Ethyl Amide Polymer Synthesis

The following describes embodiments of synthetic methods of using a carboxylic acid terminated-polymer to prepare a DTB-containing polymer (e.g., Polymer A, Polymer C, and Polymer E) and a dithioester-free (e.g., DTB-free) polymer (e.g., Polymer B, Polymer D, and Polymer F), as shown in the following scheme.

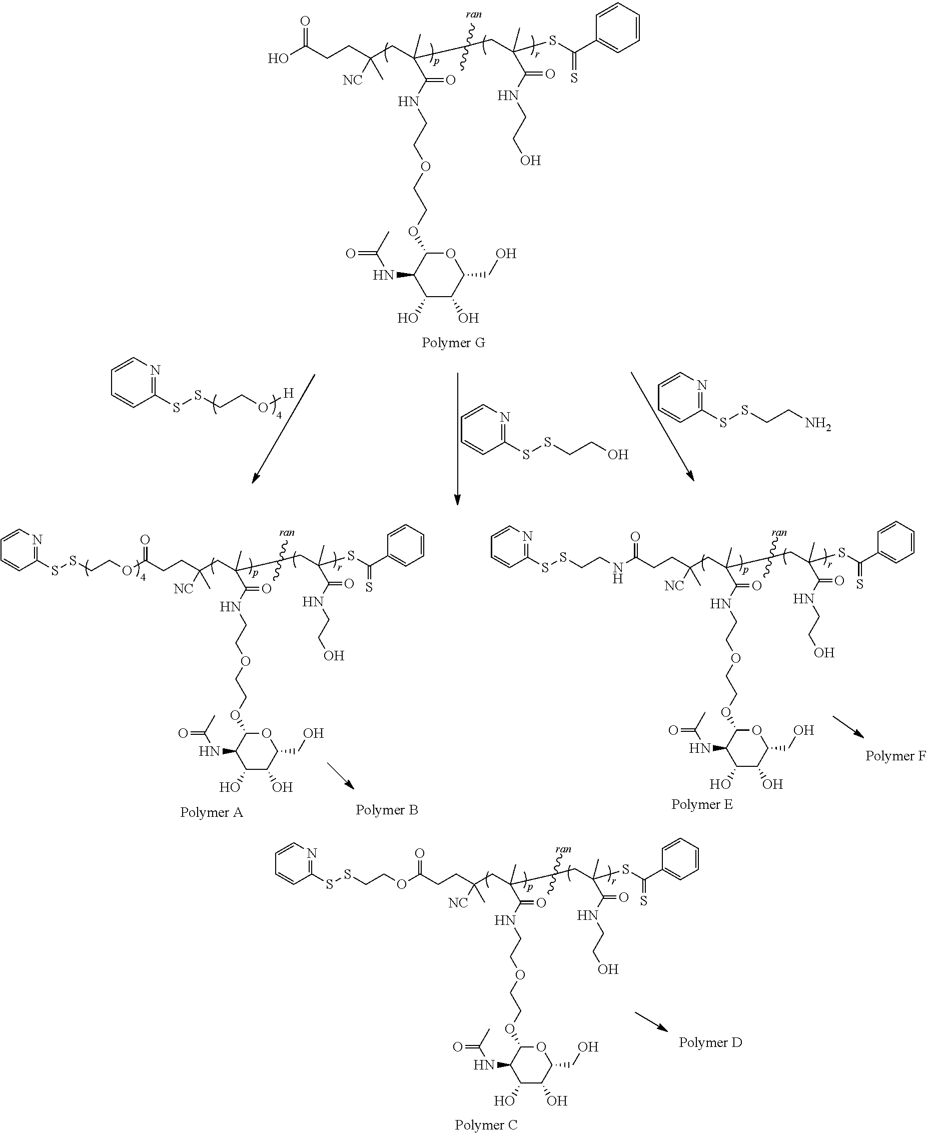

### Synthesis of Polymer A

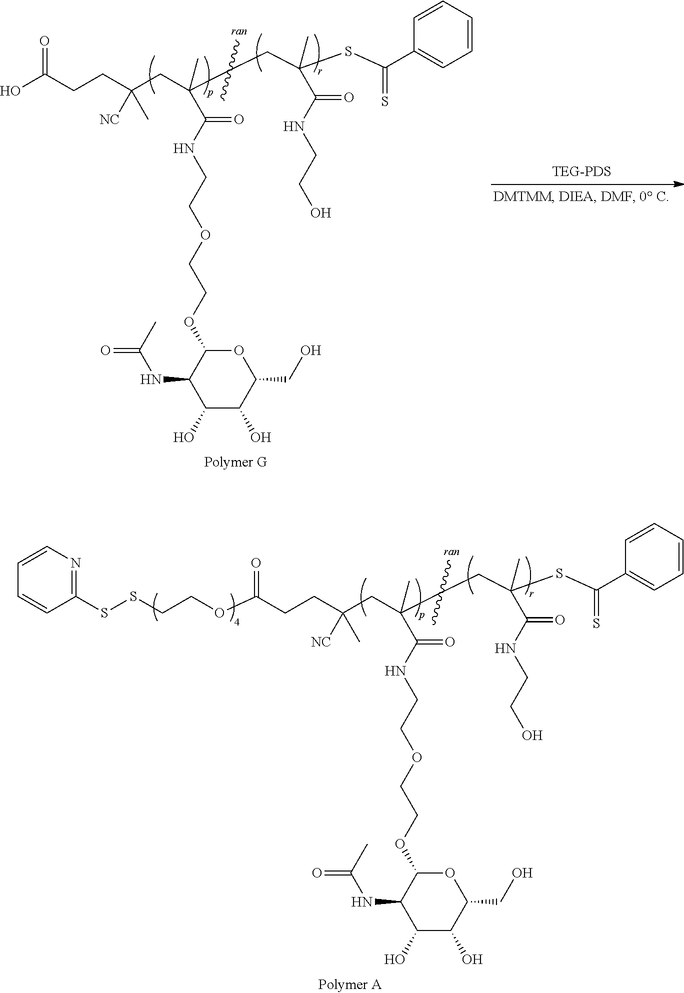

poly(GalNAc-co-HEMA)-PDS (Polymer A). To a solution of poly(GalNAc-co-HEMA)-CVA (26 mg, 1.3 μmol) in DMF (1.0 mL) at 0° C. was added 2-(2-(2-(2-(pyridin-2-yldisulfanyl)ethoxy)ethoxy)ethoxy)ethan-1-ol (TEG-PDS, 100 mg, 0.3 mmol), 4-(4,6-dimethoxy-1,3,5-triazin-2-yl)-4-methyl-morpholinium chloride (DMTMM, 15 mg, 54.3 μmol) and N, N-diisopropylethylamine (DIEA, 100 μL, 0.6 mmol). The reaction was warmed to room temperature while stirring for 15 hours. The crude product was then precipitated dropwise into EtOAc (45 mL). The precipitate and solvent mixture was centrifuged at 2000-G for 30 min with careful removal of supernatant. The solid was re-dissolved in 2.0 mL Milli-Q water, charged into a Slide-a-Lyzer dialysis cassette (10 kDa MWCO) and dialyzed against 4 L of Milli-Q water for 24 hours during which solvent exchanges were performed at t=4 hours and t=8 hours. The dialyzed aqueous solution was then dried via lyophilization to yield poly(GalNAc-co-HEMA)-PDS as a pink solid (10 mg, 33%).

### Synthesis of Polymer C

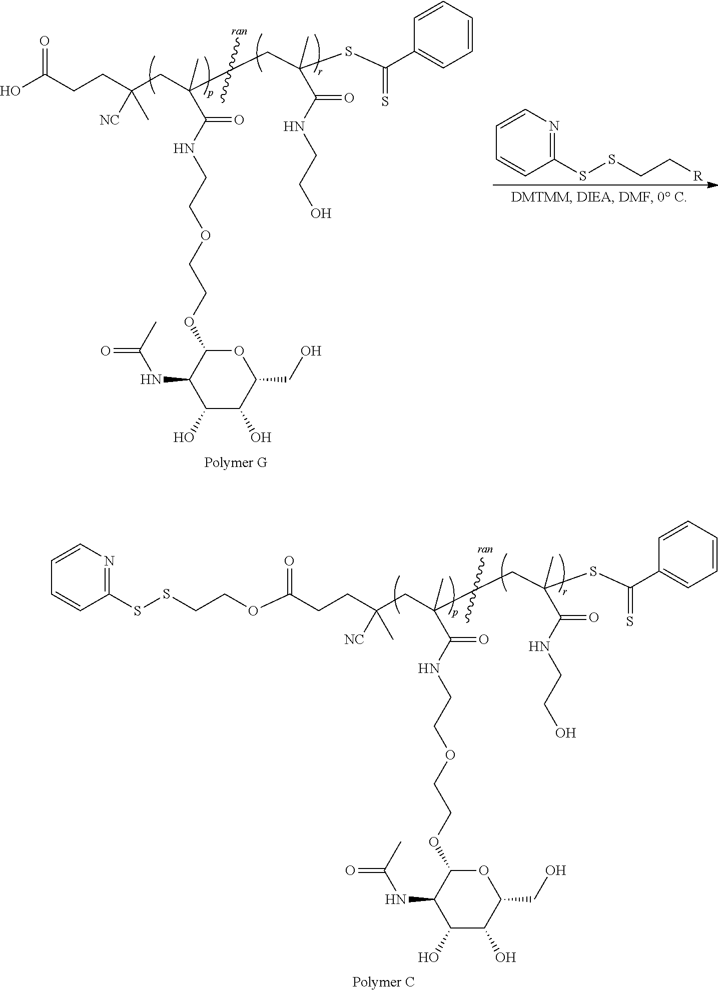

poly(GalNAc-co-HEMA)-PDS (Polymer C). To a solution of poly(GalNAc-co-HEMA)-CVA (26 mg, 1.3 μmol) in DMF (1.0 mL) at 0° C. is added 2-(2-(2-(2-(pyridin-2-yldisulfanyl)ethoxy)ethoxy)ethoxy)ethan-1-ol (TEG-PDS, 100 mg, 0.3 mmol), 4-(4,6-dimethoxy-1,3,5-triazin-2-yl)-4-methyl-morpholinium chloride (DMTMM, 15 mg, 54.3 μmol) and N, N-diisopropylethylamine (DIEA, 100 μL, 0.6 mmol). The reaction is warmed to room temperature while stirring for 15 hours. The crude product is then precipitated dropwise into EtOAc (45 mL). The precipitate and solvent mixture is centrifuged at 2000-G for 30 min with careful removal of supernatant. The solid is re-dissolved in 2.0 mL Milli-Q water, charged into a Slide-a-Lyzer dialysis cassette (10 kDa MWCO) and dialyzed against 4 L of Milli-Q water for 24 hours during which solvent exchanges are performed at t=4 hours and t=8 hours. The dialyzed aqueous solution is then dried via lyophilization to yield poly(GalNAc-co-HEMA)-PDS as a solid. This is a prophetic example. If desired, Polymer D could be synthesized from Polymer C as described above.

### Synthesis of Polymer E

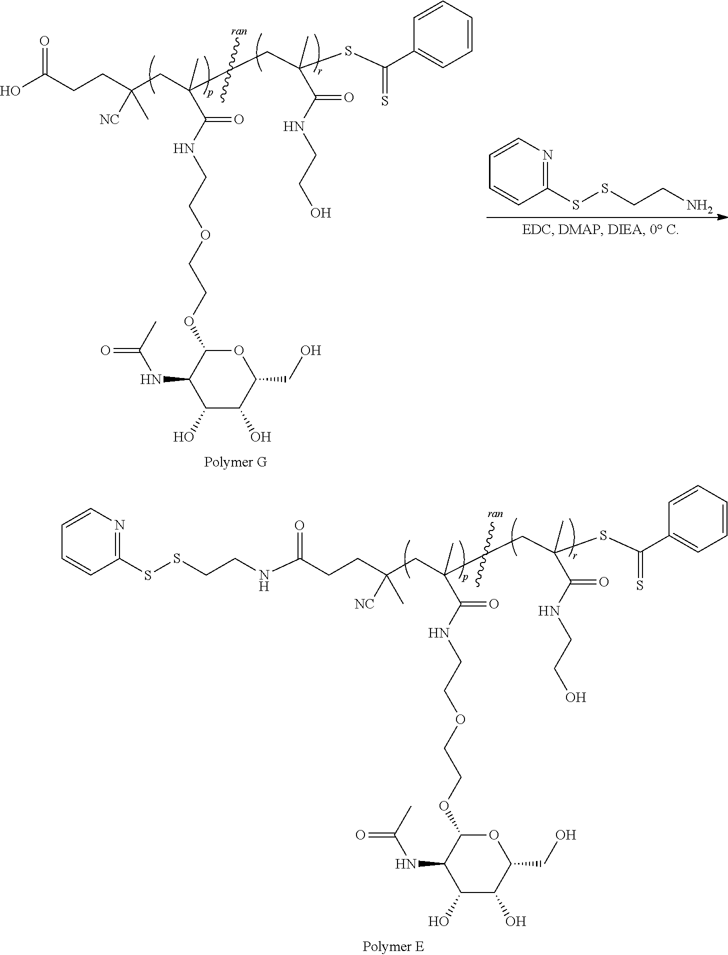

poly(GalNAc-co-HEMA)-Ethyl Amide (Polymer E). A solution of poly(GalNAc-co-HEMA)-CVA (300 mg, 15.9 μmol) in DMF (4.0 mL) was stirred at room temperature for 5 hours. To this solution was added 2-(2-Pyridinyldithio)ethylamine hydrochloride (ethylamine-PDS, 10.6 mg, 47.6 μmol) and N, N-diisopropylethylamine (DIEA, 13.8 μL, 7.9 μmol). Above mixture was cooled to 0° C. and was added EDC (7.6 mg, 39.6 μmol) and DMAP (0.5 mg, 4.1 μmol). The reaction was warmed to room temperature while stirring for 15 hours. The crude product was then precipitated dropwise into EtOAc (45 mL). The precipitate and solvent mixture was centrifuged at 2000-G for 30 min with careful removal of supernatant. The solid was re-dissolved in 2.0 mL Milli-Q water, charged into a Slide-a-Lyzer dialysis cassette (7 kDa MWCO) and dialyzed against 4 L of Milli-Q water for 24 hours during which solvent exchanges were performed at t=4 hours and t=20 hours. The dialyzed aqueous solution was then dried via lyophilization to yield poly(GalNAc-co-HEMA)-Ethyl Amide as a pink solid (178 mg, 58%). If desired, Polymer F could be synthesized from Polymer E as described elsewhere herein.

### Example 8: Additional Method for Synthesis of DTB-Free Polymers

### Synthesis of Polymer I

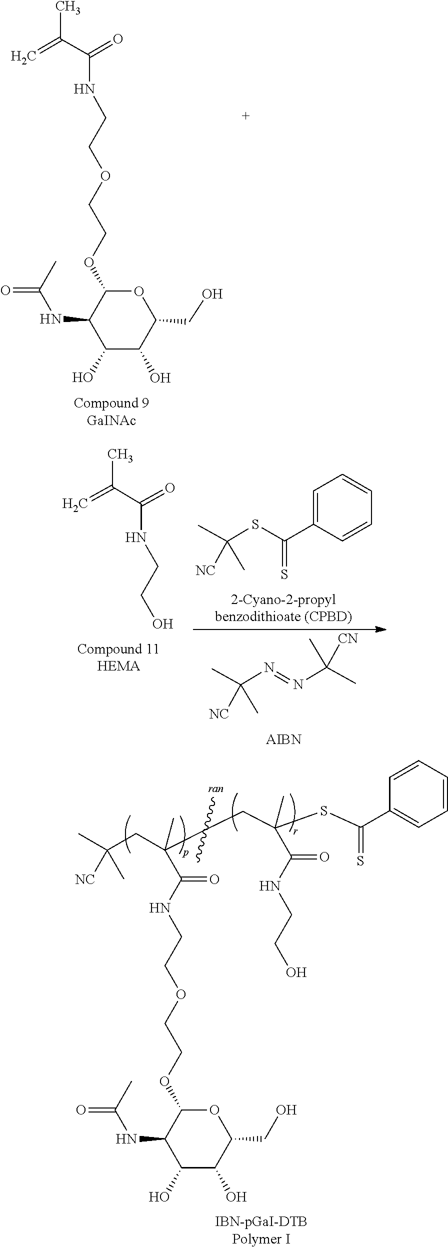

IBN-pGal-DTB (Polymer 1). A typical example synthesis of a IBN-pGal-DTB with a target molecular weight of 20.0 kDa, a target degree of polymerization of approximately 80 monomers, and a target GalNAc:HEMA monomer ratio of 50:50 is as follows: A 100 mL single-neck Schlenk flask equipped with a PTFE valve and situated in a low-light area was purged with ultra-high-purity argon (Grade 6), placed in an ice bath, and charged with a magnetic stir bar, compound 9 (15.0 g, 39.9 mmol, solid), compound 11 (5.1 g, 39.9 mmol, added as 5.0 mL neat oil), 2-cyano-2-propyl benzodithioate (176.4 mg, 0.8 mmol added as 7.0 mL of stock solution at 25.0 mg/mL), 2,2′-azobis(2-methylpropionitrile) (26.2 mg, 0.2 mmol, added as 5.2 mL of stock solution at 5.04 mg/mL), and N,N-dimethylformamide (28 mL). The flask was sealed with a rubber septum, the septum reinforced with parafilm, and the solution was sparged on ice with ultra-high-purity (Grade 6) argon for 1 hour. Following sparge, the solution is degassed according to the following procedure: The contents of the flask are stirred at ˜500 rpm and exposed to high vacuum for 2 minutes. The vacuum is then turned off and the contents are backfilled with argon. This process is repeated 10 times. After the final pump cycle, the degassed solution is overlaid with argon and allowed to stir at ˜700 rpm in a pre-heated oil bath at 75° C. for 6 hours. The RAFT polymerization was quenched by submerging the flask in an ice bath, exposing the solution to air, and allowing the solution to stir on ice at 500 rpm for 15 minutes. The crude reaction solution was then precipitated into 1000 mL 2-propanol at room temperature. The resultant precipitate was filtered via disposable polyethylene fritted funnel (40-micron, 2000 mL capacity), washed four times with 2-propanol, isolated, and dried in vacuo to yield IBN-pGal-DTB as a flaky light-pink solid (11.1 g, 55.05%). GPC: Mₚ=16.6 kDa, Ð=1.15. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer J

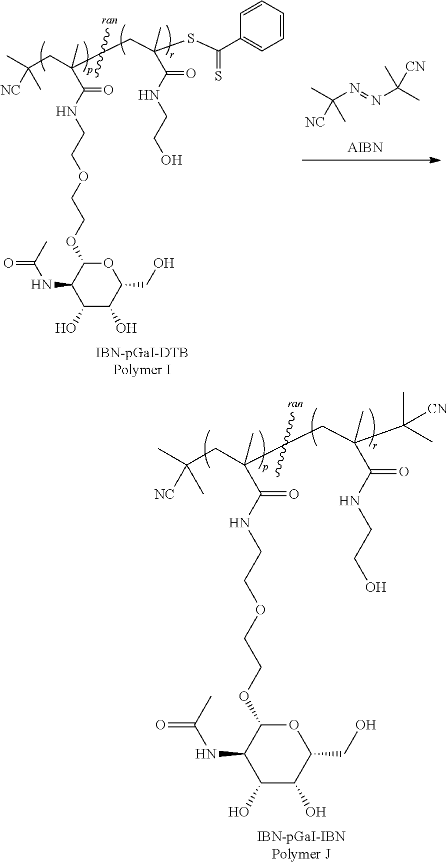

IBN-pGal-IBN (Polymer J). A 100 mL single-neck Schlenk flask equipped with a PTFE valve and situated in a low-light area is purged with high-purity argon (Grade 6) and charged with a magnetic stir bar, IBN-pGal-PDS intermediate (16.6 kDa, 10.0 g, 0.60 mmol, 1.0 Eq., solid), 2,2′-azobis(2-methylpropionitrile) (AIBN) (2.9 g, 18.07 mmol, 30 Eq., solid), and N,N-dimethylformamide (57 mL, anhydrous). The order of addition is: AIBN, pGal, DMF. The flask is sealed with a rubber septum, the septum reinforced with parafilm, and the suspension is allowed to stir in an ice bath at 700 rpm while being sparged with Argon for 60 minutes. Following sparge, the solution is degassed according to the following procedure: The contents of the flask are stirred at ˜500 rpm and exposed to high vacuum for 2 minutes. The vacuum is then turned off and the contents are backfilled with argon. This process is repeated 10 times. After the final pump cycle, the degassed solution is overlaid with argon and allowed to stir at ˜700 rpm in a pre-heated oil bath at 75° C. for 2 hours. The reaction is quenched according to the following procedure: The flask is removed from the oil bath, immediately submerged in an ice bath, and allowed to stir on ice at 700 rpm for 10 minutes. The septum and Schlenk valve is then removed from the flask, and the crude reaction solution is allowed to stir on ice at 700 rpm for an additional 15 minutes. The crude reaction solution is then precipitated into 1000 mL 2-propanol at room temperature. The resultant precipitate is filtered via disposable polyethylene fritted funnel (40-micron, 2000 mL capacity), washed four times with 2-propanol, isolated, and dried in vacuo. The dried precipitate is re-dissolved in 200 mL Milli-Q water, charged into a Slide-a-Lyzer dialysis cassette (250 mL capacity, 10.0 kDa MWCO) and dialyzed against 16 volumes of Milli-Q water for 48 hours during which solvent exchanges are performed at t=4, 12, 24 and 36 hours. The dialyzed aqueous solution is then dried via lyophilization to afford IBN-pGal-IBN final product as a flaky off-white solid. This is a prophetic example.

### Synthesis of Polymer K

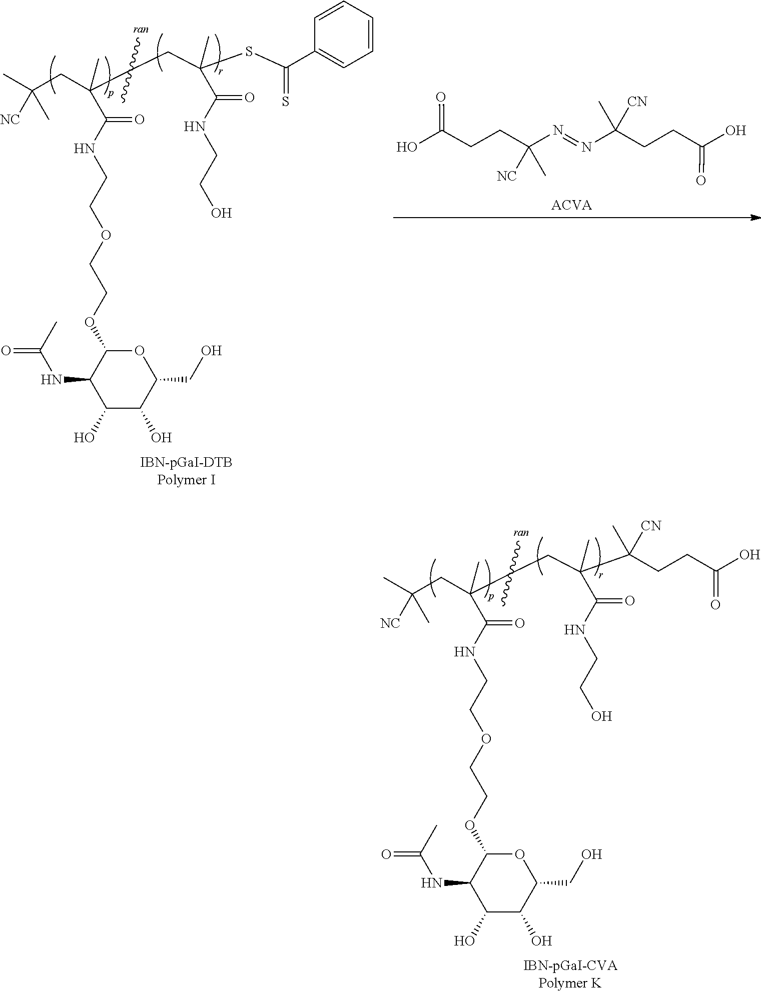

IBN-pGal-CVA (Polymer K). A 100 mL single-neck Schlenk flask equipped with a PTFE valve and situated in a low-light area was purged with high-purity argon (Grade 6) and charged with a magnetic stir bar, IBN-pGal-PDS intermediate (16.6 kDa, 10.0 g, 0.60 mmol, 1.0 Eq., solid), 4,4′-azobis(4-cyanovaleric acid) (ACVA) (5.1 g, 18.07 mmol, 30 Eq., solid), and N,N-dimethylformamide (57 mL, anhydrous). The order of addition was: AIBN, pGal, DMF. The flask was sealed with a rubber septum, the septum reinforced with parafilm, and the suspension was allowed to stir in an ice bath at 700 rpm while being sparged with Argon for 60 minutes. Following sparge, the solution is degassed according to the following procedure: The contents of the flask are stirred at ˜500 rpm and exposed to high vacuum for 2 minutes. The vacuum is then turned off and the contents are backfilled with argon. This process is repeated 10 times. After the final pump cycle, the degassed solution is overlaid with argon and allowed to stir at ˜700 rpm in a pre-heated oil bath at 75° C. for 2 hours. The reaction was quenched according to the following procedure: The flask was removed from the oil bath, immediately submerged in an ice bath, and allowed to stir on ice at 700 rpm for 10 minutes. The septum and Schlenk valve were then removed from the flask, and the crude reaction solution was allowed to stir on ice at 700 rpm for an additional 15 minutes. The crude reaction solution was then precipitated into 1000 mL 2-propanol at room temperature. The resultant precipitate was filtered via disposable polyethylene fritted funnel (40-micron, 2000 mL capacity), washed four times with 2-propanol, isolated, and dried in vacuo. The dried precipitate was re-dissolved in 200 mL Milli-Q water, charged into a Slide-a-Lyzer dialysis cassette (250 mL capacity, 10.0 kDa MWCO) and dialyzed against 16 volumes of Milli-Q water for 48 hours during which solvent exchanges were performed at t=4, 12, 24 and 36 hours. The dialyzed aqueous solution was then dried via lyophilization to afford IBN-pGal-IBN final product as a flaky off-white solid. (8.5 g, 56.5%). GPC: MP=16.6 kDa, Ð=1.15. ¹H-NMR (499.9 MHz, D₂O, 25° C., ppm): δ=0.8-1.6 (m, 3H, backbone methyl), 1.6-2.3 (m, 2H, backbone methylene), 3.2-3.45 (br. s, 4H, ethoxy methylene), 3.5-4.1 (m, sugar ring protons), 4.45-4.6 (br. s, 1H, anomeric), 7.0-8.6 (m, 9H, end-group aromatic).

### Synthesis of Polymer L

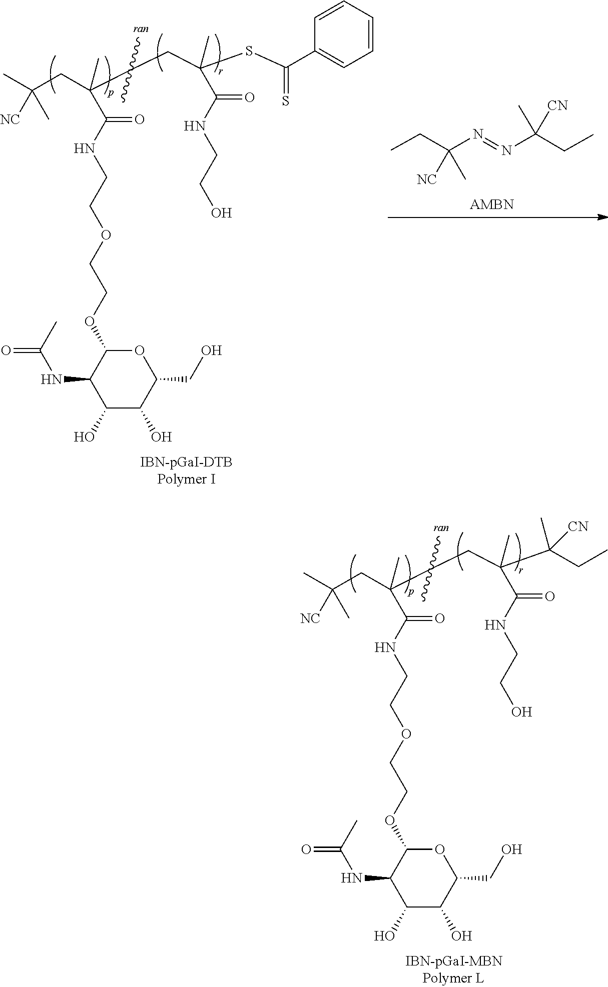

IBN-pGal-MBN (Polymer L). A 100 mL single-neck Schlenk flask equipped with a PTFE valve and situated in a low-light area is purged with high-purity argon (Grade 6) and charged with a magnetic stir bar, IBN-pGal-PDS intermediate (16.6 kDa, 10.0 g, 0.60 mmol, 1.0 Eq., solid), 2,2′-azobis(2-methylbutyronitrile) (AMBN) (3.5 g, 18.07 mmol, 30 Eq., solid), and N,N-dimethylformamide (57 mL, anhydrous). The order of addition is: AIBN, pGal, DMF. The flask is sealed with a rubber septum, the septum reinforced with parafilm, and the suspension is allowed to stir in an ice bath at 700 rpm while being sparged with Argon for 60 minutes. Following sparge, the solution is degassed according to the following procedure: The contents of the flask are stirred at ˜500 rpm and exposed to high vacuum for 2 minutes. The vacuum is then turned off and the contents are backfilled with argon. This process is repeated 10 times. After the final pump cycle, the degassed solution is overlaid with argon and allowed to stir at ˜700 rpm in a pre-heated oil bath at 75° C. for 2 hours. The reaction is quenched according to the following procedure: The flask is removed from the oil bath, immediately submerged in an ice bath, and allowed to stir on ice at 700 rpm for 10 minutes. The septum and Schlenk valve is then removed from the flask, and the crude reaction solution is allowed to stir on ice at 700 rpm for an additional 15 minutes. The crude reaction solution is then precipitated into 1000 mL 2-propanol at room temperature. The resultant precipitate is filtered via disposable polyethylene fritted funnel (40-micron, 2000 mL capacity), washed four times with 2-propanol, isolated, and dried in vacuo. The dried precipitate is re-dissolved in 200 mL Milli-Q water, charged into a Slide-a-Lyzer dialysis cassette (250 mL capacity, 10.0 kDa MWCO) and dialyzed against 16 volumes of Milli-Q water for 48 hours during which solvent exchanges are performed at t=4, 12, 24 and 36 hours. The dialyzed aqueous solution is then dried via lyophilization to afford IBN-pGal-MBN final product as a flaky off-white solid. This is a prophetic example.

### Synthesis of Polymer M

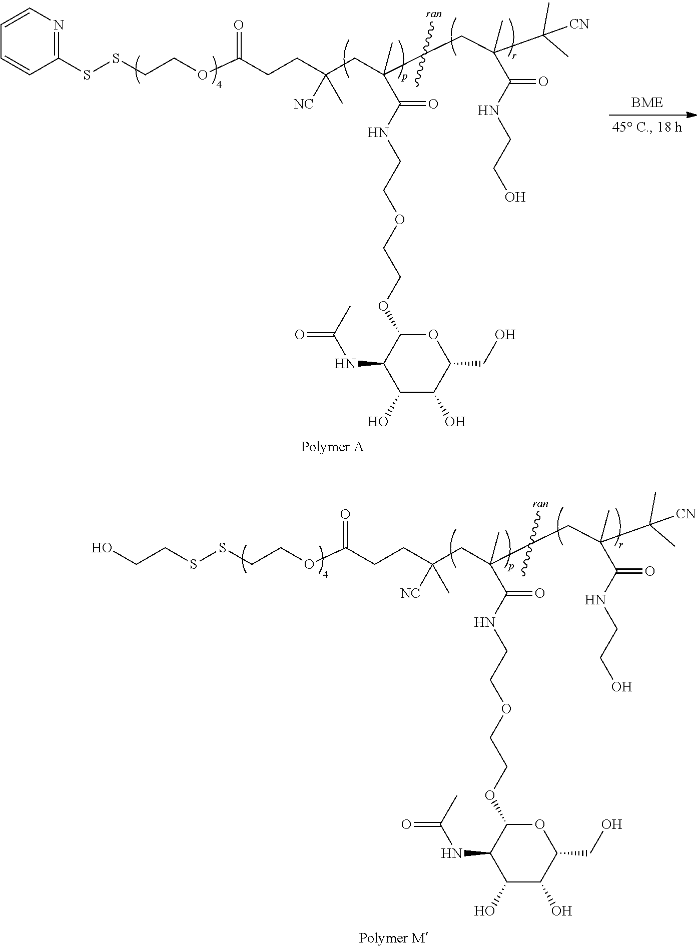

poly(GalNAc-co-HEMA)-ethanol (Polymer M′). A solution of PDS-pGal-IBN (24.7 kDa, 2 g, 80.97 mmol) in 2-mercaptoethanol (BME, 10 mL) is heated at 45° C. for 18 hours. The crude product is then precipitated dropwise into EtOAc (45 mL). The precipitate and solvent mixture is centrifuged at 2500-G for 30 minutes with careful removal of supernatant, EtOAc (45 mL) is added to the solid. The mixture is centrifuged at 2000-G for 30 minutes, this process is repeated two more times. The solid is dried under pressure overnight and re-dissolved in 5.0 mL Milli-Q water, charged into a Slide-a-Lyzer dialysis cassette (7 kDa MWCO) and dialyzed against 4 L of Milli-Q water for 24 hours during which solvent exchanges are performed at t=4 hours and t=15 hours. The dialyzed aqueous solution is then dried via lyophilization to yield of poly(GalNAc-co-HEMA)-ethanol as a white solid (24.7 kDa, 1.5 g, 75%).

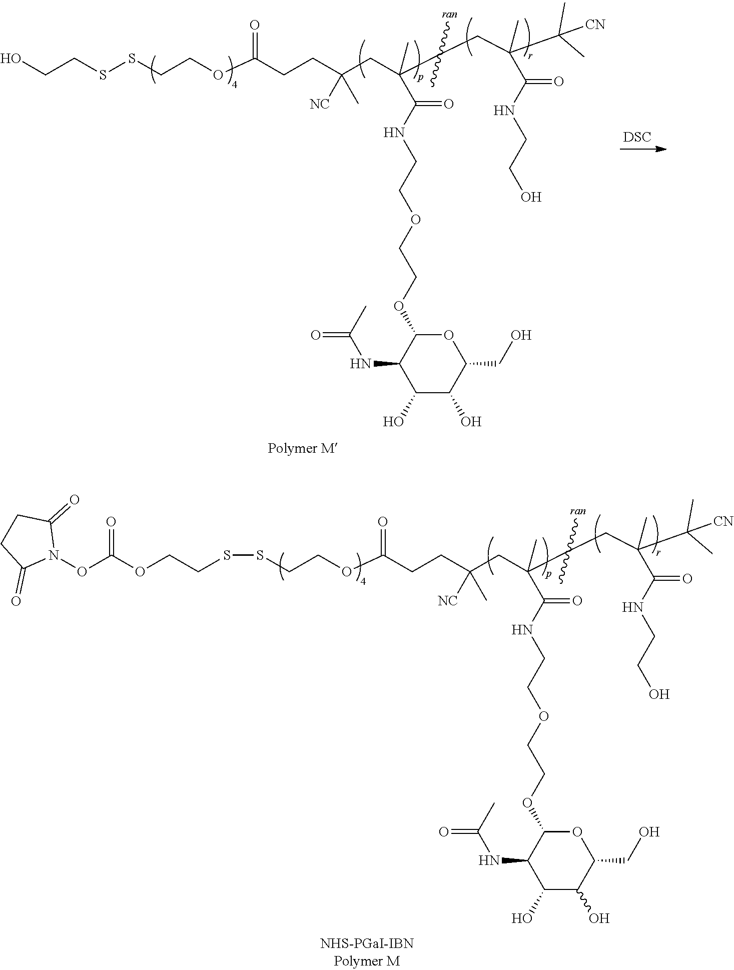

poly(GalNAc-co-HEMA)-NHS (Polymer M). To a solution of poly(GalNAc-co-HEMA)-ethanol (24.7 kDa, 1.5 g, 0.60 mmol) in DMF (15 mL) is added pyridine (19 mL, 0.24 mmol), the mixture is cooled down to 0° C. in ice bath and as N,N′-disuccinimidyl carbonate (DSC) (31 mg, 0.121 mmol) was added. The reaction is stirred at room temperature for 15 hours. The crude product is then precipitated dropwise into EtOAc (45 mL). The precipitate and solvent mixture is centrifuged at 2500-G for 30 minutes with careful removal of supernatant, EtOAc (45 mL) is added to the solid. The mixture is centrifuged at 2000-G for 30 minutes, this process is repeated two more times. The solid is dried under reduced pressure for 18 hours to yield poly(GalNAc-co-HEMA)-NHS as a white solid (1.4 g, 93%).

### Synthesis of Polymer N

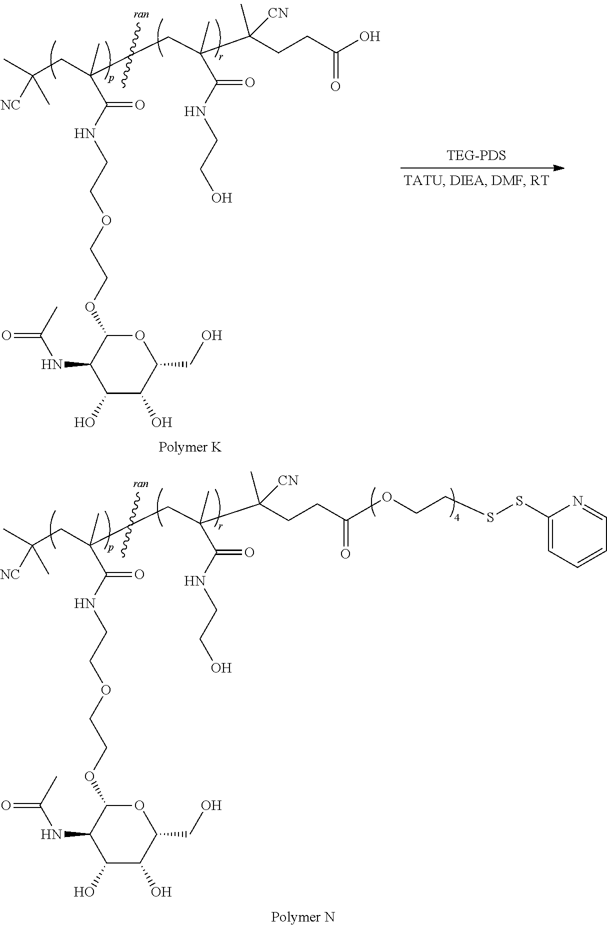

IBN-pGal-PDS (Polymer N). To a solution of IBN-pGal-COOH (15.8 kDa, 200 mg, 12.99 μmol) in DMF (2.0 mL) is added 2-(2-(2-(2-(pyridin-2-yldisulfanyl)ethoxy)ethoxy)ethoxy)ethan-1-ol (TEG-PDS, 20 mg, 0.063 mmol), O-(7-Azabenzotriazole-1-yl)-N,N,N′,N′-tetramethyluronium tetrafluoroborate (TATU, 10 mg, 31.04 μmol) and N, N-diisopropylethylamine (10 μL, 0.06 mmol). The reaction is stirred at room temperature for 15 hours. The crude product is then precipitated dropwise into EtOAc (45 mL). The precipitate and solvent mixture is centrifuged at 2500-G for 30 minutes with careful removal of supernatant, EtOAc (45 mL) is added to the solid. The slurry is centrifuged at 2000-G for 30 minutes, this process is repeated two more times. The solid is re-dissolved in 5.0 mL Milli-Q water, charged into a Slide-a-Lyzer dialysis cassette (7 kDa MWCO) and dialyzed against 4 L of Milli-Q water for 24 hours during which solvent exchanges are performed at t=4 hours and t=15 hours. The dialyzed aqueous solution is then dried via lyophilization to yield IBN-pGal-PDS as a white solid (15.8 kDa, 168 mg, 84%/).

### Example 9: Functionalization of Polymers

### KAN0032-pGal-Ethyl Amide Synthesis

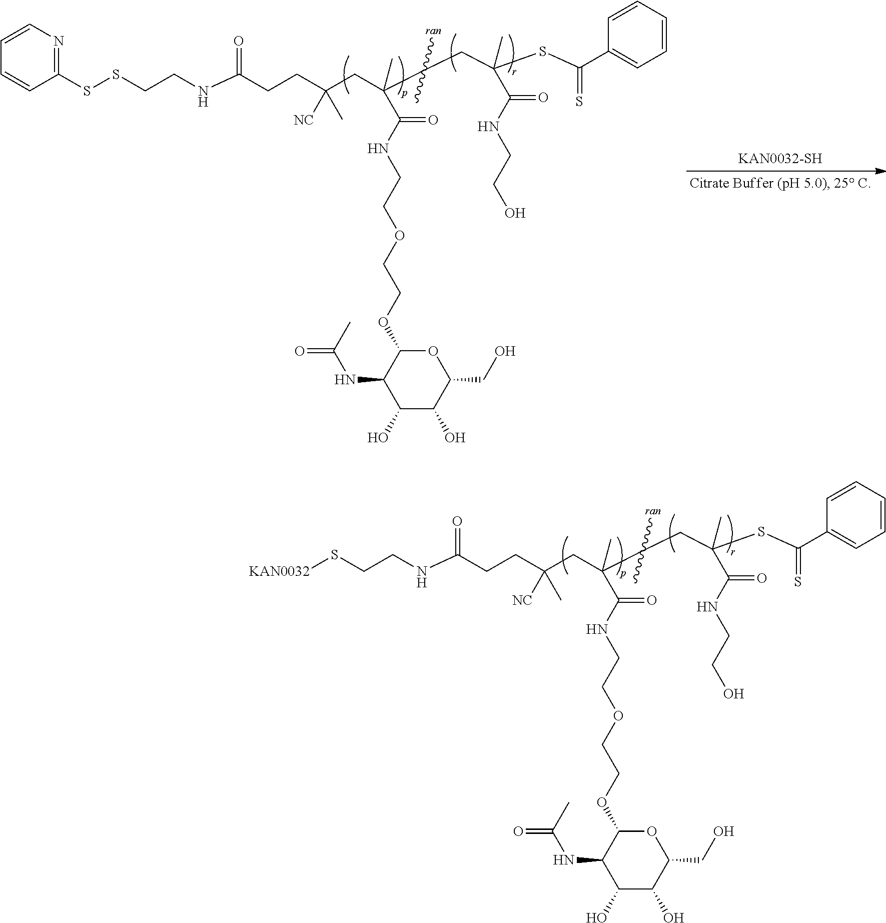

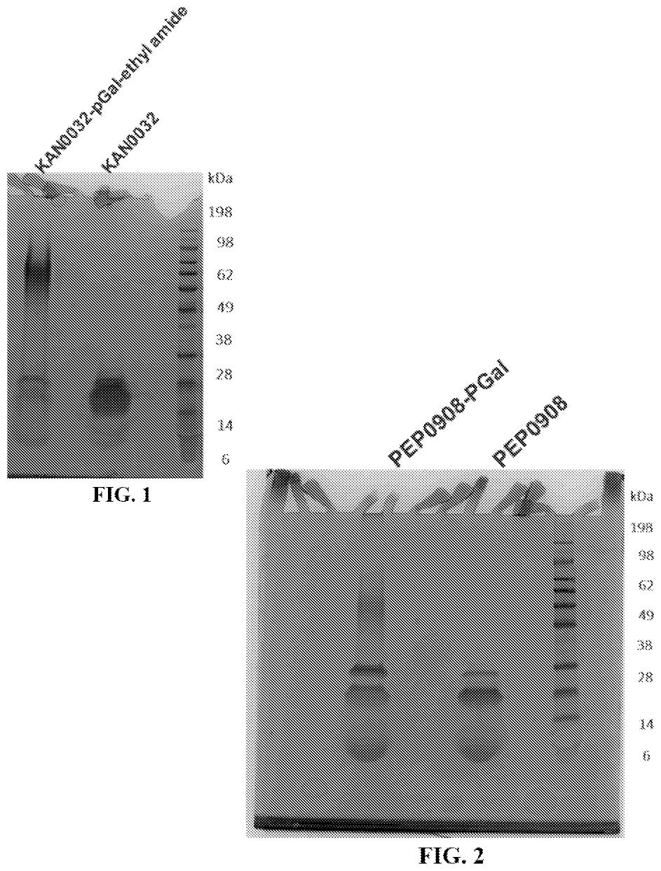

KAN0032-pGal-ethyl amide: KAN0032 (2.2 mg, 0.8 μmol) was dissolved in water (220 μL) and added to 40 mM citric buffer (220 μL). The solution was added to a vial containing poly(GalNAc-co-HEMA)-ethyl amide (17.2 mg, 0.9 μmol). The mixture was placed on an orbital shaker at 60 rpm for 3 hours. The conjugate was diluted to 1.0 mL with Milli-Q water, charged into a Slide-a-Lyzer dialysis cassette (7 kDa MWCO) and dialyzed against 4 L of Milli-Q water for 24 hours during which solvent exchanges were performed at t=4 hours, t=8 hours. The dialyzed aqueous solution was then dried via lyophilization to yield KAN0032-pGal-ethyl amide as a light pink solid (10 mg, 67%/). FIG. 1 shows an SDS-PAGE gel, stained with Coomassie SimplyBlue) performed on the product which shows a distinct band corresponding to KAN-0032-pGal conjugate—a band that is not present in the lane containing peptide alone. pGal polymer does not stain with Coomassie SimplyBlue, and therefore cannot be visualized. KAN0032 is a representative tolerogenic portion of a full-length antigen. It is a foreign antigen found in foods that can enter the body through the oral route and is representative of a food antigen that is the target of an unwanted immune response. KAN0032 is 27 amino acids in length and comprises free amine groups presented by a lysine residue along the KAN0032 peptide chain as well as a terminal free amine (for amine conjugation via amide formation or carbamate formation). KAN0032 also comprises a free sulfhyryl group presented by a cysteine residue along the KAN0032 peptide chain (for thiol-based conjugation and disulfide formation). In this instance, a disulfide is formed to form the tolerogenic construct.

### PEP0908-pGal Synthesis

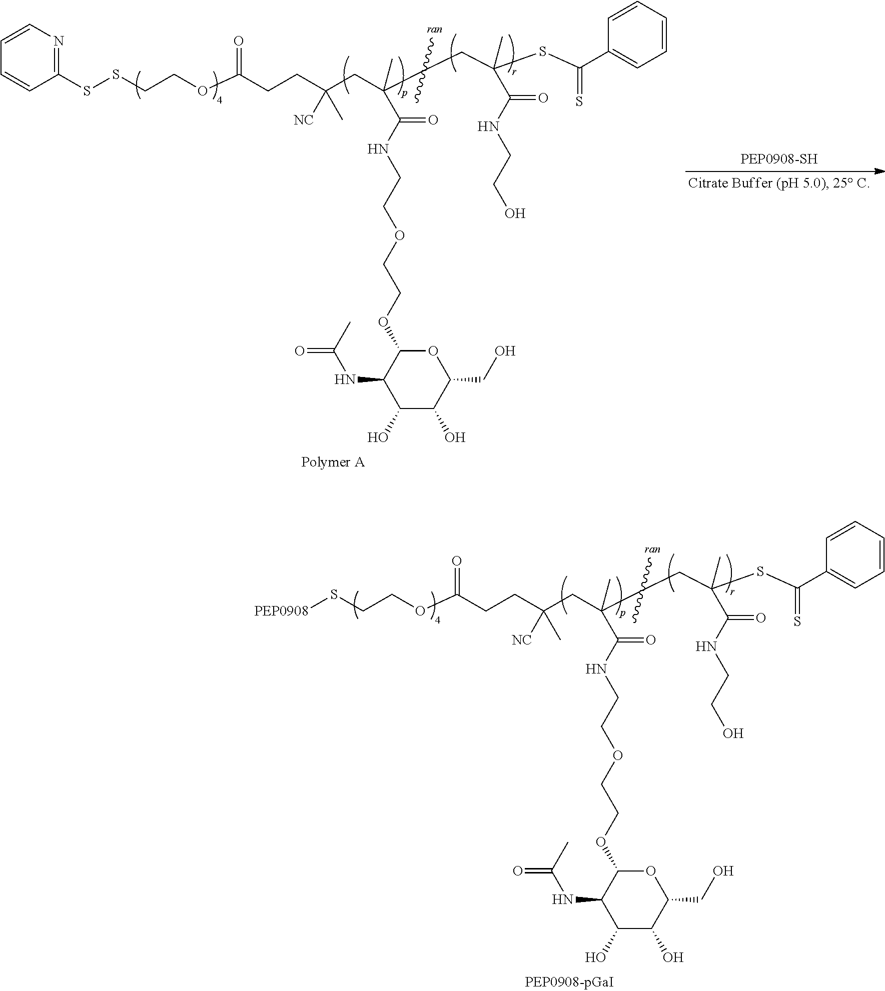

PEP0908-pGal: PEP0908 (1.0 mg, 0.2 μmol) was dissolved in 0.1 M citric buffer (100 μL). The solution was added to a vial containing poly(GalNAc-co-HEMA)-PDS (post modified) (6 mg, 0.3 μmol). The mixture was placed on an orbital shaker at 60 rpm for 3 hours. As shown in FIG. 2, conjugate formation was confirmed by SDS-PAGE gel (stained with Coomassie SimplyBlue) which shows a distinct band corresponding to PEP0908-pGal conjugate—a band that is not present in the lane containing peptide alone. pGal polymer does not stain with Coomassie SimplyBlue, and therefore cannot be visualized. PEP0908 is a representative tolerogenic portion of a full-length antigen. It is an autoantigen that is the target of an unwanted immune response in an autoimmune disease (in this case MS). PEP0908 is 45 amino acids in length and comprises free amine groups presented by lysine residues along the PEP0908 peptide chain as well as a terminal free amine (for amine conjugation via amide formation or carbamate formation). PEP0908 also comprises a free sulfhydryl group presented by a cysteine residue along the PEP0908 peptide chain (for thiol-based conjugation and disulfide formation). In this instance, a disulfide is formed to provide the tolerogenic construct.

### KAN0029 Amine Conjugation

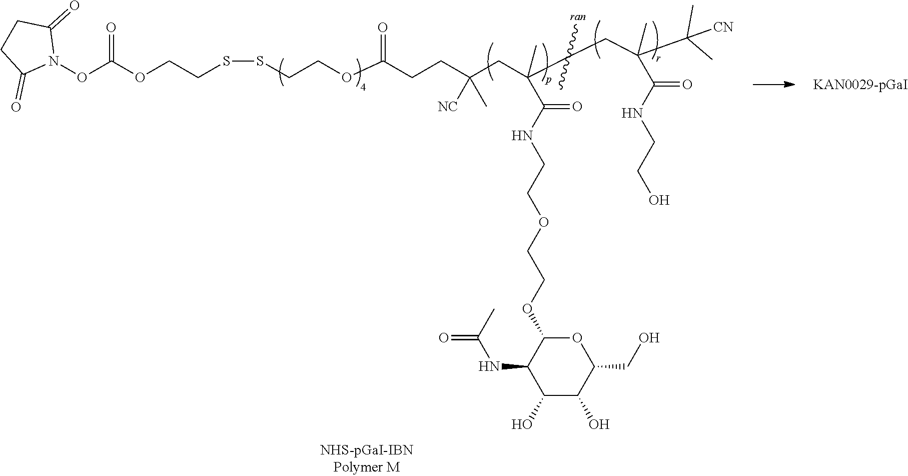

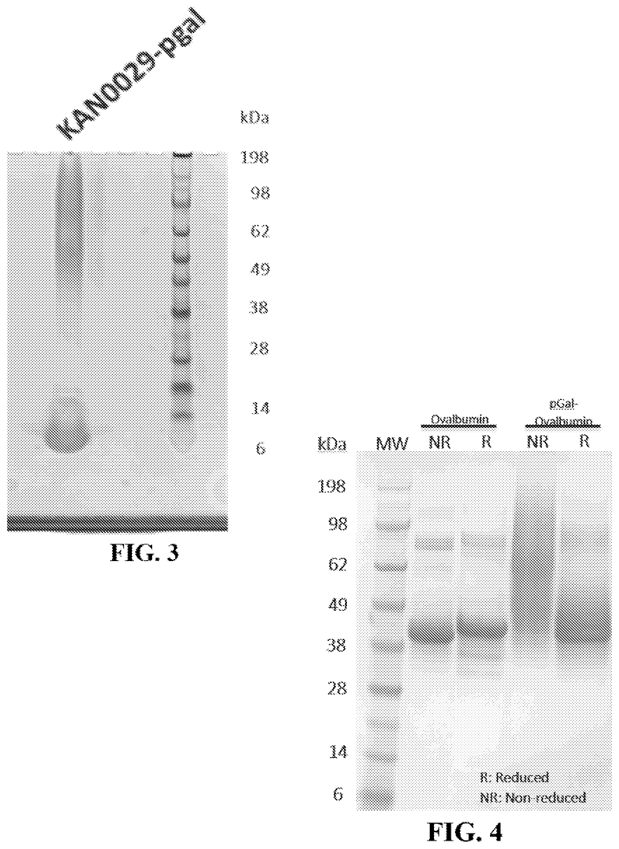

KAN0029-pGal: KAN0029 (0.3 mg, 0.106 μmol) was dissolved in PBS buffer (60 μL). The solution was added to a vial containing poly(GalNAc-co-HEMA)-NHS (NHS-pGal-IBN, 24.7 kDa, 4 mg, 0.22 μmol) and N, N-diisopropylethylamine (1 μL) was added. The mixture was placed on an orbital shaker at 60 rpm for 18 hours. As shown in FIG. 3, conjugate was confirmed by SDS-PAGE gel (stained with Coomassie SimplyBlue) which shows a distinct band corresponding to KAN0029-pGal conjugate. pGal polymer does not stain with Coomassie SimplyBlue, and therefore cannot be visualized. KAN0029 is representative of a tolerogenic portion of a full-length antigen. It is a viral antigen that is representative of viral vectors used for gene therapy, where an unwanted immune response is exhibited towards the viral vector. KAN0029 is 26 amino acids in length and comprises free amine groups presented by lysine residues along the KAN0029 peptide chain as well as a terminal free amine (for amine conjugation via amide formation or carbamate formation). In this instance, as shown above, a carbamate is formed to provide the tolerogenic construct.

### Ovalbumin Amine Conjugation

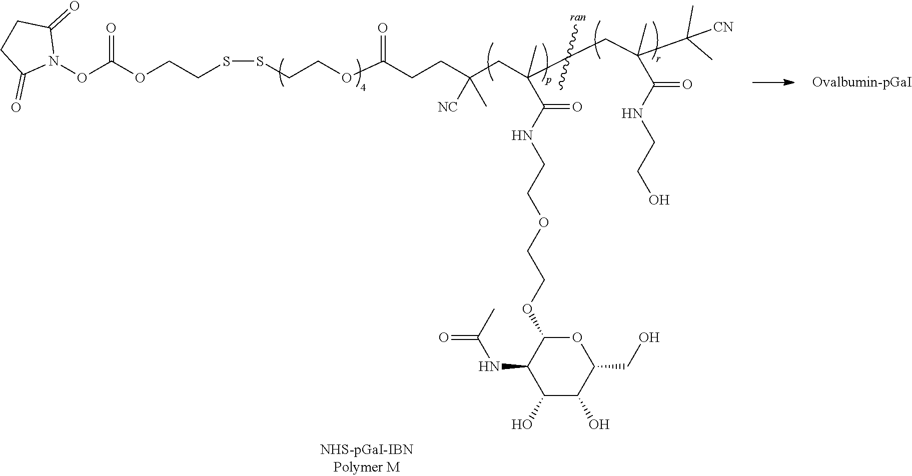

Ovalbumin-pGal: Ovalbumin (0.5 mg, 0.011 μmol) was dissolved in HEPES buffer pH 7 (200 μL). The solution was added to a vial containing poly(GalNAc-co-HEMA)-NHS (NHS-pGal-IBN, 22.6 kDa, 121 mg, 5.35 μmol). The mixture was placed on an orbital shaker at 60 rpm for 18 hours. As shown in FIG. 4, conjugate was confirmed by SDS-PAGE gel (stained with Coomassie SimplyBlue) which shows a distinct band corresponding to Ovalbumin-pGal conjugate. pGal polymer does not stain with Coomassie SimplyBlue, and therefore cannot be visualized. OVA (ovalbumin) is representative of a full-length antigen. It is a foreign antigen found in foods and is representative of a food antigen that is the target of an unwanted immune response. OVA is 385 residues in length and comprises free amine groups presented by lysine residues along the OVA polypeptide chain as well as a terminal free amine (for amine conjugation via amide formation or carbamate formation).

### KAN0029-pGal-Omega Synthesis

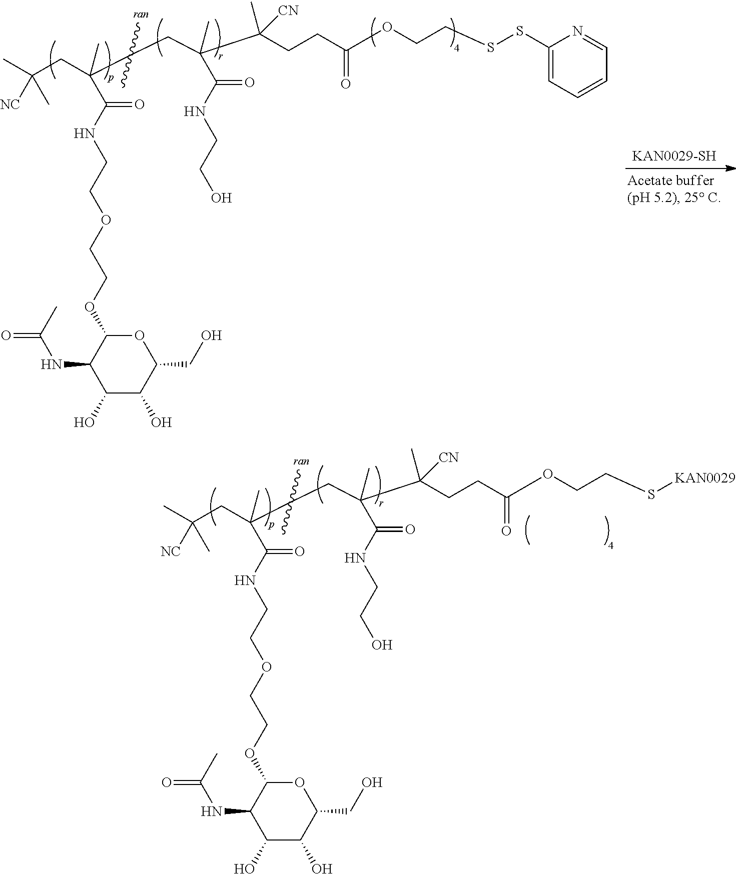

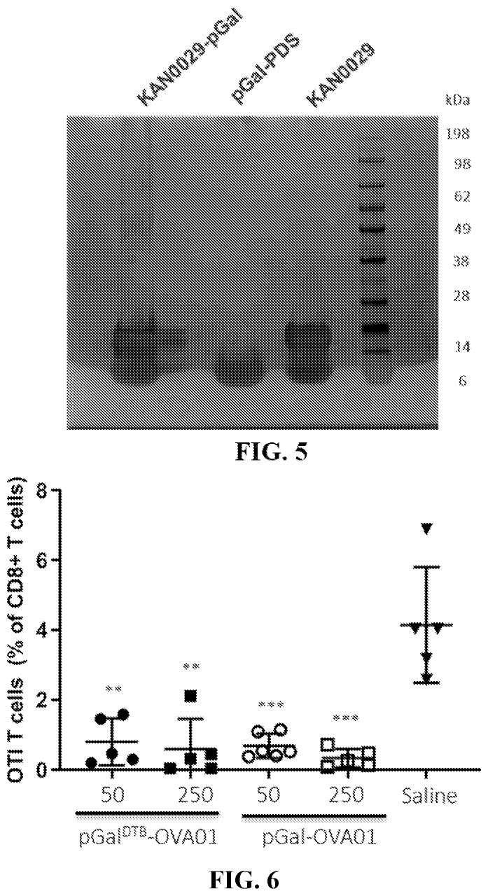

KAN0029-pGal: KAN0029 (1.0 mg, 0.35 μmol) was dissolved in 0.1 M acetic buffer (100 μL). The solution was added to a vial containing IBN-pGal-PDS (15.8 kDa, 11 mg, 0.69 μmol). The mixture was placed on an orbital shaker at 60 rpm for 18 hours. Formation of conjugate was confirmed by SDS-PAGE gel (stained with Coomassie SimplyBlue), as shown in FIG. 5, which showed a distinct band corresponding to KAN0029-pGal conjugate—a band that is not present in the lane containing peptide alone. pGal polymer does not stain with Coomassie SimplyBlue, and therefore cannot be visualized. KAN0029 is representative of a tolerogenic portion of a full-length antigen. It is a viral antigen that is representative of viral vectors used for gene therapy, where an unwanted immune response is exhibited towards the viral vector. KAN0029 is 26 amino acids in length and comprises a free sulfhydryl group presented by a cysteine residue along the KAN0029 peptide chain (for thiol-based conjugation and disulfide formation). In this instance, a disulfide is formed to provide the tolerogenic construct.

### Example 10: In Vitro

poly(GalNAc-co-HEMA)-PDS with and without a DTB group were tested for receptor binding using surface plasmon resonance conducted on a Biacore T200 instrument. Due to the multivalency of carbohydrate-receptor interactions, steady-state affinity was used to report the equilibrium dissociation constant (K_{D}) of the interaction. 2700 RU of biotinylated recombinant human receptor was immobilized onto a strepavidin-coated dextran surface. In addition to the receptor-coated surface, a blank reference surface containing only streptavidin-coated dextran was included to monitor non-specific binding to the surface and to correct for bulk refractive index changes. HEPES-buffered saline at pH 7.3 supplemented with 5 mM CaCl₂ and surfactant P20 (HBS-P), was used as the assay buffer. DTB containing-poly(GalNAc-co-HEMA)-PDS and dithioester-free (e.g., DTB-free) poly(GalNAc-co-HEMA)-PDS were sequentially injected over receptor-coated and reference surfaces using single-cycle kinetics in a 5-point concentration range spanning 9.8 nM-800 nM following a 1:3-fold dilution scheme. After each series of injections, the surface was regenerated with HBS-P at pH 6.3 without CaCl₂. The association time for the interaction with each surface was 60 seconds, and the flow rate used for the interaction was 30 μL/min. Using the steady-state fitting algorithm provided by the Biacore T200 evaluation software, the equilibrium dissociation constants were calculated as follows: K_{D}=105 nM for DTB containing-poly(GalNAc-co-HEMA)-PDS, and K_{D}=182 nM Dithioester-Free (e.g., DTB-free) poly(GalNAc-co-HEMA)-PDS. Given that these K_{D} values are within 2-fold of each other, which is the expected error margin of the these measurements, the affinities of DTB containing-poly(GalNAc-co-HEMA)-PDS and Dithioester-Free (e.g., DTB-free) poly(GalNAc-co-HEMA)-PDS to their receptor are considered equivalent.

### Example 11: In Vivo

The following experiment was conducted to demonstrate that tolerogenic compositions disclosed herein, containing or not containing DTB intermediate, can establish tolerance to a given antigen. OVA01 is an immunogenic fragment of the ovalbumin antigen that contains an epitope recognized by OTI antigen-specific CD8+ T cells. OVA01 is used in this example as a non-limiting example of a immunogenic fragment of an antigen to which tolerance is desired. OVA01 was chemically conjugated to pGal using bioconjugate techniques. In order to determine if pGal-antigen conjugates induced immune tolerance of OVA01-specific OTI CD8+ T cells, mice were first infused with OTI CD8+ T cells, then administered poly(GalNAc-co-HEMA)-OVA01 conjugates. Two weeks after poly(GalNAc-co-HEMA)-OVA01 treatment, mice were then challenged with ovalbumin antigen formulated with an adjuvant to determine the tolerogenic efficacy of poly(GalNAc-co-HEMA)-OVA01 therapy. Mice are euthanized 4 days after challenge in order to assess the frequency of antigen-specific OTI CD8+ T cells, which is one metric of demonstration of immune tolerance.

Protocol: On day 0, congenic CD45.1 C57BL/6 mice received an intravenous infusion of 770,000 OTI CD8+ T cells that were purified by magnetic negative separation. On day 1, mice (n=5 per group) were treated with either saline, poly(GalNAc-co-HEMA)-OVA01 containing DTB (dosed at 50, 250, or 1,250 μmol antigen/g body weight), or poly(GalNAc-co-HEMA)-OVA01 with DTB removed (50, 250, or 1,250 μmol antigen/g body weight). On day 15, animals were challenged with a total of 10 μg of ovalbumin and 50 ng of lipolysaccharide administered by equally distribution across all four limbs. On day 19, mice were euthanized and the frequency of OTI T cells in the spleen was assessed by flow cytometry.

Results: The results depicted in FIG. 6 illustrate the remaining OVA01-specific OTI CD8+ T cells after antigen challenge (as a percentage of total live CD3+CD8+ T cells) in the spleen. Mice treated with saline showed a high frequency of OTI CD8+ T cells in the spleen, indicative of maintenance of an inflammatory immune response specific to the OVA01 antigen. Mice treated with DTB-containing poly(GalNAc-co-HEMA)-OVA01 or dithioester-free (e.g., DTB-free) poly(GalNAc-co-HEMA)-OVA01 exhibited significant reductions in OVA01-specific OTI CD8+ T cells in the spleen, at all doses tested. Tolerogenic poly(GalNAc-co-HEMA) compositions with or without DTB intermediate were similarly efficacious. The statistical significance by an unpaired t test are indicated as compared to the saline control group: *=p<0.05, **=p<0.01, ***=p<0.001.

### Example 12: Stability Study

Samples of antigen conjugated to poly(GalNAc-co-HEMA)-PDS with a DTB end group and antigen conjugated to poly(GalNAc-co-HEMA)-PDS without a DTB end group were formulated in 10 mM sodium acetate, containing 274 mM sorbitol at a peptide concentration of 1 mg/mL (pH of about 5 to 5.5). Using aseptic techniques, aliquots of the polymer conjugate solution was filled into Type 1 glass vials, which were then sealed with 4023/50G rubber stoppers and aluminum caps. Individual vials containing each sample were placed in a calibrated 23-27° C. incubator.

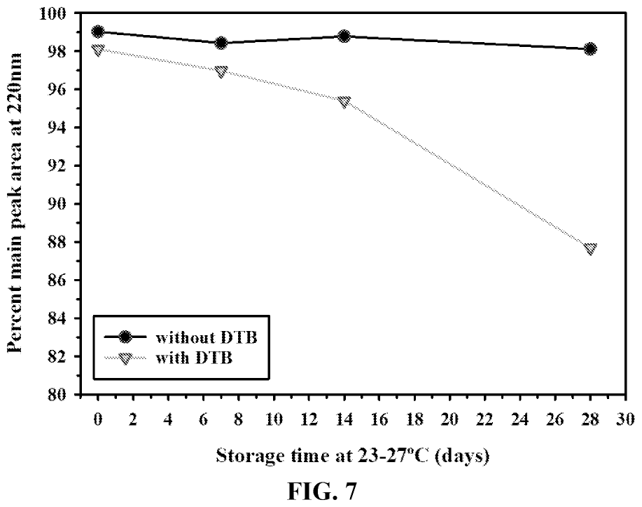

FIG. 7 shows that, surprisingly, the stability over a period of days (measured using reversed-phase HPLC) for two differing polymer conjugates having alternative end groups (e.g., one with a terminal end unit and the other a dithioester) is different. Analytical testing was performed on separate sample vials at the time points of 0, 7, 14, and 28 days, as shown in FIG. 7. Also shown, the dithioester-free (e.g., DTB-free) conjugates had remarkably higher stability than dithioester containing conjugates under the same conditions.

As noted above, samples were tested by reversed-phase HPLC. The percent main peak area was measured at 220 nm reported and graphed. The dithioester-free (e.g., DTB-free) poly(GalNAc-co-HEMA) conjugate (having an isobutyronitrile (IBN) end-group) had enhanced stability relative to conjugate having a DTB end-group.

This testing demonstrates that dithioester-free (e.g., DTB-free) constructs (and/or terminal end units comprising a carbon bond to the linker, lacking aryl functionalities, and/or lacking sulfur containing atoms, such as dithioesters) have surprisingly improved stability versus dithioester-containing constructs. In several embodiments, stability is improved by using terminal end units that lack a dithioester group, ones that are a reaction product of and/or are produced using a reaction with an azo-compound (e.g., a bis-azo compound), that lack an end-capping group with a sulfur containing unit (e.g., a dithioester), and/or that lack an aryl moiety. In several embodiments, stability of the resultant reaction product is enhanced (relative to a dithioester containing construct) by about 5%, about 10%, about 15%, about 20%, or about 25%. In several embodiments, such increases in stability result in a functional improvement of the compositions disclosed herein, with respect to induction of antigen-specific immune tolerance. In several embodiments, the increased stability of the reaction product that is then incorporated into a tolerogenic composition as disclosed herein results in a longer functional life of the composition in vivo, allowing for the more rapid, more efficient, more robust, or otherwise improved induction in tolerance to an antigen.

In several embodiments, the stability of DTB-free embodiments (versus compounds comprising DTB) is improved by equal to or at least about: 1.0%, 2.5%, 5%, 10%, 15%, 20%, or ranges including and/or spanning the aforementioned values. In several embodiments, DTB-free compounds degrade at a reduced rate, for example, their stability decreases by less than or equal to about: 0.1%, 0.5%, 1.0%, 2.0%, 2.5%, 5% over a period of 5, 10, 14, 20, 15, 28 days, or longer. In several embodiments, testing for stability may be performed using the conditions provided in Example 11. In several embodiments, stability testing may be performed over a period of equal to or at least about: 7 days, 14 days, 28 days, or ranges including and/or spanning the aforementioned values. In several embodiments, testing for stability may be performed using a solution comprising sodium acetate buffer. In several embodiments, testing for stability may be performed using a solution comprising sorbitol. In several embodiments, testing for stability may be performed using an aqueous solution at a pH of about 5. In several embodiments, testing for stability may be performed at a temperature of about 23-27° C. In some embodiments, testing is performed using a solution that is 10 mM sodium acetate containing 274 mM sorbitol at a peptide concentration of 1 mg/mL (pH of about 5). In some embodiments, testing is performed for a period of 14 days or 28 days at 23-27° C.

### Example 13: Additional Stability Studies of Dithioester-Free Embodiments

Two lots of pGal polymer final product (DTB-free) were placed on a long-term stability evaluation program under three storage condition temperatures: −20° C.±5° C., 5° C.±3° C. and at 25° C.±2° C. At the 25° C. storage condition a relative humidity (RH) of 60%±5% was maintained. In all cases, the pGal polymer was stored in high-density polyethylene (HDPE) plastic bottles, sealed with polypropylene (PP) caps, overlaid with Argon gas, and protected from light. This container closure system has been demonstrated to be compatible with pGal polymer. After initial polymer characterization immediately following manufacture (t=0), samples of pGal polymer were withdrawn from each storage temperate condition at time intervals of 3, 6, and 9 months for the −20° C.±5° C. and 5° C.±3° C. storage temperatures, and at time intervals of 3 and 9 months for the 25° C.±2° C. storage temperature. At each stability time point, the pGal polymer was evaluated for appearance, chemical identification, molecular weight, molecular weight distribution, moisture content, reactivity (thiopyridine content), and purity.

At each stability time point, appearance of pGal polymer was evaluated by visual inspection and compared to known reference material. Appearance was also confirmed to be as expected based on previous manufacturing experience at similar scale. Chemical identification of pGal polymer was also confirmed using ¹H-NMR spectroscopy. The ¹H-NMR spectrum of pGal polymer was compared to known reference material where the relative integrations and chemical shifts of protons in various relevant positions within the pGal polymer structure were confirmed. The peak molecular weight (Mₚ) and molecular weight distribution (M_{W}/M_{N}) of pGal polymer were determined via size-exclusion chromatography equipped with multiple-angle light scattering detection (SEC-MALS), affording the absolute molecular weight profile of pGal polymer based on a known refractive index increment calibration. Moisture content was measured using Karl Fischer Titration in accordance with USP <921>. The reactivity of pGal polymer was determined by evaluating the presence of the 2-thiopyridine (TP) group at the alpha terminus of the polymer chain. This method involved the reduction of the TP group from the pGal polymer at a known concentration via aqueous treatment with tris(2-carboxyethyl)phosphine hydrochloride (TCEP) reducing agent and analysis of the crude reaction mixture via reversed-phase high-performance liquid chromatography (RP-HPLC). The liberated 2-mercaptopyridine (or its tautomer pyridine-2-thione) was measured via RP-HPLC and quantified against a 2-mercaptopyridine standard curve. A mass balance was then performed to establish the molar amount of pGal polymer containing an active alpha end group functionalized with TP based on an expected 1:1 molar ratio between the TP and the pGal polymer. Purity was determined by RP-HPLC where the intact pGal polymer was well-separated from its known degradation products based on their relative retention times at a given wavelength. Using the same assay, pGal polymer with a DTB end-group can be readily separated from pGal polymer without a DTB end-group by observing the presence of the pGal polymer peak across various wavelengths, thereby confirming the presence of both degradation impurities concurrently with pGal polymer end group variations. Purity evaluation reports total polymer purity (relative to the appearance of degradation products) as well as the percentage of DTB-containing polymer present in the sample.

Tables 2-7, below, provide the results of these tests.

TABLE 2
Stability Data for pGal Polymer Stored at −20° C. ± 5° C.

| Test | Technique | Initial | 3 Months | 6 Months | 9 Months |
|---|---|---|---|---|---|
| Appearance | Visual | — | No Change | No Change | No Change |
| | Inspection | | | | |
| Identification | ¹H-NMR | — | Conforms to | Conforms to | Conforms to |
| | | | reference | reference | reference |
| Peak MW | SEC- | — | No Change | +0.4% | No Change |
| (Mₚ) | MALS | | | | |
| Molar mass | SEC- | — | −0.8% | +0.8% | +0.8% |
| dispersity | MALS | | | | |
| (M_{W}/M_{N}) | | | | | |
| Thiopyridine | RP-HPLC | — | +1.4% | No Change | −1.4% |
| content | | | | | |
| Purity (LC-UV) | RP-HPLC | — | 100% Total | 100% Total | 100% Total |
| | | | pGal polymer | pGal polymer | pGal polymer |

Abbreviations: — = Not tested; M_{N} = Number-average molecular weight; Mₚ = Peak molecular weight; MW = Molecular weight; M_{W} = Weight-average molecular weight; NAIR = Nuclear magnetic resonance; SEC-MALS = Size-exclusion chromatography with multiple-angle light scattering detection; RP-HPLC = Reversed-phased high-performance liquid chromatography.

TABLE 3
Stability Data for pGal Polymer Stored at 5° C. ± 3° C.

| Test | Technique | Initial | 3 Months | 6 Months | 9 Months |
|---|---|---|---|---|---|
| Appearance | Visual | — | No Change | No Change | No Change |
| | Inspection | | | | |
| Identification | ¹H-NMR | — | Conforms to | Conforms to | Conforms to |
| | | | reference | reference | reference |
| Peak MW | SEC- | — | No Change | No Change | No Change |
| (Mₚ) | MALS | | | | |
| Molar mass | SEC- | — | −0.8% | +0.8% | +0.8% |
| dispersity | MALS | | | | |
| (M_{W}/M_{N}) | | | | | |
| Thiopyridine | RP-HPLC | — | No Change | −1.4% | −1.4% |
| content | | | | | |
| Purity (LC-UV) | RP-HPLC | — | 100% Total | 100% Total | 100% Total |
| | | | pGal polymer | pGal polymer | pGal polymer |

Abbreviations: — = Not tested; M_{N} =Number-average molecular weight; Mₚ = Peak molecular weight; MW = Molecular weight; M_{W} = Weight-average molecular weight; NMR = Nuclear magnetic resonance; SEC-MELS = Size-exclusion chromatography with multiple-angle light scattering detection; RP-HPLC = Reversed-phased high-performance liquid chromatography.

TABLE 4
Stability Data for pGal Polymer Stored at 25°
C. ± 2° C./60% ± 5% RH

| Test | Technique | Initial | 3 Months | 9 Months |
|---|---|---|---|---|
| Appearance | Visual | — | No Change | No Change |
| | Inspection | | | |
| Identification | ¹H-NMR | — | Conforms to | Conforms to |
| | | | reference | reference |
| Peak MW (Mₚ) | SEC-MALS | — | +0.4% | No Change |
| Molar mass | SEC-MALS | — | −0.8% | +0.8% |
| dispersity | | | | |
| (M_{W}/M_{N}) | | | | |
| Thiopyridine | RP-HPLC | — | No Change | No Change |
| content | | | | |
| Purity (LC-UV) | RP-HPLC | — | 100% Total | 100% Total |
| | | | pGal polymer | pGal polymer |

Abbreviations: — = Not tested; M_{N} = Number-average molecular weight; Mₚ = Peak molecular weight; MW = Molecular weight; M_{W} = Weight-average molecular weight; NMR = Nuclear magnetic resonance; SEC-MALS = Size-exclusion chromatography with multiple-angle light scattering detection; RP-HPLC = Reversed-phased high-performance liquid chromatography; RH = Relative humidity.

TABLE 5
Stability Data for pGal Polymer Stored at −20° C. ± 5° C.

| Test | Technique | Initial | 3 Months | 6 Months | 9 Months |
|---|---|---|---|---|---|
| Appearance | Visual | — | No Change | No Change | No Change |
| | Inspection | | | | |
| Identification | ¹H-NMR | — | Conforms to | Conforms to | Conforms to |
| | | | reference | reference | reference |
| Peak MW | SEC- | — | No Change | No Change | No Change |
| (Mₚ) | MALS | | | | |
| Molar mass | SEC- | — | No Change | +1.6% | +0.8% |
| dispersity | MALS | | | | |
| (M_{W}/M_{N}) | | | | | |
| Thiopyridine | RP- | — | No Change | +1.6% | No Change |
| content | HPLC | | | | |
| Purity (LC-UV) | RP- | — | 100% Total | 100% Total | 100% Total |
| | HPLC | | pGal polymer | pGal polymer | pGal polymer |

Abbreviations: — = Not tested; M_{N} = Number-average molecular weight; Mₚ = Peak molecular weight; MW = Molecular weight; M_{W} = Weight-average molecular weight; NMR = Nuclear magnetic resonance; SEC-MALS = Size-exclusion chromatography with multiple-angle light scattering detection; RP-HPLC = Reversed-phased high-performance liquid chromatography.

TABLE 6
Stability Data for pGal Polymer Stored at 5° C. ± 3° C.

| Test | Technique | Initial | 3 Months | 6 Months | 9 Months |
|---|---|---|---|---|---|
| Appearance | Visual | — | No Change | No Change | No Change |
| | Inspection | | | | |
| Identification | ¹H-XMR | — | Conforms | Conforms | Conforms |
| | | | to reference | to reference | to reference |
| Peak MW | SEC-MALS | — | No Change | No Change | No Change |
| (Mₚ) | | | | | |
| Molar mass | SEC-MALS | — | No Change | +1.7% | +1.7% |
| dispersity | | | | | |
| (M_{W}/M_{N}) | | | | | |
| Thiopyridine | RP-HPLC | — | No Change | −1.6% | +1.6% |
| content | | | | | |
| Purity (LC-UV) | RP-HPLC | — | 100% Total | 100% Total | 100% Total |
| | | | pGal polymer | pGal polymer | pGal polymer |

Abbreviations: — = Not tested; M_{N} = Number-average molecular weight; Mₚ = Peak molecular weight; MW = Molecular weight; M_{W} = Weight-average molecular weight; NMR = Nuclear magnetic resonance; SEC-MALS = Size-exclusion chromatography with multiple-angle light scattering detection; RP-HPLC = Reversed-phased high-performance liquid chromatography.

TABLE 7
Stability Data for pGal Polymer Stored at 25° C. ± 2° C./60% ± 5% RH

| Test | Technique | Initial | | 3 Months | | 9 Months | |
|---|---|---|---|---|---|---|---|
| Appearance | Visual | — | | No Change | | No Change | |
| | Inspection | | | | | | |
| Identification | ¹H-NMR | — | | Conforms to | | Conforms to | |
| | | | | reference | | reference | |
| Peak MW (Mₚ) | SEC-MALS | — | | No Change | | No Change | |
| Molar mass | SEC-MALS | — | | No Change | | +1.7% | |
| dispersity | | | | | | | |
| (M_{W}/M_{N}) | | | | | | | |
| Moisture | KFT | 3% | w/w | 5% | w/w | 5% | w/w |
| content | | | | | | | |
| Thiopyridine | RP-HPLC | 63% | mol/mol | 63% | mol/mol | 63% | mol/mol |
| content | | | | | | | |

| | | | | |
|---|---|---|---|---|
| Purity (LC-UV) | RP-HPLC | — | 100% Total | 100% Total |
| | | | pGal polymer | pGal polymer |

Abbreviations: — = Not tested; M_{N} = Number-average molecular weight; Mₚ = Peak molecular weight; MW = Molecular weight; M_{W} = Weight-average molecular weight; NMR = Nuclear magnetic resonance; SEC-MALS = Size-exclusion chromatography with multiple-angle light scattering detection; RP-HPLC = Reversed-phased high-performance liquid chromatography; RH = Relative humidity.

### Example 14: Stability Study

This is a prophetic example. Polymers B, D, F, M, and N (from the Examples above) are functionalized to a sample antigen (a fragment of full-length antigen) thereby providing conjugate compounds of Formula (1). Polymers B, D, and F provide disulfide-based, α-end-linked antigen conjugates. Polymer M provides an amine-based (disulfanyl ethyl ester), α-end-linked antigen conjugate. Polymer N provides a disulfide-based, ω-end-linked antigen conjugate. Additionally, α-end-linked antigen conjugates (comprising the sample antigen “X”) have/may have the following terminal end:

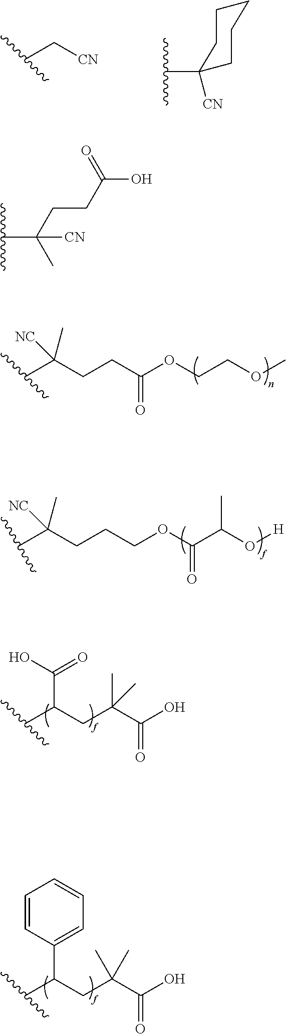

and the following polymer structure are prepared:

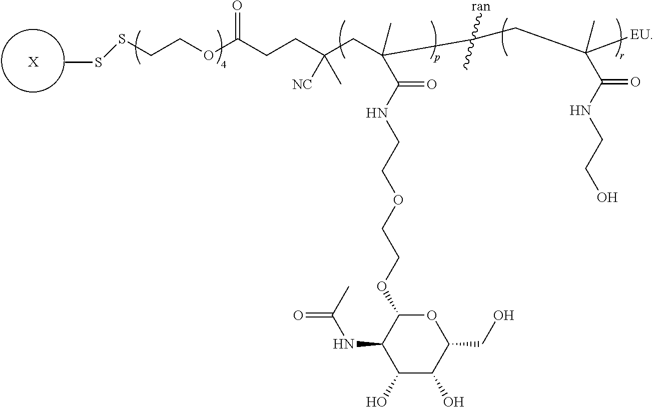

Control polymers comprising dithioester end-capping group (e.g., R²) are also prepared and functionalized to the sample antigen. Each polymer conjugate (experimental and control) is formulated in three different buffer solution conditions: 1) 1 mg/mL concentration of a conjugate in a solution of PBS (pH 7.2); 2) 1 mg/mL concentration of a conjugate in a solution of HEPES-buffered saline (pH 8.04); and 3) 1 mg/mL concentration conjugate in a solution of 10 mM sodium acetate, 274 mM sorbitol. Using aseptic techniques, aliquots of the polymer conjugate solutions are filled into Type 1 glass vials, which are then sealed with 4023/50G rubber stoppers and aluminum caps. Individual vials containing each sample are placed in a calibrated 23-27° C. incubator.

Surprisingly, the stability over a period of days (measured using reversed-phase HPLC) for the polymer conjugates of Formula (1) versus the control conjugates having a dithioester end-capping group is different. Analytical testing is performed on separate sample vials at the time points of 0, 7, 14, and 28 days. The CTA-remnant-free/dithioester-free (e.g., R²-free) conjugates have remarkably higher stability than dithioester containing conjugates under the same conditions. The percent main peak area is measured at 220 nm reported and graphed. The dithioester-free conjugates (having terminal end units) have enhanced stability relative to conjugate having a dithioester end-group. For example, after a period of about 10 days in storage (e.g., in buffered solution at room temperature), the peak corresponding to the intact construct as disclosed herein loses no more than about 1% of its area at 220 nm as measured by HPLC. Alternatively, after a period of about 10 days in storage (e.g., in buffered solution at room temperature), the peak corresponding to the intact construct comprising a dithioester loses about 4% of its area at 220 nm as measured by HPLC. After a period of about 20 days in storage (e.g., in buffered solution at room temperature), the peak corresponding to the intact construct as disclosed herein loses no more than about 1% of its area at 220 nm as measured by HPLC. Alternatively, after a period of about 20 days in storage (e.g., in buffered solution at room temperature), the peak corresponding to the intact construct comprising a dithioester loses about 6% of its area at 220 nm as measured by HPLC. After a period of about 28 days in storage (e.g., in buffered solution at room temperature), the peak corresponding to the intact construct as disclosed herein loses no more than about 2% of its area at 220 nm as measured by HPLC. Alternatively, after a period of about 28 days in storage (e.g., in buffered solution at room temperature), the peak corresponding to the intact construct comprising a dithioester loses about 10% of its area at 220 nm as measured by HPLC.

This testing demonstrates that dithioester-free constructs (and/or terminal end units comprising a carbon bond to the linker, lacking aryl functionalities, and/or lacking sulfur containing atoms, such as dithioesters) have surprisingly improved stability versus dithioester-containing constructs.

### Example 15: In Vivo Study (Fragment of Autoantigen Associated with MS)

Myelin oligodendrocyte glycoprotein (MOG) 30-60 is a fragment of an autoantigen that is associated with multiple sclerosis that is used herein as a non-limiting example of an immunogenic fragment of MOG. The mouse experimental autoimmune encephalomyelitis (EAE) model was used to demonstrate effective immune tolerance induction by pGal (e.g., Polymer B) conjugated to a fragment of an autoantigen, in that effective immune tolerance induction improves the disease outcome in the EAE model. To induce multiple sclerosis pathology (EAE disease), recipient mice were adoptively transferred on day 0 with MOG35-55-reactive T cells obtained from a separate, previously-vaccinated donor mouse. On day 0, 3, and 6, mice received via intravenous injection of either MOG30-60 peptide, MOG-30-60 peptide chemically conjugated to pGal (LT-MOG-30-60), or saline; an additional control group received administrations of a monoclonal antibody that binds VLA-4 (integrin alpha 4 beta 1). Multiple sclerosis pathology was assessed and scored daily by a pharmacology expert blinded to group identities. As illustrated in FIG. 8, mice administered with pGal-MOG30-60 were protected from multiple sclerosis pathology (EAE disease); in stark contrast, mice administered with MOG30-60 antigen alone, saline, or a monoclonal antibody that binds VLA-4 (integrin alpha 4 beta 1) were not substantively protected from disease. These data illustrate that compositions comprising an MS-related antigen, by way of example, here pGal-MOG30-60, effectively induced immune tolerance to MOG, and prevented autoimmune pathology of the central nervous system and the associated multiple sclerosis symptomology (EAE disease). It shall be appreciated that, according to additional embodiments disclosed herein, other immunogenic fragments of MOG are effective in inducing tolerance to MOG, and thus treatment of MS.

### Example 16: In Vivo Study (Mimetope of an Autoantigen for Type-1-Diabetes)

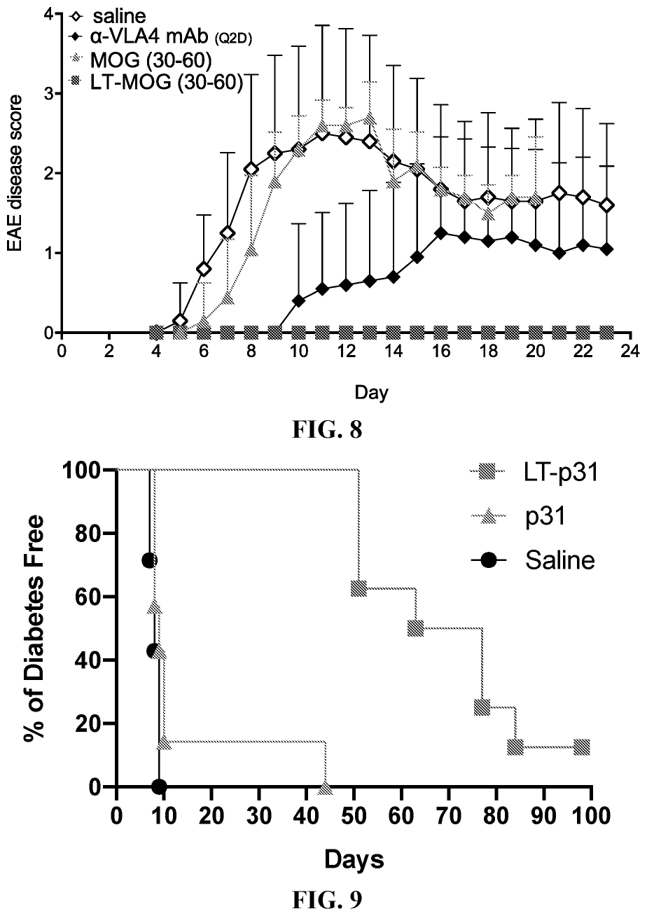

P31 is a mimetope of chromogranin-A, which is an autoantigen in type-1 diabetes. P31 is used in this example as a non-limiting example of a mimotope of an antigen to which tolerance is desired. The NOD.BDC2.5 mouse model was used to illustrate the tolerogenic efficacy of pGal (e.g., Polymer B) conjugated to a mimetope of an autoantigen. In brief, p31-specific T cells from NOD BDC2.5 transgenic mice were adoptively transferred into NOD.SCID mice to induce type-1 diabetes by driving an inflammatory autoimmune response to the islet antigen chromogranin-A. On days 0, 3, and 6, mice received intravenous administrations of either p31 peptide alone, p31 peptide chemically conjugated to pGal (LT-p31), or saline, and the onset of type-1 diabetes was monitored via blood glucose measurements. As illustrated in FIG. 9, pGal-p31 induced prolonged protection against the induction of type-1 diabetes as compared to p31 administered alone and as compared to saline administration. In this mouse model, protection against type-1 diabetes is indicative of effective immune tolerance induction to the autoantigen that drives disease. It shall be appreciated that, according to additional embodiments disclosed herein, other immunogenic fragments of Type I diabetes-related antigens, including mimotopes and/or immunogenic fragments of an antigen are effective in inducing tolerance to TID-related antigens, and thus treatment of Type 1 Diabetes.

### Example 17: In Vivo Study (Tolerogenic Portion of Human Proinsulin)

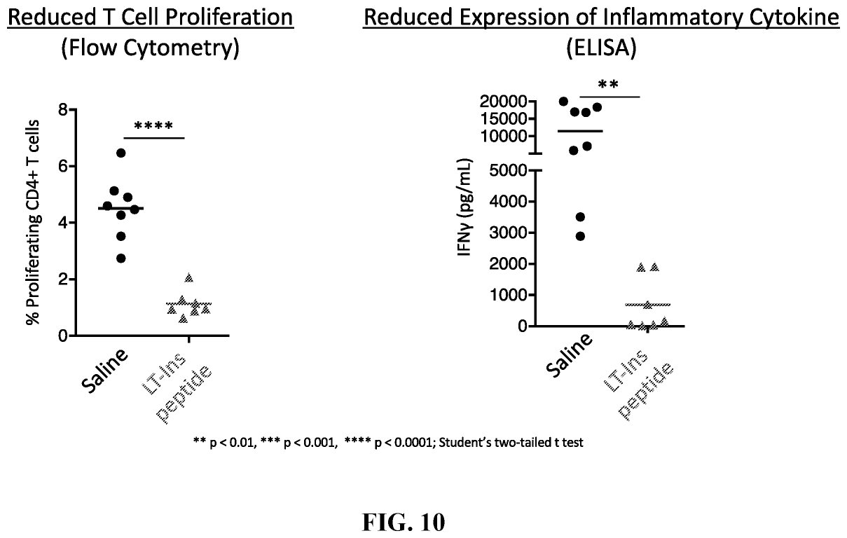

This example employs as the antigen to which tolerance is desired a tolerogenic portion of human proinsulin (LT-Ins peptide or “KAN0014”). Tolerogenic efficacy of pGal-LT-Ins peptide was characterized in HLA-DR4 transgenic mice by measuring the induction of tolerance to subsequent antigen challenge with adjuvanted KAN0014 peptide. Dosing with tolerogen (pGal-LT-Ins peptide) occurred between days −20 to −14 relative to antigen challenge. All mice were challenged on day 0 by the intradermal (i.d.) route with adjuvanted LT-Ins peptide. Mice were treated with pertussis toxin by the intraperitoneal route (i.p.) on days 0 and +1, relative to the timing of the first antigen challenge. On day 7 after antigen challenge, cells from the spleen and draining (axillary, inguinal, and iliac) lymph nodes (LN) were isolated and analyzed for T cell responses specific to the LT-Ins peptide. In FIG. 10, the left panel illustrates reduced proliferation of LT-Ins peptide-specific T cells in animals treated with saline or pGal-LT-Ins peptide, and the right panel illustrates the expression of the inflammatory cytokine interferon-gamma following in vitro stimulation of T cells with LT-Ins peptide. The results of both immunoassays illustrate a marked and statistically significant reduction in the magnitude of LT-Ins peptide-specific T cell inflammatory responses induced by administration of pGal-LT-Ins peptide, and thus effectively demonstrate immune tolerance induction to LT-Ins peptide. It shall be appreciated that, according to additional embodiments disclosed herein, other immunogenic fragments of Type I diabetes-related antigens, including mimotopes and/or immunogenic fragments of an antigen are effective in inducing tolerance to TID-related antigens, and thus treatment of Type I Diabetes.

### Example 18: Additional In Vivo Studies

This is a prophetic example. Experiments are performed as outlined in Example 11 using constructs having a terminal end unit (e.g., EU) that is not a dithiobenzoate, not a trithiocarbonate, and not a xanthate. Twelve total constructs are prepared and tested. The first construct comprises a full-length antigen associated with celiac disease. The second construct comprises a fragment of the full-length celiac disease antigen. The third construct comprises a tolerogenic portion of the full-length celiac disease antigen. The fourth construct comprises a mimetic of the full-length celiac disease antigen. The fifth construct comprises a full-length antigen associated with multiple sclerosis. The sixth construct comprises a fragment of the full-length multiple sclerosis antigen. The seventh construct comprises a tolerogenic portion of the full-length multiple sclerosis antigen. The eighth construct comprises a mimetic of the full-length multiple sclerosis antigen. The ninth construct comprises a full-length antigen associated with type-1 diabetes. The tenth construct comprises a fragment of the full-length type-1 diabetes antigen. The eleventh construct comprises a tolerogenic portion of the full-length type-1 diabetes antigen. The twelfth construct comprises a mimetic of the full-length type-1 diabetes antigen. Mice treated with saline showed a high frequency of OTI CD8+ T cells in the blood and lymphoid organs (i.e. spleen), indicative of maintenance of an inflammatory immune response specific to the antigens. Mice treated with constructs of as disclosed herein exhibited significant reductions in OVA01-specific OTI CD8+ T cells in the blood and lymphoid organs (i.e. spleen), at all doses tested. Statistical tests indicate as compared to the saline control group, the induced tolerance is statistically significant as (p<0.05). The studies are repeated in humans and the induced tolerance is again statistically significant (p<0.05) versus the control saline group.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a tolerance inducing liver targeting composition” include “instructing the administration of a tolerance inducing liver targeting composition.” In addition, where features or aspects of the disclosure are described in terms of Markush groups, those skilled in the art will recognize that the disclosure is also thereby described in terms of any individual member or subgroup of members of the Markush group. Any variables disclosed in the context of one embodiment may be applied to other embodiments. For example, when integer “v” is defined in the context of one embodiment, that definition of v may also be applied to a second embodiment (even if “v” is not defined for the second embodiment).

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%” In some embodiments, at least 95% identical in sequence includes 96%, 97%, 98%, 99%, and 100% identical in sequence to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

1.-82. (canceled)

83. A compound for the induction of antigen-specific immune tolerance in a subject, the compound comprising:
an antigen to which tolerance is desired or a tolerogenic portion thereof;
wherein the antigen to which tolerance is desired, when presented alone to the subject is capable of inducing an unwanted immune response in the subject;
a polymeric linker that does not include a dithiobenzoate, trithiocarbonate, or a xanthate, the linker comprising:
a copolymer or a random copolymer, wherein the copolymer or random copolymer comprises of first acrylyl unit and a second acrylyl unit, the first acrylyl unit comprising a first C-amido or C-carboxy functionality and the second acrylyl unit comprising a second C-amido or C-carboxy functionality;
wherein the second C-amido or C-carboxy functionality is conjugated to an aliphatic group, an alcohol, or an aliphatic alcohol;
wherein the polymeric linker is bonded to the antigen to which tolerance is desired or tolerogenic portion thereof via a disulfide bond or a disulfanyl ethyl ester,
wherein the disulfide bond or the disulfanyl ethyl ester are each configured to be cleaved upon administration of the compound to the subject and to release the antigen to which tolerance is desired or tolerogenic portion thereof from the polymeric linker;
wherein the polymeric linker comprises an end-capping group that improves the stability of the compound in solution by at least 20% as compared to a linker that includes a dithiobenzoate-containing, trithiocarbonate containing, or a xanthate-containing end-capping group; and
a liver-targeting moiety;
wherein the liver targeting moiety is connected to the first acrylyl unit through the first C-amido or C-carboxy functionality and a polyether.

84. The compound of claim 83, wherein the antigen to which tolerance is desired is a food antigen or a self antigen.

85. The compound of claim 84, wherein the antigen to which tolerance is desired is a food antigen, and wherein the food antigen is associated with Celiac disease.

86. The compound of claim 83, wherein the antigen to which tolerance is desired comprises one or more of SEQ ID NO. 55, SEQ ID NO. 54, SEQ ID NO. 56, or SEQ ID NO. 57.

87. The compound of claim 83, wherein the liver targeting moiety comprises one or more of N-acetylgalactosamine, galactose, galactosamine, glucose, glucosamine, and N-acetylglucosamine.

88. The compound of claim 87, wherein the liver-targeting moiety comprises N-acetylgalactosamine, wherein the antigen to which tolerance is desired comprises one or more of SEQ ID NO. 55, and wherein, when in a solution of 10 mM sodium acetate and 274 mM sorbitol at a compound concentration of 1 mg/mL, at a temperature of 23° C. to 27° C., the compound degrades less than 2.0% over a period of 28 days.

89. The compound of claim 84, wherein the antigen to which tolerance is desired is a self antigen, and wherein the self antigen is associated with multiple sclerosis.

90. The compound of claim 83, wherein the antigen to which tolerance is desired comprises one or more of SEQ ID NO. 27, SEQ ID NO. 28, SEQ ID NO. 30, SEQ ID NO. 43, SEQ ID NO. 44, SEQ ID NO. 45, or SEQ ID NO. 46.

91. A compound for the induction of antigen-specific immune tolerance in a subject, the compound comprising:
an antigen to which tolerance is desired;
wherein the antigen to which tolerance is desired, when presented alone to the subject is capable of inducing an unwanted immune response in the subject;
a polymeric linker comprising:
a copolymer comprising a first acrylyl unit and a second acrylyl unit, the first acrylyl unit comprising a first C-amido or C-carboxy functionality and the second acrylyl unit comprising a second C-amido or C-carboxy functionality;
wherein the second C-amido or C-carboxy functionality is conjugated to an aliphatic group, an alcohol, or an aliphatic alcohol;
wherein the polymeric linker is bonded to the antigen to which tolerance is desired via a disulfide bond or a disulfanyl ethyl ester;
wherein the polymeric linker comprises a terminal end unit lacking each of a dithioester and a dithiobenzoate (DTB) and wherein the terminal end unit confers improved stability to the compound when in solution; and
a liver-targeting moiety; and
wherein the liver targeting moiety is connected to the first acrylyl unit through the first C-amido or C-carboxy functionality and a polyether.

92. The compound of claim 91, wherein the antigen to which tolerance is desired is associated with Celiac disease.

93. The compound of claim 91, wherein the food antigen is selected from the group consisting of high molecular weight glutenin, low molecular weight glutenin, alpha-, gamma- and omega-gliadin, hordein, secalin, avenin, a tolerogenic portion of any of said antigens.

94. The compound of claim 91, wherein the antigen to which tolerance is desired is associated with food allergy, and wherein the food antigen is selected from the group consisting of conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), α-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1 lS globulin (Sin a 2), lipid transfer protein (Sin a 3), profilin (Sin a 4), profilin (Api g 4), high molecular weight glycoprotein (Api g 5), Pen a 1 allergen (Pen a 1), allergen Pen m 2 (Pen m 2), tropomyosin fast isoform, high molecular weight glutenin, low molecular weight glutenin, alpha-, gamma- and omega-gliadin, hordein, secalin, avenin, major strawberry allergy Fra a 1-E (Fra a 1), profilin (Mus xp 1), and a tolerogenic portion of any of said antigens.

95. The compound of claim 91, where the antigen to which tolerance is desired is associated with an autoimmune disease, and wherein the autoimmune disease is selected from the group consisting of multiple sclerosis, Type I diabetes, rheumatoid arthritis, vitiligo, uveitis, pemphigus vulgaris, neuromyelitis optica, Goodpasture's Disease, Parkinson's disease, myasthenia gravis, celiac disease, and anti-neutrophil cytoplasmic antibody-associated vasculitis.

96. The compound of claim 95, where the antigen to which tolerance is desired comprises one or more of a tolerogenic portion of myelin oligodendrocyte glycoprotein, a tolerogenic portion of myelin basic protein, and a tolerogenic portion of.

97. The compound of claim 95, wherein the antigen to which tolerance is desired comprises one or more of a tolerogenic portion of insulin, a tolerogenic portion of proinsulin, a tolerogenic portion of preproinsulin, a tolerogenic portion of glutamic acid decarboxylase-65 (GAD-65), a tolerogenic portion of GAD-67, and a tolerogenic portion of insulinoma associated protein 2 (IA-2).

98. The compound of claim 95, wherein the antigen to which tolerance is desired comprises one or more of a tolerogenic portion desmoglein-3, a tolerogenic portion of desmoglein-1, and a tolerogenic portion of desmoglein-4.

99. The compound of claim 95, wherein the antigen to which tolerance is desired comprises one or more of a tolerogenic portion of an acetylcholine receptor, a tolerogenic portion of muscle-specific kinase (MuSK), and a tolerogenic portion of lipoprotein receptor-related protein (LRP4).

100. A compound for the induction of antigen-specific immune tolerance in a subject, the compound comprising an antigen to which tolerance is desired, a polymeric linker comprising a polymeric portion, and a liver targeting moiety;
wherein the linker is bonded to the antigen via a disulfide bond or a disulfanyl ethyl ester;
wherein the disulfide bond or the disulfanyl ethyl ester are each configured to be cleaved upon administration of the compound to the subject and to release the antigen from the polymeric linker;
wherein the polymeric linker comprises a terminal end unit lacking each of a dithioester and a dithiobenzoate (DTB) and wherein the terminal end unit confers improved stability to the compound when in solution.

101. The compound of claim 100, wherein the antigen to which tolerance is desired comprises one or more of SEQ ID NO. 55, SEQ ID NO. 54, SEQ ID NO. 56, or SEQ ID NO. 57 and the liver targeting moiety comprises one or more of N-acetylgalactosamine, galactose, galactosamine, glucose, glucosamine, and N-acetylglucosamine.

102. The compound of claim 100, wherein the antigen to which tolerance is desired comprises one or more of and the liver targeting moiety comprises one or more of N-acetylgalactosamine, galactose, galactosamine, glucose, glucosamine, and N-acetylglucosamine.
